SEQUENCE LISTING (332 sequences)

SEQ ID NO: 1 - DNA, 1168 bp, Rattus Norvegicus
agccccgagtcttggctggccggtggtgggagcagaccgttcttgcctctgaaccggcaatcatggctccgggttggccgcggcctctgccgcagctcctcgtgttgggattcgggttggtgttgatacacgccacggccggggagcaagcaccaggcaacgccccatgctcaagcggcagctcctggagcgcggacctcgacaagtgcatggactgcgcttcttgtccagcgcgaccacacagcgacttctgcctgggatgtgagtgggggtggggacggggctggtggcaagcggatcctagatctgggtaggtggttgttggggcagaaaggaggtcgtagacttaggatataggaaaccaggaaaaactgactgaggaagggacttttataagggtgtgcgttgggaggggggtaagcttgagaccctttgagggcaggctggattggcgtcccaagggcaagatctgatttgaacccgatcactaggcgcagcagcacctcctgcccacttcaggatgctatggcccattctgggaggcgctcttagtctggccctggttttggcgctggtttctggttttcctggtctggagacgatgccgccggagagaaaagtttactacccccatagaggagactggtggagaaggctgcccaggtgtggcactgatccagtgaggagcacccgcgctggtgcccattcatcgtccattcattcattctggagccagcctggctttccagagacaagccgcgccagactcttccaaccacaagggggtggggcgaggtggtgattcacctccaaggactgggcttagggttcaggggagccttccagggtgtctaattgccctgtctctggttctggggcagacagagagcctcaagctaggtcacaaagcgactcatactaaggatctgcagcatttgcacaagggaatctcttgctccccacaagtcctggaggcctggccgacttgagggggcagacacaacactgggttccacccactcggatggactgaaatgccactaccctgagttttagccctgggagtgggttagattgagggacctgttcataacactaagggggctggccctctgggagggctggtcccagtacacacaaacacaacccatcccccaagggggtgata

SEQ ID NO: 2 - DNA, 230 bp, Artificial Sequence
  Synthetic polynucleotide
gtgagtgggggtggggacggggctggtggcaagcggatcctagatctgggtaggtggttgttggggcagaaaggaggtcgtagacttaggatataggaaaccaggaaaaactgactgaggaagggacttttataagggtgtgcgttgggaggggggtaagcttgagaccctttgagggcaggctggattggcgtcccaagggcaagatctgatttgaacccgatcactag

SEQ ID NO: 3 - DNA, 948 bp, Rattus Norvegicus
ggaggagggcagagccagaggagggacagcctgatgcagacagccctgactcaacctgccagcccccttacctgtcagccttgaggagatggaacagcccagcctactaggcctgcccccactccccaacttcccttgataatgaaccagaaactgaaactaaaacggctccgcttgatgagtcacttcccaaagtgactaaagtagctgagaagtagaaacagaggcagaagagaacccagggcactgggacaggacttcctgagggtctgaggagcatggcagacagcccagaggtggtagccatggactgtgagatggtggggctcgggcctcaaagggtgagtggcctcgcccgctgcagcattgtgaacgtccacagcgcagtcctctatgacaagtacatccagcctgagggagagatcacggactacagaacccaagtcagcgggatcacgcctcagcacatggcgagggccacgccatttgctgaagccaggctagagatcctgcagcttctgaaaggcaagctggtggtgggccatgacctgaagcatgacttcagtgccctgaaggaggacatgagaaaatacaccatctacgacacgtccacagacatgctgctgtggcacgaggccaagctgcactgctacagccgtgtgtccctgaggctgctgtgcaagcgcctgctgcacaagagcatccaggtgaggagcttcccggcccctgtgacccttctctctcttctccctcctctctctctccctccctcctcccactcaggtaaggagtaacctgcccttcctcttcctcctctcctcctccaagcaaggagtcctcctgctttctcccttagtcctccctctccttccctgattctaaaagtgaggatcttcctctccctcctccccgtgctcccactcctcccaagggcaggtcagtcccacc

SEQ ID NO: 4 - DNA, 255 bp, Artificial Sequence
  Synthetic polynucleotide
ggaggagggcagagccagaggagggacagcctgatgcagacagccctgactcaacctgccagcccccttacctgtcagccttgaggagatggaacagcccagcctactaggcctgcccccactccccaacttcccttgataatgaaccagaaactgaaactaaaacggctccgcttgatgagtcacttcccaaagtgactaaagtagctgagaagtagaaacagaggcagaagagaacccagggcactgggacag

SEQ ID NO: 5 - DNA, 249 bp, Artificial Sequence
  Synthetic polynucleotide
gtgaggagcttcccggcccctgtgacccttctctctcttctccctcctctctctctccctccctcctcccactcaggtaaggagtaacctgcccttcctcttcctcctctcctcctccaagcaaggagtcctcctgctttctcccttagtcctccctctccttccctgattctaaaagtgaggatcttcctctccctcctccccgtgctcccactcctcccaagggcaggtcagtcccacctggcctaa

SEQ ID NO: 6 - DNA, 2438 bp, Rattus Norvegicus
agcgaccggcagggtgcccgcccgacttcccagctgccccgtgcggcccgggcatgcccagtgtgggcgcagccccggctctccgagcgccggggcggagcgggcagccgggcgcggagcaggcgaaccgaacctcggtgggcgagcgccggggctggagccgcggggggtcggtggcggggccttccatgatgagtgatttgggggcacttccctctctctgtaattgatagtctgggcagtgaagagatggctgacagtgtcaaaacctttctgcaggaccttggcaggggaatcaaagactccatctggggcatctgtaccatctcaaagctagatgctcggatccagcagaagagagaggagcagcgtcgaaggagggcaagtagcctcttggcccagaggagagcccagagtgtagagcggaagcaagagagtgagccacgtattgttagtagaattttccagtgctgtgcttggaatggtggagtattctggttcagtctcctcttgttttaccgagtgtttattcctgtgcttcaatcagtaacagcccgggttattggtgatccgtcactacatggagatgtttggtcatggctggaattcttcctcacatcaattttcagtgctctttgggtgctgcccctgtttgtgcttagcaaagttgtgaatgccatttggttccaagatatagctgacttggcatttgaggtatcagggaggaaacctcatccattccccagtgtcagcaaaataattgctgacatgctcttcaaccttttgctacaggcacttttccttattcaggggatgtttgtgagtctcttccccatccatcttgtgggtcagctggttagtttgctgcatatgtctcttctctactcactctactgctttgagtaccgttggttcaacaaaggaattgaaatgcaccagcgattgtcaaacatagaaaggaactggccttactactttgggtttggcttgcccttggctttcctcacagcaatgcaatcctcctacattatcaggtaatttgggtagaaaaagaattcgtaagcatcttttgttcagctactgtaccaacattttacatcatttgaccatcaggaagatttgttgctgttaatagtgacatttggtatcattaaaatgaaaatataaaaagaaatctaggctgcctcctggaggaagatacttaggagttcagaagtgaagagatgaggcttataatactttttcctaaagtcatgccttgtataattgacctttttttcccttaaacagtggctgcctcttttctatcctgtttcctttattcatcatcagcgccaatgaagcaaagactcctggaaaagcatatcttttccagttgcgcctcttctccttggtggtctttttaagcaacagacttttccacaagacggtctacctacagtcggccctgagcagctcatcctccgcagagaaattcccttcgccacatccttctcctgccaaactgaaagctgctgcaggccactgagtcctgctgtcaaaggggacgggtgggactgggtggaggatgtggcagctcttttcttggttttcctccccctgctgtggaaggcaggacccactgccaaggggcctctgcatattccctttgtctttgaattgggaatcttcctgactccggtatgtggatttttaccaccaccctaggtctggaaggaccagttttccagctgttttttagcttttgccagctcctgtgcctggactgatttgagtactcccccctcccttgtgtcatttaccttcccacctcctcctgcctcccagcacccctgggtgaatgggctttgtaattttaactgttgtattttgtgaatttgttgttactgtttcctgtgaagcacataccttgtatgtgggaggtaaaggagcacgccagctgctccatgtcactccctctatagccatcactgtcttgtttttttgtaactcaggttaggttttggtctttactgctctgctgcagaaaaggaaaggaatgggggaaaggagcctaagagatgggacagatagacctcagccaaactggttgggttttgaggagtcatgttctttgttccccttgaaggggaaacaggttttttcactggtacatttaaagttccccagctatgggaaggtaccagttctggacaagtgccactgcatcatagcaactctgaagatgaaacttactgttggccactgccaggtcagactcgtgttttaaggaatactgggtgcttcatataggaactgaaggggtaaacttactaaaccattcaacctgtgattggtgatgtttttctgtcattttaagagtcgacacatggggtgggagggcagatgtaaaaaaacttgtacaattttaaaatcacaattaaacgtgagctggtttc

SEQ ID NO: 7 - DNA, 256 bp, Artificial Sequence
  Synthetic polynucleotide
gtaatttgggtagaaaaagaattcgtaagcatcttttgttcagctactgtaccaacattttacatcatttgaccatcaggaagatttgttgctgttaatagtgacatttggtatcattaaaatgaaaatataaaaagaaatctaggctgcctcctggaggaagatacttaggagttcagaagtgaagagatgaggcttataatactttttcctaaagtcatgccttgtataattgacctttttttcccttaaacag

SEQ ID NO: 8 - DNA, 137 bp, Artificial Sequence
  Synthetic polynucleotide
agcgaccggcagggtgcccgcccgacttcccagctgccccgtgcggcccgggcatgcccagtgtgggcgcagccccggctctccgagcgccggggcggagcgggcagccgggcgcggagcaggcgaaccgaacctcg

SEQ ID NO: 9 - DNA, 2422 bp, Rattus Norvegicus
ataaaatcaatcccactttcttgttaaaaggagaaacgatctgaattttgaaagactagaagcccaactgaaggcgtgccactgcaggatcatattttctaaagcaaaggccttctgtgctggcgctggcaatcatcgctttggagatccaagcactgaagtatgtggagttaacagaaggagtagaatgtattcagaaacattccaagataagtggccgagatctgaccttctggcaagagcttgtttccaagtgtttaactgaatattcatcaaacaagtgttccaagccgaacggtcagaagttaaaatggatcgtgtctgggcgcactgcacgacaactgaagcacagttattacaggataacccacctcccaacaattcccgaaaccatgggttagttggcaaatctggttgttatcctctgtgtacagaacatttcccagtgagatcgtttttgtgctataacttaaggattgaaatactaccttcaatataaagaatacaggatgaaaacagtaaaggaaacgtgagtttgttggtctagacagagaatactgggaggcattcactgtgtaccgcagtctgaagagaaatgagtatcaaacctctagacacatgctcatactgctgtcaaaggactagcgtagaaaagagagtcctccaaaccggaagtttaaatgtagttactaaaatagcacttctttaacttacatatccccccactgtggcttatttaaagttacagaagtccaagcagaacgacaaaagatgtgacccatatatgaacacattttaatctgttcattgattaggagagtgaatatgaacttgcatgatgcccatgttaggtttctggaaactgccggggtatcttaattctctagtattctccctctgtggcagttgggctaatacaaagtaactatacgcatgagaatataaaatcagtctctgatacatacacatttttaccatcaaaatttcttaatcatagcaaagacttacctttttatgattaggaattttttttttaatgtatggcagcacatgcctttaatcccaacactagggaggcagaggcaggtggatctctttgagttcgaagccaggctggtctttacagtgagttccaggacagctggagagctacagaatggagagacgctgtctcaaaaacactgaaaacaaacaaacaaaccataccagtttgtaggcagacttctgttgggttgggtttgtactgtttgcctatgcagtgggattacagcagcagcaacaaaaactgtccctgaagtctttctctgccactgtgacctgagtttcctatggtacgcgatttattctaagaaacctcagcccctcaccacgttagctgttggcaaatggcctcacagttgcggaaagtcccaattctaggcttgggaaagcaatgcttagatttgaattggcccatgaagcattcaaatcaaggctaaagacataaatgtgaaataaaactgtgaaccttcattttaacattgatctcacttcccagatttaatcaatatatacttaggtggtattaaaaatggtaaactgcctaatttaaatctcaaaatttaaactatgaggtttacatcaaagccaacatttcacaaatgtacttttaaggtattaaaagaggtatttaagcagtaaatggtttcttggcacccataaccaagtaatagttaagttagaggtgggacttttttattgctatgagaattacatttaaacttttgggtgttttataaaaagcagatttcacaagttttgaaaattgtgacctttactgaaatttgttacctttaatatttcttctagaggataggtatttataaaagaaaaattcgtcagaattgctgcctcaatctagtcccatttgagaaaatttgtttctactgtctcaataactggatgaaatatcactctgaaaacttgcctattgcactaaagctagtttaggcttgataaaacactccaggaggtttttaccacagactgtttctattaaaactgctgcttctcatgtacaattttgttttaaaaggagaaccgagtacatctgcaaaacctaagtcttaagggacgtcaggaggtaccttcagaattataggatcaccatggtagtggggattctccatgctggccttgaatgtttgatcttcactgctgaaatgtgggtagctcctcagcgccctgtagagcctgagtctacctagaatagctgtaaccattttgacaagtaatggataagaaaattatccattgagaagctaaaaacaaaacaaaacaaaaccaaagaacgggtgtattttattcttaacctttgtaaaccatcactgagaacacttcagttcttcctaacagctgttatgcttcgatttgaaaaaaatac

SEQ ID NO: 10 - DNA, 30 bp, Artificial Sequence
  Synthetic polynucleotide
ataaaatcaatcccactttcttgttaaaag

SEQ ID NO: 11 - DNA, 110 bp, Artificial Sequence
  Synthetic polynucleotide
ataaaatcaatcccactttcttgttaaaaggagaaacgatctgaattttgaaagactagaagcccaactgaaggcgtgccactgcaggatcatattttctaaagcaaagg

SEQ ID NO: 12 - DNA, 1111 bp, Rattus Norvegicus
agaggccatccatcaaaagaatcacaccaatcagaggcgaagatgagtcccagctagcaatcctggtgcccagcctatctccccgcggccgggcgcagtccttcaccgcacgctgaaccggaggaaggctgcgcctagtcggggcgctgagggaccctccaccgggacgccggcccctccccgggcctctgctcacttgcccccctctgcgagcccgtccccctagtcggcctctcggatcggggacgtggggcgagctgagagcaggcccggggtgggtggtcactgtggagaagacgtggctgtcaaggatgatagaagtactgacaactgactctcagaaactgctacaccagctgaacaccctgttggaacaggagtccagatgtcagccaaagggtctgtggcctgaaactgattgagtctgcacatgataatggcctcaggatgactgcaagactccgggactttgaagtcaaagatctactgagtctaactcagttctttggcttcgacacagaaacattttcccttgctgtgaatttactggacagattcttgtctaaaatgaagggtacaggcgaagcatctcggctgtgtcggactgagctgcttttatttggctgtgaaatcgattgaagaggaaaggaacgtcccgctggcaactgatttgatccggataagtcagtataggttcacagtttcagacctgatgagaatggaaaagattgtgttggagaaagtgtgctggaaagtcaaagctactactgccttccaatttctgcagctctattactccctcattcgggagaccttgccatttgaaaggagaaacgatctgaattttgaaagactagaagcccaactgaaggcgtgccactgcaggatcatattttctaaagcaaaggccttctgtgctggcgctggcaatcatcgctttggagatccaagcactgaagtatgtggagttaacagaaggagtagaatgtattcagaaacattccaaggtatgccaaggtgatagcattgatcctattagcaagctacaagagaaatgtatccactgtactcctgaagtaaaataaaatgctgtgtattgatttgaagctta

SEQ ID NO: 13 - DNA, 104 bp, Artificial Sequence
  Synthetic polynucleotide
gtatgccaaggtgatagcattgatcctattagcaagctacaagagaaatgtatccactgtactcctgaagtaaaataaaatgctgtgtattgatctgaagctta

SEQ ID NO: 14 - DNA, 203 bp, Artificial Sequence
  Synthetic polynucleotide
ccttctgtgctggcgctggcaatcatcgctttggagatccaagcactgaagtatgtggagttaacagaaggagtagaatgtattcagaaacattccaaggtatgccaaggtgatagcattgatcctattagcaagctacaagagaaatgtatccactgtactcctgaagtaaaataaaatgctgtgtattgatttgaagctta

SEQ ID NO: 15 - DNA, 1228 bp, Rattus Norvegicus
atcccgttgcgggtatagcagacggaaaagcgagctggaagtgtagacacaactctcgcgagatgtagagcgaatccctggagaggggcggatgcgcagaggtgcaccacgtgtggtgttcctccagcgcagtcaggatctcgtctagtttgggtctaggttagatttggaatcgtggagatgcggaaggaaagcccaccctccgctcgtgcccccggcggcccgcgagtggaacctgccccccaatgcgcccgcctgcatggaacggcagttggaagctgcacggtaccgctctgatggttcccttctgctcggggcttccagtctgagtggtcgctgctgggcaggatctctgtggtttttcaaggatcctagtgcggcccccaacgaaggtttctgctctgctggcgtccagacggaggctggagtagctgacctcacttgggttggggacaaaggtatcttagtggcttctgattcaggtgctgttgaattgtgggagctagatgagaacgagacacttattgtcagcaagttctgcaagtatgagcatgatgacattgtgtctactgtcactgtcctgagctccggcacacaggctgtcagtggtagcaaagacttctgcatcaaaatttgggacctggctcagcagatgtcgctgaattcataccgagctcatgcgggacaggtcacctgtgttgctgcctctccccacagagaaactgtgtttctttcatgcagtgaggacagtagaattttgctctgggatacccgctgtcccaagccggcatcacagatgggctgcaatgcctctggctacctccctacctccctggcttggcatcctcagcagagtgaaatctttgtctttggtaaggcagcatgacgtatcgactctgggaatggggcggataccttggagtacatgctcctgtcagcacacttgggcactgcatggattatgtcggttttacctagctttggagaccatcattttttcctaataggtttgcttcattgtttcagctcctttattctgggcatggttagaaatcagtgagctgcatagcaagtgcttgctgttagggaactatgttctgctgtggtgtgactacacatggcaagagtttgtagttaattaattcacagccacatagtgactgactgattctatatcacaaaggaaactgttgtctacatcttgagaaactaggattcattcaaatct

SEQ ID NO: 16 - DNA, 224 bp, Artificial Sequence
  Synthetic polynucleotide
cttggagtacatgctcctgtcagcacacttgggcactgcatggattatgtcggttttacctagctttggagaccatcattttttcctaataggtttgcttcattgtttcagctcctttattctgggcatggttagaaatcagtgagctgcatagcaagtgcttgctgttagggaactatgttctgctgtggtgtgactacacatggcaagagtttgtagttaat

SEQ ID NO: 17 - DNA, 43 bp, Artificial Sequence
  Synthetic polynucleotide
gtaaggcagcatgacgtatcgactctgggaatggggcggatac

SEQ ID NO: 18 - DNA, 17 bp, Artificial Sequence
  Synthetic polynucleotide
gtaataactttatccag

SEQ ID NO: 19 - DNA, 88 bp, Artificial Sequence
  Synthetic polynucleotide
taattcacagccacatagtgactgactgattctatatcacaaaggaaactgttgtctacatcttgagaaactaggattcattcaaatc

SEQ ID NO: 20 - DNA, 2510 bp, Rattus Norvegicus
atcccgttgcgggtatagcagacggaaaagcgagctggaagtgtagacacaactctcgcgagatgtagagcgaatccctggagaggggcggatgcgcagaggtgcaccacgtgtggtgttcctccagcgcagtcaggatctcgtctagtttgggtctaggttagatttggaatcgtggagatgcggaaggaaagcccaccctccgctcgtgcccccggcggcccgcgagtggaacctgccccccaatgcgcccgcctgcatggaacggcagttggaagctgcacggtaccgctctgatggttcccttctgctcggggcttccagtctgagtggtcgctgctgggcaggatctctgtggtttttcaaggatcctagtgcggcccccaacgaaggtttctgctctgctggcgtccagacggaggctggagtagctgacctcacttgggttggggacaaaggtatcttagtggcttctgattcaggtgctgttgaattgtgggagctagatgagaacgagacacttattgtcagcaagttctgcaagtatgagcatgatgacattgtgtctactgtcactgtcctgagctccggcacacaggctgtcagtggtagcaaagacttctgcatcaaaatttgggacctggctcagcagatgtcgctgaattcataccgagctcatgcgggacaggtcacctgtgttgctgcctctccccacagagaaactgtgtttctttcatgcagtgaggacagtagaattttgctctgggatacccgctgtcccaagccggcatcacagatgggctgcaatgcctctggctacctccctacctccctggcttggcatcctcagcagagtgaaatctttgtctttggtgatgagaatggatctgtctcccttgtggacaccaagaatgcaagctgcaccctcagctcagctgtgcactcgcagtgtgtcactagactggtgttctctccacacaggtgttcccttcctggcctctctcagtgaagactgttcacttgctgtgctggattcaagcttttctgaggtggtttagaagtcgagcccacagagactttgtgagagatgctacatggtctccactcaatcactcccttcttaccacagttggctgggaccatcaggtcatccaccatgttgtgccgttagagcctctcccagcccctggacctgacagtgttgtggagtagaatggattgcagaagagaaaagagccttctgtctgcaagtgtcgactcgcttgcccctgccttccatttgtgagagagagagcacaggagccttgtagagcatgattactccctagtcccgtgcagtaactagtcagattctcagcctgagggaggctgcatcccatagtgacttggaggaaaaagcttcctttataaatggatgtatgtgtgagtacacgtgtgagtgcagtgtatagtttggagtggagaaaattatccttcagctttcccaaagcaatgctctttacccccgacaaaatgaccagaggattttatgcttcccatgtctgggggctgactatgttgttagggatatggagtgtgggtatccctggatgcttgggaatacttcacattactcagaggtgcgtaacttatttttatatagagtttaattcactatatgggaattacttcccatatacaaaagtctagccagactaccttgagaagacagaacagttttgtacgaatgcctgttaaatggaatcaaatccactttctgggtgaatcaatttggcccttggagttcgcgctcgctcaccctgtgttccttcttttgtgttgtgtatgagagatgagtgctttcctgtcctagatttcacagttttagggatgatggagtacttaaattaaaagtgtttcagtgaagcgagaagattgacatggattgttacagagaaatctgtcattgtcatttttcccacctaaaaattccctgaggactgatctgggtactttgctctggagagctgaagtctgagcgctgtatatttggactcctaagattgaagatccggcatcagagaaactgtcaaaaatactggaaggtgaaataaagacttatcctttcatctttcctccaaagcttatttattacctgctttgcttaggtgagtcacctgagtatcaatcagtcttaactgcccactgtctttgtagtctactgttcggcactccgttagcaattatgggtcagtaactgatcttattagagctttacaactttggtttaggaaaagcacataaaatgggcgccctgaaggaccatgagcttccattctactcttgccagtcaagagcctctcgtgatcacccttgagtaaagcacttgaatttgcagccgacttggaattttactaagttccagggaaggcgtaagagggaggtagaatcagtctctgcttctcaagagataaagcttgtattttgtgttccttagggaaggacaaattatccgtttacacggcctgctt

SEQ ID NO: 21 - DNA, 171 bp, Artificial Sequence
  Synthetic polynucleotide
gcactccgttagcaattatgggtcagtaactgatcttattagagctttacaactttggtttaggaaaagcacataaaatgggcgccctgaaggaccatgagcttccattctactcttgccagtcaagagcctctcgtgatcacccttgagtaaagcacttgaatttgcagc

SEQ ID NO: 22 - DNA, 13 bp, Artificial Sequence
  Synthetic polynucleotide
ctgtgtcagtcag

SEQ ID NO: 23 - DNA, 134 bp, Artificial Sequence
  Synthetic polynucleotide
gacttggaattttactaagttccagggaaggcgtaagagggaggtagaatcagtctctgcttctcaagagataaagcttgtattttgtgttccttagggaaggacaaattatccgtttacacggcctgctttgg

SEQ ID NO: 24 - DNA, 1168 bp, Rattus Norvegicus
tttcccggggcctgggcggagagggaggaaaacttcctggctgggactctggcggtttctgcctgccaaccctccgatcccgcctaccagcgttggctcttcccgacccctcccccggcgcccccagtgtccgccatggccaaagcctacgaccacctcttcaagttgctgctgatcggggactcgggggtgggcaagacttgtctcatcattcgctttgcagaggacaacttcaacagcacttacatctccaccatcggaattgatttcaagatccgaactgtggaaatagaggggaagagaatcaaactgcaagtctgggacacagctggccaagaacgattcaagacaataaccaccgcctattaccgtggagccatgggcattatcctagtatatgacatcacagatgagaaatccttcgagaatattcagaactggatgaaaagcatcaaagagaacgcgtctgctggagtggagcgccttctgctggggaacaaatgtgacatggaagccaagcggaaggtgcagagagagcaggctgagagggtgagtcaggccgggctgctgtggccgaatgccttgcttctgccctttatccagctctctccttcctacctcatcccctacagttggcccgagagcacagaatccgattttttgagacaagtgccaaatccagtgtgaatgtggatgaggctttcagttccctggcccgtgacatcttgctcaagacaggaggccggagatcgggaaacagcagcaagccctcaagcactgacctgaaagtatctgacaagaagaacagcaacaagtgctccttgggctgaggaacatttcttgcctcctattcaccctgaacctggaggctagacctgagggaggtggactgaggtagactgatggaaaacagaggggaggagctgtggtggtgcctggaggggtggatgacaggggaggaaggaaagatgaaatgggcagggaaaggagggcgaggaaccaaggacgtgaaaagtgaagagaaggggtttgagaagagaaaaagaagaaggtctcaggtctcggaccgtccaacattaatgtcagtatgctgatctctccattcctggttcagggttcgggtcccgagaggctggctcggccctactctgagggtctctcactccac

SEQ ID NO: 25 - DNA, 83 bp, Artificial Sequence
  Synthetic polynucleotide
gtgagtcaggccgggctgctgtggccgaatgccttgcttctgccctttatccagctctctccttcctacctcatcccctacag

SEQ ID NO: 26 - DNA, 149 bp, Artificial Sequence
  Synthetic polynucleotide
gtgagtcaggccgggctgctgtggccgaatgccttgcttctgccctttatccagctctctccttcctacctcatcccctacagttggctcgagagcacagaatccgattttttgagacgagtgccaaatccagtgtgaatgtggatgag

SEQ ID NO: 27 - DNA, 1356 bp, Rattus Norvegicus
gtgacgcgcctgtgttgtttcgccattttgggaaggtcctacaggccgaatgcaatccggcgcccgttgactgccatttttagaaggtctgtaaaggctcctcgtgtcgcggttgcgtagccatggaattgatttcaagatccgaactgtggaaatagaggggaagagaatcaaactgcaagtctgggacacagctggccaagaacgattcaagacaataaccaccgcctattaccgtggagccatgggcattatcctagtatatgacatcacagatgagaaatccttcgagaatattcagaactggatgaaaagcatcaaagagaacgcgtctgctggagtggagcgccttctgctggggaacaaatgtgacatggaagccaagcggaaggtgcagagagagcaggctgagaggttggcccgagagcacagaatccgattttttgagacaagtgccaaatccagtgtgaatgtggatgaggctttcagttccctggcccgtgacatcttgctcaagacaggaggccggagatcgggaaacagcagcaagccctcaagcactgacctgaaagtatctgacaagaagaacagcaacaagtgctccttgggctgaggaacatttcttgcctcctattcaccctgaacctggaggctagacctgagggaggtggactgaggtagactgatggaaaacagaggggaggagctgtggtggtgcctggaggggtggatgacaggggaggaaggaaagatgaaatgggcagggaaaggagggcgaggaaccaaggacgtgaaaagtgaagagaaggggtttgagaagagaaaaagaagaaggtctcaggtctcggaccgtccaacattaatgtcagtatgctgatctctccattcctggttcagggttcgggtcccgagaggctggctcggccctactctgagggtctctcactccacagatctttgttagtattaaaggccactgtttggcacgaatgttccatttgcattactttcattattgtcagaattgctctttactcaaatccaatttctgtcatgccaggatattagttctcctgaatctggctgggtcccccttccttaccccaactctttaactggtgatgaaaacagcaaggagaaagggcagcctgaagaatggacatcctttttccactatgggttcctgaggacaagagtggccggaaccatgcctttagcaatagtaagccaggatgaaggatttgaaccaagcttcaggaaacagcagcacttaatatggtttacccaaggagatgggaagcatctttgattctgctgtagtcaaatggtgtttgatgggcactgttagctgctttt

SEQ ID NO: 28 - DNA, 875 bp, Artificial Sequence
  Synthetic polynucleotide
ggaaacagcagcaagccctcaagcactgacctgaaagtatctgacaagaagaacagcaacaagtgctccttgggctgaggaacatttcttgcctcctattcaccctgaacctggaggctagacctgagggaggtggactgaggtagactgatggaaaacagaggggaggagctgtggtggtgcctggaggggtggatgacaggggaggaaggaaagatgaaatgggcagggaaaggagggcgaggaaccaaggacgtgaaaagtgaagagaaggggtttgagaagagaaaaagaagaaggtctcaggtctcggacgtccaacattaatgtcagtatgctgatctctccattcctggttcagggttcgggtcccgagaggctggctcggccctactctgagggtctctcactccacagatctttgttagtattaaaggccactgtttggcacgaatgttccatttgcattactttcattattgtcagaattgctctttactcaaatccaatttctgtcatgccaggatattagttctcctgaatctggctgggtcccccttccttaccccaactctttaactggtgatgaaaacagcaaggagaaagggcagcctgaagaatggacatcctttttccactatgggttcctgaggacaagagtggccggaaccatgcctttagcaatagtaagccaggatgaaggatttgaaccaagcttcaggaaacagcagcacttaatatggtttacccaaggagatgggaagcatctttgattctgctgtagtcaaatggtgtttgatgggcactgttagctgcttttgttagaaaagaacattctcagtgttccaccaagaaacacatgaacaaagtaagag

SEQ ID NO: 29 - DNA, 125 bp, Artificial Sequence
  Synthetic polynucleotide
gtgacgcgcctgtgttgtttcgccattttgggaaggtcctacaggccgaatgcaatccggcgcccgttgactgccatttttagaaggtctgtaaaggctcctcgtgtcgcggttgcgtagccatg

SEQ ID NO: 30 - DNA, 407 bp, Rattus Norvegicus
tgggggtgggggaatctagcctctctagagccctagccctctgacgaggaggaggtgtagtgccctgtagcttttccccaggactcgccacatgtccctttgtctggggaatgatgccctcaccctctgcatctggcctgacatctgttctctctgcagaatttctatcactccaagcgccgattggtcttctgcaagagaaagccctgaagtgcccacgggaggctcgccttcttctgctgtgggtcaggaggcctcttccccatcttctgccttagccttcattcagtgtgtcttaattattatttgtgttttaatttaaacttctcctgtatatactctgcctgccccctcccagcctccaaacttagttatttaaaaaacaaaacaaaacaatgaaattagta

SEQ ID NO: 31 - DNA, 159 bp, Artificial Sequence
  Synthetic polynucleotide
tgggggtgggggaatctagcctctctagagccctagccctctgacgaggaggaggtgtagtgccctgtagcttttccccaggactcgccacatgtccctttgtctggggaatgatgccctcaccctctgcatctggcctgacatctgttctctctgcag

SEQ ID NO: 32 - DNA, 897 bp, Rattus Norvegicus
tcagtacataaaatgatataggacattaaaacatttatgtatatcgtacattaaattattttccccaagcatataagcatgtaatatatatctaatgatttaggacatacatttaaactcaactataaattcataacaacatgtctattctcaaatacattaagataatgcttgttagacatatctgtgttattagacatgcaccattaagtcataaacctttctcttccatatgactatccctgtccccaattggtctctatttctaccatcctccgtgaaatcaacaacccgcccactcgtccccctcttctcgctccgggcccattcgtcctgggggtgactatactgaaactttaccaggcatctggttcttacttcagggccatcaattggttcatcgtccatacgttccccttaaataagacatctcgatggtaacgggtctaatcagcccatgatcaacataactgtggtgatatacatttggtattttttaattttcggatgccttcctcaacatagccgtcaaggcatgaaggtcagcacaaagtcctgtggaaccttttagttaagggtcatttatcctcatagacaaagctcgaaagactattttattcatgtttgtaagacataaatatttataaatactgaaaaatctgtcaacaaacccccccaccccctacacctgaaacttcaatgccaaaccccaaaaacattaaagcaagaattaaataaaacaaaaagctacttaattcttaaaaggcttctccattctagtagaccacaaaattttaacttaaatcttagcattggtaaaatttcccgacacaaaatctttccttctaactaaaccctctttacttgcctaccctcagaaaattccacatacaccaa

SEQ ID NO: 33 - DNA, 897 bp, Artificial Sequence; identical to SEQ ID NO: 32
  Synthetic polynucleotide

SEQ ID NO: 34 - DNA, 481 bp, Rattus Norvegicus
tcatctatcttatataagataatgtctgatctaaatatgagaccctgtgaggttggccataagcacactgttagctccttccgtcagacctttttggggctggctggtgaaacagaatgaatcctgttgggtctgcttctcacagcgtatggggctgtgtgcttttactcatggtggcagcagcagttgttgtcacttctttggccaggcacagtgggtgatccttacagagcagcacagattcccattgattccagtctatgtatttacttgtggttggtacagaatttaatgcagatagagtctttaaatatgtgaagatatgcagcactgattttaaacttaaactggcatgatactttccatatgtctagacacactggtaaatttttgttacatattgttttcaaattttccgttagtcttgtttctatgagatacttttattgtgacttaaaattcttagaaattaaagttctga

SEQ ID NO: 35 - DNA, 481 bp, Artificial Sequence; identical to SEQ ID NO: 34
  Synthetic polynucleotide

SEQ ID NO: 36 - DNA, 1233 bp, Rattus Norvegicus
aagttcagtgaaatcttgaataccaaacatctgacatacctcacaaattcaggaaagcaaggatggaccttcacaagtgatgcaagtttaatcggataagttccaaacgagttttaggtctttagtattgtaaataatcattctgtttttctacttacctgtttttatcaagatacatttccatgtggagtgacttcaaaatgcttgatttcatagagccactaggtggcatctttgtaaaagtggaagcacatgaaaagaaatagcctaggagtaaatgcaagatagcaaggtagatgagtcgttttgaacaagacctaagcaagtgcacagatttaggatgaaaagttaaattaaaagctttaacactttttattttgtgattaatactatttttctattgctattttcattcaggggacattgacatttatgtagccaaacatgtttatgtatttgtgagagagaaagtgtgtgtgtgcgtgcgcgcgtgttcgtgcacttgcttttgctatgttagtaacctatgcagatagaccacctaagtacattctgtgtaaggctttttaaaattgagcatagaaagcagagttcatagtacattatcaataagttattaagatatcacgctagacttaacagtggaaagaacatgaatttttctgctgcctctattcctaaccctggtgttttcattctgtccctaacaggaggctaatgaatgataaagctttgaaggtgaaggggtgggaaagtgatgttagtgaagagatgagagtggctttgtagcaacagggtataaaaaagacaacgacgattataatgagggagatggattttacaacacgtttttaatttcactaaactttacatgccaaaggatgttaaatatagcacgtaataagtgtttaaaatctgggctcttgattcagaccgagttttaatactggatctcattcaatctttgtgttttttctttttcttttcttttttttccaatagtacggcagtgctaatagcagtcctttagtagagttactgcattgactattggaaatacagataagttgctgtgcttagtgataaaacttactaaacatttgctgttttttgtgagataaccacatgaaatttgttcagggaacatatttgatctgttctccaaaatgtcttaagactaaagaatttgatgaaagaaaaataaaagctttgcttctcctgacttagaaagca

SEQ ID NO: 37 - DNA, 1437 bp, Artificial Sequence
  Synthetic polynucleotide
aagttcagtgaaatcttgaataccaaacatctgacatacctcacaaattcaggaaagcaaggatggaccttcacaagtgatgcaagtttaatcggataagttccaaacgagttttaggtctttagtattgtaaataatcattctgtttttctacttacctgtttttatcaagatacatttccatgtggagtgacttcaaaatgcttgatttcatagagccactaggtggcatctttgtaaaagtggaagcacatgaaaagaaatagcctaggagtaaatgcaagatagcaaggtagatgagtcgttttgaacaagacctaagcaagtgcacagatttaggatgaaaagttaaattaaaagctttaacactttttattttgtgattaatactatttttctattgctattttcattcaggggacattgacatttatgtagccaaacatgtttatgtatttgtgagagagaaagtgtgtgtgtgcgtgcgcgcgtgttcgtgcacttgcttttgctatgttagtaacctatgcagatagaccacctaagtacattctgtgtaaggctttttaaaattgagcatagaaagcagagttcatagtacattatcaataagttattaagatatcacgctagacttaacagtggaaagaacatgaatttttctgctgcctctattcctaaccctggtgttttcattctgtccctaacaggaggctaatgaatgataaagctttgaaggtgaaggggtgggaaagtgatgttagtgaagagatgagagtggctttgtagcaacagggtataaaaaagacaacgacgattataatgagggagatggattttacaacacgtttttaatttcactaaactttacatgccaaaggatgttaaatatagcacgtaataagtgtttaaaatctgggctcttgattcagaccgagttttaatactggatctcattcaatctttgtgttttttctttttcttttcttttttttccaatagtacggcagtgctaatagcagtcctttagtagagttactgcattgactattggaaatacagataagttgctgtgcttagtgataaaacttactaaacatttgctgttttttgtgagataaccacatgaaatttgttcagggaacatatttgatctgttctccaaaatgtcttaagactaaagaatttgatgaaagaaaaataaaagctttgcttctcctgacttagaaagcagggtatttgagagttcttcctgttctttctgggtgccaggaagctaaagcacaaaagcaagtagatatacaacagttgtaaacttgtgttcttttataatccctgtctctcggtgttcatatttgcatttttaaagatgataacctgtgagcttagactgcttacaggactgcctttccaaaagtgtgagagagctcccagaga

SEQ ID NO: 38 - DNA, 627 bp, Rattus Norvegicus
gttataataagtatcctgggtatctaatccattagtgacatttcataggcaaccgcatggacccacatgggagtgtgaatagaacaggatcccagaggaacagttccagtaacagagcccacatctgttgctagctccttagtttagtatggacggatggtaaagaccaaagtctatagcacggaaaagattaataaatgagtaataaatgggtgttaataaatgataatttttgtggtagttatgaaggatagttttagctaatttaaagagagtgttagcttgtgttggggagaggccacagtagaggctgcacttgccactgctgtgtagcggcactctcctgactcacttaaaaattctctggaggtttaagtgaggacccagttccatctctagatatccaggctccctagaacatttccagttgattccaacatgcatttggtgttaaagaccactgcagtgtcatttgcacatagacccaagacatggagttaggttttgttttttggggtttttttttgcaattctaatgtataatatcaatcaatattcttttgtgtgataccacatttggactattgtcccttcagctgataactgacattaaagaaatacagttga

SEQ ID NO: 39 - DNA, 627 bp, Artificial Sequence; identical to SEQ ID NO: 38
  Synthetic polynucleotide

SEQ ID NO: 40 - DNA, 1654 bp, Rattus Norvegicus
gcctacattgctgcggccaccagacggaccaggggccaccagtgccagccctgtggcctgtctactggactctggtcaccaggctcagcttgttgagttcctcattgtgcactatgagcagatcttcgggatggatgagctccctctgacctctgagcccctgactcaagaccctgccttggctcctgcactcctcgaatccagtccccagcacccagccttactcgttgcccaagactcacagcccctgaccatagcctcagattccagcccagaccccaaacaacacagtgccttggagaagtgtcccaaggtcgcgcctcctgagcttacggctctgcagagtaaccgaaaggaggaggaggaggaagacaccagagatggggcgggggatgggtccagccactcccccgaggacttgctcctggtagcacaatcccggggccacttcagccgccagccagtgaagtattcccggggaggtgtgcgaccagttactcatcagctctccagcctggctctggtggcctccaagctgtgtgaggagactcctatcactgtctcagcagtgcaccgaggtagtttgagggcacgaggcctgggccctgctgctgcctcccctgaaggcggcaccctgcgccggaaccctctgcccaagcactttgagatcacccaggagacagcccggctactctccaagctggacagtgaggctgtgtccagaacctcctgttgtgctgacccagagccggaggagtctgaggagcacctctgaccacccaccatcctgagggacctaggactgaattgatggtcctatatgtctccctacctgacccaacctggtgaccaaaccaattccatggggtgtggggagagatggggttaccgagagttcaggtaatgaagattacatgcctatgggagtcctgaaacacttggttgaagtgtccctccctgccagaggagatttaaacaagcaaggcaacacaggcaattggggtcaagactcaatggtcaattcaggtcagtagatccttcaggtctacctcagtctctgacctcgagacaggcgtctgactgtttcagtcataatggcttctggtcctgctaccacttgcctccttggcagactccaattcagccctgtcacctttatttactccttccttttgtttgtttgtttgtttgtttgttttgagccaaggtctttctgtagctctggctggtctggagctccctttgcaaaccacaaatcctctgtctcccaagtgctggccaaatctgtgttcttacaacctccaaataaaaccctcttcctgcagccggcaggacggccgagcacaatggtgcttgctgtacagcctcatagaccttccttggatctcccagacacacttagtggaaggagagaatccattcttgcaaattgtctgacctatgcacatgcacacaaaaaaataaatttttaattaaaaaaatctgctgggtagtggtgccacacacctttatttaaacctaacactcaggaggtagaggcaggaggatctcttgagtttgattccagacaggtctacagattaagttccaggacagccagggctacacagagacaccctatcttgggaagcaaaataaagtaagataaatcttg

SEQ ID NO: 41 - DNA, 1259 bp, Artificial Sequence
  Synthetic polynucleotide
ggtccagccactcccccgaggacttgctcctggtagcacaatcccggggccacttcagccgccagccagtgaagtattcccggggaggtgtgcgaccagttactcatcagctctccagcctggctctggtggcctccaagctgtgtgaggagactcctatcactgtctcagcagtgcaccgaggtagtttgagggcacgaggcctgggccctgctgctgcctcccctgaaggcggcaccctgcgccggaaccctctgcccaagcactttgagatcacccaggagacagcccggctactctccaagctggacagtgaggctgtgtccagaacctcctgttgtgctgacccagagccggaggagtctgaggagcacctctgaccacccaccatcctgagggacctaggactgaattgatggtcctatatgtctccctacctgacccaacctggtgaccaaaccaattccatggggtgtggggagagatggggttaccgagagttcaggtaatgaagattacatgcctatgggagtcctgaaacacttggttgaagtgtccctccctgccagaggagatttaaacaagcaaggcaacacaggcaattggggtcaagactcaatggtcaattcaggtcagtagatccttcaggtctacctcagtctctgacctcgagacaggcgtctgactgtttcagtcataatggcttctggtcctgctaccacttgcctccttggcagactccaattcagccctgtcacctttatttactccttccttttgtttgtttgtttgtttgtttgttttgagccaaggtctttctgtagctctggctggtctggagctccctttgcaaaccacaaatcctctgtctcccaagtgctggccaaatctgtgttcttacaacctccaaataaaaccctcttcctgcagccggcaggacggccgagcacaatggtgcttgctgtacagcctcatagaccttccttggatctcccagacacacttagtggaaggagagaatccattcttgcaaattgtctgacctatgcacatgcacacaaaaaaataaatttttaattaaaaaaatctgctgggtagtggtgccacacacctttatttaaacctaacactcaggaggtagaggcaggaggatctcttgagtttgattccagacaggtctacagattaagttccaggacagccagggctacacagagacaccctatcttgggaagcaaaataaagtaagataaatcttg

SEQ ID NO: 42 - DNA, 2221 bp, Rattus Norvegicus
gcgaccggcagggtgcccgcccgacttcccagctgccccgtgcggcccgggcatgcccagtgtgggcgcagccccggctctccgagcgccggggcggagcgggcagccgggcgcggagcaggcgaaccgaacctcggtgggcgagcgccggggctggagccgcgggggtcggtggcgggagctggggaccgaacccagggccttgcgcttcttaggccttccatgatgagtgatttgggggcacttccctctctctgtaattgatagtctgggcagtgaagagatggctgacagtgtcaaaacctttctgcaggaccttggcaggggaatcaaagactccatctggggcatctgtaccatctcaaagctagatgctcggatccagcagaagagagaggagcagcgtcgaaggagggcaagtagcctcttggcccagaggagagcccagagtgtagagcggaagcaagagagtgagccacgtattgttagtagaattttccagtgctgtgcttggaatggtggagtattctggttcagtctcctcttgttttaccgagtgtttattcctgtgcttcaatcagtaacagcccgggttattggtgatccgtcactacatggagatgtttggtcatggctggaattcttcctcacatcaattttcagtgctctttgggtgctgcccctgtttgtgcttagcaaagttgtgaatgccatttggttccaagatatagctgacttggcatttgaggtatcagggaggaaacctcatccattccccagtgtcagcaaaataattgctgacatgctcttcaaccttttgctacaggcacttttccttattcaggggatgtttgtgagtctcttccccatccatcttgtgggtcagctggttagtttgctgcatatgtctcttctctactcactctactgctttgagtaccgttggttcaacaaaggaattgaaatgcaccagcgattgtcaaacatagaaaggaactggccttactactttgggtttggcttgcccttggctttcctcacagcaatgcaatcctcctacattatcagtggctgcctcttttctatcctgtttcctttattcatcatcagcgccaatgaagcaaagactcctggaaaagcatatcttttccagttgcgcctcttctccttggtggtctttttaagcaacagacttttccacaagacggtctacctacagtcggccctgagcagctcatcctccgcagagaaattcccttcgccacatccttctcctgccaaactgaaagctgctgcaggccactgagtcctgctgtcaaaggggacgggtgggactgggtggaggatgtggcagctcttttcttggttttcctcccctgctgtggaaggcaggacccactgccaaggggcctctgcatattcctttgtctttgaattggaatcttcctgactccggtatgtggatttttaccaccaccctaggtctggaaggaccagttttccagctgttttttagcttttgccagctcctgtgcctggactgatttgagtactcccccctcccttgtgtcatttaccttcccacctcctcctgcctcccagcacccctgggtgaatgggctttgtaattttaactgttgtattttgtgaatttgttgttactgtttcctgtgaagcacataccttgtatgtgggaggtaaaggagcacgccagctgctccatgtcactccctctatagccatcactgtcttgtttttttgtaactcaggttaggttttggtctttactgctctgctgcagaaaaggaaaggaatgggggaaaggagcctaagagatgggacagatagacctcagccaaactggttgggttttgaggagtcatgttctttgttccccttgaaggggaaacaggttttttcactggtacatttaaagttccccagctatgggaaggtaccagttctggacaagtgccactgcatcatagcaactctgaagatgaaacttactgttggccactgccaggtcagactcgtgttttaaggaatactgggtgcttcatataggaactgaaggggtaaacttactaaaccattcaacctgtgattggtgatgtttttctgtcattttaagagtcgacacatggggtgggagggcagatgtaaaaaaacttgtacaattttaaaatcacaattaaacgtgagctggtttcccataaaa

SEQ ID NO: 43 - DNA, 1078 bp, Artificial Sequence
  Synthetic polynucleotide
tcttttccagttgcgcctcttctccttggtggtctttttaagcaacagacttttccacaagacggtctacctacagtcggccctgagcagctcatcctccgcagagaaattcccttcgccacatccttctcctgccaaactgaaagctgctgcaggccactgagtcctgctgtcaaaggggacgggtgggactgggtggaggatgtggcagctcttttcttggttttcctcccctgctgtggaaggcaggacccactgccaaggggcctctgcatattcctttgtctttgaattggaatcttcctgactccggtatgtggatttttaccaccaccctaggtctggaaggaccagttttccagctgttttttagcttttgccagctcctgtgcctggactgatttgagtactcccccctcccttgtgtcatttaccttcccacctcctcctgcctcccagcacccctgggtgaatgggctttgtaattttaactgttgtattttgtgaatttgttgttactgtttcctgtgaagcacataccttgtatgtgggaggtaaaggagcacgccagctgctccatgtcactccctctatagccatcactgtcttgtttttttgtaactcaggttaggttttggtctttactgctctgctgcagaaaaggaaaggaatgggggaaaggagcctaagagatgggacagatagacctcagccaaactggttgggttttgaggagtcatgttctttgttccccttgaaggggaaacaggttttttcactggtacatttaaagttccccagctatgggaaggtaccagttctggacaagtgccactgcatcatagcaactctgaagatgaaacttactgttggccactgccaggtcagactcgtgttttaaggaatactgggtgcttcatataggaactgaaggggtaaacttactaaaccattcaacctgtgattggtgatgtttttctgtcattttaagagtcgacacatggggtgggagggcagatgtaaaaaaacttgtacaattttaaaatcacaattaaacgtgagctggtttcccataaaa

SEQ ID NO: 44 - DNA, 966 bp, Rattus Norvegicus
agaccgttcttgcctctgaaccggcaatcatggctccgggttggccgcggcctctgccgcagctcctcgtgttgggattcgggttggtgttgatacgcgccacggccggggagcaagcaccaggcaacgccccatgctcaagcggcagctcctggagcgcggacctcgacaagtgcatggactgcgcttcttgtccagcgcgaccacacagcgacttctgcctgggatgcgcagcagcacctcctgcccacttcaggatgctatggcccattctgggaggcgctcttagtctggccctggttttggcgctggtttctggtttcctggtctggagacgatgccgccggagagaaaagtttactacccccatagaggagactggtggagaaggctgcccaggtgtggcactgatccagtgaggagcacccgcgctggtgcccattcatcgtccattcattcattctggagccagcctggctttccagagacaagccgcgccagactcttccaaccacaagggggtggggcgaggtggtgattcacctccaaggactgggcttagggttcaggggagccttccagggtgtctaattgccctgtctctggttctggggcagacagagagcctcaagctaggtcacaaagcgactcatactaaggatctgcagcatttgcacaagggaatctcttgctccccacaagtcctggaggcctggccgacttgaggggcagacacaacactgggttccacccactcggatggactgaaatgccactaccctgagttttagccctgggagtgggttagattgagggacctgttcataacactaagggggctggccctctgggagggctggtcccagtacacacaaacacaacccatcccccaagggggtgatatttattttggggataaagtttggaggggagggagacttattaataaaagaatctttaacttta

SEQ ID NO: 45 - DNA, 603 bp, Artificial Sequence
  Synthetic polynucleotide
cccccatagaggagactggtggagaaggctgcccaggtgtggcactgatccagtgaggagcacccgcgctggtgcccattcatcgtccattcattcattctggagccagcctggctttccagagacaagccgcgccagactcttccaaccacaagggggtggggcgaggtggtgattcacctccaaggactgggcttagggttcaggggagccttccagggtgtctaattgccctgtctctggttctggggcagacagagagcctcaagctaggtcacaaagcgactcatactaaggatctgcagcatttgcacaagggaatctcttgctccccacaagtcctggaggcctggccgacttgaggggcagacacaacactgggttccacccactcggatggactgaaatgccactaccctgagttttagccctgggagtgggttagattgagggacctgttcataacactaagggggctggccctctgggagggctggtcccagtacacacaaacacaacccatcccccaagggggtgatatttattttggggataaagtttggaggggagggagacttattaataaaagaatctttaacttta

SEQ ID NO: 46 - DNA, 443 bp, Rattus Norvegicus
aagcactgttagtttgctggtctgtctcctctgtgctcttgtctgtgtctgtgtcctcgatcctgactcaagcaatcccgctctgttttgctgactcctgagcagacatagcttgtgggccaggaccttgccagccttgagtggcagtggcttcagaagtgaaatgatctggctgtctgcattcgaatgactccccagtcccagcctagcctctcttttgcactgctggttcagcccactgggcctccgtcctttcctctggaagggacttggccttgggtgacaaattcctctttgatgaatgtaccctgtgggaatgtttcatactgacagattatttttatttattcaatgtcgtatttaagatatttattttttatactgaaggagcaccctttttttaagagaaataaatgaaataataaagaacccattcttgtcga

SEQ ID NO: 47 - DNA, 443 bp, Artificial Sequence; identical to SEQ ID NO: 46
  Synthetic polynucleotide

SEQ ID NO: 48 - DNA, 808 bp, Rattus Norvegicus
cagaagagaacccagggcactgggacaggacttcctgagggtctgaggagcatggcagacagcccagaggtggtagccatggactgtgagatggtggggctcgggcctcaaagggtgagtggcctcgcccgctgcagcattgtgaacgtccacagcgcagtcctctatgacaagtacatccagcctgagggagagatcacggactacagaacccaagtcagcgggatcacgcctcagcacatggcgagggccacgccatttgctgaagccaggctagagatcctgcagcttctgaaaggcaagctggtggtgggccatgacctgaagcatgacttcagtgccctgaaggaggacatgagaaaatacaccatctacgacacgtccacagacatgctgctgtggcacgaggccaagctgcactgctacagccgtgtgtccctgaggctgctgtgcaagcgcctgctgcacaagagcatccagaacaactggcggggccactgctctgtagaagatgccagggccacaatggagctctacaaaatctctcagcgactcagagcccagcgagggctgccctgcctgggaacatcagcctgaacttcatcctcatccaggatcagaagcagctactccttgaaggaccatagttttccatgaatgagacctgtttctagctacaacaacaacagcaacaaccacaaaatgcaaaagcaagaacactccgccattacagcaattctcttgctttggagcaaagaaaaaaaaaaccaaactttagtaataaatgactttgattttgtctaatcaa

SEQ ID NO: 49 - DNA, 328 bp, Artificial Sequence
  Synthetic polynucleotide
aacaactggcggggccactgctctgtagaagatgccagggccacaatggagctctacaaaatctctcagcgactcagagcccagcgagggctgccctgcctgggaacatcagcctgaacttcatcctcatccaggatcagaagcagctactccttgaaggaccatagttttccatgaatgagacctgtttctagctacaacaacaacagcaacaaccacaaaatgcaaaagcaagaacactccgccattacagcaattctcttgctttggagcaaagaaaaaaaaaaccaaactttagtaataaatgactttgattttgtctaatcaa

SEQ ID NO: 50 - DNA, 1491 bp, Rattus Norvegicus
gcaaagtcacactcaggacacaagcgacatggccctgcgcgcgctccatgcccagcctacaggcggtcctcagctgaggttcctgctgttcctgctgctcttgctgcttctgctgtcatggccatctcagggggacgccctggcattgcctgagcagcgacgctccctctctgagtcccaactcaacccggacgagctacggggtcgcttccaggacctgctgagccgactgcatgccaaccagagccgggaggactcgaactcagaaccaacccctgacccagctgtccggatactcagtccagaggtgcgattggggtcccacggccggctgctgctccgcgtcaaccgggcgtcgctgactcagggtctccccgaagcctaccgcgtgcaccgagcgctgctcctgctgacgccttcgtccaggccctgggacatcaccaggcccctgcaacgtgcgatcagcctccagggaccccacgctcgcgcactgcgcttgcgcctggcgccgcctcccgacctagccgtgctgccctctggcggcgcgcgcctggaactgcacttacgatcggccgccggcagggggcgccgaagcgcgcatttgcacccaagagactcgtgcccgctgggtcccgggcgctgctgtcacctggagactgtgcaggcaactctcgaggacctaggttggagcgactgggtactgtccccgcgtcagctgcaactgagcatgtgcgtgggcgagtgcccccacctctaccggtcggccaacacgcatgcgcagatcaaagcacgcctgcatggcctgcagcccgacagagtgccggccccgtgctgtgtcccctccagctacaccccggtggttcttatgcacaggacagacagcggcgtgtcactgcagacttatgatgatctggtagcccagggctgccactgcgcttgagcactgggccctgctccttacctacactccccttccatgatgctatttatatttttatttaataatattattaatttattggggttgggctgggtgggtggattgtgtatttatttaaaactctgctaataaaggtgagcttggtttctatgcggcgtctcagtgatctcggcatgattcccatcaccctagcctcagtttcccttgtcacctgagaagtggagcagaggacgggcctccattaggctggcctggggtgattttattgactgacagctgatgtgagtgaccagccagggcctggtggtcctgccatgtttgagccgcaaggaggagcaggacagtcagcagttcattgctgcagcctctgcttcagcgcccttctaggttccctctggacattcctcccatccccatttcaagccaggcctaacatcccagaatcagggggcctctgcaatccagatagtccgacaacggctggctgggaatgaaaagtgtaagaagccagtggctgctcagtcaacaagactgggtgtctcggctgctct

SEQ ID NO: 51 - DNA, 1183 bp, Artificial Sequence
  Synthetic polynucleotide
tgcgattggggtcccacggccggctgctgctccgcgtcaaccgggcgtcgctgactcagggtctccccgaagcctaccgcgtgcaccgagcgctgctcctgctgacgccttcgtccaggccctgggacatcaccaggcccctgcaacgtgcgatcagcctccagggaccccacgctcgcgcactgcgcttgcgcctggcgccgcctcccgacctagccgtgctgccctctggcggcgcgcgcctggaactgcacttacgatcggccgccggcagggggcgccgaagcgcgcatttgcacccaagagactcgtgcccgctgggtcccgggcgctgctgtcacctggagactgtgcaggcaactctcgaggacctaggttggagcgactgggtactgtccccgcgtcagctgcaactgagcatgtgcgtgggcgagtgcccccacctctaccggtcggccaacacgcatgcgcagatcaaagcacgcctgcatggcctgcagcccgacagagtgccggccccgtgctgtgtcccctccagctacaccccggtggttcttatgcacaggacagacagcggcgtgtcactgcagacttatgatgatctggtagcccagggctgccactgcgcttgagcactgggccctgctccttacctacactccccttccatgatgctatttatatttttatttaataatattattaatttattggggttgggctgggtgggtggattgtgtatttatttaaaactctgctaataaaggtgagcttggtttctatgcggcgtctcagtgatctcggcatgattcccatcaccctagcctcagtttcccttgtcacctgagaagtggagcagaggacgggcctccattaggctggcctggggtgattttattgactgacagctgatgtgagtgaccagccagggcctggtggtcctgccatgtttgagccgcaaggaggagcaggacagtcagcagttcattgctgcagcctctgcttcagcgcccttctaggttccctctggacattcctcccatccccatttcaagccaggcctaacatcccagaatcagggggcctctgcaatccagatagtccgacaacggctggctgggaatgaaaagtgtaagaagccagtggctgctcagtcaacaagactgggtgtctcggctgctct

SEQ ID NO: 52 - DNA, 3218 bp, Rattus Norvegicus
caatcctggtgcccagcctatctccccgcggccgggcgcagtccttcaccgcacgctgaaccggaggaaggctgcgcctagtcggggcgctgagggaccctccaccgggacgccggcccctccccgggcctctgctcacttgcccccctgcgagcccgtccccctagtcggcctctcggatcggggacgtggggcgagctgagagcaggcccggggtgggtggtcactgtggagaagacgtggctgtcaagatgatagaagtactgacaactgactctcagaaactgctacaccagctgaacaccctgttggaacaggagtccagatgtcagccaaaggtctgtggcctgaaactgattgagtctgcacatgataatggcctcaggatgactgcaagactccgggactttgaagtcaaagatctactgagtctaactcagttctttggcttcgacacagaaacattttcccttgctgtgaatttactggacagattcttgtctaaaatgaaggtacaggcgaagcatctcggctgtgtcggactgagctgcttttatttggctgtgaaatcgattgaagaggaaaggaacgtcccgctggcaactgatttgatccggataagtcagtataggttcacagtttcagacctgatgagaatggaaaagattgtgttggagaaagtgtgctggaaagtcaaagctactactgccttccaatttctgcagctctattactccctcattcgggagaccttgccatttgaaaggagaaacgatctgaattttgaaagactagaagcccaactgaaggcgtgccactgcaggatcatattttctaaggcaaagccttctgtgctggcgctggcaatcatcgctttggagatccaagcactgaagtatgtggagttaacagaaggagtagaatgtattcagaaacattccaagataagtggccgagatctgaccttctggcaagagcttgtttccaagtgtttaactgaatattcatcaaacaagtgttccaagccgaacggtcagaagttaaaatggatcgtgtctgggcgcactgcacgacaactgaagcacagttattacaggataacccacctcccaacaattcccgaaaccatgggttagttggcaaatctggttgttatcctctgtgtacagaacatttcccagtgagatcgtttttgtgctataacttaaggattgaaatactaccttcaatataaagaatacaggatgaaaacagtaaaggaaacgtgagtttgttggtctagacagagaatactgggaggcattcactgtgtaccgcagtctgaagagaaatgagtatcaaacctctagacacatgctcatactgctgtcaaaggactagcgtagaaaagagagtcctccaaaccggaagtttaaatgtagttactaaaatagcacttctttaacttacatatccccccactgtggcttatttaaagttacagaagtccaagcagaacgacaaaagatgtgacccatatatgaacacattttaatctgttcattgattaggagagtgaatatgaacttgcatgatgcccatgttaggtttctggaaactgccggggtatcttaattctctagtattctccctctgtggcagttgggctaatacaaagtaactatacgcatgagaatataaaatcagtctctgatacatacacatttttaccatcaaaatttcttaatcatagcaaagacttacctttttatgattaggaattttttttttaatgtatggcagcacatgcctttaatcccaacactagggaggcagaggcaggtggatctctttgagttcgaagccaggctggtctttacagtgagttccaggacagctggagagctacagaatggagagacgctgtctcaaaaacactcaaaacaaacaaacaaaccataccagtttgtaggcagacttctgttgggttgggtttgtactgtttgcctatgcagtgggattacagcagcagcaacaaaaactgtccctgaagtctttctctgccactgtgacctgagtttcctatggtacgcgatttactctaagaaacctcagcccctcaccacgttagctgttggcaaatggcctcacagttgcggaaagtcccaattctaggcttgggaaagcaatgcttagatttgaattggcccatgaagcattcaaatcaaggctaaagacataaatgtgaaataaaactgtgaaccttcattttaacattgatctcacttcccagatttaatcaatatatacttaggtggtattaaaaatggtaaactgcctaatttaaatctcaaaatttaaactatgaggtttacatcaaagccaacatttcacaaatgtacttttaaggtattaaaagaggtatttaagcagtaaatggtttcttggcacccataaccaagtaatagttaagttagaggtgggacttttttattgctatgagaattacatttaaacttttgggtgttttataaaaagcagatttcacaagttttgaaaattgtgacctttactgaaatttgttacctttaatatttcttctagaggataggtatttataaaagaaaaattcgtcagaattgctgcctcaatctagtcccatttgagaaaatttgtttctactgtctcaataactggatgaaatatcactctgaaaacttgcctattgcactaaagctagtttaggcttgataaaacactccaggaggtttttaccacagactgtttctattaaaactgctgcttctcatgtacaattttgttttaaaaggaaccgagtacatctgcaaaacctaagtcttaagggacgtcaggaggtaccttcagaattataggatcaccatggtagtggggattctccatgctggccttgaatgtttgatcttcactgctgaaatgtgggtagctcctcagcgccctgtagagcctgagtctacctagaatagctgtaaccattttgacaagtaatggataagaaaattatccattgagaagctaaaaacaaaacaaaacaaaaccaaagaacgggtgtattttattcttaacctttgtaaaccatcactgagaacacttcagttcttcctaacagctgttatgcttcgatttgaaaaaaatactgagtggataaccaactaccatcatgctttgggtacacctttcaataaaattactgaaatgca

SEQ ID NO: 53 - DNA, 1558 bp, Artificial Sequence
  Synthetic polynucleotide
cagtaaaggaaacgtgagtttgttggtctagacagagaatactgggaggcattcactgtgtaccgcagtctgaagagaaatgagtatcaaacctctagacacatgctcatactgctgtcaaaggactagcgtagaaaagagagtcctccaaaccggaagtttaaatgtagttactaaaatagcacttctttaacttacatatccccccactgtggcttatttaaagttacagaagtccaagcagaacgacaaaagatgtgacccatatatgaacacattttaatctgttcattgattaggagagtgaatatgaacttgcatgatgcccatgttaggtttctggaaactgccggggtatcttaattctctagtattctccctctgtggcagttgggctaatacaaagtaactatacgcatgagaatataaaatcagtctctgatacatacacatttttaccatcaaaatttcttaatcatagcaaagacttacctttttatgattaggaattttttttttaatgtatggcagcacatgcctttaatcccaacactagggaggcagaggcaggtggatctctttgagttcgaagccaggctggtctttacagtgagttccaggacagctggagagctacagaatggagagacgctgtctcaaaaacactcaaaacaaacaaacaaaccataccagtttgtaggcagacttctgttgggttgggtttgtactgtttgcctatgcagtgggattacagcagcagcaacaaaaactgtccctgaagtctttctctgccactgtgacctgagtttcctatggtacgcgatttactctaagaaacctcagcccctcaccacgttagctgttggcaaatggcctcacagttgcggaaagtcccaattctaggcttgggaaagcaatgcttagatttgaattggcccatgaagcattcaaatcaaggctaaagacataaatgtgaaataaaactgtgaaccttcattttaacattgatctcacttcccagatttaatcaatatatacttaggtggtattaaaaatggtaaactgcctaatttaaatctcaaaatttaaactatgaggtttacatcaaagccaacatttcacaaatgtacttttaaggtattaaaagaggtatttaagcagtaaatggtttcttggcacccataaccaagtaatagttaagttagaggtgggacttttttattgctatgagaattacatttaaacttttgggtgttttataaaaagcagatttcacaagttttgaaaattgtgacctttactgaaatttgttacctttaatatttcttctagaggataggtatttataaaagaaaaattcgtcagaattgctgcctcaatctagtcccatttgagaaaatttgtttctactgtctcaataactggatgaaatatcactctgaaaacttgcctattgcactaaagctagtttaggcttgataaaacactccaggaggtttttaccacagactgtttctattaaaactgctgcttctcatgtacaattttgttttaaaag

SEQ ID NO: 54 - DNA, 2030 bp, Rattus Norvegicus
ggggcggatgcgcagaggtgcaccacgtgtggtgttcctccagcgcagtcaggatctcgtctagtttgggtctaggttagatttggaatcgtggagatgcggaaggaaagcccacctccgctcgtgcccccggcggcccgcgagtggaacctgccccccaatgcgcccgcctgcatggaacggcagttggaagctgcacggtaccgctctgatggttcccttctgctcggggcttccagtctgagtggtcgctgctgggcaggatctctgtggtttttcaaggatcctagtgcggcccccaacgaaggtttctgctctgctggcgtccagacggaggctggagtagctgacctcacttgggttggggacaaaggtatcttagtggcttctgattcaggtgctgttgaattgtgggagctagatgagaacgagacacttattgtcagcaagttctgcaagtatgagcatgatgacattgtgtctactgtcactgtcctgagctccggcacacaggctgtcagtggtagcaaagacttctgcatcaaaatttgggacctggctcagcagatgtcgctgaattcataccgagctcatgcgggacaggtcacctgtgttgctgcctctccccacagagaaactgtgtttctttcatgcagtgaggacagtagaattttgctctgggatacccgctgtcccaagccggcatcacagatgggctgcaatgcctctggctacctccctacctccctggcttggcatcctcagcagagtgaaatctttgtctttggtgatgagaatggatctgtctcccttgtggacaccaagaatgcaagctgcaccctcagctcagctgtgcactcgcagtgtgtcactagactggtgttctctccacacagtgttcccttcctggcctctctcagtgaagactgttcacttgctgtgctggattcaagcttttctgaggtgtttagaagtcgagcccacagagactttgtgagagatgctacatggtctccactcaatcactcccttcttaccacagttggctgggaccatcaggtcatccaccatgttgtgccgttagagcctctcccagcccctggacctgacagtgttgtggagtagaatggattgcagaagaaaagagccttctgtctgcaagtgtcgactcgcttgcccctgccttccatttgtgagagagagagcacaggagccttgtagagcatgattactccctagtcccgtgcagtaactagtcagattctcagcctgagggaggctgcatcccatagtgacttggaggaaaaagcttcctttataaatggatgtatgtgtgagtacacgtgtgagtgcagtgtatagtttggagtggagaaaattatccttcagctttcccaaagcaatgctctttacccccgacaaaatgaccagaggattttatgcttcccatgtctgggggctgactatgttgttagggatatggagtgtgggtatccctggatgcttgggaatacttcacattactcagaggtgcgtaacttatttttatatagagtttaattcactatatgggaattacttcccatatacaaaagtctagccagactaccttgagaagacagaacagttttgtacgaatgcctgttaaatggaatcaaatccactttctgggtgaatcaatttggcccttggagttcgcgctcgctcaccctgtgttccttcttttgtgttgtgtatgagagatgagtgctttcctgtcctagatttcacagttttagggatgatggagtacttaaattaaaagtgtttcagtgaagcgagaagattgacatggattgttacagagaaatctgtcattgtcatttttcccacctaaaaattccctgaggactgatctgggtactttgctctggagagctgaagtctgagcgctgtatatttggactcctaagattgaagatccggcatcagagaaactgtcaaaaatactggaaggtgaaataaagacttatcctttcatctttcctcca

SEQ ID NO: 55 - DNA, 1146 bp, Artificial Sequence
  Synthetic polynucleotide
gtttagaagtcgagcccacagagactttgtgagagatgctacatggtctccactcaatcactcccttcttaccacagttggctgggaccatcaggtcatccaccatgttgtgccgttagagcctctcccagcccctggacctgacagtgttgtggagtagaatggattgcagaagaaaagagccttctgtctgcaagtgtcgactcgcttgcccctgccttccatttgtgagagagagagcacaggagccttgtagagcatgattactccctagtcccgtgcagtaactagtcagattctcagcctgagggaggctgcatcccatagtgacttggaggaaaaagcttcctttataaatggatgtatgtgtgagtacacgtgtgagtgcagtgtatagtttggagtggagaaaattatccttcagctttcccaaagcaatgctctttacccccgacaaaatgaccagaggattttatgcttcccatgtctgggggctgactatgttgttagggatatggagtgtgggtatccctggatgcttgggaatacttcacattactcagaggtgcgtaacttatttttatatagagtttaattcactatatgggaattacttcccatatacaaaagtctagccagactaccttgagaagacagaacagttttgtacgaatgcctgttaaatggaatcaaatccactttctgggtgaatcaatttggcccttggagttcgcgctcgctcaccctgtgttccttcttttgtgttgtgtatgagagatgagtgctttcctgtcctagatttcacagttttagggatgatggagtacttaaattaaaagtgtttcagtgaagcgagaagattgacatggattgttacagagaaatctgtcattgtcatttttcccacctaaaaattccctgaggactgatctgggtactttgctctggagagctgaagtctgagcgctgtatatttggactcctaagattgaagatccggcatcagagaaactgtcaaaaatactggaaggtgaaataaagacttatcctttcatctttcctccaaagcttatttattacctgctttgcttaggtgagtcacctgagtatcaatcagtcttaactgcccactgtctttgtagtcta

SEQ ID NO: 56 - DNA, 1971 bp, Rattus Norvegicus
ggtaaccaggagttaagcgggctccctgccagcgcgagggctttaaaaggggtgatgcaacgcgctcccagtcacagtctcactcagcgagacgcgcggtgcacggtgcttccccggcggagccgaccgaccaacccgcgctccggcagagtccttggcgctcgcctgccggcgggacagacagcccgcctctagccgctctctggaccctggccgccccgagcgaagactggagcaaaatgatgcttcaacatccaggccaggtctctgcctcagaagtcagcgcgaccgccatcgtcccctgcctctcacctcctgggtcactggtgtttgaggattttgctaacctgacaccttttgtcaaggaagagctgagattcgccatccagaacaagcacctttgccatcggatgtcctctgcgctggagtcagtcaccatcaacaacagacctctggagatgtcagtcaccaagtctgaggtggcccctgaagaagatgagagaaaaaggaggcggcgggaaagaaacaaaattgctgctgccaagtgtcgaaacaagaaaaaagagaagacagagtgcctgcagaaggagtcagagaaactggagagtgtgaatgccgaactgaaggcccagatcgaggagctgaagaatgagaagcagcatctgatttacatgctcaacctgcaccggcccacgtgtatcgtccgggctcagaacgggcggacgccggaagacgagaggaacctttttatccaacagataaaagaaggaacattgcagagctaagcagaggtggcatgggggcaattggggagtccttactgaatcctccttttccaccccaaaccctgaagccattggaaaactggcttcctgtgcacttctagaatctcagcagccacgagctgttgggtcaggagggcctgcggtgactactgcgttgtcccactctgtccccgagtgaaccgtggagcaggcaggagcatcctttgtctcaccggctccaggatttaggccttaccatcccggccattctcagatgacctagctggccccaggctggggtcccatgcaaagcaggatcgcactaatgggatgcaggcagaagtgtctaccttgacaggtggggtgggaccacgtcctccactgcggctgacaacatccctcctagggaagatggagtgagaacattcatcattgaagttgtccaatggccagggtatgctttctagaaactatgctgttctgtcctagactgactgtgcatagggcattcatttctgagcctggtgttgtgctatttagatgtttgtcttgcacaacattggcgtgatttttttccgggagtttcatcagacctgatttccgagagtttgggggtctgccactgtggacaatatcccccaaaagtgtttgggtggccatgtaaactggctgatgaccagctgtgctactctgtgctgaccgaggactgatgcctccttcccctgtacccactgctgaggaagaacccgggcacagcagctgtccttggctacaaactgttacaatgtcacagaacgaaggcacaaagtcccgctttcaaagggcgtaggactccacactcagtgacagggcaggaagagccaaggattctccgttttcccttccttcccaccaaaaaccacagcccgtggagactggtatttgaagccaggagtggggcaaggaaggtgtctgcactgtgggatgttaactgcgcttttgtcttgaagctattttgagatgcggtccagagtatttcagctgggaggtccctcccactggccaccagggctctggctactgttaaaattctgatgtttctgtgaaatcctcagtgttcaatccagactcagtagtatattacagttttctgtaagagagaacgttacttatttatcccagtattcctagcctgtcaacgtaataaaatatcagaatgagacctggt

SEQ ID NO: 57 - DNA, 1384 bp, Artificial Sequence
  Synthetic polynucleotide
gagtcagagaaactggagagtgtgaatgccgaactgaaggcccagatcgaggagctgaagaatgagaagcagcatctgatttacatgctcaacctgcaccggcccacgtgtatcgtccgggctcagaacgggcggacgccggaagacgagaggaacctttttatccaacagataaaagaaggaacattgcagagctaagcagaggtggcatgggggcaattggggagtccttactgaatcctccttttccaccccaaaccctgaagccattggaaaactggcttcctgtgcacttctagaatctcagcagccacgagctgttgggtcaggagggcctgcggtgactactgcgttgtcccactctgtccccgagtgaaccgtggagcaggcaggagcatcctttgtctcaccggctccaggatttaggccttaccatcccggccattctcagatgacctagctggccccaggctggggtcccatgcaaagcaggatcgcactaatgggatgcaggcagaagtgtctaccttgacaggtggggtgggaccacgtcctccactgcggctgacaacatccctcctagggaagatggagtgagaacattcatcattgaagttgtccaatggccagggtatgctttctagaaactatgctgttctgtcctagactgactgtgcatagggcattcatttctgagcctggtgttgtgctatttagatgtttgtcttgcacaacattggcgtgatttttttccgggagtttcatcagacctgatttccgagagtttgggggtctgccactgtggacaatatcccccaaaagtgtttgggtggccatgtaaactggctgatgaccagctgtgctactctgtgctgaccgaggactgatgcctccttcccctgtacccactgctgaggaagaacccgggcacagcagctgtccttggctacaaactgttacaatgtcacagaacgaaggcacaaagtcccgctttcaaagggcgtaggactccacactcagtgacagggcaggaagagccaaggattctccgttttcccttccttcccaccaaaaaccacagcccgtggagactggtatttgaagccaggagtggggcaaggaaggtgtctgcactgtgggatgttaactgcgcttttgtcttgaagctattttgagatgcggtccagagtatttcagctgggaggtccctcccactggccaccagggctctggctactgttaaaattctgatgtttctgtgaaatcctcagtgttcaatccagactcagtagtatattacagttttctgtaagagagaacgttacttatttatcccagtattcctagcctgtcaacgtaataaaatatcagaatgagacctggt

SEQ ID NO: 58 - DNA, 1120 bp, Rattus Norvegicus
ggcctgggcggagagggaggaaaacttcctggctgggactctggcggtttctgcctgccaaccctccgatccgcctaccagcgttggctcttcccgacccctcccccggcgcccccagtgtccgccatggccaaagcctacgaccacctcttcaagttgctgctgatcggggactcgggggtgggcaagacttgtctcatcattcgctttgcagaggacaacttcaacagcacttacatctccaccatcggaattgatttcaagatccgaactgtggaaatagaggggaagagaatcaaactgcaagtctgggacacagctggccaagaacgattcaagacaataaccaccgcctattaccgtggagccatgggcattatcctagtatatgacatcacagatgagaaatccttcgagaatattcagaactggatgaaaagcatcaaagagaacgcgtctgctggagtggagcgccttctgctggggaacaaatgtgacatggaagccaagcggaaggtgcagagagagcaggctgagaggttggcccgagagcacagaatccgattttttgagacaagtgccaaatccagtgtgaatgtggatgaggctttcagttccctggcccgtgacatcttgctcaagacaggaggccggagatcgggaaacagcagcaagccctcaagcactgacctgaaagtatctgacaagaagaacagcaacaagtgctccttgggctgaggaacatttcttgcctcctattcaccctgaacctggaggctagacctgagggaggtggactgaggtagactgatggaaaacagaggggaggagctgtggtggtgcctggaggggtggatgacaggggaggaaggaaagatgaaatgggcagggaaaggagggcgaggaaccaaggacgtgaaaagtgaagagaaggggtttgagaagagaaaaagaagaaggtctcaggtctcggaccgtccaacattaatgtcagtatgctgatctctccattcctggttcagggttcgggtcccgagaggctggctcggccctactctgagggtctctcactccacagatctttgttagtattaaaggccactgtttggcacgaatgttc

SEQ ID NO: 59 - DNA, 120 bp, Artificial Sequence
  Synthetic polynucleotide
ttggcccgagagcacagaatccgattttttgagacaagtgccaaatccagtgtgaatgtggatgaggctttcagttccctggcccgtgacatcttgctcaagacaggaggccggagatcg

SEQ ID NO: 60 - DNA, 1877 bp, Rattus Norvegicus
gatgggatgcatctatcttgtgatatgtaccagccacaggcaccatgtccgatcctggtgatgtccgacctgttccacacaggagcaaagtatgccgtcgtctgttcggtcccgtggacagtgagcagttgagccgcgattgcgatgcgctcatggcgagctgtctccaggaggcccgagaacggtggaactttgacttcgccactgagacgccactggagggcaactacgtctgggagcgtgttcggagcccagggctgcccaagatctacctgagccctgggtcccgccgccgtgatgacctgggaggggacaagaggcccagtacctcctcggccctgctgcaggggccagggccagctccggaggaccacgtggccttgtcgctgtcttgcactctggtgtctcacgcccctgagaggcctgaagactccccgggcgggaccgggacatctcagggccgaaaacggaggcagaccagcctaacagatttctatcactccaagcgccgattggtcttctgcaagagaaagccctgaagtgcccacgggaggctcgccttcttctgctgtgggtcaggaggcctcttccccatcttctgccttagccttcattcagtgtgtcttaattattatttgtgttttaatttaaacttctcctgtatatactctgcctgccccctcccagcctccaaacttagttatttaaaaaacaaaacaaaacaatgaaattagtaggacggtaggctccttagtgctgtttttttttttttttatgtagaccattatttaagtccttctcaacccaagctgtgtttctgatcctggcgggatggtcctggcgggatggtcctggcgggatggtcgcactggcctcatgccatctgcatctcgcatctcgcccaatccccgcccctcccccctagctcctcccccgcccccactccctgcctggttccttgccacttcttacctgggggcgatcctcagacctgaatagcactttggacaactgagtaggacttcggggtctccttgtcatctctaaggcccgctaggatgacagtgaagcagtcacagcctagaacaaagatgcccggttaggacctaagcgtaccgtccagagccttgacatttactcagacctgtgaagatcctttgccactcctggggaccccgcctcccctgtgggtctctgccagctgcccctctatttttgagggttaatctggtgatctgctgctctcccttcctcagacccttcccctccccaggttggcaggaggcatcctttccctcgccacggctcagtggaccagaagggaacgggtacacagggtacactaagtggggtttcctggtcctacctcaggcagctccagtggcaactgcccttcttatgggtctagggtaggtccttggtgacgagacgggcttcccagagcatccctgtgtgtgtggtgtgtggtggtggtggggggtggacttatctgggatggggaccccagccgctgaagtcctcagtgacttgtcccatttcttagtagttgtacaaggagtcaggccaagatggtgcctcgggggctgagggagctcacaggaactgagcagtgactggtcctttcccagtattgaatactgagcccctgtgggtgtcgaagcacttagtgggtctggccccaaccccaaacacccctgtttctgtaacaccctgagctggactgtttatctttagccgggagaacatgtattttggtcccctccctgtctccgctcggattgtaaacctcccacgtgtggggatcacaccctgcactgtcccgaatctttacaccctatcccaaagctggtgctcaataaatacttctagatgatttg

SEQ ID NO: 61 - DNA, 1109 bp, Artificial Sequence
  Synthetic polynucleotide
ttttttatgtagaccattatttaagtccttctcaacccaagctgtgtttctgatcctggcgggatggtcctggcgggatggtcctggcgggatggtcgcactggcctcatgccatctgcatctcgcatctcgcccaatccccgcccctcccccctagctcctcccccgcccccactccctgcctggttccttgccacttcttacctgggggcgatcctcagacctgaatagcactttggacaactgagtaggacttcggggtctccttgtcatctctaaggcccgctaggatgacagtgaagcagtcacagcctagaacaaagatgcccggttaggacctaagcgtaccgtccagagccttgacatttactcagacctgtgaagatcctttgccactcctggggaccccgcctcccctgtgggtctctgccagctgcccctctatttttgagggttaatctggtgatctgctgctctcccttcctcagacccttcccctccccaggttggcaggaggcatcctttccctcgccacggctcagtggaccagaagggaacgggtacacagggtacactaagtggggtttcctggtcctacctcaggcagctccagtggcaactgcccttcttatgggtctagggtaggtccttggtgacgagacgggcttcccagagcatccctgtgtgtgtggtgtgtggtggtggtggggggtggacttatctgggatggggaccccagccgctgaagtcctcagtgacttgtcccatttcttagtagttgtacaaggagtcaggccaagatggtgcctcgggggctgagggagctcacaggaactgagcagtgactggtcctttcccagtattgaatactgagcccctgtgggtgtcgaagcacttagtgggtctggccccaaccccaaacacccctgtttctgtaacaccctgagctggactgtttatctttagccgggagaacatgtattttggtcccctccctgtctccgctcggattgtaaacctcccacgtgtggggatcacaccctgcactgtcccgaatctttacaccctatcccaaagctggtgctcaataaatacttctagatgatttg

SEQ ID NO: 62 - protein, 70 aa, Rattus Norvegicus
TGNHGSGLAAASAAAPRVGIRVGVDTRHGRGASTRQRPMLKRQLLERGPRQVHGLRFLSSATTQRLLPGM

SEQ ID NO: 63 - protein, 171 aa, Rattus Norvegicus
MADSPEVVAMDCEMVGLGPQRVSGLARCSIVNVHSAVLYDKYIQPEGEITDYRTQVSGITPQHMARATPFAEARLEILQLLKGKLVVGHDLKHDFSALKEDMRKYTIYDTSTDMLLWHEAKLHCYSRVSLRLLCKRLLHKSIQVRSFPAPVTLLSLLPPLSLPPSSHSGKE

SEQ ID NO: 64 - protein, 262 aa, Rattus Norvegicus
MADSVKTFLQDLGRGIKDSIWGICTISKLDARIQQKREEQRRRRASSLLAQRRAQSVERKQESEPRIVSRIFQCCAWNGGVFWFSLLLFYRVFIPVLQSVTARVIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIIR

SEQ ID NO: 65 - protein, 56 aa, Rattus Norvegicus
MIRNFFFNVWQHMPLIPTLGRQRQVDLFEFEARLVFTVSSRTAGELQNGETLSQKH

SEQ ID NO: 66 - protein, 38 aa, Rattus Norvegicus
MRPSIKRITPIRGEDESQLAILVPSLSPRGRAQSFTAR

SEQ ID NO: 67 - protein, 208 aa, Rattus Norvegicus
MERQLEAARYRSDGSLLLGASSLSGRCWAGSLWFFKDPSAAPNEGFCSAGVQTEAGVADLTWVGDKGILVASDSGAVELWELDENETLIVSKFCKYEHDDIVSTVTVLSSGTQAVSGSKDFCIKIWDLAQQMSLNSYRAHAGQVTCVAASPHRETVFLSCSEDSRILLWDTRCPKPASQMGCNASGYLPTSLAWHPQQSEIFVFGKAA

SEQ ID NO: 68 - protein, 249 aa, Rattus Norvegicus
MERQLEAARYRSDGSLLLGASSLSGRCWAGSLWFFKDPSAAPNEGFCSAGVQTEAGVADLTWVGDKGILVASDSGAVELWELDENETLIVSKFCKYEHDDIVSTVTVLSSGTQAVSGSKDFCIKIWDLAQQMSLNSYRAHAGQVTCVAASPHRETVFLSCSEDSRILLWDTRCPKPASQMGCNASGYLPTSLAWHPQQSEIFVFGDENGSVSLVDTKNASCTLSSAVHSQCVTRLVFSPHRCSLPGLSQ

SEQ ID NO: 69 - protein, 183 aa, Rattus Norvegicus
MAKAYDHLFKLLLIGDSGVGKTCLIIRFAEDNFNSTYISTIGIDFKIRTVEIEGKRIKLQVWDTAGQERFKTITTAYYRGAMGIILVYDITDEKSFENIQNWMKSIKENASAGVERLLLGNKCDMEAKRKVQREQAERVSQAGLLWPNALLLPFIQLSPSYLIPYSWPESTESDFLRQVPNPV

SEQ ID NO: 70 - protein, 122 aa, Rattus Norvegicus
MGIILVYDITDEKSFENIQNWMKSIKENASAGVERLLLGNKCDMEAKRKVQREQAERLAREHRIRFFETSAKSSVNVDEAFSSLARDILLKTGGRRSGNSSKPSSTDLKVSDKKNSNKCSLG

SEQ ID NO: 71 - protein, 257 aa, Rattus Norvegicus
PTLLRPPDGPGATSASPVACLLDSGHQAQLVEFLIVHYEQIFGMDELPLTSEPLTQDPALAPALLESSPQHPALLVAQDSQPLTIASDSSPDPKQHSALEKCPKVAPPELTALQSNRKEEEEEDTRDGAGDGSSHSPEDLLLVAQSRGHFSRQPVKYSRGGVRPVTHQLSSLALVASKLCEETPITVSAVHRGSLRARGLGPAAASPEGGTLRRNPLPKHFEITQETARLLSKLDSEAVSRTSCCADPEPEESEEHL

SEQ ID NO: 72 - protein, 340 aa, Rattus Norvegicus [sequence omitted]

SEQ ID NO: 73 - protein, 138 aa, Rattus Norvegicus
TVLASEPAIMAPGWPRPLPQLLVLGFGLVLIRATAGEQAPGNAPCSSGSSWSADLDKCMDCASCPARPHSDFCLGCAAAPPAHFRMLWPILGGALSLALVLALVSGFLVWRRCRRREKFTTPIEETGGEGCPGVALIQ

SEQ ID NO: 74 - protein, 198 aa, Rattus Norvegicus
QKRTQGTGTGLPEGLRSMADSPEVVAMDCEMVGLGPQRVSGLARCSIVNVHSAVLYDKYIQPEGEITDYRTQVSGITPQHMARATPFAEARLEILQLLKGKLVVGHDLKHDFSALKEDMRKYTIYDTSTDMLLWHEAKLHCYSRVSLRLLCKRLLHKSIQNNWRGHCSVEDARATMELYKISQRLRAQRGLPCLGTSA

SEQ ID NO: 75 - protein, 312 aa, Rattus Norvegicus
QSHTQDTSDMALRALHAQPTGGPQLRFLLFLLLLLLLLSWPSQGDALALPEQRRSLSESQLNPDELRGRFQDLLSRLHANQSREDSNSEPTPDPAVRILSPEVRLGSHGRLLLRVNRASLTQGLPEAYRVHRALLLLTPSSRPWDITRPLQRAISLQGPHARALRLRLAPPPDLAVLPSGGARLELHLRSAAGRGRRSAHLHPRDSCPLGPGRCCHLETVQATLEDLGWSDWVLSPRQLQLSMCVGECPHLYRSANTHAQIKARLHGLQPDRVPAPCCVPSSYTPVVLMHRTDSGVSLQTYDDLVAQGCHCA

SEQ ID NO: 76 - protein, 294 aa, Rattus Norvegicus
MIEVLTTDSQKLLHQLNTLLEQESRCQPKVCGLKLIESAHDNGLRMTARLRDFEVKDLLSLTQFFGFDTETFSLAVNLLDRFLSKMKVQAKHLGCVGLSCFYLAVKSIEEERNVPLATDLIRISQYRFTVSDLMRMEKIVLEKVCWKVKATTAFQFLQLYYSLIRETLPFERRNDLNFERLEAQLKACHCRIIFSKAKPSVLALAIIALEIQALKYVELTEGVECIQKHSKISGRDLTFWQELVSKCLTEYSSNKCSKPNGQKLKWIVSGRTARQLKHSYYRITHLPTIPETMG

SEQ ID NO: 77 - protein, 342 aa, Rattus Norvegicus
MRKESPPPLVPPAAREWNLPPNAPACMERQLEAARYRSDGSLLLGASSLSGRCWAGSLWFFKDPSAAPNEGFCSAGVQTEAGVADLTWVGDKGILVASDSGAVELWELDENETLIVSKFCKYEHDDIVSTVTVLSSGTQAVSGSKDFCIKIWDLAQQMSLNSYRAHAGQVTCVAASPHRETVFLSCSEDSRILLWDTRCPKPASQMGCNASGYLPTSLAWHPQQSEIFVFGDENGSVSLVDTKNASCTLSSAVHSQCVTRLVFSPHSVPFLASLSEDCSLAVLDSSFSEVFRSRAHRDFVRDATWSPLNHSLLTTVGWDHQVIHHVVPLEPLPAPGPDSVVE

SEQ ID NO: 78 - protein, 124 aa, Rattus Norvegicus
MSSALESVTINNRPLEMSVTKSEVAPEEDERKRRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 79 - protein, 203 aa, Rattus Norvegicus
MAKAYDHLFKLLLIGDSGVGKTCLIIRFAEDNFNSTYISTIGIDFKIRTVEIEGKRIKLQVWDTAGQERFKTITTAYYRGAMGIILVYDITDEKSFENIQNWMKSIKENASAGVERLLLGNKCDMEAKRKVQREQAERLAREHRIRFFETSAKSSVNVDEAFSSLARDILLKTGGRRSGNSSKPSSTDLKVSDKKNSNKCSLG

SEQ ID NO: 80 - protein, 164 aa, Rattus Norvegicus
MSDPGDVRPVPHRSKVCRRLFGPVDSEQLSRDCDALMASCLQEARERWNFDFATETPLEGNYVWERVRSPGLPKIYLSPGSRRRDDLGGDKRPSTSSALLQGPGPAPEDHVALSLSCTLVSHAPERPEDSPGGTGTSQGRKRRQTSLTDFYHSKRRLVFCKRKP

SEQ ID NO: 81 - DNA, 2077 bp, Homo Sapiens
tccgctccgttcggccggttctcccgggaagctattaatagcattacgtcagcctgggactggcaacacggagtaaacgaccgcgccgccagcctgagggctataaaaggggtgatgcaacgctctccaagccacagtcgcacgcagccaggcgcgcactgcacagctctcttctctcgccgccgcccgagcgcacccttcagcccgcgcgccggccgtgagtcctcggtgctcgcccgccggccagacaaacagcccgcccgaccccgtcccgaccctggccgccccgagcggagcctggagcaaaatgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaagagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaagcagtcgtggtatgggggcgactggggagtcctcattgaatcctcattttatacccaaaaccctgaagccattggagagctgtcttcctgtgtacctctagaatcccagcagcagagaaccatcaaggcgggagggcctgcagtgattcagcaggcccttcccattctgccccagagtgggtcttggaccagggcaagtgcatctttgcctcaactccaggatttaggccttaacacactggccattcttatgttccagatggcccccagctggtgtcctgcccgcctttcatctggattctacaaaaaaccaggatgcccaccgttaggattcaggcagcagtgtctgtacctcgggtgggagggatggggccatctccttcaccgtggctaccattgtcactcgtaggggatgtggagtgagaacagcatttagtgaagttgtgcaacggccagggttgtgctttctagcaaatatgctgttatgtccagaaattgtgtgtgcaagaaaactaggcaatgtactcttccgatgtttgtgtcacacaacactgatgtgacttttatatgctttttctcagatctggtttctaagagttttggggggcggggctgtcaccacgtgcagtatctcaagatattcaggtggccagaagagcttgtcagcaagaggaggacagaattctcccagcgttaacacaaaatccatgggcagcatgatggcaggtcctctgttgcaaactcagttccaaagtcacaggaagaaagcagaaagttcaacttccaaagggttaggactctccactcaatgtcttaggtcaggagttgtgtctaggctggaagagccaaagaatattccattttcctttccttgtggttgaaaaccacagtcagtggagagatgtttggaaaccacagtcagtggagcctgggtggtacccaggctttagcattattggatgtcaatagcattgtttttgtcatgtagctgttttaagaaatctggcccagggtgtttgcagctgtgagaagtcactcacactggccacaaggacgctggctactgtctattaaaattctgatgtttctgtgaaattctcagagtgtttaattgtactcaatggtatcattacaattttctgtaagagaaaatattacttatttatcctagtattcctaacctgtcagaataataaatattggaaccaagacatggtaaacatg

SEQ ID NO: 82 - DNA, 1925 bp, Homo Sapiens
gggcggaggtggggttagcttcagttgaccaaccatgccttgaggataaattggatgggatcagatgggaagatgtgacaagaagagaaatcctcctctatataggatgctctgctgtttcctaaggattttcagcaccttgccccaaaatcaaaatgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaagagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaagcagtcgtggtatgggggcgactggggagtcctcattgaatcctcattttatacccaaaaccctgaagccattggagagctgtcttcctgtgtacctctagaatcccagcagcagagaaccatcaaggcgggagggcctgcagtgattcagcaggcccttcccattctgccccagagtgggtcttggaccagggcaagtgcatctttgcctcaactccaggatttaggccttaacacactggccattcttatgttccagatggcccccagctggtgtcctgcccgcctttcatctggattctacaaaaaaccaggatgcccaccgttaggattcaggcagcagtgtctgtacctcgggtgggagggatggggccatctccttcaccgtggctaccattgtcactcgtaggggatgtggagtgagaacagcatttagtgaagttgtgcaacggccagggttgtgctttctagcaaatatgctgttatgtccagaaattgtgtgtgcaagaaaactaggcaatgtactcttccgatgtttgtgtcacacaacactgatgtgacttttatatgctttttctcagatctggtttctaagagttttggggggcggggctgtcaccacgtgcagtatctcaagatattcaggtggccagaagagcttgtcagcaagaggaggacagaattctcccagcgttaacacaaaatccatgggcagcatgatggcaggtcctctgttgcaaactcagttccaaagtcacaggaagaaagcagaaagttcaacttccaaagggttaggactctccactcaatgtcttaggtcaggagttgtgtctaggctggaagagccaaagaatattccattttcctttccttgtggttgaaaaccacagtcagtggagagatgtttggaaaccacagtcagtggagcctgggtggtacccaggctttagcattattggatgtcaatagcattgtttttgtcatgtagctgttttaagaaatctggcccagggtgtttgcagctgtgagaagtcactcacactggccacaaggacgctggctactgtctattaaaattctgatgtttctgtgaaattctcagagtgtttaattgtactcaatggtatcattacaattttctgtaagagaaaatattacttatttatcctagtattcctaacctgtcagaataataaatattggaaccaagacatggtaaacatg

SEQ ID NO: 83 - DNA, 2454 bp, Homo Sapiens
atctgtgagaacgggatcggcgaggaggcgacggggaggaggcgacgcggaggggtgaggggtcctctccgccgccaggcggagttgggagccggacgatcccgcacgcagcgcgaggcgggaccccggactgactacatttcccagtctccgtcgctccgcgcggcccaccccttcggctctgggccccgcctcgtggtgccggctggttcttcgcgctcgcccgacttcccagcggccccgtgcggcccgggcatgcccagtgcgggcgcagcggccccggccctggaagcgccccggcggagctggcccgcggtgggctaggggcagggccggagccgcggcggcggagctgtggatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 84 - DNA, 3460 bp, Homo Sapiens
atctgtgagaacgggatcggcgaggaggcgacggggaggaggcgacgcggaggggtgaggggtcctctccgccgccaggcggagttgggagccggacgatcccgcacgcagcgcgaggcgggaccccggactgactacatttcccagtctccgtcgctccgcgcggcccaccccttcggctctgggccccgcctcgtggtgccggctggttcttcgcgctcgcccgacttcccagcggccccgtgcggcccgggcatgcccagtgcgggcgcagcggccccggccctggaagcgccccggcggagctggcccgcggtgggctaggggcagggccggagccgcggcggcggagctgtggatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggtaagtccatctaaagaaatccagaaaatggaggcccagtttggaaatgctactgctaagcagacttcttaaactactcatagtcaggcttttggcaaacaatcctttgagggaagcagcctcattggcaagagggagcttaccttcggcttagtgggtttaactctcaactttgaggctgggggatgtggtagtatggcattatctttttggtgcttaacatggtatatctctgactgggttgtaagatggcataaatgagcatgagttgctctaaattagagaagtcaaattgatgtcagcatctggatccttttgggggtgttttagaaagaccttcagatatttaccagggtgtgcgtggttgaaagaggttaactgatacttacaggatggcatccttttgatttgctatcttgcttagttccagactaatcctgacaaaggatgctggtgctgaaattcttaattcacttagcctgtcagctttgaaattacgattatagaattctaagaaactttgcatgctttatatcagatttgtacacttctaatttatatgctgtagctctgttctaaatctgtacctaggctgggtgtggtggctcacacctgtaatcccaacactttgggaggccaaggtgggtggatcgcctgaggtcaggagttcaagaccagcttggccaatatggtgaaaccctgtctctactaaaaatacaaaaattagctgggcgaggtggcaggcgcctgtaatcccagctacttgggaggctgaggtgggagaatcgcttgaaaccaggaggtggaggttgcagtgagctgagattgcaccaccgcactcccgcctgggcatcaaagcaagactccatctcaaataataataataataataataataataataataaaatctgtacctaaaactcatggtttgtctaccatagttacttagtcacttgactaaaaggaaatataatatctaattgtattggctttcttgttttaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 85 - DNA, 2560 bp, Homo Sapiens
atctgtgagaacgggatcggcgaggaggcgacggggaggaggcgacgcggaggggtgaggggtcctctccgccgccaggcggagttgggagccggacgatcccgcacgcagcgcgaggcgggaccccggactgactacatttcccagtctccgtcgctccgcgcggcccaccccttcggctctgggccccgcctcgtggtgccggctggttcttcgcgctcgcccgacttcccagcggccccgtgcggcccgggcatgcccagtgcgggcgcagcggccccggccctggaagcgccccggcggagctggcccgcggtgggctaggggcagggccggagccgcggcggcggagctgtggttgtgcctagggagaccagttttctgccgaattgcctaggagctgtggggagatgggcctgaatggacccgtggactcagaggcagcccgtggagccccgtgggaaatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 86 - DNA, 2179 bp, Homo Sapiens
ggctaccggcgaggaccccttatgggaaggggccggagggagcgggacggggccgggcacatccccgggcgaggctgccccagatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 87 - DNA, 2583 bp, Homo Sapiens
atctgtgagaacgggatcggcgaggaggcgacggggaggaggcgacgcggaggggtgaggggtcctctccgccgccaggcggagttgggagccggacgatcccgcacgcagcgcgaggcgggaccccggactgactacatttcccagtctccgtcgctccgcgcggcccaccccttcggctctgggccccgcctcgtggtgccggctggttcttcgcgctcgcccgacttcccagcggccccgtgcggcccgggcatgcccagtgcgggcgcagcggccccggccctggaagcgccccggcggagctggcccgcggtgggctaggggcagggccggagccgcggcggcggagctgtggatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagacggattcttgccctgtcaccaagctgtcacctgagattgccgtggtgcaatctcagcttactgcagcctctgcctcccaggtccaagcaattcttctgcctcagcgaaccggctggctggaagtacagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 88 - DNA, 2906 bp, Homo Sapiens
atctgtgagaacgggatcggcgaggaggcgacggggaggaggcgacgcggaggggtgaggggtcctctccgccgccaggcggagttgggagccggacgatcccgcacgcagcgcgaggcgggaccccggactgactacatttcccagtctccgtcgctccgcgcggcccaccccttcggctctgggccccgcctcgtggtgccggctggttcttcgcgctcgcccgacttcccagcggccccgtgcggcccgggcatgcccagtgcgggcgcagcggccccggccctggaagcgccccggcggagctggcccgcggtgggctaggggcagggccggagccgcggcggcggagctgtggatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagacggattcttgccctgtcaccaagctgtcacctgagattgccgtggtgcaatctcagcttactgcagcctctgcctcccaggtccaagcaattcttctgcctcagcgaaccggctggctggaagtacaggtgcgcaccaccacgcctggctaattttttatattctttagtggagacagggttttgccatgttggccaggctggtctcgaactcctgacctcagttgatcgcctaccacggcctcccaaagtgttaggattacagccgtgagccactgcgcttgaccatttttttagtcttttctcattgactgacattagaacattgggagaacatgaggcactgaaaaataatacagtacccacaaaaggtaagatataaaatgaactactgaatgacaattaggaaataacttatgctttataaactaaatacatgtgatcttttccagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 89 - DNA, 3016 bp, Homo Sapiens
atctgtgagaacgggatcggcgaggaggcgacggggaggaggcgacgcggaggggtgaggggtcctctccgccgccaggcggagttgggagccggacgatcccgcacgcagcgcgaggcgggaccccggactgactacatttcccagtctccgtcgctccgcgcggcccaccccttcggctctgggccccgcctcgtggtgccggctggttcttcgcgctcgcccgacttcccagcggccccgtgcggcccgggcatgcccagtgcgggcgcagcggccccggccctggaagcgccccggcggagctggcccgcggtgggctaggggcagggccggagccgcggcggcggagctgtggatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagtgagccacgtattgttagtagaattttccagtgttgtgcttggaatggtggagtgttctggttcagtctcctcttgttttatcgagtatttattcctgtgcttcagtcggtaacagcccgaattatcggtaagtgtataccctgctccttgtctgttggggacagagtgggagatgatgtttcagtctccctcagtttactgaacatttcctgtgtaccaggagctttcactgtttattttcttttttgttgttttattctttttaaaaatttatatttcgttattctcctagaggccctttaagaatgggtactgttgttctatccattttatgaatgaggaaatgcaagttaccaaggttaaaccattagtaagaggcaaagctgagatttaaacccagatggtatcctgcagatagcgtactggtaatcactgtggcatctcgttaccatcatgtaagcaggtttgtgtcaagcagcacatatctatggtttctttgaattggaattgtggccagaatgtggtgcgtctattatgagttagactctttgagataggtatagtgatgtgctggcaagaactagaaagatccaggaggagacagagaatagtcacgagatgggggttccgcatttggtcttctggatttccatttctccatttctagtaactcgtttcctctttcttaggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagaccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccacaaagtg

SEQ ID NO: 90 - DNA, 2665 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtaagtgtgaagtggaatcctttgggaatcagggaaggggagcagtgggtgggcgagtcattttcctcttcagaaaactgtgttgtgcttttggagaacagattctccatctgtatccgaagggacttggtaatggaagcaggctggctgttgaccctgtccattcctggggcacattcacaaccccaaggacaagccgtgtccttggtagaggggggtggtaggtgaaatttttaagtctagccctccagcgctctcccacaagggggcggcagtctaccctctcacataaagaactgcgtatgaaatgcagcctgctacagggtaaaaggcagcacctggaagacctgaattgtacttccctccttgccgtgatttgctgcctgttactgcccctcatcactgtcttgttctatccagcatttcttcatctatgaaaatagaagactggattggatgactgatgacatccactgtggatagctacactgctatcctggcgatgaggccatacagttggtgaaatgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttg

SEQ ID NO: 91 - DNA, 2507 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttgaagcatgtgtgttatgtgtagttagatatatttcacttatgtgagtcatcatggtgttggtcttgtagcccattatttttcctgtgcttccccagcttcccaaagtagctagttagaacttaaggtaaatatttattcttgggttggtggagtggatattgccagttaggagtcatggatcaattactgattatattgaaagtaaatataatcaattatgtacttttgagctttgcaggttcaatttaggtaaaaatcacattatgaaactgggaaagtctgaaggaatatgggcaaaatatttctcagtaaagcttccatgcttcacccttgacatgattacccttgagtaaaacatgggaatttgt

SEQ ID NO: 92 - DNA, 2198 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattgtgaggctctgctaaccccattctgtgcaatgaataccaagaggaagggccagttcctgggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttg

SEQ ID NO: 93 - DNA, 3984 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttgctgttctctggtccaccaagggctatgaggctcaccccttggatggtgctcagaaccacaaggaatactgaagaagccttgcctatgatttcaggtggcatcagacattgggctaattttcaaggtgtctctccttcccaacaggacagtcggagtctggttctttaataaatagctatatggatcttacttactggttcacaatcatattacagcagcaaaaacacccaagctctaactgctctgggcctccagccatgtttcctccagatagcatgttagaaaggacaggagattttaaaaatcagattggacttcagagggctccaaattttcttgccacatctagacctgaaagtaaagagctctgattattaagaggcagattctcgttgcatttggacagcgtgagtaatggtcacactgaacacattagaatcagttgtgaaattgattttgtttcaaaaaaggataagcaagctgtagtctcaattttgagttaattgaaatttctcaagaacttcatcagcagctcacatgatacagttttggaagagtaaacaaggtaagcagacaaccctgggaccccaaacacattttcttttttagttctccatcaaaaataggcatttctggagttttggtctcactgagtgatcacaggactgctgcaatccagggaactgagggagctgtgagcatctgagaagaaaggttagctcttgttttaagttgtagagacaacttaaaacaagttgtgacacaattagtgatcccataatcttttatcatctggtacaacatttaatatagtagcattctacttactgtggaaactccttgtttcacaaatgatgttctgcagcttcaatatgtaatggatggaccatcaaagttcaaaatataataaggaatttaattcccaaacaacatcctttaacattctggtgaaagcccagccagtgtcactggttgaaagtgccttcagtggatatagctatatttcacctgtccctgtgatcatgaggtaccagattccggtcaagcatttcactttgggaggcaccatgttgtaaacattaggaatttcaggtgcaaactaattttccattctggtagtaactcttacctggagtgtcagctaagtcatgaaaattgtttagattagaaaatgcactttttatatatagcctaaattagcttcatgttctgtagtttctgcagcttgtctcagaaatcctctgagtagaactgatgtatgagaccctttaagaccattatccaactgcagtttgcttggcagagttagcattaatgcaccctttgatctaggcacctggaggcaggtaagacaggctctgcgtgcaccaccgtccctggacctgttgtggcggactccttgaagcaagatcaacttgggctttgtttgagtttttaaagtcttttttttttaatgtaaaatttgtcattttaactttttaagtatacagttcagtggcattaagtttatttgcatttttgtgcagctttcaccacttccatctccaaaactttttcatcatcccaaactgaaactgtacttattaaacagtaactccccttcctcaagaccctggcaatcaccattcatcttcatttctataaatttgactattctagacatacgtgtaagtgggagcatacaatatttggtttttttagaggggggttctggcttaattttataaggtgttaacttgttcctcaaaatgtgttctataaactggcagcatttgcatatcttggaacttgcttgaaatacagaatgtccagatctactgagtcagaatttacattttcaaaagcttcctacgtgactcatgcatattaaagtttgggaagcactgacttagattaccttttgagaattccagatgggtcagaaaccagacagaaatactcagtagtgagaagctatggtgtatcagaagctgttaggcatttcatggtttggtagtgagcaagacagatagttttcctgtattcagcgacttagtctagagagagacaggatggaattaagtgtttaggtgctagccaaaagtaaagattcgtagaaaacaagggttcatatcccagtcatcaaagtgataaattttccctgcttaacatttagattaaaaagtaataattaggccgggtgtgagccctgcgtctggcccatgactatcatttctttcttatggggtttattgtggtcactgcctagcttgtatctgattgatcagggaactgattc

SEQ ID NO: 94 - DNA, 2389 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccggtgagccgagatcccgggcctgggagctcccttaccccggcccagggcgcccctccccggagactgaccctcccagcccatccccgccctgcatgttatcctgaccctgggtcgcgtcaccgagctgtccccagccctgccccgcgttcctgagctcagatccagacccggccggaacgctgttctgtgcggtctccatggaggcgaacccttctacccttcacgcgctgacagcttgtaccccccgtagatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttg

SEQ ID NO: 95 - DNA, 2429 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctggtgggtccctgttctcattgctcctgtctctaagcctcctgtgggtgcctttcctatcctgttaacctcattaggtaacattttgggcttattctaagaatccagtgacttattttagcatgtctataaggtttccttagggattttgcagtccatcgtaagtagaccatagcagcagtccaagaggcaaagacttggggaatgttgattacaggaagttcttgtttgaacacgtttttatgttgatagtgtaggtcaagattaaatgtctgtttgcttttgcttcttagcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttg

SEQ ID NO: 96 - DNA, 1563 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttcatttcttcatctatgaaaatagaagactggattggatgactgatgacatccactgtggatagctacactgctatcctggcgatgaggccatacagttggtgaaatgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttcccta

SEQ ID NO: 97 - DNA, 2289 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgagtcactatcccattatccacccccacccccgggtggaacggtagaatggagacttggggtaccgtagggttgagctcaatatctaacgtcctatactcagtgcttggaggagcctgagagtgttggccccgaatgcaaactcttgtgtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttg

SEQ ID NO: 98 - DNA, 1118 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttggctggttcttgtgctgaaacaccacgatggtgctgcccataaactcgtgtgttatttcaccaactgggcacacagtcggccaggccctgcctcgatcttgccccatgac

SEQ ID NO: 99 - DNA, 2105 bp, Homo Sapiens
aattctcgcgagaagtagagttgcggttccctcgggagaggggcggaggctcagaggtgcaccacgtggggtttgctgccggagcggagtctccggccggcgtccagtttgagtctaggttggagttggaaccgtggagatgcggaaggaaaccccaccccccctagtgcccccggcggcccgggagtggaatcttcccccaaatgcgcccgcctgcatggaacggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttg

SEQ ID NO: 100 - DNA, 1720 bp, Homo Sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 101 - DNA, 1396 bp, Homo Sapiens
agaaagcaactagcacagccatgcccaggccaggacccgtgacacagcgatgctctcagatgctgctgatgttacagatgttgttgggggcacccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 102 - DNA, 3148 bp, Homo Sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgaggtctgggctgtggtgggacccaggagggggcaggcagaggggcacactcttagcccgccctaaacccatctgtgccgtgtgactccggcaaacctctcacccttgctaagcctagcaactaggttaaggcaccctcaaaccccagtctccccagtctccctaacacttgtaccagcctgaggggctgtgattcctagaggctcagtagtctggaatgtgatgggtttggcactcttcatttctcgggattacacctgtaatcccagcactttaggaggccaaggtgggagcatcgctttaggccagaacattgcagcctgggcagtatagcgagaatccatctctaccaaaaatgtttaaaaaaaattagccagacatgatggtgtgtgcctgtagtctcagttattcaggaggctgaggcgggaggagcacttgaatccaggagattgaggctgcactgagccatgatgcagtggcacgatcttggctcactgcaacctccaccgccccgattcaagccattcttctgcctcagcctcccgagtagctgggatcacaggcgccagccaccatgcccagctaatttttggtagttttagtagaagcgggcgtttcaccatattggccacatatcggccaggctggtcttgaactcctgacctcaagtgatccacctgcatcggcctcccaaagtgctgggattacaggcgtgagccaccgcgcccgcacggagaaatgctcttggtaggggaaataacttgtgcaaaggcgccgccgccgtggtgagatccagcttggcgtgttaggggaaatgggcaccctcgttcctggaaaacggtaggcctgtgggtgaccagcttccctatctgtcttcccacagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 103 - DNA, 2268 bp, Homo Sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 104 - DNA, 1925 bp, Homo Sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgaggtctgggctgtggtgggacccaggagggggcaggcagaggggcacactcttagcccgccctaaacccatctgtgccgtgtgactccggcaaacctctcacccttgctaagcctagcaactaggttaaggcaccctcaaaccccagtctccccagtctccctaacacttgtaccagcctgaggggctgtgattcctagaggctcagtagtctggaatgtgatgggtttggcactcttcatttctcgggattacacctgtaatcccagcactttaggaggccaaggtgggagcatcgctttaggccagaacattgcagcctgggcagtatagcgagaatccatctctaccaaaaatgtttaaaaaaaattagccagacatgatggtgtgtgcctgtagtctcagttattcaggaggctgaggcgggaggagcacttgaatccaggagattgaggctgcactgagccatgagtgccccactgcactccagcctgggtaacagcaagaccataataaataaataaataagtgaagcaagtaagtttttgactg

SEQ ID NO: 105 - DNA, 1034 bp, Homo Sapiens
aaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaactttaaat

SEQ ID NO: 106 - DNA, 1757 bp, Homo Sapiens
aaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccaggtacgcgggactccggggtcggggaaccccgggccggggctcgggagccggactgggtgtctggagaacagcgccgaactgggggaacaggcggatgtggggatctggggtcaggtggccttcaaacagctcgctcccggctgtgggaagttccatcactctccaagcttcattcacaatccgggcctcagtttctcccaccgtccaattggggttggggggggtcttcagttccacaagtccgaagtacttcgaatggagagagaaagtgagtgagggcgggatgtttagtctaggaaggggcacgcccccagcctggtccgtacagggtctaactagccccagtgatcgacgagttggggaggccgtcgagaggcactagggtctgtgccctctataccatgtggggtgcccccttccatccggtgacttcagtgcccagctatgcggggggagggactggggtcgggcccggtgcccaggagtgagtcacccgccggcggcaggggggttgtggggacctgaccccccccacccccagcgtcccgcgctacgccctccctcccggagctgccgtgtcccggtcgggccgtgcggtcacctgtgaggaatgcgcggggagggcgccggtgactcacgttattcgagccggccgaccctgacctcagaccccagaatagccgagtcccgcccccagcctctgacccgaggccccctccccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaactttaaat

SEQ ID NO: 107 - DNA, 1060 bp, Homo Sapiens
aaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccagcctctgacccgaggccccctccccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaactttaaat

SEQ ID NO: 108 - DNA, 1269 bp, Homo Sapiens
aaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgtgagtggggggcagggccagtggccagcggaccccagactgggagggagggtggctggtgcgcagagcggggccgagagctttgcatctgggaaatcattcgggaggaggtgggagctgggagggggctccggtcagggaggaggccacgtttgggagaaggcagaaggctcagtcttagggggcggggctggcctggagaggggcgaggtctgactccgagtcccgcccccaggcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaactttaaat

SEQ ID NO: 109 - DNA, 2774 bp, Homo Sapiens
aatccggctggcagccgattgacctgacccaggcgagggttgctgcaccgagtatttagtttggccccctactgaatgaagacactttgttaaattctccaatgagagacgcttagaggtaggggcgtggaagggggccggggaatttgaggccagataacgggaagtgtgagccgattggccggacttctcactccacggcccagccgtttacggatcgcggaggccatccatcaaaagaattacaccaatcagcggcgaaaatgcccccttctcgcgagaaagccccgccctccaatatattcctcgttagggcaggcgcggccccttcggctccgagctgaccctgatcagggccgagttgtctcggcggcgctgccgaggcctccacccaggacagtccccctccccgggcctctctcctcttgcctacgagtcccctctcctcgtaggcctctcggatctgatatcgtggggtgaggtgagcaggcccggggagggtggttaccgctgaggagctgcagtctctgtcaagatgatagaggtactgacaacaactgactctcagaaactgctacaccagctgaatgccctgttggaacaggagtctagatgtcagccaaaggtctgtggtttgagactaattgagtctgcacacgataatggcctcagaatgactgcaagactaagggactttgaagtaaaagatcttcttagtctaactcagttctttggctttgacacagagacattttctctagctgtgaatttactggacagattcctgtctaaaatgaaggtacagcccaagcaccttgggtgtgttggactgagctgcttttatttggctgtaaaatcaatagaagaggaaaggaatgtcccattggcaactgacttgatccgaataagtcaatataggtttacggtttcagacttgatgagaatggaaaagattgtattggagaaggtgtgttggaaagtcaaagctactactgcctttcaatttctgcaactgtattattcactccttcaagagaacttgccacttgaaaggagaaatagcattaattttgaaagactagaagctcaactgaaggcatgtcattgcaggatcatattttctaaagcaaagccttctgtgttggcattgtctatcattgcattagagatccaagcacagaagtgtgtagagttaacagaaggaatagaatgtcttcagaaacattccaagataaatggcagagatctgaccttctggcaagagcttgtatccaaatgtttaactgaatattcatcaaataagtgttccaaaccaaatgttcagaagttgaaatggattgtttctgggcgtactgcacggcaattgaagcatagctactacagaataactcaccttccaacaattcctgaaatggtcccttaactggattattacagcaccaaaaaacttctctgaagcctttctccacaaccttgttctatggattccataatgttacaatggatttaagctatgaagcctcaaaacatcacgagataagcatgatggtctcagacttgggaaaactgcctaatattatgctgtagtggaattatgtttagatttgaattcatctgtgaagcattcaaatcaaagctaaaagcctaaatgtgaaatgctaatgacaagcctgagaaggtaaactgtgaatcttcatttctatcattgatctaactttagatattggatcaatatatttaggtggtattgaaaatgctattggaggagtcacactaatactatcaactatcagtcttcccacagcttcaatcactgtcattattctaatcctactcctacttaaattttaagttatgaggtttatgtcaaaagcaacatttcacaaatgtacttttaaggcataataagggttaacattctaggcagtataaacacaccccataatgcaagtaataggtaatctagagatgtggactttattgctatatgggaattacatttaaatttgagggcattttatataaagaaaaatacagacctataaagtttggcatattcattaagttatcttttaatatttttttctagaaaacaggtgacatttgtatctacgataaaaatttttatacagaacctactgcctcaaactgaatcccatcaagaaaactagtttctattgtattagtaactcaaaataaattatcacttcgaaaacttgctttcccacactaaggtaagttcagactagattgaacactccagaattttttactacagactgtttttaagttagaagtgatggcaattttataaatagagaatatacttccactgatgcccttactgtgccaaaacaaaaatcttaagaaaagcaagtagacaccttcataactatgaatgaagctgctgaagtagtgtttaggatcctccatggcagttagtgaatgtaagaagtacagtgttaaagtgttgtaaacagttactcagtgcaatgtatagcctgagtctatccatgatggctatatccaatttgacatcacgttatggatcagtacacaatgaaaaaccaaagaaccacagtatatcttattcttaacttttgtaaaccatgttttatgggtaactttttagttttcccaaaaggctgataaatttcaatattttgaatacatcattgttaattttgagttggcagaggtaaactaaccaactaccattatgttttagtactaagggatatacctttcaataaagttaatgaaattcaatcctt

SEQ ID NO: 110 - DNA, 881 bp, Homo Sapiens
aatccggctggcagccgattgacctgacccaggcgagggttgctgcaccgagtatttagtttggccccctactgaatgaagacactttgttaaattctccaatgagagacgcttagaggtaggggcgtggaagggggccggggaatttgaggccagataacgggaagtgtgagccgattggccggacttctcactccacggcccagccgtttacggatcgcggaggccatccatcaaaagaattacaccaatcagcggcgaaaatgcccccttctcgcgagaaagccccgccctccaatatattcctcgttagggcaggcgcggccccttcggctccgagctgaccctgatcagggccgagttgtctcggcggcgctgccgaggcctccacccaggacagtccccctccccgggcctctctcctcttgcctacgagtcccctctcctcgtaggcctctcggatctgatatcgtggggtgaggtgagcaggcccggggagggtggttaccgctgaggagctgcagtctctgtcaagatgatagaggtactgacaacaactgactctcagaaactgctacaccagctgaatgccctgttggaacaggagtctagatgtcagccaaaggtctgtggtttgagactaattgagtctgcacacgataatggcctcagaatgactgcaagactaagggactttgaagtaaaagatcttcttagtctaactcagttctttggctttgacacagagacattttctctagctgtgaatttactggacagattcctgtctaaaatgaaggtatgtttgaagctacatttttgtaattttgctcagtgtgttttggagatggaattgttaccttttggctggtattcataaa

SEQ ID NO: 111 - DNA, 1951 bp, Homo Sapiens
ggcatagtactattcattttaattttttggcccaaatcatctaaaactcccatatggtgcctttagaagttagtatctctgatggaaggggaattcctttcaatagttcatttgctatgttaagtgtgcataagctttactttaaaaattgacttcaatgtttttctaaaaacatcttttgactagatgtagtattacctaataaaagtaataccattttctttttaaaggagaaatagcattaattttgaaagactagaagctcaactgaaggcatgtcattgcaggatcatattttctaaagcaaagccttctgtgttggcattgtctatcattgcattagagatccaagcacagaagtgtgtagagttaacagaaggaatagaatgtcttcagaaacattccaagataaatggcagagatctgaccttctggcaagagcttgtatccaaatgtttaactgaatattcatcaaataagtgttccaaaccaaatgttcagaagttgaaatggattgtttctgggcgtactgcacggcaattgaagcatagctactacagaataactcaccttccaacaattcctgaaatggtcccttaactggattattacagcaccaaaaaacttctctgaagcctttctccacaaccttgttctatggattccataatgttacaatggatttaagctatgaagcctcaaaacatcacgagataagcatgatggtctcagacttgggaaaactgcctaatattatgctgtagtggaattatgtttagatttgaattcatctgtgaagcattcaaatcaaagctaaaagcctaaatgtgaaatgctaatgacaagcctgagaaggtaaactgtgaatcttcatttctatcattgatctaactttagatattggatcaatatatttaggtggtattgaaaatgctattggaggagtcacactaatactatcaactatcagtcttcccacagcttcaatcactgtcattattctaatcctactcctacttaaattttaagttatgaggtttatgtcaaaagcaacatttcacaaatgtacttttaaggcataataagggttaacattctaggcagtataaacacaccccataatgcaagtaataggtaatctagagatgtggactttattgctatatgggaattacatttaaatttgagggcattttatataaagaaaaatacagacctataaagtttggcatattcattaagttatcttttaatatttttttctagaaaacaggtgacatttgtatctacgataaaaatttttatacagaacctactgcctcaaactgaatcccatcaagaaaactagtttctattgtattagtaactcaaaataaattatcacttcgaaaacttgctttcccacactaaggtaagttcagactagattgaacactccagaattttttactacagactgtttttaagttagaagtgatggcaattttataaatagagaatatacttccactgatgcccttactgtgccaaaacaaaaatcttaagaaaagcaagtagacaccttcataactatgaatgaagctgctgaagtagtgtttaggatcctccatggcagttagtgaatgtaagaagtacagtgttaaagtgttgtaaacagttactcagtgcaatgtatagcctgagtctatccatgatggctatatccaatttgacatcacgttatggatcagtacacaatgaaaaaccaaagaaccacagtatatcttattcttaacttttgtaaaccatgttttatgggtaactttttagttttcccaaaaggctgataaatttcaatattttgaatacatcattgttaattttgagttggcagaggtaaactaaccaactaccattatgttttagtactaagggatatacctttcaataaagttaatgaaattcaatcctt

SEQ ID NO: 112 - DNA, 1023 bp, Homo Sapiens
atccactccacgaacgtcaaatgcgtcatctgaggagggggagggccggggcaaaggaggggcaccctgacatggagcctgccagctccgtcagccctgactcggcccggagctgagctccccacctgccggtagcccaggagatggagcagcccagcccacgtgcccggccttccgcccctgacttcacttgataacaaactagaaactgaaacagggtcgggatgccgatgccggcttggagttagagatgagtcaccgctgagagcagctgcagtagctgagcagtggcagcagagaggcagacgtgagctgagggcgcagaggcaggcagcatctctgagggtccccaaggaacatggctgggagccgtgaggtggtggccatggactgcgagatggtggggctggggccccaccgggagagtggcctggctcgttgcagcctcgtgaacgtccacggtgctgtgctgtacgacaagttcatccggcctgagggagagatcaccgattacagaacccgggtcagcggggtcacccctcagcacatggtgggggccacaccatttgccgtggccaggctagagatcctgcagctcctgaaaggcaagctggtggtgggtcatgacctgaagcacgacttccaggcactgaaagaggacatgagcggctacacaatctacgacacgtccactgacaggctgttgtggcgtgaggccaagctggaccactgcaggcgtgtctccctgcgggtgctgagtgagcgcctcctgcacaagagcatccagaacagcctgcttggacacagctcggtggaagatgcgagggcaacgatggagctctatcaaatctcccagagaatccgagcccgccgagggctgccccgcctggctgtgtcagactgaagccccatccagcccgttccgcagggactagaggctttcggctttttgggacagcaactaccttgcttttggaaaatacatttttaatagtaaagtggctctatattttctctacgcca

SEQ ID NO: 113 - DNA, 1856 bp, Homo Sapiens
atccactccacgaacgtcaaatgcgtcatctgaggagggggagggccggggcaaaggaggggcaccctgacatggagcctgccagctccgtcagccctgactcggcccggagctgagctccccacctgccggtagcccaggagatggagcagcccagcccacgtgcccggccttccgcccctgacttcacttgataacaaactagaaactgaaacagggtcgggatgccgatgccggcttggagttagagatgagtcaccgctgagagcagctgcagtagctgagcagtggcagcagagaggcagacgtgagctgagggcgcagaggcaggcagcatctctgagggtccccaaggaacatggctgggagccgtgaggtggtggccatggactgcgagatggtggggctggggccccaccgggagagtggcctggctcgttgcagcctcgtgaacgtccacggtgctgtgctgtacgacaagttcatccggcctgagggagagatcaccgattacagaacccgggtcagcggggtcacccctcagcacatggtgggggccacaccatttgccgtggccaggctagagatcctgcagctcctgaaaggcaagctggtggtgggtcatgacctgaagcacgacttccaggcactgaaagaggacatgagcggctacacaatctacgacacgtccactgacaggctgttgtggcgtgaggccaagctggaccactgcaggcgtgtctccctgcgggtgctgagtgagcgcctcctgcacaagagcatccagaacagcctgcttggacacagctcggtggaagatgcgagggcaacgatggagctctatcaaatctcccagagaatccgagcccgccgagggctgccccgcctggctgtgtcagactgaagccccatccagcccgttccgcagggactagaggctttcggctttttgggacagcaactaccttgcttttggaaaatacatttttaatagtaaagtggctctatattttctctacgccatcactgggtcctcttcttattcttctctccaagctgggttaacagtagacaggacccatttctgtgtgatgttaggagggaatgaagtcttatgctggggaggtgggcaagtatcaatttccttaatatcttgaatcctgtgggtccaaaatgtggcttggaaatctaagtagcatgtggcttaattactaatcccaccctttgctgttgcatcccagccctattcctggtgcatttatgcccagagaggtggcattatttcctggggtggcattcagctcctcttgagttggtgccacagcatttgtgggctttgaagcaaaggtacaggaaatgtcaagggtgccaccccggcaaccttgagcaagtcacccctcctatttgtaaaatgaggaaggaaaggtaacaaactgtggagtcagagagaagtaggttggaatcctctttgtcatttagtagctgtttgacctaaggtggtttactgaacttctcagtttctccatctgtaaaatgagaattctagcaactcgtagggtatttgtgagatgttgcatgcaaagcccccagcaccatgcctgtcctagcttaagcacccaccaggtgtcgataagtaattgttcttccctggactgcctgcacatctagggcaccccaggaagagtcaccgcactctgtttcggggctcggctctctgaggggagggcatcctgtatggggaggagggtctggaccagagctgtccctgatcgctagaatcaacggcagaattcttgttattttaataataattaataatttagtgcttccactaaataaaaatcattggaatggtg

SEQ ID NO: 114 - DNA, 2964 bp, Homo Sapiens
gcattgatggtaggtcctgctgtgcctggtccctggctggtagagtgaggatgagggcagggaagggaaaacacccaacaaaggtttgcttttgttgttgaaatcaagccagttaccctgtgggaagtgaagcttgctcccaccatagaactctgggcaatgatgtaaagcacacacctcacctcagatatgtctcacagcaggggccggggaactgggatatttgtacaccagttcccatcagccaccagctgctgctgctcctgcgcactgattccgcagcacttctggctggtgggcaaagcaggcaggaaaaggcatacggatatggggggttgggagttcaccctgtgggaagggcagagggagaggcgccagacactaagagccagggctgtacacagggacccttcaggtcaacagctctttattaaacacctgctgcctgctctaggccagatcctagagacgcagggtgagcaagacccrtctctgtccctgagctcttggttggttgggaggtggcccttaaccagatcatcagcacacatggtaaggagtgtgtgacggagggaagggcagtgccctgggagcaggatgaaggatggggctggcagggcaggagaccagatctgaacagagcctggagggtcttagtcaaggagggcagaagaaggcatacagagtggggccaggtgagtcaaagcctgaggctcggggaggggctggggagcacggagcccgcccctagcctgctggcaagagctgtaggaatcatgtccggagtagggaacagggctgagtctgggcctgttagcctcaaagcacataaggcacataagggccaggctgggcttttcctggagcccttgccgctctgtggctctcagtctggggccttaagcctgacccagggccttctcccaggctctgtccctgggctcattaagcctggttgttccagaagctcagtaccccaccaggcagctcctgcctgcactgatgcccctagaaaccagccctcccgcccgcccccgctcacatggacagtggtgctactgatcactgtgaccagaactgacccacaggacacraggagctaacatggtagagctgtcacagttgggcagtggtgttactgcccctcgagggagtgaaatagcaggcctgtggctccctgccaagctgcgcgcatggtagaccgggctgcacaggaaaggaaagaccctgccatttatgaagccctgtggagggtgctgggcacagttctctcattccatcctttaaggcaggtataatcaggcccatttcacagaagaggaaacagaggcccagaaaggaaaagtgacttgcccacggtcacagagctcataagcaggggagctgggaattgcagcctggggtttgtctgattccaaatccggggctctttacctccccacttggtaataaagggtgctggtgctgcattctgcttctgttcttggcttccttctccatagagcagagtggtagaacacataacctccctgtgcctcagtttcctcatctgtcaaggatggtgacaggggcaggtatgcagaggcacagcrcagacgacgccaccatgctacctgcttgctgattaatgatagggtgtccaagccaaaaaataaaactaaacattaaaaaataaaacaatacagcgtaataacggcaacaagaaaaagcctttttgagctgggccgtctaaccaggtgtccccaacaccctgtctggagtgcctggaaaaggagcccctgtgagaacaggaagtcctctctcgacagagggacaccaagaaaacgcagccaggtccaacagcccgctggctcccatgctgggtaccggccaggacaccagggctgagcggggcatgcgaagggagatgaaaggtgactgcagtgaggatttgccctgtgtactcgcctctgcaagatccacatctcagctgcacaactggctggcttgtgtgtgagcctcagttttatcatcagtaaaatgggggaataatcatatccagctcagaggtttaacttcagcctagtgcacggaagacatggtaaatggtcatcgcttctcccttgaatggaccgagggcccccgactcccatagcactaccccataaagtctaggctcccgacttcggcattcaaggccttcagtgataaatccaggcccaccatccgtccccacaccacctttcacacaccccctgcccccaccctcgcaggcctcttattttagctgcacgtgctctccctgcctccgaaatgcttgcggcctttcctttccatttattgacgcccttgtgttctctgtgagcagcccttccctgtgtccctagctggagttcctcactccctcctcagtgtcctgaagcttcttgttagagctgctgcagccactgcgtgtgtctgacagcaagtctgcagcctctgcctcccttgcgggactgcaggtgacttgaaggcacagcctggttcccaggaggctggctttcgtgccactggccccagggctgaaggcaggccagctccagccacctgctgagtctcctaagtccagagtgtcagagacctcaagagactctcacccactgacccacctggggactctggaaatcgggatgtgaccagggaagaccctgtcacggggcctctgaattcagcctcatcccaagtccccactctatacttgtgtctgcagaacagcctgcttggacacagctcggtggaagatgcgagggcaacgatggagctctatcaaatctcccagagaatccgagcccgccgagggctgccccgcctggctgtgtcagactgaagccccatccagcccgttccgcagggactagaggctttcggctttttgggacagcaactaccttgcttttggaaaatacatttttaatagtaaagtggctctatattttctctacgcca

SEQ ID NO: 115 - DNA, 1205 bp, Homo Sapiens
ctctttcccggagcctgggcggagagggaggaaaacttcttcctggcctgggctccgtgccgctctgtttgccaaccgtccagtcccgcctaccagtgccgggcgctccccacccctcccccggctcccccggtgtccgccatggccaaagcctacgaccacctcttcaagttgctgctgatcggggactcgggggtgggcaagacttgtctgatcattcgctttgcagaggacaacttcaacaacacttacatctccaccatcggaattgatttcaagatccgcactgtggatatagaggggaagaagatcaaactacaagtctgggacacggctggccaagagcggttcaagacaataactactgcctactaccgtggagccatgggcattatcctagtatacgacatcacggatgagaaatctttcgagaatattcagaactggatgaaaagcatcaaggagaatgcctcggctggggtggagcgcctcttgctggggaacaaatgtgacatggaggccaagaggaaggtgcagaaggagcaggccgataagttggctcgagagcatggaatccgatttttcgaaactagtgctaaatccagtatgaatgtggatgaggcttttagttccctggcccgggacatcttgctcaagtcaggaggccggagatcaggaaacggcaacaagcctcccagtactgacctgaaaacttgtgacaagaagaacaccaacaagtgctccctgggctgaggaccctttcttgcctccccaccccggaagctgaacctgagggagacaacggcagagggagtgagcaggggagaaatagcagaggggcttggagggtcacataggtagatggtaaagagaatgaggagaaaaaggagaaaagggaaaagcagaaaggaaaaaaaggaagagagaggaagggagaagggagaggaatgaattgaggaagtgaaagaaggcaaggaggtaggaagagagggaggaggaaaggaaggagagatgcctcaggcttcagaccttacctgggttttcagggcaaacataaatgtaaatacactgatttattctgttactagatcaggttttagggtcctgcaaaaggctagctcggcactacactagggaatttgctcctgttctgtcacttgtcatggtctttcttggtattaaaggccaccatttgcacaaatgtt

SEQ ID NO: 116 - DNA, 1323 bp, Homo Sapiens
ctctttcccggagcctgggcggagagggaggaaaacttcttcctggcctgggctccgtgccgctctgtttgccaaccgtccagtcccgcctaccagtgccgggcgctccccacccctcccccggctcccccggtgtccgccatggccaaagcctacgaccacctcttcaagttgctgctgatcggggactcgggggtgggcaagacttgtctgatcattcgctttgcagaggacaacttcaacaacacttacatctccaccatcggaattgatttcaagatccgcactgtggatatagaggggaagaagatcaaactacaagtctgggacacggctggccaagagcggttcaagacaataactactgcctactaccgtggagccatgggcattatcctagtatacgacatcacggatgagaaatctttcgagaatattcagaactggatgaaaagcatcaaggagaatgcctcggctggggtggagcgcctcttgctggggaacaaatgtgacatggaggccaagaggaaggtgcagaaggagcaggccgataaggtgagggccaggctgagcaatgttctagaactgggctgggagggccaagataggttgcattgcagaggacttggctgctgtccttaattcaattctcttcccacttcaccccttatagttggctcgagagcatggaatccgatttttcgaaactagtgctaaatccagtatgaatgtggatgaggcttttagttccctggcccgggacatcttgctcaagtcaggaggccggagatcaggaaacggcaacaagcctcccagtactgacctgaaaacttgtgacaagaagaacaccaacaagtgctccctgggctgaggaccctttcttgcctccccaccccggaagctgaacctgagggagacaacggcagagggagtgagcaggggagaaatagcagaggggcttggagggtcacataggtagatggtaaagagaatgaggagaaaaaggagaaaagggaaaagcagaaaggaaaaaaaggaagagagaggaagggagaagggagaggaatgaattgaggaagtgaaagaaggcaaggaggtaggaagagagggaggaggaaaggaaggagagatgcctcaggcttcagaccttacctgggttttcagggcaaacataaatgtaaatacactgatttattctgttactagatcaggttttagggtcctgcaaaaggctagctcggcactacactagggaatttgctcctgttctgtcacttgtcatggtctttcttggtattaaaggccaccatttgcacaaatgtt

SEQ ID NO: 117 - DNA, 1301 bp, Homo Sapiens
ctctttcccggagcctgggcggagagggaggaaaacttcttcctggcctgggctccgtgccgctctgtttgccaaccgtccagtcccgcctaccagtgccgggcgctccccacccctcccccggctcccccggtgtccgccatggccaaagcctacgaccacctcttcaagttgctgctgatcggggactcgggggtgggcaagacttgtctgatcattcgctttgcagaggacaacttcaacaacacttacatctccaccatcggtgagcccgctggccatgtcccagaccccgactctcctgtgacctccttctccggctcccggattactagtgaccccacttttcagtccccttagagaattgatttcaagatccgcactgtggatatagaggggaagaagatcaaactacaagtctgggacacggctggccaagagcggttcaagacaataactactgcctactaccgtggagccatgggcattatcctagtatacgacatcacggatgagaaatctttcgagaatattcagaactggatgaaaagcatcaaggagaatgcctcggctggggtggagcgcctcttgctggggaacaaatgtgacatggaggccaagaggaaggtgcagaaggagcaggccgataagttggctcgagagcatggaatccgatttttcgaaactagtgctaaatccagtatgaatgtggatgaggcttttagttccctggcccgggacatcttgctcaagtcaggaggccggagatcaggaaacggcaacaagcctcccagtactgacctgaaaacttgtgacaagaagaacaccaacaagtgctccctgggctgaggaccctttcttgcctccccaccccggaagctgaacctgagggagacaacggcagagggagtgagcaggggagaaatagcagaggggcttggagggtcacataggtagatggtaaagagaatgaggagaaaaaggagaaaagggaaaagcagaaaggaaaaaaaggaagagagaggaagggagaagggagaggaatgaattgaggaagtgaaagaaggcaaggaggtaggaagagagggaggaggaaaggaaggagagatgcctcaggcttcagaccttacctgggttttcagggcaaacataaatgtaaatacactgatttattctgttactagatcaggttttagggtcctgcaaaaggctagctcggcactacactagggaatttgctcctgttctgtcacttgtcatggtctttcttggtattaaaggccaccatttgcacaaatgtt

SEQ ID NO: 118 - DNA, 1197 bp, Homo Sapiens
ctctttcccggagcctgggcggagagggaggaaaacttcttcctggcctgggctccgtgccgctctgtttgccaaccgtccagtcccgcctaccagtgccgggcgctccccacccctcccccggctcccccggtgtccgccatggccaaagcctacgaccacctcttcaagttgctgctgatcggggactcgggggtgggcaagacttgtctgatcattcgctttgcagaggacaacttcaacaacacttacatctccaccatcggaattgatttcaagatccgcactgtggatatagaggggaagaagatcaaactacaagtctgggacacggctggccaagagcggttcaagacaataactactgcctactaccgtggagccatgggcattatcctagtatacgacatcacggatgagaaatctttcgagaatattcagaactggatgaaaagcatcaaggagaatgcctcggctggggtggagcgcctcttgctggggaacaaatgtgacatggaggccaagaggaaggtgcagaaggagcaggccgataagttggctcgagagcatggaatccgatttttcgaaactagtgctaaatccagtatgaatgtggatgagttccctggcccgggacatcttgctcaagtcaggaggccggagatcaggaaacggcaacaagcctcccagtactgacctgaaaacttgtgacaagaagaacaccaacaagtgctccctgggctgaggaccctttcttgcctccccaccccggaagctgaacctgagggagacaacggcagagggagtgagcaggggagaaatagcagaggggcttggagggtcacataggtagatggtaaagagaatgaggagaaaaaggagaaaagggaaaagcagaaaggaaaaaaaggaagagagaggaagggagaagggagaggaatgaattgaggaagtgaaagaaggcaaggaggtaggaagagagggaggaggaaaggaaggagagatgcctcaggcttcagaccttacctgggttttcagggcaaacataaatgtaaatacactgatttattctgttactagatcaggttttagggtcctgcaaaaggctagctcggcactacactagggaatttgctcctgttctgtcacttgtcatggtctttcttggtattaaaggccaccatttgcacaaatgtt

SEQ ID NO: 119 - DNA, 1849 bp, Homo Sapiens
ctcctcattttcttggtaccatcgccttagtctccccaaatccacaagaactcttctctatttttctctgttcccctcttcccctacccccaccacctagaactctctcgatcctatctccatgtttcctgtatccctgtccgccacatctttttctcccgatccgatgctagcctctttctgtcttgctgagcttcaaactctcccactgtgtccccacctcaccatctacttcccaggttccctgccccagtctccctgcacctcagctgaacaagggtttgggaagggtggaacaaggtggctgggtgtgctggagggtggcccaaggactgtgatcatgaatatgcagccagcttggtgaggaacgtggaaatgaggggacttttggggtctctgagtcagcctggtgactcatcctcctcccaggggcccccagcaggcttgggggaggtgcccaagtttctactaagaagaacattgtctcccctcctcccttctcttctttcttcatctcctctggcttgggaaatagacaggaagagtctcacgacaattctggttacaaaggaaaaggaaaaatgcccctctttcctaattgtctcaaaagtaatcctgtttagcctcagactttcctagtgccaagtttggcccactagttaattcacatagactggcttcagctacttttttccttactctctactaggagggtaggtgggataccaagagtagagggctctaagaaaacagagaagaggaatgtgaaagtaatatctggggggagacagagactgaattcagagagttgtcagctggtataactgaatggacccaagaggcagcctaaggattttaagcctccatgcagaattttcgtcaccttttccctcccaaaatccataaaatgaattgatttcaagatccgcactgtggatatagaggggaagaagatcaaactacaagtctgggacacggctggccaagagcggttcaagacaataactactgcctactaccgtggagccatgggcattatcctagtatacgacatcacggatgagaaatctttcgagaatattcagaactggatgaaaagcatcaaggagaatgcctcggctggggtggagcgcctcttgctggggaacaaatgtgacatggaggccaagaggaaggtgcagaaggagcaggccgataagttggctcgagagcatggaatccgatttttcgaaactagtgctaaatccagtatgaatgtggatgaggcttttagttccctggcccgggacatcttgctcaagtcaggaggccggagatcaggaaacggcaacaagcctcccagtactgacctgaaaacttgtgacaagaagaacaccaacaagtgctccctgggctgaggaccctttcttgcctccccaccccggaagctgaacctgagggagacaacggcagagggagtgagcaggggagaaatagcagaggggcttggagggtcacataggtagatggtaaagagaatgaggagaaaaaggagaaaagggaaaagcagaaaggaaaaaaaggaagagagaggaagggagaagggagaggaatgaattgaggaagtgaaagaaggcaaggaggtaggaagagagggaggaggaaaggaaggagagatgcctcaggcttcagaccttacctgggttttcagggcaaacataaatgtaaatacactgatttattctgttactagatcaggttttagggtcctgcaaaaggctagctcggcactacactagggaatttgctcctgttctgtcacttgtcatggtctttcttggtattaaaggccaccatttgcacaaatgtt

SEQ ID NO: 120 - DNA, 2184 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 121 - DNA, 2088 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 122 - DNA, 2325 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 123 - DNA, 2634 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggtgaatgacgggcgtgggtcggtgcgcgctcggcttgcgcacacggtgtctctaagtgcgcgggtgacgagagtcgggatgtgccggagaccccggggcggagagcgggattacaagtacaggaatccctggtcacgctccccgcccctggaaacccagctggggcgagggagggcgtggacgggaccgttctgggagctcgcctttggctgcggttggctccaggccccaggcgcagtttgctcgcggcgtggggatgaagtccgtgtccctggaggggcccaggaagggcgaggaaagcggagtggagcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 124 - DNA, 2392 bp, Homo Sapiens
aacatgttgagctctggcatagaagaggctggtggctattttgtccttgggctgcctgttttcaggtgaggaaggggatggtaggagacaggagacctctaaagaccccagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 125 - DNA, 2300 bp, Homo Sapiens
aacatgttgagctctggcatagaagaggctggtggctattttgtccttgggctgcctgttttcaggtgaggaaggggatggtaggagacaggagacctctaaagaccccaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 126 - DNA, 2186 bp, Homo Sapiens
aacatgttgagctctggcatagaagaggctggtggctattttgtccttgggctgcctgttttcaggtgaggaaggggatggtaggagacaggagacctctaaagaccccagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 127 - DNA, 2304 bp, Homo Sapiens
aacatgttgagctctggcatagaagaggctggtggctattttgtccttgggctgcctgttttcaggtgaggaaggggatggtaggagacaggagacctctaaagaccccagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 128 - DNA, 2140 bp, Homo Sapiens
aacatgttgagctctggcatagaagaggctggtggctattttgtccttgggctgcctgttttcagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 129 - DNA, 2108 bp, Homo Sapiens
aacatgttgagctctggcatagaagaggctggtggctattttgtccttgggctgcctgttttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 130 - DNA, 2275 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggagaaactgcaagttgggacttgtccctaggaaaatccagttgctgccaaggtcgtgcagtcactcagccctggagtcaagccagagcaggcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 131 - DNA, 2248 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggaaaatccagttgctgccaaggtcgtgcagtcactcagccctggagtcaagccagagcaggcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 132 - DNA, 2154 bp, Homo Sapiens
aacatgttgagctctggcatagaagaggctggtggctattttgtccttgggctgcctgttttcaggtgaggaaggggatggtaggagacaggagacctctaaagaccccaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 133 - DNA, 2413 bp, Homo Sapiens
gaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacaggtgcggacatgtgcacggaaggactttgtaagggaccaggattctcagaatccatggtccaagggctgacctgtctggtcctggtccagcatgctccaggtagaaggaaacaggcccagagaggggaagcaacctccctgaggtcacacagcaagtaggcagcaaagaccaactagctaacatttattgggaatgttcattatgccaggccctttgccaagcttctaagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatatt

SEQ ID NO: 134 - DNA, 3542 bp, Homo Sapiens
cgtggcctccgctgggcagggcgacgcggaaccgaggcggcggcggcggctgcggcggcaggaaatcggggctcggccccgccggcgcgcgaccccatccccatcccggtccctgcagagcgatccccgggcccagatatggacgcagcagagccgggactccccccaggtcctgagggcaggaagaggtacagtgacatcttccggagcctggacaacctcgaaatctcactggggaacgtgacccttgagatgctggctggagaccctctactctcagaagacccagaacctgacaagacccctacagccactgttaccaacgaagccagctgttggagcggcccctccccagagggtcctgtacccctcacaggggaggaactggacttgcggctcattcggacaaaggggggtgtggacgcagccctggaatatgccaagacctggagccgctatgccaaggaactgcttgcctggactgaaaagagagccagctatgagctggagtttgctaagagcaccatgaagatcgctgaagctggcaaggtgtccattcaacagcagagccacatgcctctgcagtacatctacaccctgtttctggagcacgatctcagcctgggaaccctggccatggagacagtggcccagcagaaaagagactactaccagcccctcgccgccaaacggactgagattgagaagtggcggaaggagttcaaggagcagtggatgaaggagcagaagcggatgaatgaggcggtgcaggcactgcggcgcgcccagctgcagtatgtgcaacgcagcgaggacctgcgggcacgctcccaggggtcccctgaggactcggccccccaggcctcgccgggacctagcaagcagcaggagcggcggcggcgctcgcgagaggaggcccaggccaaggcgcaggaggccgaggcgctgtaccaggcctgtgtccgcgaggccaacgcgcggcagcaggacctggagatcgccaagcagcgaatcgtgtcgcacgtgcgcaagctggtgtttcagggggatgaagtgctgaggcgggtgacgctgagtctcttcgggctgcggggggcgcaggcagagcgtggcccccgcgccttcgccgccctggccgagtgctgtgcgccctttgagccgggccagcgctaccaggagtttgtacgggcgctgcggcccgaggccccgccgcccccgccgcccgccttctccttccaggagttccttccctccttgaacagctcccctctggacatcagaaagaagctctctgggcctcttcctccaaggctggatgagaattcagctgagccaggcccttgggaggatccgggcacaggctggcgctggcaagggactccaggccccactccgggcagcgatgtggacagcgtgggtggcggcagcgagtctcggtccctggactcacccacttccagcccaggcgctggcacgaggcagctggtgaaggcttcgtccacaggcactgagtcctcagatgactttgaggagcgagaccctgacctgggagacgggctggagaatgggctgggcagccccttcgggaagtggacactgtccagcgcggctcagacccaccagctgcggcgactgcggggcccagccaagtgccgcgagtgcgaagccttcatggtcagcgggacggagtgtgaggagtgctttctgacctgccacaagcgctgcctggagactctcctgatcctctgtggacacaggcggctcccagcccggacacccctttttggggttgacttcctgcagctacccagggacttcccggaggaggtaccctttgtggtcacgaagtgcacggctgagatagaacaccgtgccctggatgtgcagggcatttaccgggtcagcgggtcccgggtccgtgtggagcggctgtgccaggctttcgagaatggccgagcgttggtggagctgtcggggaactcgcctcatgacgtctcgagtgtcctcaagcgatttcttcaggagctcaccgagcccgtgatccccttccacctctacgacgccttcatctctctggctaagaccttgcatgcagaccctggggacgaccctgggacccccagccccagccctgaggttatccgctcgctgaagaccctcttggtacagctgcctgactctaactacaacaccctgcggcacctggtggcccatctgttcagggtggctgcacgatttatggaaaacaagatgtctgccaacaacctgggcattgtgtttgggccgacactgctgcggccgccggacggcccgcgggcagccagcgccatccctgtcacctgcctgctggactctgggcatcaggcccagcttgtggagttcctcatcgtgcactacgagcagatctttgggatggatgagctcccccaggccactgagcccccgccccaagactccagcccagcccctgggcccctcacaaccagctcccaaccgccacccccgcaccttgacccagactcccagcccccagtcctagcctcagaccccggcccagacccccagcaccacagtaccctggagcagcatcccacggccacacctaccgagattccaactccacagagtgaccagagagaggacgtggctgaagacaccaaagatgggggaggggaagtgtccagccaaggcccagaggactcactcctggggacacagtctcgtggccacttcagccgccagccagtgaagtatccccggggcggtgtgaggcctgtaacccaccagctgtccagtctggccctggtggcttccaagctgtgcgaggagacccccatcacatcagtgcccagagggagtttgcgggggcgggggcccagccctgcagctgcctcccctgagggcagccccctgcgccgcaccccgctgcccaagcattttgagattacccaggagacagcccggctactctcgaaattggacagcgaggctgtgcccagggccacctgctgcccggacgtccagcctgaggaagccgaggaccatctctgaccaccctggcaccttaaataaggaagaggcccagattgtgaacacggacccatatatccctacctcccaccacctagtggccaaacaccccgccaggagttcaatgctgggagaggtccagagggttcctataaggaaaaactatttaatacatgacctaggggaggcctaaaacccttttggggataatgtcccagagtccccccactagacacaggtcactgccaagcatcagggccactcgggctcagaggtcactcagggtcaatactcagggtcagtgcaggttatgagatccttggggtccaccctagtctctgacacctgggacaggggtgcttttgctactttggttgtggtcactcccccacacctgcctgcctccttcacggactcgaagtgaccttcctggaggaggtgggcagctcagactccacatgctggtggtgcctgaggtctgatggcctctaataaactgtgtcctatatgcc

SEQ ID NO: 135 - DNA, 3616 bp, Homo Sapiens
cgtggcctccgctgggcagggcgacgcggaaccgaggcggcggcggcggctgcggcggcaggaaatcggggctcggccccgccggcgcgcgaccccatccccatcccggtccctgcagagcgatccccgggcccagatatggacgcagcagagccgggactccccccaggtcctgagggcaggaagaggtacagtgacatcttccggagcctggacaacctcgaaatctcactggggaacgtgacccttgagatgctggctggagaccctctactctcagaagacccagaacctgacaagacccctacagccactgttaccaacgaagccagctgttggagcggcccctccccagagggtcctgtacccctcacaggggaggaactggacttgcggctcattcggacaaaggggggtgtggacgcagccctggaatatgccaagacctggagccgctatgccaaggaactgcttgcctggactgaaaagagagccagctatgagctggagtttgctaagagcaccatgaagatcgctgaagctggcaaggtgtccattcaacagcagagccacatgcctctgcagtacatctacaccctgtttctggagcacgatctcagcctgggaaccctggccatggagacagtggcccagcagaaaagagactactaccagcccctcgccgccaaacggactgagattgagaagtggcggaaggagttcaaggagcagtggatgaaggagcagaagcggatgaatgaggcggtgcaggcactgcggcgcgcccagctgcagtatgtgcaacgcagcgaggacctgcgggcacgctcccaggggtcccctgaggactcggccccccaggcctcgccgggacctagcaagcagcaggagcggcggcggcgctcgcgagaggaggcccaggccaaggcgcaggaggccgaggcgctgtaccaggcctgtgtccgcgaggccaacgcgcggcagcaggacctggagatcgccaagcagcgaatcgtgtcgcacgtgcgcaagctggtgtttcagggggatgaagtgctgaggcgggtgacgctgagtctcttcgggctgcggggggcgcaggcagagcgtggcccccgcgccttcgccgccctggccgagtgctgtgcgccctttgagccgggccagcgctaccaggagtttgtacgggcgctgcggcccgaggccccgccgcccccgccgcccgccttctccttccaggagttccttccctccttgaacagctcccctctggacatcagaaagaagctctctgggcctcttcctccaaggctggatgagaattcagctgagccaggcccttgggaggatccgggcacaggctggcgctggcaagggactccaggccccactccgggcagcgatgtggacagcgtgggtggcggcagcgagtctcggtccctggactcacccacttccagcccaggcgctggcacgaggcagctggtgaaggcttcgtccacaggcactgagtcctcagatgactttgaggagcgagaccctggtgaggctgaagcagggtaggtcaggatgggacagggcagggcaccaggcactcctgaccctgtctccctgcagacctgggagacgggctggagaatgggctgggcagccccttcgggaagtggacactgtccagcgcggctcagacccaccagctgcggcgactgcggggcccagccaagtgccgcgagtgcgaagccttcatggtcagcgggacggagtgtgaggagtgctttctgacctgccacaagcgctgcctggagactctcctgatcctctgtggacacaggcggctcccagcccggacacccctttttggggttgacttcctgcagctacccagggacttcccggaggaggtaccctttgtggtcacgaagtgcacggctgagatagaacaccgtgccctggatgtgcagggcatttaccgggtcagcgggtcccgggtccgtgtggagcggctgtgccaggctttcgagaatggccgagcgttggtggagctgtcggggaactcgcctcatgacgtctcgagtgtcctcaagcgatttcttcaggagctcaccgagcccgtgatccccttccacctctacgacgccttcatctctctggctaagaccttgcatgcagaccctggggacgaccctgggacccccagccccagccctgaggttatccgctcgctgaagaccctcttggtacagctgcctgactctaactacaacaccctgcggcacctggtggcccatctgttcagggtggctgcacgatttatggaaaacaagatgtctgccaacaacctgggcattgtgtttgggccgacactgctgcggccgccggacggcccgcgggcagccagcgccatccctgtcacctgcctgctggactctgggcatcaggcccagcttgtggagttcctcatcgtgcactacgagcagatctttgggatggatgagctcccccaggccactgagcccccgccccaagactccagcccagcccctgggcccctcacaaccagctcccaaccgccacccccgcaccttgacccagactcccagcccccagtcctagcctcagaccccggcccagacccccagcaccacagtaccctggagcagcatcccacggccacacctaccgagattccaactccacagagtgaccagagagaggacgtggctgaagacaccaaagatgggggaggggaagtgtccagccaaggcccagaggactcactcctggggacacagtctcgtggccacttcagccgccagccagtgaagtatccccggggcggtgtgaggcctgtaacccaccagctgtccagtctggccctggtggcttccaagctgtgcgaggagacccccatcacatcagtgcccagagggagtttgcgggggcgggggcccagccctgcagctgcctcccctgagggcagccccctgcgccgcaccccgctgcccaagcattttgagattacccaggagacagcccggctactctcgaaattggacagcgaggctgtgcccagggccacctgctgcccggacgtccagcctgaggaagccgaggaccatctctgaccaccctggcaccttaaataaggaagaggcccagattgtgaacacggacccatatatccctacctcccaccacctagtggccaaacaccccgccaggagttcaatgctgggagaggtccagagggttcctataaggaaaaactatttaatacatgacctaggggaggcctaaaacccttttggggataatgtcccagagtccccccactagacacaggtcactgccaagcatcagggccactcgggctcagaggtcactcagggtcaatactcagggtcagtgcaggttatgagatccttggggtccaccctagtctctgacacctgggacaggggtgcttttgctactttggttgtggtcactcccccacacctgcctgcctccttcacggactcgaagtgaccttcctggaggaggtgggcagctcagactccacatgctggtggtgcctgaggtctgatggcctctaataaactgtgtcctatatgcc

SEQ ID NO: 136 - DNA, 3540 bp, Homo Sapiens
cgtggcctccgctgggcagggcgacgcggaaccgaggcggcggcggcggctgcggcggcaggaaatcggggctcggccccgccggcgcgcgaccccatccccatcccggtccctgcagagcgatccccgggcccagatatggacgcagcagagccgggactccccccaggtcctgagggcaggaagaggtacagtgacatcttccggagcctggacaacctcgaaatctcactggggaacgacccttgagatgctggctggagaccctctactctcagaagacccagaacctgacaagacccctacagccactgttaccaacgaagccagctgttggagcggcccctccccagagggtcctgtacccctcacaggggaggaactggacttgcggctcattcggacaaaggggggtgtggacgcagccctggaatatgccaagacctggagccgctatgccaaggaactgcttgcctggactgaaaagagagccagctatgagctggagtttgctaagagcaccatgaagatcgctgaagctggcaaggtgtccattcaacagcagagccacatgcctctgcagtacatctacaccctgtttctggagcacgatctcagcctgggaaccctggccatggagacagtggcccagcagaaaagagactactaccagcccctcgccgccaaacggactgagattgagaagtggcggaaggagttcaaggagcagtggatgaaggagcagaagcggatgaatgaggcggtgcaggcactgcggcgcgcccagctgcagtatgtgcaacgcagcgaggacctgcgggcacgctcccaggggtcccctgaggactcggccccccaggcctcgccgggacctagcaagcagcaggagcggcggcggcgctcgcgagaggaggcccaggccaaggcgcaggaggccgaggcgctgtaccaggcctgtgtccgcgaggccaacgcgcggcagcaggacctggagatcgccaagcagcgaatcgtgtcgcacgtgcgcaagctggtgtttcagggggatgaagtgctgaggcgggtgacgctgagtctcttcgggctgcggggggcgcaggcagagcgtggcccccgcgccttcgccgccctggccgagtgctgtgcgccctttgagccgggccagcgctaccaggagtttgtacgggcgctgcggcccgaggccccgccgcccccgccgcccgccttctccttccaggagttccttccctccttgaacagctcccctctggacatcagaaagaagctctctgggcctcttcctccaaggctggatgagaattcagctgagccaggcccttgggaggatccgggcacaggctggcgctggcaagggactccaggccccactccgggcagcgatgtggacagcgtgggtggcggcagcgagtctcggtccctggactcacccacttccagcccaggcgctggcacgaggcagctggtgaaggcttcgtccacaggcactgagtcctcagatgactttgaggagcgagaccctgacctgggagacgggctggagaatgggctgggcagccccttcgggaagtggacactgtccagcgcggctcagacccaccagctgcggcgactgcggggcccagccaagtgccgcgagtgcgaagccttcatggtcagcgggacggagtgtgaggagtgctttctgacctgccacaagcgctgcctggagactctcctgatcctctgtggacacaggcggctcccagcccggacacccctttttggggttgacttcctgcagctacccagggacttcccggaggaggtaccctttgtggtcacgaagtgcacggctgagatagaacaccgtgccctggatgtgcagggcatttaccgggtcagcgggtcccgggtccgtgtggagcggctgtgccaggctttcgagaatggccgagcgttggtggagctgtcggggaactcgcctcatgacgtctcgagtgtcctcaagcgatttcttcaggagctcaccgagcccgtgatccccttccacctctacgacgccttcatctctctggctaagaccttgcatgcagaccctggggacgaccctgggacccccagccccagccctgaggttatccgctcgctgaagaccctcttggtacagctgcctgactctaactacaacaccctgcggcacctggtggcccatctgttcagggtggctgcacgatttatggaaaacaagatgtctgccaacaacctgggcattgtgtttgggccgacactgctgcggccgccggacggcccgcgggcagccagcgccatccctgtcacctgcctgctggactctgggcatcaggcccagcttgtggagttcctcatcgtgcactacgagcagatctttgggatggatgagctcccccaggccactgagcccccgccccaagactccagcccagcccctgggcccctcacaaccagctcccaaccgccacccccgcaccttgacccagactcccagcccccagtcctagcctcagaccccggcccagacccccagcaccacagtaccctggagcagcatcccacggccacacctaccgagattccaactccacagagtgaccagagagaggacgtggctgaagacaccaaagatgggggaggggaagtgtccagccaaggcccagaggactcactcctggggacacagtctcgtggccacttcagccgccagccagtgaagtatccccggggcggtgtgaggcctgtaacccaccagctgtccagtctggccctggtggcttccaagctgtgcgaggagacccccatcacatcagtgcccagagggagtttgcgggggcgggggcccagccctgcagctgcctcccctgagggcagccccctgcgccgcaccccgctgcccaagcattttgagattacccaggagacagcccggctactctcgaaattggacagcgaggctgtgcccagggccacctgctgcccggacgtccagcctgaggaagccgaggaccatctctgaccaccctggcaccttaaataaggaagaggcccagattgtgaacacggacccatatatccctacctcccaccacctagtggccaaacaccccgccaggagttcaatgctgggagaggtccagagggttcctataaggaaaaactatttaatacatgacctaggggaggcctaaaacccttttggggataatgtcccagagtccccccactagacacaggtcactgccaagcatcagggccactcgggctcagaggtcactcagggtcaatactcagggtcagtgcaggttatgagatccttggggtccaccctagtctctgacacctgggacaggggtgcttttgctactttggttgtggtcactcccccacacctgcctgcctccttcacggactcgaagtgaccttcctggaggaggtgggcagctcagactccacatgctggtggtgcctgaggtctgatggcctctaataaactgtgtcctatatgcc

SEQ ID NO: 137 - DNA, 3345 bp, Homo Sapiens
cgtggcctccgctgggcagggcgacgcggaaccgaggcggcggcggcggctgcggcggcaggaaatcggggctcggccccgccggcgcgcgaccccatccccatcccggtccctgcagagcgatccccgggcccagatatggacgcagcagagccgggactccccccaggtcctgagggcaggaagaggtacagtgacatcttccggagcctggacaacctcgaaatctcactggggaacgtgacccttgagatgctggctggagaccctctactctcagaagacccagaacctgacaagacccctacagccactgttaccaacgaagccagctgttggagcggcccctccccagagggtcctgtacccctcacaggggaggaactggacttgcggctcattcggacaaaggggggtgtggacgcagccctggaatatgccaagacctggagccgctatgccaaggaactgcttgcctggactgaaaagagagccagctatgagctggagtttgctaagagcaccatgaagatcgctgaagctggcaaggtgtccattcaacagcagagccacatgcctctgcagtacatctacaccctgtttctggagcacgatctcagcctgggaaccctggccatggagacagtggcccagcagaaaagagactactaccagcccctcgccgccaaacggactgagattgagaagtggcggaaggagttcaaggagcagtggatgaaggagcagaagcggatgaatgaggcggtgcaggcactgcggcgcgcccagctgcagtatgtgcaacgcagcgaggacctgcgggcacgctcccaggggtcccctgaggactcggccccccaggcctcgccgggacctagcaagcagcaggagcggcggcggcgctcgcgagaggaggcccaggccaaggcgcaggaggccgaggcgctgtaccaggcctgtgtccgcgaggccaacgcgcggcagcaggacctggagatcgccaagcagcgaatcgtgtcgcacgtgcgcaagctggtgtttcagggggatgaagtgctgaggcgggtgacgctgagtctcttcgggctgcggggggcgcaggcagagcgtggcccccgcgccttcgccgccctggccgagtgctgtgcgccctttgagccgggccagcgctaccaggagtttgtacgggcgctgcggcccgaggccccgccgcccccgccgcccgccttctccttccaggagttccttccctccttgaacagctcccctctggacatcagaaagaagctctctgggcctcttcctccaaggctggatgagaattcagctgagccaggcccttgggaggatccgggcacaggctggcgctggcaagggactccaggccccactccgggcagcgatgtggacagcgtgggtggcggcagcgagtctcggtccctggactcacccacttccagcccaggcgctggcacgaggcagctggtgaaggcttcgtccacaggcactgagtcctcagatgactttgaggagcgagaccctgacctgggagacgggctggagaatgggctgggcagccccttcgggaagtggacactgtccagcgcggctcagacccaccagctgcggcgactgcggggcccagccaagtgccgcgagtgcgaagccttcatggtcagcgggacggagtgtgaggagtgctttctgacctgccacaagcgctgcctggagactctcctgatcctctgtggacacaggcggctcccagcccggacacccctttttggggttgacttcctgcagctacccagggacttcccggaggaggtaccctttgtggtcacgaagtgcacggctgagatagaacaccgtgccctggatgtgcagggcatttaccgggtcagcgggtcccgggtccgtgtggagcggctgtgccaggctttcgagaatggccgagcgttggtggagctgtcggggaactcgcctcatgacgtctcgagtgtcctcaagcgatttcttcaggagggtggctgcacgatttatggaaaacaagatgtctgccaacaacctgggcattgtgtttgggccgacactgctgcggccgccggacggcccgcgggcagccagcgccatccctgtcacctgcctgctggactctgggcatcaggcccagcttgtggagttcctcatcgtgcactacgagcagatctttgggatggatgagctcccccaggccactgagcccccgccccaagactccagcccagcccctgggcccctcacaaccagctcccaaccgccacccccgcaccttgacccagactcccagcccccagtcctagcctcagaccccggcccagacccccagcaccacagtaccctggagcagcatcccacggccacacctaccgagattccaactccacagagtgaccagagagaggacgtggctgaagacaccaaagatgggggaggggaagtgtccagccaaggcccagaggactcactcctggggacacagtctcgtggccacttcagccgccagccagtgaagtatccccggggcggtgtgaggcctgtaacccaccagctgtccagtctggccctggtggcttccaagctgtgcgaggagacccccatcacatcagtgcccagagggagtttgcgggggcgggggcccagccctgcagctgcctcccctgagggcagccccctgcgccgcaccccgctgcccaagcattttgagattacccaggagacagcccggctactctcgaaattggacagcgaggctgtgcccagggccacctgctgcccggacgtccagcctgaggaagccgaggaccatctctgaccaccctggcaccttaaataaggaagaggcccagattgtgaacacggacccatatatccctacctcccaccacctagtggccaaacaccccgccaggagttcaatgctgggagaggtccagagggttcctataaggaaaaactatttaatacatgacctaggggaggcctaaaacccttttggggataatgtcccagagtccccccactagacacaggtcactgccaagcatcagggccactcgggctcagaggtcactcagggtcaatactcagggtcagtgcaggttatgagatccttggggtccaccctagtctctgacacctgggacaggggtgcttttgctactttggttgtggtcactcccccacacctgcctgcctccttcacggactcgaagtgaccttcctggaggaggtgggcagctcagactccacatgctggtggtgcctgaggtctgatggcctctaataaactgtgtcctatatgcc

SEQ ID NO: 138 - protein, 124 aa, Homo Sapiens
MSSALESVTVSDRPLGVSITKAEVAPEEDERKKRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 139 - protein, 181 aa, Homo Sapiens
MMLQHPGQVSASEVSASAIVPCLSPPGSLVFEDFANLTPFVKEELRFAIQNKHLCHRMSSALESVTVSDRPLGVSITKAEVAPEEDERKKRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 140 - protein, 340 aa, Homo Sapiens
MADSVKTFLQDLARGIKDSIWGICTISKLDARIQQKREEQRRRRASSVLAQRRAQSIERKQESEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIISGCLFSILFPLFIISANEAKTPGKAYLFQLRLFSLVVFLSNRLFHKTVYLQSALSSSTSAEKFPSPHPSPAKLKATAGH

SEQ ID NO: 141 - protein, 228 aa, Homo Sapiens
MADSVKTFLQDLARGIKDSIWGICTISKLDARIQQKREEQRRRRASSVLAQRRAQSIERKQESEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGKSI

SEQ ID NO: 142 - protein, 342 aa, Homo Sapiens
MADSVKTFLQDLARGIKDSIWGICTISKLDARIQQKREEQRRRRASSVLAQRRAQSIERKQERRILALSPSCHLRLPWCNLSLLQPLPPRSKQFFCLSEPAGWKYSEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIISGCLFSILFPLFIISANEAKTLAKHISSSCASSPWWSS

SEQ ID NO: 143 - protein, 152 aa, Homo Sapiens
MADSVKTFLQDLARGIKDSIWGICTISKLDARIQQKREEQRRRRASSVLAQRRAQSIERKQERRILALSPSCHLRLPWCNLSLLQPLPPRSKQFFCLSEPAGWKYRCAPPRLANFLYSLVETGFCHVGQAGLELLTSVDRLPRPPKVLGLQP

SEQ ID NO: 144 - protein, 163 aa, Homo Sapiens [sequence omitted]

SEQ ID NO: 145 - protein, 342 aa, Homo Sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELFRSQAHRDFVRDATWSPLNHSLLTTVGWDHQVVHHVVPTEPLPAPGPASVTE

SEQ ID NO: 146 - protein, 290 aa, Homo Sapiens [sequence omitted]

SEQ ID NO: 147 - protein, 302 aa, Homo Sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSEFISSSMKIEDWIG

SEQ ID NO: 148 - protein, 210 aa, Homo Sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIVRLC

SEQ ID NO: 149 - protein, 179 aa, Homo Sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDIWWVPVLIAPVSKPPVGAFPILLTSLGNILGLF

SEQ ID NO: 150 - protein, 123 aa, Homo Sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGESLSHYPPPPPGGTVEWRLGVP

SEQ ID NO: 151 - protein, 303 aa, Homo Sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELAGSCAETPRWCCP

SEQ ID NO: 152 - protein, 72 aa, Homo Sapiens
MRKETPPPLVPPAAREWNLPPNAPACMERQLEAARYRSGEPRSRAWELPYPGPGRPSPETDPPSPSPPCMLS

SEQ ID NO: 153 - protein, 37 aa, Homo Sapiens
MRKETPPPLVPPAAREWNLPPNAPACMERRFSSGPPA

SEQ ID NO: 154 - protein, 308 aa, Homo Sapiens [sequence omitted]

SEQ ID NO: 155 - protein, 331 aa, Homo Sapiens
MTPGPRSCRNATRTFRAPVRQGEPGGAGPQPHPKAQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 156 - protein, 94 aa, Homo Sapiens [sequence omitted]

SEQ ID NO: 157 - protein, 129 aa, Homo Sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 158 - protein, 121 aa, Homo Sapiens
MARGSLRRLLRLLVLGLWLALLRSVAGEQAPGTRDSGVGEPRAGAREPDWVSGEQRRTGGTGGCGDLGSGGLQTARSRLWEVPSLSKLHSQSGPQFLPPSNWGWGGSSVPQVRSTSNGERK

SEQ ID NO: 159 - protein, 94 aa, Homo Sapiens
MARGSLRRLLRLLVLGLWLALLRSVAGEQAPASDPRPPPQAPPPAPAAAPGARTWTSAWTARLAGRDRTATSAWAALQHLLPPSGCFGPSLGAL

SEQ ID NO: 160 - protein, 250 aa, Homo Sapiens
MARGSLRRLLRLLVLGLWLALLRSVAGEQAPGTAPCSRGSSWSADLDKCMDCASCRARPHSDFCLGCEWGAGPVASGPQTGREGGWCAERGRELCIWEIIREEVGAGRGLRSGRRPRLGEGRRLSLRGRGWPGEGRGLTPSPAPRRCSTSCPLPAALAHPWGRSEPDLRAGAAFWLFGLETMPQEREVHHPHRGDRRRGLPSCGADPVTMCPLPAGARPLIIHSSILEPVSASQTRREPSSSNHKGGGGR

SEQ ID NO: 161 - protein, 295 aa, Homo Sapiens
MIEVLTTTDSQKLLHQLNALLEQESRCQPKVCGLRLIESAHDNGLRMTARLRDFEVKDLLSLTQFFGFDTETFSLAVNLLDRFLSKMKVQPKHLGCVGLSCFYLAVKSIEEERNVPLATDLIRISQYRFTVSDLMRMEKIVLEKVCWKVKATTAFQFLQLYYSLLQENLPLERRNSINFERLEAQLKACHCRIIFSKAKPSVLALSIIALEIQAQKCVELTEGIECLQKHSKINGRDLTFWQELVSKCLTEYSSNKCSKPNVQKLKWIVSGRTARQLKHSYYRITHLPTIPEMVP

SEQ ID NO: 162 - protein, 114 aa, Homo Sapiens [sequence omitted]

SEQ ID NO: 163 - protein, 181 aa, Homo Sapiens
MAGSREVVAMDCEMVGLGPHRESGLARCSLVNVHGAVLYDKFIRPEGEITDYRTRVSGVTPQHMVGATPFAVARLEILQLLKGKLVVGHDLKHDFQALKEDMSGYTIYDTSTDRLLWREAKLDHCRRVSLRVLSERLLHKSIQNSLLGHSSVEDARATMELYQISQRIRARRGLPRLAVSD

SEQ ID NO: 164 - protein, 125 aa, Homo Sapiens
MSHSRGRGTGIFVHQFPSATSCCCSCALIPQHFWLVGKAGRKRHTDMGGWEFTLWEGQRERRQTLRARAVHRDPSGQQLFIKHLLPALGQILETQGEQDPSLSLSSWLVGRWPLTRSSAHMVRSV

SEQ ID NO: 165 - protein, 203 aa, Homo Sapiens
MAKAYDHLFKLLLIGDSGVGKTCLIIRFAEDNFNNTYISTIGIDFKIRTVDIEGKKIKLQVWDTAGQERFKTITTAYYRGAMGIILVYDITDEKSFENIQNWMKSIKENASAGVERLLLGNKCDMEAKRKVQKEQADKLAREHGIRFFETSAKSSMNVDEAFSSLARDILLKSGGRRSGNGNKPPSTDLKTCDKKNTNKCSLG

SEQ ID NO: 166 - protein, 122 aa, Homo Sapiens
MGIILVYDITDEKSFENIQNWMKSIKENASAGVERLLLGNKCDMEAKRKVQKEQADKLAREHGIRFFETSAKSSMNVDEAFSSLARDILLKSGGRRSGNGNKPPSTDLKTCDKKNTNKCSLG

SEQ ID NO: 167 - protein, 166 aa, Homo Sapiens
MAKAYDHLFKLLLIGDSGVGKTCLIIRFAEDNFNNTYISTIGIDFKIRTVDIEGKKIKLQVWDTAGQERFKTITTAYYRGAMGIILVYDITDEKSFENIQNWMKSIKENASAGVERLLLGNKCDMEAKRKVQKEQADKVRARLSNVLELGWEGQDRLHCRGLGCCP

SEQ ID NO: 168 - protein, 72 aa, Homo Sapiens [sequence omitted]

SEQ ID NO: 169 - protein, 184 aa, Homo Sapiens
MAKAYDHLFKLLLIGDSGVGKTCLIIRFAEDNFNNTYISTIGIDFKIRTVDIEGKKIKLQVWDTAGQERFKTITTAYYRGAMGIILVYDITDEKSFENIQNWMKSIKENASAGVERLLLGNKCDMEAKRKVQKEQADKLAREHGIRFFETSAKSSMNVDEFPGPGHLAQVRRPEIRKRQQASQY

SEQ ID NO: 170 - protein, 164 aa, Homo Sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 171 - protein, 163 aa, Homo Sapiens
SEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 172 - protein, 200 aa, Homo Sapiens [sequence omitted]

SEQ ID NO: 173 - protein, 970 aa, Homo Sapiens
MDAAEPGLPPGPEGRKRYSDIFRSLDNLEISLGNVTLEMLAGDPLLSEDPEPDKTPTATVTNEASCWSGPSPEGPVPLTGEELDLRLIRTKGGVDAALEYAKTWSRYAKELLAWTEKRASYELEFAKSTMKIAEAGKVSIQQQSHMPLQYIYTLFLEHDLSLGTLAMETVAQQKRDYYQPLAAKRTEIEKWRKEFKEQWMKEQKRMNEAVQALRRAQLQYVQRSEDLRARSQGSPEDSAPQASPGPSKQQERRRRSREEAQAKAQEAEALYQACVREANARQQDLEIAKQRIVSHVRKLVFQGDEVLRRVTLSLFGLRGAQAERGPRAFAALAECCAPFEPGQRYQEFVRALRPEAPPPPPPAFSFQEFLPSLNSSPLDIRKKLSGPLPPRLDENSAEPGPWEDPGTGWRWQGTPGPTPGSDVDSVGGGSESRSLDSPTSSPGAGTRQLVKASSTGTESSDDFEERDPDLGDGLENGLGSPFGKWTLSSAAQTHQLRRLRGPAKCRECEAFMVSGTECEECFLTCHKRCLETLLILCGHRRLPARTPLFGVDFLQLPRDFPEEVPFVVTKCTAEIEHRALDVQGIYRVSGSRVRVERLCQAFENGRALVELSGNSPHDVSSVLKRFLQELTEPVIPFHLYDAFISLAKTLHADPGDDPGTPSPSPDVIRSLKTLLVQLPDSNYNTLRHLVAHLFRVAARFMENKMSANNLGIVFGPTLLRPPDGPRAASAIPVTCLLDSGHQAQLVEFLIVHYEQIFGMDELPQATEPPPQDFSPAPGPLTTSSQPPPPHLDPDSQPPVLASDPGPDPQHHSTLEQHPTATPTEIPTPQSDQREDVAEDTKDGGGEVSSQGPEDSLLGTQSRGHFSRQPCKYPRGGVRPVTHQLSSLALVASKLCEETPITSVPRGSLRGRGPSPAAASPEGSPLRRTPLPKHFEITQETARLLSKLDSEAVPRATCCPDVQPEEAEDHL

SEQ ID NO: 174 - protein, 474 aa, Homo Sapiens
MDAAEPGLPPGPEGRKRYSDIFRSLDNLEISLGNVTLEMLAGDPLLSEDPEPDKTPTATVTNEASCWSGPSPEGPVPLTGEELDLRLIRTKGGVDAALEYAKTWSRYAKELLAWTEKRASYELEFAKSTMKIAEAGKVSIQQQSHMPLQYIYTLFLEHDLSLGTLAMETVAQQKRDYYQPLAAKRTEIEKWRKEFKEQWMKEQKRMNEAVQALRRAQLQYVQRSEDLRARSQGSPEDSAPQASPGPSKQQERRRRSREEAQAKAQEAEALYQACVREANARQQDLEIAKQRIVSHVRKLVFQGDEVLRRVTLSLFGLRGAQAERGPRAFAALAECCAPFEPGQRYQEFVRALRPEAPPPPPPAFSFQEFLPSLNSSPLDIRKKLSGPLPPRLDENSAEPGPWEDPGTGWRWQGTPGPTPGSDVDSVGGGSESRSLDSPTSSPGAGTRQLVKASSTGTESSDDFEERDPGEAEAG

SEQ ID NO: 175 - protein, 36 aa, Homo Sapiens
MDAAEPGLPPGPEGRKRYSDIFRSLDNLEISLGNDP

SEQ ID NO: 176 - protein, 694 aa, Homo Sapiens
MDAAEPGLPPGPEGRKRYSDIFRSLDNLEISLGNVTLEMLAGDPLLSEDPEPDKTPTATVTNEASCWSGPSPEGPVPLTGEELDLRLIRTKGGVDAALEYAKTWSRYAKELLAWTEKRASYELEFAKSTMKIAEAGKVSIQQQSHMPLQYIYTLFLEHDLSLGTLAMETVAQQKRDYYQPLAAKRTEIEKWRKEFKEQWMKEQKRMNEAVQALRRAQLQYVQRSEDLRARSQGSPEDSAPQASPGPSKQQERRRRSREEAQAKAQEAEALYQACVREANARQQDLEIAKQRIVSHVRKLVFQGDEVLRRVTLSLFGLRGAQAERGPRAFAALAECCAPFEPGQRYQEFVRALRPEAPPPPPPAFSFQEFLPSLNSSPLDIRKKLSGPLPPRLDENSAEPGPWEDPGTGWRWQGTPGPTPGSDVDSVGGGSESRSLDSPTSSPGAGTRQLVKASSTGTESSDDFEERDPDLGDGLENGLGSPFGKWTLSSAAQTHQLRRLRGPAKCRECEAFMVSGTECEECFLTCHKRCLETLLILCGHRRLPARTPLFGVDFLQLPRDFPEEVPFVVTKCTAEIEHRALDVQGIYRVSGSRVRVERLCQAFENGRALVELSGNSPHDVSSVLKRFLQEGGCTIYGKQDVCQQPGHCVWADTAAAAGRPAGSQRHPCHLPAGLWASGPACGVPHRALRADLWDG

SEQ ID NO: 177 - DNA, 1977 bp, Mus Musculus
ctgggattggtaacctggagttaagcgggctccctgccaacgcgagggctttaaaaggggtgatgcaacgcgctcccagccacagtctcactcagcgagacgccgcgcacggtgcttccccagtggagccaatcggctaacccgcgctccggcagagtccttggcgctcgcccgccggcgggacagaccacccgcctctggccgctctctggaccctggccgccccgagcgaagactggagcaaaatgatgcttcaacatccaggccaggtctctgcctcagaagtcagtgcgaccgccattgtcccctgcctctcacctcctgggtcactggtatttgaggattttgctaacctgacaccctttgtcaaggaagagctgagattcgccatccagaataaacacctctgccatcggatgtcctctgcgctggagtcagttaccgtcaacaacagacccctggagatgtcagtcaccaagtctgaggcggcccctgaagaagatgagaggaaaaggaggcggcgagaaagaaataaaattgctgctgccaagtgtcgaaacaagaaaaaggagaagacagagtgcctgcagaaagagtcagagaaactggagagtgtgaatgctgagctgaaggcccagattgaggagctgaagaatgagaaacagcatttgatatacatgctcaacctgcaccggcccacctgcatcgtccgggctcagaatggacggacaccggaagacgagaggaacctctttatccaacagataaaagaaggaacattgcagagctaagcagaggtggcacggaggcaattggggagttcttactgaatcctccttttccaccccacaccctgaagccattggaaaactggcttcctgtgcacttctagaatcccagcagccaagagccgttggggcaggagggcctgtggtgacctactgcattgacccactctgcccccgagtgaaccgtggagcaggcaggagcatcctttgtctcaccaattccaggatttaggccttatcatcccggccagtctcagatgacctagctggccccaggctggggtcctatgcaaagcaggatcccactaatgggattcaggcagaagtgtctaccttgataggtggggtgggaccacatcctccactgtggctgacaacgcccttccaagggaatatggaatgagaacattcattattgaggttgtccaatggccagggtatgctttctagaaaatatgctgttctgtcccagaatgactgtgcatagggtatccgtttcagagcctggtgttgtgctatttagatgtttgtcttgcacaacattggcatgatttttccgggagtttcatcagatctgatttctgagagtctggggatctgccatggtggaaagtgcccctcaaaagcatttgtgtggccacatgaactggctggcaccaggggagtgaaactggctgatgaccagctgagccactttgtgccaacagaggatggacgacacctttccctgtacccactgcagaggaagaaccctgggcacagcagctttgtccttggctacaaactgttacaacgtcacacaatgaaggcacaaagtccaactttcaaagggtgtaggactccatactcagtgacagggcaggaagagccaaagataaccacagccacagcctgtggagaccagggttggaagccaggtgcagggccaggcatctgcattgtgggatgttaatggcacttttgtcttgtagctattttgagatgtggtccagagcatttcagctgggagatctccctctggccaccaggactctggctactgttaaaatcctgatgtttctgtggaatcctcagtgtttaatcccactcaatagtatcattacagttttctgtaagagaaaatattacttatttatcccagtattcctagcctgtcaacataataaatatcggaacaaaacctggtaaa

SEQ ID NO: 178 - DNA, 1871 bp, Mus Musculus
ctgggattggtaacctggagttaagcgggctccctgccaacgcgagggctttaaaaggggtgatgcaacgcgctcccagccacagtctcactcagcgagacgccgcgcacggtgcttccccagtggagccaatcggctaacccgcgctccggcagagtccttggcgctcgcccgccggcgggacagaccacccgcctctggccgctctctggaccctggccgccccgagcgaagactggagcaaaatgatgcttcaacatccaggccagagctgagattcgccatccagaataaacacctctgccatcggatgtcctctgcgctggagtcagttaccgtcaacaacagacccctggagatgtcagtcaccaagtctgaggcggcccctgaagaagatgagaggaaaaggaggcggcgagaaagaaataaaattgctgctgccaagtgtcgaaacaagaaaaaggagaagacagagtgcctgcagaaagagtcagagaaactggagagtgtgaatgctgagctgaaggcccagattgaggagctgaagaatgagaaacagcatttgatatacatgctcaacctgcaccggcccacctgcatcgtccgggctcagaatggacggacaccggaagacgagaggaacctctttatccaacagataaaagaaggaacattgcagagctaagcagaggtggcacggaggcaattggggagttcttactgaatcctccttttccaccccacaccctgaagccattggaaaactggcttcctgtgcacttctagaatcccagcagccaagagccgttggggcaggagggcctgtggtgacctactgcattgacccactctgcccccgagtgaaccgtggagcaggcaggagcatcctttgtctcaccaattccaggatttaggccttatcatcccggccagtctcagatgacctagctggccccaggctggggtcctatgcaaagcaggatcccactaatgggattcaggcagaagtgtctaccttgataggtggggtgggaccacatcctccactgtggctgacaacgcccttccaagggaatatggaatgagaacattcattattgaggttgtccaatggccagggtatgctttctagaaaatatgctgttctgtcccagaatgactgtgcatagggtatccgtttcagagcctggtgttgtgctatttagatgtttgtcttgcacaacattggcatgatttttccgggagtttcatcagatctgatttctgagagtctggggatctgccatggtggaaagtgcccctcaaaagcatttgtgtggccacatgaactggctggcaccaggggagtgaaactggctgatgaccagctgagccactttgtgccaacagaggatggacgacacctttccctgtacccactgcagaggaagaaccctgggcacagcagctttgtccttggctacaaactgttacaacgtcacacaatgaaggcacaaagtccaactttcaaagggtgtaggactccatactcagtgacagggcaggaagagccaaagataaccacagccacagcctgtggagaccagggttggaagccaggtgcagggccaggcatctgcattgtgggatgttaatggcacttttgtcttgtagctattttgagatgtggtccagagcatttcagctgggagatctccctctggccaccaggactctggctactgttaaaatcctgatgtttctgtggaatcctcagtgtttaatcccactcaatagtatcattacagttttctgtaagagaaaatattacttatttatcccagtattcctagcctgtcaacataataaatatcggaacaaaacctggtaaa

SEQ ID NO: 179 - DNA, 3932 bp, Mus Musculus
ggggcgacaccactgggtcggcagaggcaggaaatcgggctggtcccgggcggcgcgcagccctgtccctttctcagcattgccgcgcgttccccaccccaccccctgcccccgggcatcgccatggactcagcggagacagagctaaccccagctcctgagggcaggaagagatacagtgacatcttccagagcctggacaacctcgagatatcattggggaacgtaacttttgatcccctggctggagaccctgtacgcagagaagacctggagcctgacaaagctgacacagccacagtggtgactgaggagaacagtgaagccagtagttggcgggatctctccccagaaggtcctgcacccctcacagaggaagaactggatttgcgactcattcggaccaagggtggtgtagatgctgccctggagtatgctaaagcatggagccgctatgccaaggaactgctggcctggacagacaagagagccaactacgagctcgagtttgctaagagtatcatgaagctcgctgaggctggcaaggtgtccattctccagcagagccaaatgccactccagtacatctacaccttgtttctggagcatgacctcagcctgggagccctggccctggagacagtggcccaacagaagagagactactaccagcctctggcggccaaaaggatggaaattgagaagtggaggaaggaattcaaggaacagtggctgaaggagcagaagcgcatgaatgaggcagtgcaggcactgcggcgctccgagctccagtacattcaacgcagagaggacctgagggcacgctcccaggggtcccctgaggaccctccttcccaggcatctcctggatccaacaagcaacaggagcgcagacggcgctcccgagaggaggcacaggccaaggcgcatgaggcggaggctctgtaccaggcctgtgtccgggaggccaattctcgccagcaggatctggagaccaccaagcggcggatagtgtcacatgtgcgcaagctggtgctgcaaggagacgaagtgcttaggcgggtgacgctgggtctgtttgagctgcgaggggcgcaggcagagcgaggaccccgctccttctctgctctggctgagtgctgtgtgccctttgaacctggccagcgctaccaggagtttgttcggacactgcagcctggggccccaccgccgccatctccggccttctgcttccaggagttcacagctgtggtgcacagtttccctcaggacacaaaaaagaagttttcggggcctctacctccaaggctggaggaggagggttcccctgagcctggcccttgggaggttgccagcttaggcagccagggaatcccaggcagtgatgtggacagtgtaggtggtggcagtgagtcccggtctttggattctcccacttccagcccaggtgctggggcccgccgacttgtaaaggcttcgtccacaggcacggagtcctccgatgactttgaggagcgagaccctgatctgggggatgggatagagaatggagtaggcagcccgttcaggaagtggacactgtccacagctgctcaaacccaccgactgcggcggctgcggggcccagccaagtgcagagaatgtgaagccttcatggtcagcgggacagaatgtgaagagtgctttttgacctgtcacaagcgctgcctggagaccctcctcatcctttgtggacaccggcggcttccggcccggatgtccctctttggggttgacttcctacagctccccagagatttccctgaggaggttccctttgtgattaccagatgcacagctgagatagagcaccgtgccctgggcttgcagggtatctatcgggtcagcgggtctcgggtacgtgtggagcggctgtgccaggcctttgagaatggccgagcactggtcgagctgtccgggaactctcctcacgatatcaccagcgttctcaagcgatttcttcaggagctcactgatcccgtggtccccttccacttgtacgacgccttcatctctctggcaaagaccctgcatgcagaccctggggacgaccctgggacccccaaccccagccctgagattattcgctcgctgaagaccctcttggtgcagctgcctgactctaactacagtaccctgcgacacctggtggcccatctgttcagggtggctgctcggtttgaagaaaacaagatgtctgccaacaatctgggaattgtatttgggcctacactgctgcggccaccagatggacccagggccaccggtgccagccctgtggcctgtctgctggactccggtcaccaggctcagctggttgagttcctcattgtgcactatgagcagatctttggaatggatgagctccctctggcctcggagcctctgacccaagaccctggcttggctcctgcatgccttgaatccagtccccagcacccagcctcacttcttgcccaagacacacagcccctgaccatagccttggattccagcccagaccccaaacaccacagtgccttggagaagtgccccgaggtcacacctcctgagcttgcaactctgcagagggaccagagggaggaggaggtggaagacaccagagatggggcaggggatgggtccagccactgccctgaggacttggccctgggagcacaatcccggggccacttcagccgccagccagtgaagtattcccggggaggtgtgaggccagttactcatcaactctccagcctggctctggtggcctccaagctgtgtgaggagactcctgtcactgtctcagcagtgcaccgaggtagtttgagggtacgaggtttgggccctgctgctgcctgccctgaaggcagccccctgcgccggaaccctctgcccaagcactttgagatcacccaggagacagcccgacttctctccaagctgaacagtgatgctgtgtccaggaccacctgttgtgctgacccagagcctgaggagtctgaagagcacctctgaccacctaccatcctgagggacccagaactgaattgatggtccctgacccaacctggtgaccaaaccaattcagtggggtgtggggagagacggggttaccagtgagttccggtaatgaagattacctacctgtgggagtcccaagacacttggttgaagtgtcccatcatctccctgccagaggagatttaaacagctaggcaacacaggtgcaggtgtttcggggtcaagactcaatggtcaatacagatcagtagatccttggggactacccgagtctctgacctctgagacgggtgtcttgactatttcagtcataatggctcctggccctactacacttgcagcagtaacagactcccaagttagcaggtggctgcagcccagccctgtctccttttaatttgtttgtttgtttgtttgttttaagctaagtctcactgtgtagctctggctggtctggagctccctttgtgagccacaaatcctctgcctcccaagtgctggctaaatctgtgttcttacaacctccaaataaaaccatatcctgctgccggcaggagggctgaccgcagtggtgcttgctgtaaagcctcacatttgttggacctcccagagccacttagtggaaggagagagcccatccctacaaattgtctgacctgtgcacatgcacaaataaaagaaaattttaatttaaaaaaaaaaatccgctgggcggtagtgccacacacctctacttgatcctagcactcaggagatagaggcaggaggatctctgagttttatgccagccaggtgtacagagaaagttccaggacagccagggctacacagagaaaccctgtttcagaaaaacaaaataaaataagataaatcttgtctgttcttgc

SEQ ID NO: 180 - DNA, 3922 bp, Mus Musculus
ggggcgacaccactgggtcggcagaggcaggaaatcgggctggtcccgggcggcgcgcagccctgtccctttctcagcattgccgcgcgttccccaccccaccccctgcccccgggcatcgccatggactcagcggagacagagctaaccccagctcctgagggcaggaagagatacagtgacatcttccagagcctggacaacctcgagatatcattggggaacgtaacttttgatcccctggctggagaccctgtacgcagagaagacctggagcctgacaaagctgacacagccacagtggtgactgaggagaacagtgaagccagtagttggcgggatctctccccagaaggtcctgcacccctcacagaggaagaactggatttgcgactcattcggaccaagggtggtgtagatgctgccctggagtatgctaaagcatggagccgctatgccaaggaactgctggcctggacagacaagagagccaactacgagctcgagtttgctaagagtatcatgaagctcgctgaggctggcaaggtgtccattctccagcagagccaaatgccactccagtacatctacaccttgtttctggagcatgacctcagcctgggagccctggccctggagacagtggcccaacagaagagagactactaccagcctctggcggccaaaaggatggaaattgagaagtggaggaaggaattcaaggaacagtggctgaaggagcagaagcgcatgaatgaggcagtgcaggcactgcggcgctccgagctccagtacattcaacgcagagaggacctgagggcacgctcccaggggtcccctgaggaccctccttcccaggcatctcctggatccaacaagcaacaggagcgcagacggcgctcccgagaggaggcacaggccaaggcgcatgaggcggaggctctgtaccaggcctgtgtccgggaggccaattctcgccagcaggatctggagaccaccaagcggcggatagtgtcacatgtgcgcaagctggtgctgcaaggagacgaagtgcttaggcgggtgacgctgggtctgtttgagctgcgaggggcgcaggcagagcgaggaccccgctccttctctgctctggctgagtgctgtgtgccctttgaacctggccagcgctaccaggagtttgttcggacactgcagcctggggccccaccgccgccatctccggccttctgcttccaggagttcacagctgtggtgcacaggacacaaaaaagaagttttcggggcctctacctccaaggctggaggaggagggttcccctgagcctggcccttgggaggttgccagcttaggcagccagggaatcccaggcagtgatgtggacagtgtaggtggtggcagtgagtcccggtctttggattctcccacttccagcccaggtgctggggcccgccgacttgtaaaggcttcgtccacaggcacggagtcctccgatgactttgaggagcgagaccctgatctgggggatgggatagagaatggagtaggcagcccgttcaggaagtggacactgtccacagctgctcaaacccaccgactgcggcggctgcggggcccagccaagtgcagagaatgtgaagccttcatggtcagcgggacagaatgtgaagagtgctttttgacctgtcacaagcgctgcctggagaccctcctcatcctttgtggacaccggcggcttccggcccggatgtccctctttggggttgacttcctacagctccccagagatttccctgaggaggttccctttgtgattaccagatgcacagctgagatagagcaccgtgccctgggcttgcagggtatctatcgggtcagcgggtctcgggtacgtgtggagcggctgtgccaggcctttgagaatggccgagcactggtcgagctgtccgggaactctcctcacgatatcaccagcgttctcaagcgatttcttcaggagctcactgatcccgtggtccccttccacttgtacgacgccttcatctctctggcaaagaccctgcatgcagaccctggggacgaccctgggacccccaaccccagccctgagattattcgctcgctgaagaccctcttggtgcagctgcctgactctaactacagtaccctgcgacacctggtggcccatctgttcagggtggctgctcggtttgaagaaaacaagatgtctgccaacaatctgggaattgtatttgggcctacactgctgcggccaccagatggacccagggccaccggtgccagccctgtggcctgtctgctggactccggtcaccaggctcagctggttgagttcctcattgtgcactatgagcagatctttggaatggatgagctccctctggcctcggagcctctgacccaagaccctggcttggctcctgcatgccttgaatccagtccccagcacccagcctcacttcttgcccaagacacacagcccctgaccatagccttggattccagcccagaccccaaacaccacagtgccttggagaagtgccccgaggtcacacctcctgagcttgcaactctgcagagggaccagagggaggaggaggtggaagacaccagagatggggcaggggatgggtccagccactgccctgaggacttggccctgggagcacaatcccggggccacttcagccgccagccagtgaagtattcccggggaggtgtgaggccagttactcatcaactctccagcctggctctggtggcctccaagctgtgtgaggagactcctgtcactgtctcagcagtgcaccgaggtagtttgagggtacgaggtttgggccctgctgctgcctgccctgaaggcagccccctgcgccggaaccctctgcccaagcactttgagatcacccaggagacagcccgacttctctccaagctgaacagtgatgctgtgtccaggaccacctgttgtgctgacccagagcctgaggagtctgaagagcacctctgaccacctaccatcctgagggacccagaactgaattgatggtccctgacccaacctggtgaccaaaccaattcagtggggtgtggggagagacggggttaccagtgagttccggtaatgaagattacctacctgtgggagtcccaagacacttggttgaagtgtcccatcatctccctgccagaggagatttaaacagctaggcaacacaggtgcaggtgtttcggggtcaagactcaatggtcaatacagatcagtagatccttggggactacccgagtctctgacctctgagacgggtgtcttgactatttcagtcataatggctcctggccctactacacttgcagcagtaacagactcccaagttagcaggtggctgcagcccagccctgtctccttttaatttgtttgtttgtttgtttgttttaagctaagtctcactgtgtagctctggctggtctggagctccctttgtgagccacaaatcctctgcctcccaagtgctggctaaatctgtgttcttacaacctccaaataaaaccatatcctgctgccggcaggagggctgaccgcagtggtgcttgctgtaaagcctcacatttgttggacctcccagagccacttagtggaaggagagagcccatccctacaaattgtctgacctgtgcacatgcacaaataaaagaaaattttaatttaaaaaaaaaaatccgctgggcggtagtgccacacacctctacttgatcctagcactcaggagatagaggcaggaggatctctgagttttatgccagccaggtgtacagagaaagttccaggacagccagggctacacagagaaaccctgtttcagaaaaacaaaataaaataagataaatcttgtctgttcttgc

SEQ ID NO: 181 - DNA, 3865 bp, Mus Musculus
ggggcgacaccactgggtcggcagaggcaggaaatcgggctggtcccgggcggcgcgcagccctgtccctttctcagcattgccgcgcgttccccaccccaccccctgcccccgggcatcgccatggactcagcggagacagagctaaccccagctcctgagggcaggaagagatacagtgacatcttccagagcctggacaacctcgagatatcattggggaacgtaacttttgatcccctggctggagaccctgtacgcagagaagacctggagcctgacaaagctgacacagccacagtggtgactgaggagaacagtgaagccagtagttggcgggatctctccccagaaggtcctgcacccctcacagaggaagaactggatttgcgactcattcggaccaagggtggtgtagatgctgccctggagtatgctaaagcatggagccgctatgccaaggaactgctggcctggacagacaagagagccaactacgagctcgagtttgctaagagtatcatgaagctcgctgaggctggcaaggtgtccattctccagcagagccaaatgccactccagtacatctacaccttgtttctggagcatgacctcagcctgggagccctggccctggagacagtggcccaacagaagagagactactaccagcctctggcggccaaaaggatggaaattgagaagtggaggaaggaattcaaggaacagtggctgaaggagcagaagcgcatgaatgaggcagtgcaggcactgcggcgctccgagctccagtacattcaacgcagagaggacctgagggcacgctcccaggggtcccctgaggaccctccttcccaggcatctcctggatccaacaagcaacaggagcgcagacggcgctcccgagaggaggcacaggccaaggcgcatgaggcggaggctctgtaccaggcctgtgtccgggaggccaattctcgccagcaggatctggagaccaccaagcggcggatagtgtcacatgtgcgcaagctggtgctgcaaggagacgaagtgcttaggcgggtgacgctgggtctgtttgagctgcgaggggcgcaggcagagcgaggaccccgctccttctctgctctggctgagtgctgtgtgccctttgaacctggccagcgctaccaggagtttgttcggacactgcagcctggggccccaccgccgccatctccggccttctgcttccaggagttcacagctgtggtgcacagtttccctcaggacacaaaaaagaagttttcggggcctctacctccaaggctggaggaggagggttcccctgagcctggcccttgggaggttgccagcttaggcagccagggaatcccaggcagtgatgtggacagtgtaggtggtggcagtgagtcccggtctttggattctcccacttccagcccaggtgctggggcccgccgacttgtaaaggcttcgtccacaggcacggagtcctccgatgactttgaggagcgagaccctgatctgggggatgggatagagaatggagtaggcagcccgttcaggaagtggacactgtccacagctgctcaaacccaccgactgcggcggctgcggggcccagccaagtgcagagaatgtgaagccttcatggtcagcgggacagaatgtgaagagtgctttttgacctgtcacaagcgctgcctggagaccctcctcatcctttgtggacaccggcggcttccggcccggatgtccctctttggggttgacttcctacagctccccagagatttccctgaggaggttccctttgtgattaccagatgcacagctgagatagagcaccgtgccctgggcttgcagggtatctatcgggtcagcgggtctcgggtacgtgtggagcggctgtgccaggcctttgagaatggccgagcactggtcgagctgtccgggaactctcctcacgatatcaccagcgttctcaagcgatttcttcaggagctcactgatcccgtggtccccttccacttgtacgacgccttcatctctctggcaaagaccctgcatgcagaccctggggacgaccctgggacccccaaccccagccctgagattattcgctcgctgaagaccctcttggtgcagctgcctgactctaactacagtaccctgcgacacctggtggcccatctgttcagggtggctgctcggtttgaagaaaacaagatgtctgccaacaatctgggaattgtatttgggcctacactgctgcggccaccagatggacccagggccaccggtgccagccctgtggcctgtctgctggactccggtcaccaggctcagctggttgagttcctcattgtgcactatgagcagatctttggaatggatgagctccctctggcctcggagcctctgacccaagaccctggcttggctcctgcatgccttgaatccagtccccagcacccagcctcacttcttgcccaagacacacagcccctgaccatagccttggattccagcccagaccccaaacaccacagtgccttggagaagtgccccgaggtcacacctcctgagggtccagccactgccctgaggacttggccctgggagcacaatcccggggccacttcagccgccagccagtgaagtattcccggggaggtgtgaggccagttactcatcaactctccagcctggctctggtggcctccaagctgtgtgaggagactcctgtcactgtctcagcagtgcaccgaggtagtttgagggtacgaggtttgggccctgctgctgcctgccctgaaggcagccccctgcgccggaaccctctgcccaagcactttgagatcacccaggagacagcccgacttctctccaagctgaacagtgatgctgtgtccaggaccacctgttgtgctgacccagagcctgaggagtctgaagagcacctctgaccacctaccatcctgagggacccagaactgaattgatggtccctgacccaacctggtgaccaaaccaattcagtggggtgtggggagagacggggttaccagtgagttccggtaatgaagattacctacctgtgggagtcccaagacacttggttgaagtgtcccatcatctccctgccagaggagatttaaacagctaggcaacacaggtgcaggtgtttcggggtcaagactcaatggtcaatacagatcagtagatccttggggactacccgagtctctgacctctgagacgggtgtcttgactatttcagtcataatggctcctggccctactacacttgcagcagtaacagactcccaagttagcaggtggctgcagcccagccctgtctccttttaatttgtttgtttgtttgtttgttttaagctaagtctcactgtgtagctctggctggtctggagctccctttgtgagccacaaatcctctgcctcccaagtgctggctaaatctgtgttcttacaacctccaaataaaaccatatcctgctgccggcaggagggctgaccgcagtggtgcttgctgtaaagcctcacatttgttggacctcccagagccacttagtggaaggagagagcccatccctacaaattgtctgacctgtgcacatgcacaaataaaagaaaattttaatttaaaaaaaaaaatccgctgggcggtagtgccacacacctctacttgatcctagcactcaggagatagaggcaggaggatctctgagttttatgccagccaggtgtacagagaaagttccaggacagccagggctacacagagaaaccctgtttcagaaaaacaaaataaaataagataaatcttgtctgttcttgc

SEQ ID NO: 182 - DNA, 2764 bp, Mus Musculus
ggggcgacaccactgggtcggcagaggcaggaaatcgggctggtcccgggcggcgcgcagccctgtccctttctcagcattgccgcgcgttccccaccccaccccctgcccccgggcatcgccatggactcagcggagacagagctaaccccagctcctgagggcaggaagagatacagtgacatcttccagagcctggacaacctcgagatatcattggggaacgtaacttttgatcccctggctggagaccctgtacgcagagaagacctggagcctgacaaagctgacacagccacagtggtgactgaggagaacagtgaagccagtagttggcgggatctctccccagaaggtcctgcacccctcacagaggaagaactggatttgcgactcattcggaccaagggtggtgtagatgctgccctggagtatgctaaagcatggagccgctatgccaaggaactgctggcctggacagacaagagagccaactacgagctcgagtttgctaagagtatcatgaagctcgctgaggctggcaaggtgtccattctccagcagagccaaatgccactccagtacatctacaccttgtttctggagcatgacctcagcctgggagccctggccctggagacagtggcccaacagaagagagactactaccagcctctggcggccaaaaggatggaaattgagaagtggaggaaggaattcaaggaacagtggctgaaggagcagaagcgcatgaatgaggcagtgcaggcactgcggcgctccgagctccagtacattcaacgcagagaggacctgagggcacgctcccaggggtcccctgaggaccctccttcccaggcatctcctggatccaacaagcaacaggagcgcagacggcgctcccgagaggaggcacaggccaaggcgcatgaggcggaggctctgtaccaggcctgtgtccgggaggccaattctcgccagcaggatctggagaccaccaagcggcggatagtgtcacatgtgcgcaagctggtgctgcaaggagacgaagtgcttaggcgggtgacgctgggtctgtttgagctgcgaggggcgcaggcagagcgaggaccccgctccttctctgctctggctgagtgctgtgtgccctttgaacctggccagcgctaccaggagtttgttcggacactgcagcctggggccccaccgccgccatctccggccttctgcttccaggagttcacagctgtggtgcacagtttccctcaggacacaaaaaagaagttttcggggcctctacctccaaggctggaggaggagggttcccctgagcctggcccttgggaggttgccagcttaggcagccagggaatcccaggcagtgatgtggacagtgtaggtggtggcagtgagtcccggtctttggattctcccacttccagcccaggtgctggggcccgccgacttgtaaaggcttcgtccacaggcacggagtcctccgatgactttgaggagcgagaccctgatctgggggatgggatagagaatggagtaggcagcccgttcaggaagtggacactgtccacagctgctcaaacccaccgactgcggcggctgcggggcccagccaagtgcagagaatgtgaagccttcatggtcagcgggacagaatgtgaagagtgctttttgacctgtcacaagcgctgcctggagaccctcctcatcctttgtggacaccggcggcttccggcccggatgtccctctttggggttgacttcctacagctccccagagatttccctgaggaggttccctttgtgattaccagatgcacagctgagatagagcaccgtgccctgggcttgcagggtatctatcgggtcagcgggtctcgggtacgtgtggagcggctgtgccaggcctttgagaatggccgagcactggtcgagctgtccgggaactctcctcacgatatcaccagcgttctcaagcgatttcttcaggagctcactgatcccgtggtccccttccacttgtacgacgccttcatctctctggcaaagaccctgcatgcagaccctggggacgaccctgggacccccaaccccagccctgagattattcgctcgctgaagaccctcttggtgcagctgcctgactctaactacagtaccctgcgacacctggtggcccatctgttcagggtggctgctcggtttgaagaaaacaagatgtctgccaacaatctgggaattgtatttgggcctacactgctgcggccaccagatggacccagggccaccggtgccagccctgtggcctgtctgctggactccggtcaccaggctcagctggttgagttcctcattgtgcactatgagcagatctttggaatggatgagctccctctggcctcggagcctctgacccaagaccctggcttggctcctgcatgccttgaatccagtccccagcacccagcctcacttcttgcccaagacacacagcccctgaccatagccttggattccagcccagaccccaaacaccacagtgccttggagaagtgccccgaggtcacacctcctgagttttggtttttcgagacagggtttctctgtgtagccctggctgtcctggaactcactctgtagaccaggctggcctcgaactcaaaaatctgcctgcctctgcctcccgagtgctgggattaaaggcctgcgccaccaccgcccagctgttctgttgttttatctgactttg

SEQ ID NO: 183 - DNA, 3735 bp, Mus Musculus
ggggcgacaccactgggtcggcagaggcaggaaatcgggctggtcccgggcggcgcgcagccctgtccctttctcagcattgccgcgcgttccccaccccaccccctgcccccgggcatcgccatggactcagcggagacagagctaaccccagctcctgagggcaggaagagatacagtgacatcttccagagcctggacaacctcgagatatcattggggaacgtaacttttgatcccctggctggagaccctgtacgcagagaagacctggagcctgacaaagctgacacagccacagtggtgactgaggagaacagtgaagccagtagttggcgggatctctccccagaaggtcctgcacccctcacagaggaagaactggatttgcgactcattcggaccaagggtggtgtagatgctgccctggagtatgctaaagcatggagccgctatgccaaggaactgctggcctggacagacaagagagccaactacgagctcgagtttgctaagagtatcatgaagctcgctgaggctggcaaggtgtccattctccagcagagccaaatgccactccagtacatctacaccttgtttctggagcatgacctcagcctgggagccctggccctggagacagtggcccaacagaagagagactactaccagcctctggcggccaaaaggatggaaattgagaagtggaggaaggaattcaaggaacagtggctgaaggagcagaagcgcatgaatgaggcagtgcaggcactgcggcgctccgagctccagtacattcaacgcagagaggacctgagggcacgctcccaggggtcccctgaggaccctccttcccaggcatctcctggatccaacaagcaacaggagcgcagacggcgctcccgagaggaggcacaggccaaggcgcatgaggcggaggctctgtaccaggcctgtgtccgggaggccaattctcgccagcaggatctggagaccaccaagcggcggatagtgtcacatgtgcgcaagctggtgctgcaaggagacgaagtgcttaggcgggtgacgctgggtctgtttgagctgcgaggggcgcaggcagagcgaggaccccgctccttctctgctctggctgagtgctgtgtgccctttgaacctggccagcgctaccaggagtttgttcggacactgcagcctggggccccaccgccgccatctccggccttctgcttccaggagttcacagctgtggtgcacagtttccctcaggacacaaaaaagaagttttcggggcctctacctccaaggctggaggaggagggttcccctgagcctggcccttgggaggttgccagcttaggcagccagggaatcccaggcagtgatgtggacagtgtaggtggtggcagtgagtcccggtctttggattctcccacttccagcccaggtgctggggcccgccgacttgtaaaggcttcgtccacaggcacggagtcctccgatgactttgaggagcgagaccctgatctgggggatgggatagagaatggagtaggcagcccgttcaggaagtggacactgtccacagctgctcaaacccaccgactgcggcggctgcggggcccagccaagtgcagagaatgtgaagccttcatggtcagcgggacagaatgtgaagagtgctttttgacctgtcacaagcgctgcctggagaccctcctcatcctttgtggacaccggcggcttccggcccggatgtccctctttggggttgacttcctacagctccccagagatttccctgaggaggttccctttgtgattaccagatgcacagctgagatagagcaccgtgccctgggcttgcagggtatctatcgggtcagcgggtctcgggtacgtgtggagcggctgtgccaggcctttgagaatggccgagcactggtcgagctgtccgggaactctcctcacgatatcaccagcgttctcaagcgatttcttcaggagggtggctgctcggtttgaagaaaacaagatgtctgccaacaatctgggaattgtatttgggcctacactgctgcggccaccagatggacccagggccaccggtgccagccctgtggcctgtctgctggactccggtcaccaggctcagctggttgagttcctcattgtgcactatgagcagatctttggaatggatgagctccctctggcctcggagcctctgacccaagaccctggcttggctcctgcatgccttgaatccagtccccagcacccagcctcacttcttgcccaagacacacagcccctgaccatagccttggattccagcccagaccccaaacaccacagtgccttggagaagtgccccgaggtcacacctcctgagcttgcaactctgcagagggaccagagggaggaggaggtggaagacaccagagatggggcaggggatgggtccagccactgccctgaggacttggccctgggagcacaatcccggggccacttcagccgccagccagtgaagtattcccggggaggtgtgaggccagttactcatcaactctccagcctggctctggtggcctccaagctgtgtgaggagactcctgtcactgtctcagcagtgcaccgaggtagtttgagggtacgaggtttgggccctgctgctgcctgccctgaaggcagccccctgcgccggaaccctctgcccaagcactttgagatcacccaggagacagcccgacttctctccaagctgaacagtgatgctgtgtccaggaccacctgttgtgctgacccagagcctgaggagtctgaagagcacctctgaccacctaccatcctgagggacccagaactgaattgatggtccctgacccaacctggtgaccaaaccaattcagtggggtgtggggagagacggggttaccagtgagttccggtaatgaagattacctacctgtgggagtcccaagacacttggttgaagtgtcccatcatctccctgccagaggagatttaaacagctaggcaacacaggtgcaggtgtttcggggtcaagactcaatggtcaatacagatcagtagatccttggggactacccgagtctctgacctctgagacgggtgtcttgactatttcagtcataatggctcctggccctactacacttgcagcagtaacagactcccaagttagcaggtggctgcagcccagccctgtctccttttaatttgtttgtttgtttgtttgttttaagctaagtctcactgtgtagctctggctggtctggagctccctttgtgagccacaaatcctctgcctcccaagtgctggctaaatctgtgttcttacaacctccaaataaaaccatatcctgctgccggcaggagggctgaccgcagtggtgcttgctgtaaagcctcacatttgttggacctcccagagccacttagtggaaggagagagcccatccctacaaattgtctgacctgtgcacatgcacaaataaaagaaaattttaatttaaaaaaaaaaatccgctgggcggtagtgccacacacctctacttgatcctagcactcaggagatagaggcaggaggatctctgagttttatgccagccaggtgtacagagaaagttccaggacagccagggctacacagagaaaccctgtttcagaaaaacaaaataaaataagataaatcttgtctgttcttgc

SEQ ID NO: 184 - DNA, 3874 bp, Mus Musculus
ggggcgacaccactgggtcggcagaggcaggaaatcgggctggtcccgggcggcgcgcagccctgtccctttctcagcattgccgcgcgttccccaccccaccccctgcccccgggcatcgccatggactcagcggagacagagctaaccccagctcctgagggcaggaagagatacagtgacatcttccagagcctggacaacctcgagatatcattggggaacgtaacttttgatcccctggctggagaccctgtacgcagagaagacctggagcctgacaaagctgacacagccacagtggtgactgaggagaggaagaactggatttgcgactcattcggaccaagggtggtgtagatgctgccctggagtatgctaaagcatggagccgctatgccaaggaactgctggcctggacagacaagagagccaactacgagctcgagtttgctaagagtatcatgaagctcgctgaggctggcaaggtgtccattctccagcagagccaaatgccactccagtacatctacaccttgtttctggagcatgacctcagcctgggagccctggccctggagacagtggcccaacagaagagagactactaccagcctctggcggccaaaaggatggaaattgagaagtggaggaaggaattcaaggaacagtggctgaaggagcagaagcgcatgaatgaggcagtgcaggcactgcggcgctccgagctccagtacattcaacgcagagaggacctgagggcacgctcccaggggtcccctgaggaccctccttcccaggcatctcctggatccaacaagcaacaggagcgcagacggcgctcccgagaggaggcacaggccaaggcgcatgaggcggaggctctgtaccaggcctgtgtccgggaggccaattctcgccagcaggatctggagaccaccaagcggcggatagtgtcacatgtgcgcaagctggtgctgcaaggagacgaagtgcttaggcgggtgacgctgggtctgtttgagctgcgaggggcgcaggcagagcgaggaccccgctccttctctgctctggctgagtgctgtgtgccctttgaacctggccagcgctaccaggagtttgttcggacactgcagcctggggccccaccgccgccatctccggccttctgcttccaggagttcacagctgtggtgcacagtttccctcaggacacaaaaaagaagttttcggggcctctacctccaaggctggaggaggagggttcccctgagcctggcccttgggaggttgccagcttaggcagccagggaatcccaggcagtgatgtggacagtgtaggtggtggcagtgagtcccggtctttggattctcccacttccagcccaggtgctggggcccgccgacttgtaaaggcttcgtccacaggcacggagtcctccgatgactttgaggagcgagaccctgatctgggggatgggatagagaatggagtaggcagcccgttcaggaagtggacactgtccacagctgctcaaacccaccgactgcggcggctgcggggcccagccaagtgcagagaatgtgaagccttcatggtcagcgggacagaatgtgaagagtgctttttgacctgtcacaagcgctgcctggagaccctcctcatcctttgtggacaccggcggcttccggcccggatgtccctctttggggttgacttcctacagctccccagagatttccctgaggaggttccctttgtgattaccagatgcacagctgagatagagcaccgtgccctgggcttgcagggtatctatcgggtcagcgggtctcgggtacgtgtggagcggctgtgccaggcctttgagaatggccgagcactggtcgagctgtccgggaactctcctcacgatatcaccagcgttctcaagcgatttcttcaggagctcactgatcccgtggtccccttccacttgtacgacgccttcatctctctggcaaagaccctgcatgcagaccctggggacgaccctgggacccccaaccccagccctgagattattcgctcgctgaagaccctcttggtgcagctgcctgactctaactacagtaccctgcgacacctggtggcccatctgttcagggtggctgctcggtttgaagaaaacaagatgtctgccaacaatctgggaattgtatttgggcctacactgctgcggccaccagatggacccagggccaccggtgccagccctgtggcctgtctgctggactccggtcaccaggctcagctggttgagttcctcattgtgcactatgagcagatctttggaatggatgagctccctctggcctcggagcctctgacccaagaccctggcttggctcctgcatgccttgaatccagtccccagcacccagcctcacttcttgcccaagacacacagcccctgaccatagccttggattccagcccagaccccaaacaccacagtgccttggagaagtgccccgaggtcacacctcctgagcttgcaactctgcagagggaccagagggaggaggaggtggaagacaccagagatggggcaggggatgggtccagccactgccctgaggacttggccctgggagcacaatcccggggccacttcagccgccagccagtgaagtattcccggggaggtgtgaggccagttactcatcaactctccagcctggctctggtggcctccaagctgtgtgaggagactcctgtcactgtctcagcagtgcaccgaggtagtttgagggtacgaggtttgggccctgctgctgcctgccctgaaggcagccccctgcgccggaaccctctgcccaagcactttgagatcacccaggagacagcccgacttctctccaagctgaacagtgatgctgtgtccaggaccacctgttgtgctgacccagagcctgaggagtctgaagagcacctctgaccacctaccatcctgagggacccagaactgaattgatggtccctgacccaacctggtgaccaaaccaattcagtggggtgtggggagagacggggttaccagtgagttccggtaatgaagattacctacctgtgggagtcccaagacacttggttgaagtgtcccatcatctccctgccagaggagatttaaacagctaggcaacacaggtgcaggtgtttcggggtcaagactcaatggtcaatacagatcagtagatccttggggactacccgagtctctgacctctgagacgggtgtcttgactatttcagtcataatggctcctggccctactacacttgcagcagtaacagactcccaagttagcaggtggctgcagcccagccctgtctccttttaatttgtttgtttgtttgtttgttttaagctaagtctcactgtgtagctctggctggtctggagctccctttgtgagccacaaatcctctgcctcccaagtgctggctaaatctgtgttcttacaacctccaaataaaaccatatcctgctgccggcaggagggctgaccgcagtggtgcttgctgtaaagcctcacatttgttggacctcccagagccacttagtggaaggagagagcccatccctacaaattgtctgacctgtgcacatgcacaaataaaagaaaattttaatttaaaaaaaaaaatccgctgggcggtagtgccacacacctctacttgatcctagcactcaggagatagaggcaggaggatctctgagttttatgccagccaggtgtacagagaaagttccaggacagccagggctacacagagaaaccctgtttcagaaaaacaaaataaaataagataaatcttgtctgttcttgc

SEQ ID NO: 185 - DNA, 845 bp, Mus Musculus
agcagttaaagagcactagctttgctttcagaggactcagatttggttcttagcatccacatggtggttcacagtcatcagtgactctagtcccaggggattaaataccctttcagacctccatgggcactgactgcaggcacatggtgaaggaaaaacacttccatacataaaaataattactacaatttaagctaaaagggcaccaaacactttatagatcttgatgagaggctatatcaaattttatacattgtgacaccggttttttatagattttctagcatatattttagatctatttatcatctatctatctatctatctatctatctttctatcatctatcttatataaaataaagtctgacctaaatatgagaccctgtatggttgtctaaaaacatactgttagctccttcagtcacacctgtttggggctggctggtgaatcagaaggaaccttgttgggtctgctgctcaccacctatggggctgtgggcatggtggcagcagcagttgttgccacttctgctttggccaagcacagtaggtgatcctgatagtcagcagcacagattcccattgattctagtctatgtatttgcttatggttggtacaatatttaatgtatataggcagaagtattatagggtctttaaatatgtgaacatatgtagcactgcttttaaacttaaaacggatatgatatttttcatatgtcaagacacactggtaaatttctgttacatattgttttcaagttttctgttagtcttgtttctatgaggtacttttattgtaacttaaaattcttagaaattaaagttctgagaaaaacctta

SEQ ID NO: 186 - DNA, 1871 bp, Mus Musculus
gtcttccccagaggccctaactacagccactaccctttcagggcccaggctgaccgtggatggggctcctggcaccttcatcctcctcctccatcttctatctgagcctgagacgaactgcccaccccctccagccacctgccgcctcctgtttggaaggaggcccgcagcagatttcctcttccctccattcccaagaggaacccctggtctggggatactgaggcaggcacagggcagggcacgtgtgagcatccagtcatcctgaaaggtaaatctggttcctgaagcgacaactgttcgtacagggttttgaaggattggtaggagttccacagaggacaccgaagaggtgcagtccctggtttgtccttatgtcccagcccatttgggcacagctgtaatgtctactcctgggtcactgaggcataggggtgacttgcctgaacttgccttttctaggttttccttggcttcacctttgatgggggtggagggaggagggagggaaagaggaggaaagagagagggagagagtcctggtcccaagaggaaggaagaagccacttgaacttcagtctggactccatcacatgttctgtggggacggtcagccctcactcaagtcccagacccggcggtttccctccctcccaccccctccccctgcggagccctgggattcccgcccgaattagcctggtcacccgagttggcatcgcacacgggggcggagcggacagagtccagtgggtataaaggcagctgcagagggcttaactcaaagacacactcaggacacaagcgacatggccccgcccgcgctccaggcccagcctccaggcggctctcaactgaggttcctgctgttcctgctgctgttgctgctgctgctgtcatggccatcgcagggggacgccctggcaatgcctgaacagcgaccctccggccctgagtcccaactcaacgccgacgagctacggggtcgcttccaggacctgctgagccggctgcatgccaaccagagccgagaggactcgaactcagaaccaagtcctgacccagctgtccggatactcagtccagaggtgagattggggtcccacggccagctgctactccgcgtcaaccgggcgtcgctgagtcagggtctccccgaagcctaccgcgtgcaccgagcgctgctcctgctgacgccgacggcccgcccctgggacatcactaggcccctgaagcgtgcgctcagcctccagggaccccgtgctcccgcattacgcctgcgcctgacgccgcctccggacctggctatgctgccctctggcggcacacagctggaactgcgcttacgggtagccgccggcagggggcgccgaagcgcgcatgcgcacccaagagactcgtgcccactgggtccggggcgctgctgtcacctggagactgtgcaggcaactcttgaagacttgggctggagcgactgggtgctgtccccgcgccagctgcagctgagcatgtgcgtgggcgagtgtccccacctgtatcgctccgcgaacacgcatgcgcagatcaaagcacgcctgcatggcctgcagcctgacaaggtgcctgccccgtgctgtgtcccctccagctacaccccggtggttcttatgcacaggacagacagtggtgtgtcactgcagacttatgatgacctggtggcccggggctgccactgcgcttgagcaccgggccctgctcctcacctacactccccttcaaggatgctatttatatttgtatttattaatattattaatttattggggtcgggctgggtggatggattgtgtatttatttaaaactctgctaataaaggtgagcttggtttcta

SEQ ID NO: 187 - DNA, 1916 bp, Mus Musculus
tgcagcagccgagaggtgtgagccgccgcggtgtcagagtctaggggaattggagtcaggcgcagatccacagcgatatccagacattcagagccacaggcaccatgtccaatcctggtgatgtccgacctgttccgcacaggagcaaagtgtgccgttgtctcttcggtcccgtggacagtgagcagttgcgccgtgattgcgatgcgctcatggcgggctgtctccaggaggcccgagaacggtggaactttgacttcgtcacggagacgccgctggagggcaacttcgtctgggagcgcgttcggagcctagggctgcccaaggtctacctgagccctgggtcccgcagccgtgacgacctgggaggggacaagaggcccagtacttcctctgccctgctgcaggggccagctccggaggaccacgtggccttgtcgctgtcttgcactctggtgtctgagcggcctgaagattccccgggtgggcccggaacatctcagggccgaaaacggaggcagaccagcctgacagatttctatcactccaagcgcagattggtcttctgcaagagaaaaccctgaagtgcccacgggagccccgccctcttctgctgtgggtcaggaggcctcttccccatcttcggccttagccctcactctgtgtgtcttaattattatttgtgttttaatttaaacgtctcctgtatatacgctgcctgccctctcccagtctccaaacttaaagttatttaaaaaaagaacaaaacaaaacaaaaaaaaccaaaacaaaacaaacctaaattagtaggacggtagggcccttagtgtgggggatttctattatgtagattattattatttaagcccctcccaacccaagctctgtgtttcctataccggaggaacagtcctactgatatcaacccatctgcatccgtttcacccaacccccctccccccattccctgcctggttccttgccacttcttacctgggggtgatcctcagacctgaatagcactttggaaaaatgagtaggactttggggtctccttgtcacctctaaggccagctaggatgacagtgaagcagtcacagcctagaacagggatggcagttaggactcaaccgtaatatcccgactcttgacattgctcagacctgtgaagacaggaatggtccccactctggatcccctttgccactcctggggagcccacctctcctgtgggtctctgccagctgcccctctattttggagggttaatctggtgatctgctgctcttttcccccaccccatacttccccttctgcaggtcggcaggaggcatatctaggcacttgccccacagctcagtggactggaagggaatgtatatgcagggtacactaagtgggattccctggtcttaccttaggcagctccagtggcaaccccctgcattgtgggtctagggtgggtccttggtggtgagacaggcctcccagagcattctatggtgtgtggtggtgggggtgggcttatctgggatggggaccccagttggggttctcagtgacttctcccatttcttagtagcagttgtacaaggagccaggccaagatggtgtcttgggggctaagggagctcacaggacactgagcaatggctgatcctttctcagtgttgaataccgtgggtgtcaaagcacttagtgggtctgactccagccccaaacatccctgtttctgtaacatcctggtctggactgtctacccttagcccgcaccccaagaacatgtattgtggctccctccctgtctccactcagattgtaagcgtctcacgagaagggacagcaccctgcattgtcccgagtcctcacacccgaccccaaagctggtgctcaataaatacttctcgatgatttat

SEQ ID NO: 188 - DNA, 3512 bp, Mus Musculus
agagaccatccatcaaaagaaccacaccaatcagcgacgtagatgcgtcccagctagagagccaggtctccagcttgttcccccactgcccggcgcagtccttcgctgcgcgctgaccccgaggaaggcagtacttggtcagggcgctgagggaccctccaccgggacgccggcccctccccgggcctctgctcacttgacccccactccctagtccgtccccttagtaggcctgtcggatcggggacgtggggcgagctgagagcaggcccggggtgggtggtcaccgtggtgaagacgtggctgtcaagatgatagaagtactgacaactgactctcagaaactgctacaccagctgaacaccctgttggaacaggagtctagatgtcagccaaaggtctgcggcttgaaactaattgagtcggcccatgataatggcctcagaatgactgcaagattacgggactttgaagtaaaagatctacttagtctaactcagttctttggctttgacacggagacattttccctagctgtgaatttactggacagattcttgtctaaaatgaaggtacaggcgaagcatcttgggtgtgttggactgagctgcttttatttggctgtgaaagcgactgaagaggaaaggaatgtcccactggcgactgatttgatccgaataagtcagtataggttcacggtttcagacctgatgagaatggagaagattgtgttggagaaagtgtgttggaaagtcaaagctactactgcctttcaatttctgcagctctattattcactcgttcacgacaccttgccatttgagaggagaaacgatctgaattttgaaagactagaagcccaacttaaggcctgccactgcaggatcatattttctaaggcaaagccttctgtgctggcgctatctatccttgcgttggagatccaagcactgaaatacgtagagttaacagaaggagtagaatgtattcagaaacattccaagataagtggccgagatttgaccttctggcaagagcttgtttccaagtgtttaactgaatattcatcaaacaagtgctccaaacctaacggtcagaagttgaaatggattgtgtctggacgcactgcacggcaactgaagcacagttattatagaataactcacctcccaacgattcctgagaccatttgttagttgataaatctggttgttattctctgtatacagaaaattttccagtatgatcattttctgctacaactgaagaattgaaatactatcttcaatataaagaatatgggatgaaaacataaaggaaaagtgaattgttgactggtctagatagagaatactggaaggcattcactgtgtacagtgcgtagcagttttaagagaaaagacatatcaaacccctagatatacgctaatacttttcatcaaaagattagcgtagtagcaaagagaatactttaaactcgaattttaaaagtagttactgaaatagcacttctttaaattacgtaccaccccactgtagcttatttaaagttgcataagcccatgcagaacaacaagcaatgtgacccatatatgaacaaattttaatctgcccatcgactatgaaaatgaagtacaaacctgggtgatggacttacaaagtaatatagggcatgcccatgttaggtttctggaaactgccagagtgtcttaattctatagctagtattttacctctatagcatttggactaatacaaagtaattatatgcatgaaaatataaaattggtctctgatacatacacatttttgacatctaaatttcttaatcatagcaaagatttatctttttatgattaaaaacaattttttccttaatgcatggcagcacatgcctttaatcccagcactagagaggcagagacaggtagatctctgagttcaaagccaggctggtctacacagtgagttccaagacagctagagaccttgtctcaaaaacactcaaaacaaccaaacaccaggggtaaatgttgctgggaagtcagggaagatggtttagaagggaagcctgacaacctgacttcattcttcaggacccacatgatggaaggagagaacgaagaactcccaagttctctcacatatgcacatacctcaccaccccccgcaggaaatacatgatcatgcgtctgagatatcaccagttcacctttagcagctcgcagtttgtaggcagatttctgttaagttgggtctgtgttgtttgcctatgtagcaggattacagcagcagcaaaaacggtccctcaagtctttctgccactctgacctgagtttcctacggtacaggatttactctgagaaacctcagcaccttgccacagtagccttggcagaatggcctcacggttagggaaactcctgattctaagcttgggagagctacgcttagatttgaattcacccaggaagcattcaaatcaaggctaaagacataaatgtgaaataaaactgtgaaccttcattctaaagttcatctgacttcccagatttgatcaatatattcttaggtggtattaaaaatggtaaactgcttaatttaaatctcaaaatttaaattatgaggtttacataaaaaccaacatttcatgaatgcacttttaaggtattaaaaggggtacttaagcggtaaatggtttcttggcacccataaccaagtaatagttaatttacaggtgggatttttttttattgctatgagaattacatttaaaattgtgggtgttttatataaagcagatatcacaagttttgaaaatttgttacctttaatatttcttctagagaataggtgtttgtatccataataaaagaaaaatttgtcagaactgctgcttcaatctaatcccatttgagagaattgtctttactgtcttaataactggatgaattatcactctgaaaatgtatttattgcactaaagttagtttaggcttgataaaacactccagacatttttactacagactgtttctataaaactgccattgcttctaatggagaattttattttaaaagaaataaaattgctgagtttatctgcaaaacctttctctaagtcttatgggacgtaaggagacacctccataattataagagccgttgtgctcagagtctccatgctggcttgaatgtatgatccacactgttaaaacataggcagcagctcagggccctggacagcctgagtcaacctagagtagctggaaccattttgacatgtaatggataagaaaattatccattgagaagctgaacaataaaccaaagaacgggtgtattttatccttaacctctgtaaaccacgttacactgagaacacttcagttcttcctaaaggttgataggcttcagtctgaaaacaatattgatttggagtggacagaagttaactaaccaactaccattatgttttgaatacacctttcaataaaatgattgaaatgcaa

SEQ ID NO: 189 - DNA, 3433 bp, Mus Musculus
agagaccatccatcaaaagaaccacaccaatcagcgacgtagatgcgtcccagctagagagccaggtctccagcttgttcccccactgcccggcgcagtccttcgctgcgcgctgaccccgaggaaggcagtacttggtcagggcgctgagggaccctccaccgggacgccggcccctccccgggcctctgctcacttgacccccactccctagtccgtccccttagtaggcctgtcggatcggggacgtggggcgagctgagagcaggcccggggtgggtggtcaccgtggtgaagacgtggctgtcaagatgatagaagtactgacaactgactctcagaaactgctacaccagctgaacaccctgttggaacaggagtctagatgtcagccaaaggtctgcggcttgaaactaattgagtcggcccatgataatggcctcagaatgactgcaagattacgggactttgaagtaaaagatctacttagtctaactcagttctttggctttgacacggagacattttccctagctgtgaatttactggacagattcttgtctaaaatgaaggtacaggcgaagcatcttgggtgtgttggactgagctgcttttatttggctgtgaaagcgactgaagaggaaaggaatgtcccactggcgactgatttgatccgaataagtcagtataggttcacggtttcagacctgatgagaatggagaagattgtgttggagaaagtgtgttggaaagtcaaagctactactgcctttcaatttctgcagctctattattcactcgttcacgacaccttgccatttgagagccttctgtgctggcgctatctatccttgcgttggagatccaagcactgaaatacgtagagttaacagaaggagtagaatgtattcagaaacattccaagataagtggccgagatttgaccttctggcaagagcttgtttccaagtgtttaactgaatattcatcaaacaagtgctccaaacctaacggtcagaagttgaaatggattgtgtctggacgcactgcacggcaactgaagcacagttattatagaataactcacctcccaacgattcctgagaccatttgttagttgataaatctggttgttattctctgtatacagaaaattttccagtatgatcattttctgctacaactgaagaattgaaatactatcttcaatataaagaatatgggatgaaaacataaaggaaaagtgaattgttgactggtctagatagagaatactggaaggcattcactgtgtacagtgcgtagcagttttaagagaaaagacatatcaaacccctagatatacgctaatacttttcatcaaaagattagcgtagtagcaaagagaatactttaaactcgaattttaaaagtagttactgaaatagcacttctttaaattacgtaccaccccactgtagcttatttaaagttgcataagcccatgcagaacaacaagcaatgtgacccatatatgaacaaattttaatctgcccatcgactatgaaaatgaagtacaaacctgggtgatggacttacaaagtaatatagggcatgcccatgttaggtttctggaaactgccagagtgtcttaattctatagctagtattttacctctatagcatttggactaatacaaagtaattatatgcatgaaaatataaaattggtctctgatacatacacatttttgacatctaaatttcttaatcatagcaaagatttatctttttatgattaaaaacaattttttccttaatgcatggcagcacatgcctttaatcccagcactagagaggcagagacaggtagatctctgagttcaaagccaggctggtctacacagtgagttccaagacagctagagaccttgtctcaaaaacactcaaaacaaccaaacaccaggggtaaatgttgctgggaagtcagggaagatggtttagaagggaagcctgacaacctgacttcattcttcaggacccacatgatggaaggagagaacgaagaactcccaagttctctcacatatgcacatacctcaccaccccccgcaggaaatacatgatcatgcgtctgagatatcaccagttcacctttagcagctcgcagtttgtaggcagatttctgttaagttgggtctgtgttgtttgcctatgtagcaggattacagcagcagcaaaaacggtccctcaagtctttctgccactctgacctgagtttcctacggtacaggatttactctgagaaacctcagcaccttgccacagtagccttggcagaatggcctcacggttagggaaactcctgattctaagcttgggagagctacgcttagatttgaattcacccaggaagcattcaaatcaaggctaaagacataaatgtgaaataaaactgtgaaccttcattctaaagttcatctgacttcccagatttgatcaatatattcttaggtggtattaaaaatggtaaactgcttaatttaaatctcaaaatttaaattatgaggtttacataaaaaccaacatttcatgaatgcacttttaaggtattaaaaggggtacttaagcggtaaatggtttcttggcacccataaccaagtaatagttaatttacaggtgggatttttttttattgctatgagaattacatttaaaattgtgggtgttttatataaagcagatatcacaagttttgaaaatttgttacctttaatatttcttctagagaataggtgtttgtatccataataaaagaaaaatttgtcagaactgctgcttcaatctaatcccatttgagagaattgtctttactgtcttaataactggatgaattatcactctgaaaatgtatttattgcactaaagttagtttaggcttgataaaacactccagacatttttactacagactgtttctataaaactgccattgcttctaatggagaattttattttaaaagaaataaaattgctgagtttatctgcaaaacctttctctaagtcttatgggacgtaaggagacacctccataattataagagccgttgtgctcagagtctccatgctggcttgaatgtatgatccacactgttaaaacataggcagcagctcagggccctggacagcctgagtcaacctagagtagctggaaccattttgacatgtaatggataagaaaattatccattgagaagctgaacaataaaccaaagaacgggtgtattttatccttaacctctgtaaaccacgttacactgagaacacttcagttcttcctaaaggttgataggcttcagtctgaaaacaatattgatttggagtggacagaagttaactaaccaactaccattatgttttgaatacacctttcaataaaatgattgaaatgcaa

SEQ ID NO: 190 - DNA, 4172 bp, Mus Musculus
agccctgcttctccttctaacaacacctttcccaccaccgtgtcaggctccatctgtggtgtgggtgctctacctctctggtcctcagcttgggggtggggtggaagtggggggagcctcaggccaccaacttcagacggaagtcaggtggggggcagggtgaggagccaacgccagaggagcctcaggaggagagggaagcagaggggcccaacaacgatggtgtcaccctgaagactggacaactgtgcagactgtggagggctgcgagaccacggctccagtatataaatcaggcaaattccccatttgagcatgaacttctgaaaacggcctgcatctaaggtctcctccaaggccctctggagtccagcccataatgaaggtcttggccgcagggattgtgcccttactgctgctggttctgcactggaaacacggggcagggagccctcttcccatcacccctgtaaatgccacctgtgccatacgccacccatgccacggcaacctcatgaaccagatcaagaatcaactggcacagctcaatggcagcgccaatgctctcttcatttcctattacacagctcaaggggagccgtttcccaacaacgtggaaaagctatgtgcgcctaacatgacagacttcccatctttccatggcaacgggacagagaagaccaagttggtggagctgtatcggatggtcgcatacctgagcgcctccctgaccaatatcacccgggaccagaaggtcctgaaccccactgccgtgagcctccaggtcaagctcaatgctactatagacgtcatgaggggcctcctcagcaatgtgctttgccgtctgtgcaacaagtaccgtgtgggccacgtggatgtgccacctgtccccgaccactctgacaaagaagccttccaaaggaaaaagttgggttgccagcttctggggacatacaagcaagtcataagtgtggtggtccaggccttctagagaggaggtcttgaatgtaccatggactgagggacctcaggagcaggatccggaggtggggagggggctcaaaatgtgctggggtttgggacattgttaaatgcaaaacggggctgctggcagaccccagggatttccaggtactcactgcactctgggctgggccatgatggaatctggcaaagttgaaacttccataggcagagcttctatacagcccagcaccagctagaaatggcaatgagggtgttggtctgagagatttctgtctcactcactcactcactcactctcactcactcactcactcactcactcagccccttgcttgctgggtgtatgaacaagctgcacaagttgtctacagcagacagcaaagggctgggaagtgtcctagacccctacagagtcaccatcatctggtcctttgctgtctctcagagaaactttggaaggcttggttgggatgtgagagagctaaggggactgggatccagaaggaatccttttattttattttattttattttattttattttattttataagttttgtgggtggaagggtaccctggggtggaatgatggaatgtgtcttctcttgagttggatgagagagttcaggcttagagactgtcagatggaagagtctaggtcaccagtgttcaggctcccacagaagcacagcggccagcttccagttgtcaaagcctgacgaactcggttagcttctatgcagttccccccacagcctggcgtggttggggtctgccagctggacctagaggtgaggtgtgtgcaggcaggaagaggcaggctgcaaaggcaggttcccagagtcctcccggggaaggacctctaactgtctaggagtcagggaaggagcaaggcagccagccattgctgaggcagtagccgactgcagctctcatctgcttctcaacccctgagaacaggtgatcttgagcagacagacaggtagcataaagtagaatgtcgggtctgaggccccggaggtcgcaaaggtacttgaaggggaccagagggctgtcttgggtccctggagcatggagaagcagaacttgaggtcagggtctcagggaagatgaggcccagagtgctgtgtttgatccagcacagctgtctatttattactatgtcctatttatattaacttattggtgctttaaatggcaaagttaattccccgaaatggtatgaggctccttccatgggagctggggccgagactctccacctagtggggcctggtctggaggcacatgattgttacaggtgcagctcatgggtcaaatcagagagctggctagctcctctgtctcccactgtgactcacttttagggtgtcagggtcccccagaaaaagctgggccagtttgtctctctgcttctgtctctgtctctccgagtctgtctctgtctgtctctgtgtctctgtctctttgtctctctctgtctctgtctctctctctctctccccgccccccccccttctccctctggtctccaagggggtggaacagtttcttgttgttttgtcccactgagctctctggcaccccctagattcctgctatgcggtgcaccattcataatgaagtgaatggctctggaaccttgggcaaaactgattccttcctcaaatcgtagctgaggagtgctgaaacatcctgacccggcacccagcgtgctttcgaccagcatggaagctcctcgggtggcccgaacacccacagagggtgaatacaggaggttggagcagtgcaggccctgaactgggcctgaacagctgcccagtgcgccagagaaggggagatcaaggcccgagacgcctgggacacagaccaggaagctgtggtccttgcttcatcgctgccttcccactcccgcccatgtctgggctcccaggcagggaatccgatctgatctctcctttgtgctgaggccaggcaagcagaggaacgccctcgatctgggagcagggtagggaggaaggcagccaagctggggcagtggctgactacagagctagctgcctgcctctcaggctctgaacagggcggtccttagcagttcagcagtgggattctgcttcacgcggttttgcacctttctctgtcactctctaagcactttacctggacggcaggtggacaggccctggagctctggcttaggaaaggcctggaaccatagatgcagcaaggagactatggtgggggccacgcgtgtcagcgacaaagttactccaccgtactcctgttgctgcgtcaggctcatctcaggactggctgcccttctccaagctgagagtcaatttgtctaaaagccaagatgatgccacagcctggggcctgttgggctttgtcatcacttcacatttgtatggacttggactctctgggctccgcccacctggcagctttgaaggctcagggaccaatggactctctccgtgcacgcccccgtccccccaacgcaaccacctacctgcgtcttactccatcagttgcccagcatcccagaaccattgagcctttggggaaaacagactttaggggcaggtagttgctcacctgacatctttcacctggaagcattgacttccaccgagcatagtaggtagtgtgtctggaccagagaaaaagggatggggcattttgcagtttatccagagagaagcaaaggggcctttatttattatttaaaacttcaaacctgaaagcactgagagtttactggtctgcccccctccccccactcttgtctatttctgtgtccttgatcccgactcaagcaacccagctctgctttgcctgctctctggagcagacatggtatgtgggccaggaccccggagtcttgcatggtagcggcttcagaagggaaatgatatggctgtctgcattcggatgactccccagtcccagcccagcctctcctttgcactgctgctctccctctttcctttcctttggaagggacttggccttgggtgacaaattcctctttgatgaatgtaccctgtgggaatgtttcatactgacagattatttttatttattcaatgtcatatttaaaatatttattttttatactgaaggagtgtctttttttttttaaagaaaaaatgaaataataaagaactcattcttgttga

SEQ ID NO: 191 - DNA, 2111 bp, Mus Musculus
ggtggggctgagggagggaaacaaacaaaatcccgctgcggggacaggagacggaaaagcaggctggaagggtagacacaattctcgcgagaagtagagcgaattccctcggagaggggcggttgcgcagaggtacaccacgtgtgcggtccctccggcgccgagtcagggtctctcgtcgagtctgggtctaggttagatttggaatcgtggagatgcggaaggacacccctcctccgctcgtgcccccggcggcccgcgagtggaacctgccccccaatgcgcccgcatgcatggaacgtcaattggaggctgcacggtaccggtctgatggttcccttctgctcggggtctccagcctgagtggtcgctgctgggtaggttctctgtggtttttcaaggatcctagtgcggcccccaacgaaggtttctgctctgctggcgtccagaccgaggctggagtagctgacctcacttgggtgggggacaaaggtatcctagtggcttctgattcaggtgctgttgaattgtgggagctagatgagaacgagacacttatagtcagcaagttctgcaagtatgagcatgatgacattgtgtctactgtcactgtcctgagctctggcacacaagctgtcagtggtagcaaagactgctgcatcaaaatttgggacctggctcagcaggtatcactgaattcataccgagctcacgctggacaggttacctgtgttgctgcctctccccacaaagactctgtgtttctttcatgtagtgaggacagtagaattttgctctgggatacccgctgtcccaagccggcatcacagatggcctgcaatgcctctggctacctccctaccgctttggcttggcatcctcagcagagtgaagtctttgtttttggtgacgagaatggatctgtctcccttgtggacaccaagaatgcaagctgtaccctcagctcagctgtgcactcccagggtgtcactagactggtattctccccacacagtgtccccctcctgacttctctcagtgaagactgttcacttgctgtgctggattcaagcctttctgaggtgtttagaagtcgagcccacagagactttgtgagagatgctacgtggtctccactcaatcactcccttcttaccacagttggctgggaccatcaggtcatccaccatgttgtgcccttagagcctctcccaaaccctggacctgacagtgttgtggagtagaatggatttcagaaaaaccaaaacaagccctccgtctgtaagcgactactcgattgcccctgccttccatgtgtgagagcacaggagccttgtagagcatgtttcctccctagccccgtgcagtaacaggcagattctcagcctgagggaggctgcatcccatagtgactcagaggaagaaacttcctctgtaaatggatgtatgtgagtacacgtgtgagtgcggtatatagtttggagtggagaaaattattcttcagctttcccaaagcaatgctctttacccctgacaaagtgaccagaggattttaatgcttcccatgtctgggaattggcgatgttagggatatggaatgtgggtatccctagatttttgggaatacttcatactactcagaggtgcgtaacttatttttatatagagtttaattcactattatgggaattacttccatatacaaaagtctagcccactgtaccttgagaagacaaaacagttttgtacaagtccctgttatactgagtcaaatccattttctgggtgaataaatttggccctgcagtgtagctcttgttcaccctttccttccttggtgttgtgtgtgagagatgagtgttttcctgtcctagatttcacagataagttgtagggatgatggaccacttaaaagtgtttcagtgaagtgggaaaattgacatggattgttacagagaaatctgtcatttttcccacctaaaaattgatctgggtaccttcctctggagagctgaagtctgaatgtatttttggactcctaagatttgagatccagcatcagggaaactgtcggaaatactaaaaggtgaaataaagacttttatccttga

SEQ ID NO: 192 - DNA, 2594 bp, Mus Musculus
ggtggggctgagggagggaaacaaacaaaatcccgctgcggggacaggagacggaaaagcaggctggaagggtagacacaattctcgcgagaagtagagcgaattccctcggagaggggcggttgcgcagaggtacaccacgtgtgcggtccctccggcgccgagtcagggtctctcgtcgagtctgggtctaggttagatttggaatcgtggagatgcggaaggacacccctcctccgctcgtgcccccggcggcccgcgagtggaacctgccccccaatgcgcccgcatgcatggaacgtcaattggaggctgcacggtaccggtctgatggttcccttctgctcggggtctccagcctgagtggtcgctgctgggtaggttctctgtggtttttcaaggatcctagtgcggcccccaacgaaggtttctgctctgctggcgtccagaccgaggctggagtagctgacctcacttgggtgggggacaaaggtatcctagtggcttctgattcaggtgctgttgaattgtgggagctagatgagaacgagacacttatagtcagcaagttctgcaagtatgagcatgatgacattgtgtctactgtcactgtcctgagctctggcacacaagctgtcagtggtagcaaagactgctgcatcaaaatttgggacctggctcagcaggtatcactgaattcataccgagctcacgctggacaggttacctgtgttgctgcctctccccacaaagactctgtgtttctttcatgtagtgaggacagtagaattttgctctgggatacccgctgtcccaagccggcatcacagatggcctgcaatgcctctggctacctccctaccgctttggcttggcatcctcagcagagtgaagtctttgtttttggtgacgagaatggatctgtctcccttgtggacaccaagaatgcaagctgtaccctcagctcagctgtgcactcccagggtgtcactagactggtattctccccacacaggtgctgtgtgtcaccaggaacttggaagggatgggtggggacagtggtgaaggagtaggaatggagtgacacagggaggaggtatctatttggcaaagctgcttccattggtccttagctggggcagtactctaacttggctttcccaagcttgtcagtgtagtctttatttggtttgtatagtttgagaagacctaaaatataatctcattcctgctcaaagcctgtgagtctgagtaactgttgctaatgtagagaaggagccattgggctggtgggctgctagtgtggtttctgtgagccctgttccattttctttgcctttccttgggattgtaatggtagtaagacctaaacctcaagtggccatcaggatgccttctcccctgtctcagtttacagtggggtcagggctggccctgatagattttggttgtggaatctatggactttatcttcttttccttttgtgtcctctcctagtgtccccctcctgacttctctcagtgaagactgttcacttgctgtgctggattcaagcctttctgaggtgtttagaagtcgagcccacagagactttgtgagagatgctacgtggtctccactcaatcactcccttcttaccacagttggctgggaccatcaggtcatccaccatgttgtgcccttagagcctctcccaaaccctggacctgacagtgttgtggagtagaatggatttcagaaaaaccaaaacaagccctccgtctgtaagcgactactcgattgcccctgccttccatgtgtgagagcacaggagccttgtagagcatgtttcctccctagccccgtgcagtaacaggcagattctcagcctgagggaggctgcatcccatagtgactcagaggaagaaacttcctctgtaaatggatgtatgtgagtacacgtgtgagtgcggtatatagtttggagtggagaaaattattcttcagctttcccaaagcaatgctctttacccctgacaaagtgaccagaggattttaatgcttcccatgtctgggaattggcgatgttagggatatggaatgtgggtatccctagatttttgggaatacttcatactactcagaggtgcgtaacttatttttatatagagtttaattcactattatgggaattacttccatatacaaaagtctagcccactgtaccttgagaagacaaaacagttttgtacaagtccctgttatactgagtcaaatccattttctgggtgaataaatttggccctgcagtgtagctcttgttcaccctttccttccttggtgttgtgtgtgagagatgagtgttttcctgtcctagatttcacagataagttgtagggatgatggaccacttaaaagtgtttcagtgaagtgggaaaattgacatggattgttacagagaaatctgtcatttttcccacctaaaaattgatctgggtaccttcctctggagagctgaagtctgaatgtatttttggactcctaagatttgagatccagcatcagggaaactgtcggaaatactaaaaggtgaaataaagacttttatccttga

SEQ ID NO: 193 - DNA, 2723 bp, Mus Musculus
ggtggggctgagggagggaaacaaacaaaatcccgctgcggggacaggagacggaaaagcaggctggaagggtagacacaattctcgcgagaagtagagcgaattccctcggagaggggcggttgcgcagaggtacaccacgtgtgcggtccctccggcgccgagtcagggtctctcgtcgagtctgggtctaggttagatttggaatcgtggagatgcggaaggacacccctcctccgctcgtgcccccggcggcccgcgagtggaacctgccccccaatgcgcccgcatgcatggaacgtcaattggaggctgcacggtaccggtctgatggttcccttctgctcggggtctccagcctgagtggtcgctgctgggtaggttctctgtggtttttcaaggatcctagtgcggcccccaacgaaggtttctgctctgctggcgtccagaccgaggctggagtagctgacctcacttgggtgggggacaaaggtatcctagtggcttctgattcaggtgctgttgaattgtgggagctagatgagaacgagacacttatagtcagcaagttctgcaagtatgagcatgatgacattgtgtctactgtcactgtcctgagctctggcacacaagctgtcagtggtagcaaagactgctgcatcaaaatttgggacctggctcagcaggtatcactgaattcataccgagctcacgctggacaggttacctgtgttgctgcctctccccacaaagactctgtgtttctttcatgtagtgaggacagtagaattttgctctgggatacccgctgtcccaagccggcatcacagatggcctgcaatgcctctggctacctccctaccgctttggcttggcatcctcagcagagtgaagtctttgtttttggtaaggcagcatgacgtaccgtagactctgggaatggggaggatgacaaagtgctaccttcaatggagtacatgcccctgtcagcacatgcaggcactgcatgaattatgccagtttgaggtagctatggagagcatctttttttccctagtgggtttgtttcctcattgttccagcttctttattctgggcatggttagaagtcagtgagctgtggagcaagtgcttgctgttatggaattatgttctgctgtggtgtgactgcatatggtaagagtttgtagttaattgtcccagccacatagtgacttactgattctataccacaaaggaaactgttgtctacattttgagagactaggattcattcaaatccaggtctggctcacatcaaagtatggtttttgttgttgttttttttttttttcttcctcaagacagggtctgtatgaagtttgggctgtcttagaacctgatgagtagaccaggctgaccttgaactcaaagatccttctgcttcagcctcccacatactagaattaaaggtgtatactatacttagccaggagcgtgaattcttagccactgtgacgtgctttatttcttctgcaggtgacgagaatggatctgtctcccttgtggacaccaagaatgcaagctgtaccctcagctcagctgtgcactcccagggtgtcactagactggtattctccccacacagtgtccccctcctgacttctctcagtgaagactgttcacttgctgtgctggattcaagcctttctgaggtgtttagaagtcgagcccacagagactttgtgagagatgctacgtggtctccactcaatcactcccttcttaccacagttggctgggaccatcaggtcatccaccatgttgtgcccttagagcctctcccaaaccctggacctgacagtgttgtggagtagaatggatttcagaaaaaccaaaacaagccctccgtctgtaagcgactactcgattgcccctgccttccatgtgtgagagcacaggagccttgtagagcatgtttcctccctagccccgtgcagtaacaggcagattctcagcctgagggaggctgcatcccatagtgactcagaggaagaaacttcctctgtaaatggatgtatgtgagtacacgtgtgagtgcggtatatagtttggagtggagaaaattattcttcagctttcccaaagcaatgctctttacccctgacaaagtgaccagaggattttaatgcttcccatgtctgggaattggcgatgttagggatatggaatgtgggtatccctagatttttgggaatacttcatactactcagaggtgcgtaacttatttttatatagagtttaattcactattatgggaattacttccatatacaaaagtctagcccactgtaccttgagaagacaaaacagttttgtacaagtccctgttatactgagtcaaatccattttctgggtgaataaatttggccctgcagtgtagctcttgttcaccctttccttccttggtgttgtgtgtgagagatgagtgttttcctgtcctagatttcacagataagttgtagggatgatggaccacttaaaagtgtttcagtgaagtgggaaaattgacatggattgttacagagaaatctgtcatttttcccacctaaaaattgatctgggtaccttcctctggagagctgaagtctgaatgtatttttggactcctaagatttgagatccagcatcagggaaactgtcggaaatactaaaaggtgaaataaagacttttatccttga

SEQ ID NO: 194 - DNA, 2094 bp, Mus Musculus
ggtggggctgagggagggaaacaaacaaaatcccgctgcggggacaggagacggaaaagcaggctggaagggtagacacaattctcgcgagaagtagagcgaattccctcggagaggggcggttgcgcagaggtacaccacgtgtgcggtccctccggcgccgagtcagggtctctcgtcgagtctgggtctaggttagatttggaatcgtggagatgcggaaggacacccctcctccgctcgtgcccccggcggcccgcgagtggaacctgccccccaatgcgcccgcatgcatggaacgtcaattggaggctgcacggtaccggtctgatggttcccttctgctcggggtctccagcctgagtggtcgctgctgggtaggttctctgtggtttttcaaggatcctagtgcggcccccaacgaaggtttctgctctgctggcgtccagaccgaggctggagtagctgacctcacttgggtgggggacaaaggtatcctagtggcttctgattcaggtgctgttgaattgtgggagctagatgagaacgagacacttatagtcagcaagttctgcaagtatgagcatgatgacattgtgtctactgtcactgtcctgagctctggcacacaagctgtcagtggtagcaaagactgctgcatcaaaatttgggacctggctcagcaggtatcactgaattcataccgagctcacgctggacaggttacctgtgttgctgcctctccccacaaagactctgtgtttctttcatgtagtgaggacagtagaattttgctctgggatacccgctgtcccaagccggcatcacagatggcctgcaatgcctctggctacctccctaccgctttggcttggcatcctcagcagagtgaagtctttgtttttggtgacgagaatggatctgtctcccttgtggacaccaagaatgcaagctgtaccctcagctcagctgtgcactcccagggtgtcactagactgtgtccccctcctgacttctctcagtgaagactgttcacttgctgtgctggattcaagcctttctgaggtgtttagaagtcgagcccacagagactttgtgagagatgctacgtggtctccactcaatcactcccttcttaccacagttggctgggaccatcaggtcatccaccatgttgtgcccttagagcctctcccaaaccctggacctgacagtgttgtggagtagaatggatttcagaaaaaccaaaacaagccctccgtctgtaagcgactactcgattgcccctgccttccatgtgtgagagcacaggagccttgtagagcatgtttcctccctagccccgtgcagtaacaggcagattctcagcctgagggaggctgcatcccatagtgactcagaggaagaaacttcctctgtaaatggatgtatgtgagtacacgtgtgagtgcggtatatagtttggagtggagaaaattattcttcagctttcccaaagcaatgctctttacccctgacaaagtgaccagaggattttaatgcttcccatgtctgggaattggcgatgttagggatatggaatgtgggtatccctagatttttgggaatacttcatactactcagaggtgcgtaacttatttttatatagagtttaattcactattatgggaattacttccatatacaaaagtctagcccactgtaccttgagaagacaaaacagttttgtacaagtccctgttatactgagtcaaatccattttctgggtgaataaatttggccctgcagtgtagctcttgttcaccctttccttccttggtgttgtgtgtgagagatgagtgttttcctgtcctagatttcacagataagttgtagggatgatggaccacttaaaagtgtttcagtgaagtgggaaaattgacatggattgttacagagaaatctgtcatttttcccacctaaaaattgatctgggtaccttcctctggagagctgaagtctgaatgtatttttggactcctaagatttgagatccagcatcagggaaactgtcggaaatactaaaaggtgaaataaagacttttatccttga

SEQ ID NO: 195 - DNA, 2596 bp, Mus Musculus
ggaagtgctcctgcggctccggttgccggcgtgactttgagctcttcccgctctgctgccggcgctgagcacaaccgatcacctcagagcaggagccttggaagtggctgttccaggcttgccatgcggctgctgcgggtcgccgcagccctggggcgtggaccgttccctcgggtccctgcagtcctgggctggcaggggaagcaggctgattggaagacccgccgatggtgttcgtcagggccagttcccaatgaaaaaatacggaacattgggatctcagctcacattgattctgggaagacgacgctaacagaacgggtgctatactacactggcaggattgccacgatgcacgaggtgaaaggtaaagatggagttggcgctgtcatggattccatggagctggagaggcagcgagggatcacgatccagtcagcagctacgtataccatgtggaaagacatcaatatcaacatcatagacacgccaggccacgtggactttaccatagaagtggaaagagccctgcgggtcctggacggtgcagtccttgttctctgtgcggttggcggagtccagtgccagaccatgacagtcagtcgtcagatgaagcgctacaacgttccctttttaaccttcattaacaaactggaccgaatgggctctaacccatccagggccctccaacaaatgagatctaaactaaatcacaatgctgcatttgtacaaatacccattggtttagagggtgattttaagggaattatagacctcattgaagaacgagctatttactttgatggagactttggtcagattgtccgatatgacgagattccggctggattgcgggccgcagccgctgaccaccggcaggagctcattgagtgcgttgctaactcagatgagcagcttggtgagctcttcctggaagaaaagatcccctcagtttccgacttaaagcgagcaatccggagagctactctgagtcgatccttcactcctgtatttttgggaagtgctttgaaaaacaaaggagttcaacctcttttggacgctgttttagaatacctgcccaatccatctgaggtccagaattacgctatactcaatcagaatgactcaaaagagaagaccaaaatcttaatgaacccccaaagagatgattcgcacccatttgtaggcctggcttttaaactggaggcgggccgttttggacagctgacttatgtccgaaattatcaaggtgaactgaagaagggaagcaccatctacaacacgaggaccgggaagaaagtgcgcgtgcagcggctagtgcgcatgcacgccgacatgatggaggatgttgaagaagtatatgctggagacatctgtgcattgtttggcattgactgtgcaagtggagatacattcacaaacaaagataacagtgacctttctatggaatcgattcatgttcctgagcctgtcatttcaatagcaatgaggccttctaacaagaatgatctggagaaattttccaaaggtattggcaggtttacacgggaagaccccacatttaaagtccactttgaccctgagagcaaggagaccattgtgtcagggatgggagagctgcacctggaaatctatgctcagagaatggagagagaatatggctgcccttgtatcacaggaaaacccaaagtagcctttagagagaccattgttgccccagtgccgtttgattttacacataaaaagcagtcgggtggtgccggccagtttggaaaagtaataggagtgctggagcccttgcccccagaggactacacgaagttggagttttctgatgagacatttgggtccaatgttccaaagcagtttgtccccgctgttgaaaaggggtttctggatgcctgcgaaaaaggccctctttctggtcacaagctttctgggctacggtttgttctgcaagatggagcacaccacatggttgattcgaatgaaatctctttcatccgagctggagaaggtgctcttaaacaagccttggcaaatggcacactatgtattattgagcctattatgtctgtggaagttatagcccccaatgaattccagggaacagtgttcgcagggattaaccggcggcatggagtgattacagggcaggacggcattgaggactacttcacactgtatgcagatgttcccctaaataatatgtttggttattccactgagcttaggtcttgcacagagggaaaaggagagtacacgatggagtactgcaggtaccagccgtgttcaccatccacacaggaagagctcatcaataagtatttggaagctacaggtcagcttcccgtaaaaaaagggaaagccaagaactgactctcctcactcaggatatactgactatttgactaccacacttggggatgatggagtctgcttgaatgaattctgacggcagaaacaaatttaggagcccctgctggcccacgttcaggagcttctgtatgtacgccaaggataactgtgtatttaaactgtattgactatttttattgtttagtaaaagaccttaaataaaattatattattactg

SEQ ID NO: 196 - DNA, 1971 bp, Mus Musculus
ggaagtgctcctgcggctccggttgccggcgtgactttgagctcttcccgctctgctgccggcgctgagcacaaccgatcacctcagagcaggagccttggaagtggctgttccaggcttgccatgcggctgctgcgggtcgccgcagccctggggcgtggaccgttccctcgggtccctgcagtcctgggctggcaggggaagcaggctgattggaagacccgccgatggtgttcgtcagggccagttcccaatgaaaaaatacggaacattgggatctcagctcacattgattctgggaagacgacgctaacagaacgggtgctatactacactggcaggattgccacgatgcacgaggtgaaaggtaaagatggagttggcgctgtcatggattccatggagctggagaggcagcgagggatcacgatccagtcagcagctacgtataccatgtggaaagacatcaatatcaacatcatagacacgccaggccacgtggactttaccatagaagtggaaagagccctgcgggtcctggacggtgcagtccttgttctctgtgcggttggcggagtccagtgccagaccatgacagtcagtcgtcagatgaagcgctacaacgttccctttttaaccttcattaacaaactggaccgaatgggctctaacccatccagggccctccaacaaatgagatctaaactaaatcacaatgctgcatttgtacaaatacccattggtttagagggtgattttaagggaattatagacctcattgaagaacgagctatttactttgatggagactttggtcagattgtccgatatgacgagattccggctggattgcgggccgcagccgctgaccaccggcaggagctcattgagtgcgttgctaactcagatgagcagcttggtgagctcttcctggaagaaaagatcccctcagtttccgacttaaagcgagcaatccggagagctactctgagtcgatccttcactcctgtatttttgggaagtgctttgaaaaacaaaggagttcaacctcttttggacgctgttttagaatacctgcccaatccatctgaggtccagaattacgctatactcaatcagaatgactcaaaagagaagaccaaaatcttaatgaacccccaaagagatgattcgcacccatttgtaggcctggcttttaaactggaggcgggccgttttggacagctgacttatgtccgaaattatcaaggtgaactgaagaagggaagcaccatctacaacacgaggaccgggaagaaagtgcgcgtgcagcggctagtgcgcatgcacgccgacatgatggaggatgttgaagaagtatatgctggagacatctgtgcattgtttggcattgactgtgcaagtggagatacattcacaaacaaagataacagtgacctttctatggaatcgattcatgttcctgagcctgtcatttcaatagcaatgaggccttctaacaagaatgatctggagaaattttccaaaggtattggcaggtttacacgggaagaccccacatttaaagtccactttgaccctgagagcaaggagaccattgtgtcagggatgggagagctgcacctggaaatctatgctcagagaatggagagagaatatggctgcccttgtatcacaggaaaacccaaagtagcctttagagagaccattgttgccccagtgccagtggcatcaaataaagaaaagttgtatatgttacctgcgatgcaatgtcgtggagtagaatggtgtaatctaactcaatgaagtcatcgtttctttgctacaacaacaacaacaacaacaaaatgcttagacaacatcatggcaaaaatgacatcttaatttcttttttgcattaatgttgatttattcacttcaggttagcctggcagaaaactagtttcttaaaataataacaattcttctttc

SEQ ID NO: 197 - DNA, 1001 bp, Mus Musculus
agccctgagtctggcttgcgggtggtgggagcagacagttcttgcctcgggaccggcaatcatggcttcggcttggccgcggtctctgccgcagatcctcgtgttgggattcggcttggtgttgatgcgcgccgcggccggggagcaagcaccaggcacctccccatgctctagcggcagctcctggagcgcggacctcgacaagtgcatggactgcgcttcttgtccagcgcgaccacacagcgacttctgcctgggatgcgccgcagcacctcctgcccacttcaggctactgtggcccattctggggggcgctcttagtctggtcctggttttggcgctggtttctagtttcctggtctggagaagatgccgccggagagaaaagtttactacccccatagaggagactggtggagagggctgcccaggtgtggcactgatccagtgaggagcacccgcgctggggcccactcgtcgtccattcattcattctggagtcagcctggccttccagacagaagccgagccagactctttcaaccacaagggggtggggcgaggtggtgattcacctccaaggactgggcttagagttcagggagccttccaaggtgtccaattgccctatctctgggtctggggcagacagagagcctcaatctgggtcacaaagcgacccatactaaggaactgcagcatttgcacaagggaatctcttgttccccacaagtcctggcagtctggctgacttgaggggcagacacaacactgggtcccacccactcggaggggctataatgccactccctgagttttagccctaggagtgggtcagattgacagacctgttcttaacactaagggggctggccctctgggagggctggccccagtacacacggaaacaaccatctcccaaggggggtgatatttattgtggggataatgtttggaggggagggagacttattaataaaagaatctttaactttaagata

SEQ ID NO: 198 - DNA, 1078 bp, Mus Musculus
agccctgagtctggcttgcgggtggtgggagcagacagttcttgcctcgggaccggcaatcatggcttcggcttggccgcggtctctgccgcagatcctcgtgttgggattcggcttggtgttgatgcgcgccgcggccggggagcaagcaccaggcagcaagttccaagtgggaaaggtctcaagttttatccgcagtccgagcttcagtttcgctacctgtccaattgaggcacctccccatgctctagcggcagctcctggagcgcggacctcgacaagtgcatggactgcgcttcttgtccagcgcgaccacacagcgacttctgcctgggatgcgccgcagcacctcctgcccacttcaggctactgtggcccattctggggggcgctcttagtctggtcctggttttggcgctggtttctagtttcctggtctggagaagatgccgccggagagaaaagtttactacccccatagaggagactggtggagagggctgcccaggtgtggcactgatccagtgaggagcacccgcgctggggcccactcgtcgtccattcattcattctggagtcagcctggccttccagacagaagccgagccagactctttcaaccacaagggggtggggcgaggtggtgattcacctccaaggactgggcttagagttcagggagccttccaaggtgtccaattgccctatctctgggtctggggcagacagagagcctcaatctgggtcacaaagcgacccatactaaggaactgcagcatttgcacaagggaatctcttgttccccacaagtcctggcagtctggctgacttgaggggcagacacaacactgggtcccacccactcggaggggctataatgccactccctgagttttagccctaggagtgggtcagattgacagacctgttcttaacactaagggggctggccctctgggagggctggccccagtacacacggaaacaaccatctcccaaggggggtgatatttattgtggggataatgtttggaggggagggagacttattaataaaagaatctttaactttaagata

SEQ ID NO: 199 - DNA, 910 bp, Mus Musculus
agccctgagtctggcttgcgggtggtgggagcagacagttcttgcctcgggaccggcaatcatggcttcggcttggccgcggtctctgccgcagatcctcgtgttgggattcggcttggtgttgatgcgcgccgcggccggggagcaagcaccaggcacctccccatgctctagcggcagctcctggagcgcggacctcgacaagtgcatggactgcgcttcttgtccagcgcgaccacacagcgacttctgcctgggattttcctggtctggagaagatgccgccggagagaaaagtttactacccccatagaggagactggtggagagggctgcccaggtgtggcactgatccagtgaggagcacccgcgctggggcccactcgtcgtccattcattcattctggagtcagcctggccttccagacagaagccgagccagactctttcaaccacaagggggtggggcgaggtggtgattcacctccaaggactgggcttagagttcagggagccttccaaggtgtccaattgccctatctctgggtctggggcagacagagagcctcaatctgggtcacaaagcgacccatactaaggaactgcagcatttgcacaagggaatctcttgttccccacaagtcctggcagtctggctgacttgaggggcagacacaacactgggtcccacccactcggaggggctataatgccactccctgagttttagccctaggagtgggtcagattgacagacctgttcttaacactaagggggctggccctctgggagggctggccccagtacacacggaaacaaccatctcccaaggggggtgatatttattgtggggataatgtttggaggggagggagacttattaataaaagaatctttaactttaagata

SEQ ID NO: 200 - DNA, 952 bp, Mus Musculus
gacgcacgacacaacctctcgattccggacgtgcctgatggtggctcttctcgttacgccccaagacctagcactgcctgtcgcccagcaactaaggcggcaggtccgtgctgtctcgaaccacagaaacctaggagaactacacgaagcacccttatgctaaaaagcgcccagaactatccagccacgccggatcccctcccccaccgcgccgacctgtcccgccttctaacctctaacctgattcctgtcttccgcgcctgcgtaaaacgtctccagtgcgcacgccggtaggcccgcccctcgcgctgtaccacgccctcaaaggctccgcctctgctgcttgaatcccacccctctatctgcaggaccctgagccgtaatgatcctgcaacagcccctggagcgaggtcccccaagtcgggacccacgggccaccactggggtaactcgcggcctaaacgccagtctttcccccagggaacctttgcacaagcagttcctgtctgaggagaacatggccacccatttctcccgactcagcctacataatgaccacccctactgcagcccccctgtgacttttcccgaagccctgccaccactcaggagcccttgcccagagctgcttctctggcgctatcccgggagcctgattcctgaagccctcaggctgctgaggctgggggatacccccagtccctactaccctgcgtccccagctggggatatcgtggagctctgagtattgatggcctgtgtccctccctatcttctttctgacaacaaagaccagcaactcccagaaagggatgaagtccttctgcaaacctggccaccaggaccttccctctgacaagaggacactagctcggtggaaccctcagtgaaaggcagcctgggaaagggaaaatgttaccccattagtgaataaagacctgtgagcagttga

SEQ ID NO: 201 - DNA, 1108 bp, Mus Musculus
gggaggaaaacttcctggctgggactccggcggtttctgcctgccaaccctcggatcccgcctaccagtgttggctcttcccgacccctcccccggcgcccccagtgtccgccatggccaaagcctacgaccacctcttcaagttgctgctcatcggggactcgggggtgggcaagacttgtctgatcattcgctttgcagaggacaacttcaacagcacttacatctctaccatcggaattgatttcaagatccgaaccgtggacatagaggggaagaggatcaaactgcaagtgtgggacacggctggccaagaacgattcaagacaataactaccgcctattaccgtggagccatgggcattatcctcgtatatgacatcacagatgagaaatccttcgagaatattcagaactggatgaaaagcatcaaagagaatgcctctgcgggagtggagcgcctcctgctgggaaacaagtgtgacatggaggccaagcggcaggtgcagagagagcaggcggagaagttggctcgagagcacagaatccgattttttgagacgagtgccaaatccagtgtgaatgtggatgaggctttcagttccctggcccgtgacatcttgctcaagacaggaggccggagatcgggaaccaacagtaagccctcaagcactggcctgaaaacatctgacaagaagaagaacaagtgcttgttaggctgagagcatttcttgcctcctattcaccctgaacctggaggctagacctgagggagtcggactgagggattgcagatgggagaactgtggtggcacctcaaggggagatgaggggaataaggagaccggcgaggacgagacggaagaaaggggcagggaaaggagggggaggaaccaaggatgtgaaaggtgaacagaagggatttgagaagaggaaaggaagaagaaatgaatggctcaggccttggacagtccaacattaaagtcaacatgctgatctctccattcctggttcagggttagggtcctgagaggctggctcggcactactccgagggtccctcactctacaaggtctttgttagtattaaaggccactgttttgcatg

SEQ ID NO: 202 - DNA, 1037 bp, Mus Musculus
gaaagggaggagtagggcagggccagaggagggacagcctgacgcggccaccagccctgactcaacctgccagcccccttacctggccagccttgaggagatggaacagcccaggctacaaggcctgcccccactcctcaacttcccttgataatgaaccagaaactgaaactaaaacaggcaggctccccgttagagatgagtcacttcccaaagtgactgaagtagccgagaagtggaaacagaggggcagaagagaacccagggcactgagacagggctttctgagggtcgccaaggagcatggcaggcatcccagaggtggtggccatggactgtgagatggtggggcttgggcctcaaagggtgagtggcctcgcccgctgcagcattgtgaacatccatggcgcagtcctgtatgacaagtacatccgacccgagggagagatcacggactacagaacccaagtcagcggggtcacgcctcagcacatggtgagggccacgccatttggtgaagccaggctagagatcctgcagcttctgaaaggcaagctggtggtgggccatgacctgaagcacgacttcaatgccctgaaggaggatatgagcaagtacaccatctatgacacgtccacagacaggctgctgtggcatgaggccaagctgcagtactacagccgagtgtccctgaggctgctgtgtaagcgcctgctacacaagaacatccagaacaactggcggggccactgctctgtggaagatgccagggccacaatggagctctacaaaatctctcagcgactcagagcccagcgagggctgccttgccctgggacgtcagactgaacttcatcctcatccagggttagaagctgccactcctcaagttctccatgaatgagacctgtttctaacaaccactgccaccaaccaccgcaatgtgtaaaaccaagaacactcccccattacagcaactctcttgctttgaggcaaaaacaacaacaacaacaacaacaacaacaacagcaaaacca

SEQ ID NO: 203 - DNA, 1017 bp, Mus Musculus
gaaagggaggagtagggcagggccagaggagggacagcctgacgcggccaccagccctgactcaacctgccagcccccttacctggccagccttgaggagatggaacagcccaggctacaaggcctgcccccactcctcaacttcccttgataatgaaccagaaactgaaactaaaacaggcaggctccccgttagagatgagtcacttcccaaagtgactgaagtagccgagaagtggaaacagaggggcagaagagaacccagggcactgagacagggctttctgagggtcgccaaggagcatggcaggcatcccagaggtggtggccatggactgtgagatggtggggcttgggcctcaaagggtgagtggcctcgcccgctgcagcattgtgaacatccatggcgcagtcctgtatgacaagtacatccgacccgagggagagatcacggactacagaacccaagtcagcggggtcacgcctcagcacatggtgagggccacgccatttggtgaagccaggctagagatcctgcagcttctgaaaggcaagctggtggtgggccatgacctgaagcacgacttcaatgccctgaaggaggatatgagcaagtacaccatctatgacacgtccacagacaggctgctgtggcatgaggccaagctgcagtactacagccgagtgtccctgaggctgctgtgtaagcgcctgctacacaagaacatccaggtgaggagtctcccggcccccatgacccttcttgtctctctctcttctccctcctccctctcgttctccctcttcccactcagatgctctttctctttctccttcctctcctactcctccaagcaaggagttctcctcctttcttccttagtcctccctctccttccttgcttctaaaagtgagggtcttcctctccctcctccccacactcccactcctcccaagggcagatcagtcccacctgcctaagctggctccaggtaaggaacctctgcagcctgctg

SEQ ID NO: 204 - DNA, 1164 bp, Mus Musculus
agaacccaggaggaagaacagcctggcttgaggccctctaaaggtctgctgctgtttgtcaactctcccagggctttctgagggtcgccaaggagcatggcaggcatcccagaggtggtggccatggactgtgagatggtggggcttgggcctcaaagggtgagtggcctcgcccgctgcagcattgtgaacatccatggcgcagtcctgtatgacaagtacatccgacccgagggagagatcacggactacagaacccaagtcagcggggtcacgcctcagcacatggtgagggccacgccatttggtgaagccaggctagagatcctgcagcttctgaaaggcaagctggtggtgggccatgacctgaagcacgacttcaatgccctgaaggaggatatgagcaagtacaccatctatgacacgtccacagacaggctgctgtggcatgaggccaagctgcagtactacagccgagtgtccctgaggctgctgtgtaagcgcctgctacacaagaacatccaggtcctgcctggcagcctcttgggggttggaggatgcatcttgccaggcactgacatccttcatcttctgctctatgtcggaatggtcaggattgctgatctccgcttgctgacgccctttctacctccgagctgcctcgcttgcccactgctcccggagagtcttgcttctgctcgctcgcacgctgttatttctgccctgtccagctctagtcacctgctgacacccctcccaaacccgagtcagggacctcagggacacgtggacagactgagcggccagctccaggactggggaggcagccccttagctcctgctctccctgtgtctgcagaacaactggcggggccactgctctgtggaagatgccagggccacaatggagctctacaaaatctctcagcgactcagagcccagcgagggctgccttgccctgggacgtcagactgaacttcatcctcatccagggttagaagctgccactcctcaagttctccatgaatgagacctgtttctaacaaccactgccaccaaccaccgcaatgtgtaaaaccaagaacactcccccattacagcaactctcttgctttgaggcaaaaacaacaacaacaacaacaacaacaacaacagcaaaacca

SEQ ID NO: 205 - DNA, 2224 bp, Mus Musculus
agcgaccggcaggctgcccgcccgacttcccagctgccccgtgcggcccgggcatgcccagtgtgggcgcagccccggctctccgagcgcccgggcggagcgggcagccgggcgcggagcaggcgaaccgagcctcggtgggcgagcgccctggctggagccgcggcggtagggtggcggggccctccatgatgaatggtttgggggcacttccctctcgctgtatttgatagtctgggcagtggagagatggctgacagtgtcaaaacctttctgcaggaccttggcaggggaatcaaagactccatctggggcatctgtaccatctcaaagctagatgctcggatccagcagaagagagaggaacagcgtcgaagaagggcaagtagcctcttggcccagaggagaccccagagtgtagagcggaagcaagagagtgaaccacgtattgttagtagaattttccagtgttgtgcttggaatggtggagtattctggttcagtctcctcttgttttatcgagtgtttattcctgtacttcagtcagtaacagcccggattattggagatccatcacttcatggagatgtttggtcatggctggaattcttcctcacatcaattttcagtgctctttgggtgctccccctgtttgtgcttagcaaagttgtgaatgccatttggttccaagatatagctgacttggcatttgaagtatcagggaggaaacctcatccattccccagtgtcagcaaaataattgctgacatgctcttcaaccttttgctacaggcacttttccttattcaggggatgtttgtgagtctcttccccatccatcttgtgggtcagctggttagtctgctgcatatgtctcttctctattcactgtactgctttgagtaccgttggttcaacaaaggaattgaaatgcaccagcgattgtcgaacatagaaaggaattggccttactactttgggtttggcttgcccttggctttcctcacagcaatgcaatcctcctacattatcagtggctgcctcttttctatcctgtttcctttattcatcatcagcgccaatgaagcaaagactcctggaaaagcatatcttttccagttgcgcctattctccttggtggtctttttaagcaacagacttttccacaagaccgtctacctgcagtcagccctgagcagctcgtcctctgcagagaaattcccttcgccacatccttctcctgccaaactgaaagctgctgcaggccactgagccctgctgtcaaaggggtgggtgggactgggtggaggatgtggcagctcttttctctgttttcctccccctgccgtggaaggcagaacccactgccaagggccctctgcatagtcccttgtctttgaattggaatcttcctgactccagtatatggatttttaccaccaccctaggtctgtaaggaccagagttttccagctgtttttttagcacttgccagctcctgtgcctggactgattgatttgagtacttttttccccctttccttgtgtcatttaccctcccacttcctcctgccttccagcacccctggatgaatgggctttgtaattttaactgttgtattttgtgaatttgttgttactgtttttctgtgaagcacatacattgtatgtgggaggtaaaggggcattccagttgctccagtcactccctctatagccatcactgtcttgttttctgtaactcaggttaggttttggtctctattgctctgctgcagaaaaggaaagaaaggagtgggggaaatggagcctgaagagttggggcagatagacctcagccaaactggctgggttttgaggagtcatgttctttcttccccttgaaggggaaagagttttttccactggtccatttaaagtttcccagctatggggtggtaccagttctggacaagtgccactgcatcatagtatgctcggagaatctgaaccttactctgaagatgaaatttactgttggccactgccaggtcagactggtgttttaaggaatactgggtgcttcatataggaactgaaggggtaaacttactaaaccattcaacctgtgattggtgatgttttcctgtcattttaagagtcgacacatgggtgggggggcagatgtaaaaaaacttgtacaattttaaaatatcacaattaaacgtgagctggtttcccaaaa

SEQ ID NO: 206 - DNA, 2144 bp, Mus Musculus
agcgaccggcaggctgcccgcccgacttcccagctgccccgtgcggcccgggcatgcccagtgtgggcgcagccccggctctccgagcgcccgggcggagcgggcagccgggcgcggagcaggcgaaccgagcctcggtgggcgagcgccctggctggagccgcggcggtagggtggcggggccctccatgatgaatggtttgggggcacttccctctcgctgtatttgatagtctgggcagtggagagatggctgacagtgtcaaaacctttctgcaggaccttggcaggggaatcaaagactccatctggggcatctgtaccatctcaaagctagatgctcggatccagcagaagagagaggaacagcgtcgaagaagggcaagtagcctcttggcccagaggagaccccagagtgtagagcggaagcaagagagtgaaccacgtattgttagtagaattttccagtgttgtgcttggaatggtggagtattctggttcagtctcctcttgttttatcgagtgtttattcctgtacttcagtcagtaacagcccggattattggagatccatcacttcatggagatgtttggtcatggctggaattcttcctcacatcaattttcagtgctctttgggtgctccccctgtttgtgcttagcaaagttgtgaatgccatttggttccaagatatagctgacttggcatttgaagtatcagggaggaaacctcatccattccccagtgtcagcaaaataattgctgacatgctcttcaaccttttgctacaggcacttttccttattcaggggatgtttgtgagtctcttccccatccatcttgtgggtcagctggttagtctgctgcatatgtctcttctctattcactgtactgctttgagtaccgttggttcaacaaaggaattgaaatgcaccagcgattgtcgaacatagaaaggaattggccttactactttgggtttggcttgcccttggctttcctcacagcaatgcaatcctcctacattatcagtggctgcctcttttctatcctgtttcctttattcatcatcagcgccaatgaagcaaagactcctggaaaagcatatcttttccagttgcgcctattctccttggtggtctttttaagcaacagacttttccacaagaccgtctacctgcagccactgagccctgctgtcaaaggggtgggtgggactgggtggaggatgtggcagctcttttctctgttttcctccccctgccgtggaaggcagaacccactgccaagggccctctgcatagtcccttgtctttgaattggaatcttcctgactccagtatatggatttttaccaccaccctaggtctgtaaggaccagagttttccagctgtttttttagcacttgccagctcctgtgcctggactgattgatttgagtacttttttccccctttccttgtgtcatttaccctcccacttcctcctgccttccagcacccctggatgaatgggctttgtaattttaactgttgtattttgtgaatttgttgttactgtttttctgtgaagcacatacattgtatgtgggaggtaaaggggcattccagttgctccagtcactccctctatagccatcactgtcttgttttctgtaactcaggttaggttttggtctctattgctctgctgcagaaaaggaaagaaaggagtgggggaaatggagcctgaagagttggggcagatagacctcagccaaactggctgggttttgaggagtcatgttctttcttccccttgaaggggaaagagttttttccactggtccatttaaagtttcccagctatggggtggtaccagttctggacaagtgccactgcatcatagtatgctcggagaatctgaaccttactctgaagatgaaatttactgttggccactgccaggtcagactggtgttttaaggaatactgggtgcttcatataggaactgaaggggtaaacttactaaaccattcaacctgtgattggtgatgttttcctgtcattttaagagtcgacacatgggtgggggggcagatgtaaaaaaacttgtacaattttaaaatatcacaattaaacgtgagctggtttcccaaaa

SEQ ID NO: 207 - DNA, 2507 bp, Mus Musculus
agcgaccggcaggctgcccgcccgacttcccagctgccccgtgcggcccgggcatgcccagtgtgggcgcagccccggctctccgagcgcccgggcggagcgggcagccgggcgcggagcaggcgaaccgagcctcggtgggcgagcgccctggctggagccgcggcggtagggtggcggggccctccatgatgaatggtttgggggcacttccctctcgctgtatttgatagtctgggcagtggagagatggctgacagtgtcaaaacctttctgcaggaccttggcaggggaatcaaagactccatctggggcatctgtaccatctcaaagctagatgctcggatccagcagaagagagaggaacagcgtcgaagaagggcaagtagcctcttggcccagaggagaccccagagtgtagagcggaagcaagagagtgaaccacgtattgttagtagaattttccagtgttgtgcttggaatggtggagtattctggttcagtctcctcttgttttatcgagtgtttattcctgtacttcagtcagtaacagcccggattattggagatccatcacttcatggagatgtttggtcatggctggaattcttcctcacatcaattttcagtgctctttgggtgctccccctgtttgtgcttagcaaagttgtgaatgccatttggttccaagtaggtgcaggacaaaatggaactgggttctacatgctgggttgactaggttacacattttagcctatgaagttagtggcctataaaacttaatccttaacgtttttaagccaagcagtcaggtaatatttgtggttttgtaaaatgaaattactgtcttgggttactgagacagacttttctcacctggcagttctagcagcaaaccagtggccttaactctggggatgaacactgggattatttaatccatattttgctatattattgttctttgtttcaggatatagctgacttggcatttgaagtatcagggaggaaacctcatccattccccagtgtcagcaaaataattgctgacatgctcttcaaccttttgctacaggcacttttccttattcaggggatgtttgtgagtctcttccccatccatcttgtgggtcagctggttagtctgctgcatatgtctcttctctattcactgtactgctttgagtaccgttggttcaacaaaggaattgaaatgcaccagcgattgtcgaacatagaaaggaattggccttactactttgggtttggcttgcccttggctttcctcacagcaatgcaatcctcctacattatcagtggctgcctcttttctatcctgtttcctttattcatcatcagcgccaatgaagcaaagactcctggaaaagcatatcttttccagttgcgcctattctccttggtggtctttttaagcaacagacttttccacaagaccgtctacctgcagtcagccctgagcagctcgtcctctgcagagaaattcccttcgccacatccttctcctgccaaactgaaagctgctgcaggccactgagccctgctgtcaaaggggtgggtgggactgggtggaggatgtggcagctcttttctctgttttcctccccctgccgtggaaggcagaacccactgccaagggccctctgcatagtcccttgtctttgaattggaatcttcctgactccagtatatggatttttaccaccaccctaggtctgtaaggaccagagttttccagctgtttttttagcacttgccagctcctgtgcctggactgattgatttgagtacttttttccccctttccttgtgtcatttaccctcccacttcctcctgccttccagcacccctggatgaatgggctttgtaattttaactgttgtattttgtgaatttgttgttactgtttttctgtgaagcacatacattgtatgtgggaggtaaaggggcattccagttgctccagtcactccctctatagccatcactgtcttgttttctgtaactcaggttaggttttggtctctattgctctgctgcagaaaaggaaagaaaggagtgggggaaatggagcctgaagagttggggcagatagacctcagccaaactggctgggttttgaggagtcatgttctttcttccccttgaaggggaaagagttttttccactggtccatttaaagtttcccagctatggggtggtaccagttctggacaagtgccactgcatcatagtatgctcggagaatctgaaccttactctgaagatgaaatttactgttggccactgccaggtcagactggtgttttaaggaatactgggtgcttcatataggaactgaaggggtaaacttactaaaccattcaacctgtgattggtgatgttttcctgtcattttaagagtcgacacatgggtgggggggcagatgtaaaaaaacttgtacaattttaaaatatcacaattaaacgtgagctggtttcccaaaa

SEQ ID NO: 208 - protein, 181 aa, Mus Musculus
MMLQHPGQVSASEVSATAIVPCLSPPGSLVFEDFANLTPFVKEELRFAIQNKHLCHRMSSALESVTVNNRPLEMSVTKSEAAPEEDERKRRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 209 - protein, 971 aa, Mus Musculus
MDSAETELTPAPEGRKRYSDIFQSLDNLEISLGNVTFDPLAGDPVRREDLEPDKADTATVVTEENSEASSWRDLSPEGPAPLTEEELDLRLIRTKGGVDAALEYAKAWSRYAKELLAWTDKRANYELEFAKSIMKLAEAGKVSILQQSQMPLQYIYTLFLEHDLSLGALALETVAQQKRDYYQPLAAKRMEIEKWRKEFKEQWLKEQKRMNEAVQALRRSELQYIQRREDLRARSQGSPEDPPSQASPGSNKQQERRRRSREEAQAKAHEAEALYQACVREANSRQQDLETTKRRIVSHVRKLVLQGDEVLRRVTLGLFELRGAQAERGPRSFSALAECCVPFEPGQRYQEFVRTLQPGAPPPPSPAFCFQEFTAVVHSFPQDTKKKFSGPLPPRLEEEGSPEPGPWEVASLGSQGIPGSDVDSVGGGSESRSLDSPTSSPGAGARRLVKASSTGTESSDDFEERDPDLGDGIENGVGSPFRKWTLSTAAQTHRLRRLRGPAKCRECEAFMVSGTECEECFLTCHKRCLETLLILCGHRRLPARMSLFGVDFLQLPRDFPEEVPFVITRCTAEIEHRALGLQGIYRVSGSRVRVERLCQAFENGRALVELSGNSPHDITSVLKRFLQELTDPVVPFHLYDAFISLAKTLHADPGDDPGTPNPSPEIIRSLKTLLVQLPDSNYSTLRHLVAHLFRVAARFEENKMSANNLGIVFGPTLLRPPDGPRATGASPVACLLDSGHQAQLVEFLIVHYEQIFGMDELPLASEPLTQDPGLAPACLESSPQHPASLLAQDTQPLTIALDSSPDPKHHSALEKCPEVTPPELATLQRDQREEEVEDTRDGAGDGSSHCPEDLALGAQSRGHFSRQPVKYSRGGVRPVTHQLSSLALVASKLCEETPVTVSAVHRGSLRVRGLGPAAACPEGSPLRRNPLPKHFEITQETARLLSKLNSDAVSRTTCCADPEPEESEEHL

SEQ ID NO: 210 - protein, 408 aa, Mus Musculus
MDSAETELTPAPEGRKRYSDIFQSLDNLEISLGNVTFDPLAGDPVRREDLEPDKADTATVVTEENSEASSWRDLSPEGPAPLTEEELDLRLIRTKGGVDAALEYAKAWSRYAKELLAWTDKRANYELEFAKSIMKLAEAGKVSILQQSQMPLQYIYTLFLEHDLSLGALALETVAQQKRDYYQPLAAKRMEIEKWRKEFKEQWLKEQKRMNEAVQALRRSELQYIQRREDLRARSQGSPEDPPSQASPGSNKQQERRRRSREEAQAKAHEAEALYQACVREANSRQQDLETTKRRIVSHVRKLVLQGDEVLRRVTLGLFELRGAQAERGPRSFSALAECCVPFEPGQRYQEFVRTLQPGAPPPPSPAFCFQEFTAVVHRTQKRSFRGLYLQGWRRRVPLSLALGRLPA

SEQ ID NO: 211 - protein, 846 aa, Mus Musculus
MDSAETELTPAPEGRKRYSDIFQSLDNLEISLGNVTFDPLAGDPVRREDLEPDKADTATVVTEENSEASSWRDLSPEGPAPLTEEELDLRLIRTKGGVDAALEYAKAWSRYAKELLAWTDKRANYELEFAKSIMKLAEAGKVSILQQSQMPLQYIYTLFLEHDLSLGALALETVAQQKRDYYQPLAAKRMEIEKWRKEFKEQWLKEQKRMNEAVQALRRSELQYIQRREDLRARSQGSPEDPPSQASPGSNKQQERRRRSREEAQAKAHEAEALYQACVREANSRQQDLETTKRRIVSHVRKLVLQGDEVLRRVTLGLFELRGAQAERGPRSFSALAECCVPFEPGQRYQEFVRTLQPGAPPPPSPAFCFQEFTAVVHSFPQDTKKKFSGPLPPRLEEEGSPEPGPWEVASLGSQGIPGSDVDSVGGGSESRSLDSPTSSPGAGARRLVKASSTGTESSDDFEERDPDLGDGIENGVGSPFRKWTLSTAAQTHRLRRLRGPAKCRECEAFMVSGTECEECFLTCHKRCLETLLILCGHRRLPARMSLFGVDFLQLPRDFPEEVPFVITRCTAEIEHRALGLQGIYRVSGSRVRVERLCQAFENGRALVELSGNSPHDITSVLKRFLQELTDPVVPFHLYDAFISLAKTLHADPGDDPGTPNPSPEIIRSLKTLLVQLPDSNYSTLRHLVAHLFRVAARFEENKMSANNLGIVFGPTLLRPPDGPRATGASPVACLLDSGHQAQLVEFLIVHYEQIFGMDELPLASEPLTQDPGLAPACLESSPQHPASLLAQDTQPLTIALDSSPDPKHHSALEKCPEVTPPEGPATALRTWPWEHNPGATSAASQ

SEQ ID NO: 212 - protein, 843 aa, Mus Musculus
MDSAETELTPAPEGRKRYSDIFQSLDNLEISLGNVTFDPLAGDPVRREDLEPDKADTATVVTEENSEASSWRDLSPEGPAPLTEEELDLRLIRTKGGVDAALEYAKAWSRYAKELLAWTDKRANYELEFAKSIMKLAEAGKVSILQQSQMPLQYIYTLFLEHDLSLGALALETVAQQKRDYYQPLAAKRMEIEKWRKEFKEQWLKEQKRMNEAVQALRRSELQYIQRREDLRARSQGSPEDPPSQASPGSNKQQERRRRSREEAQAKAHEAEALYQACVREANSRQQDLETTKRRIVSHVRKLVLQGDEVLRRVTLGLFELRGAQAERGPRSFSALAECCVPFEPGQRYQEFVRTLQPGAPPPPSPAFCFQEFTAVVHSFPQDTKKKFSGPLPPRLEEEGSPEPGPWEVASLGSQGIPGSDVDSVGGGSESRSLDSPTSSPGAGARRLVKASSTGTESSDDFEERDPDLGDGIENGVGSPFRKWTLSTAAQTHRLRRLRGPAKCRECEAFMVSGTECEECFLTCHKRCLETLLILCGHRRLPARMSLFGVDFLQLPRDFPEEVPFVITRCTAEIEHRALGLQGIYRVSGSRVRVERLCQAFENGRALVELSGNSPHDITSVLKRFLQELTDPVVPFHLYDAFISLAKTLHADPGDDPGTPNPSPEIIRSLKTLLVQLPDSNYSTLRHLVAHLFRVAARFEENKMSANNLGIVFGPTLLRPPDGPRATGASPVACLLDSGHQAQLVEFLIVHYEQIFGMDELPLASEPLTQDPGLAPACLESSPQHPASLLAQDTQPLTIALDSSPDPKHHSALEKCPEVTPPEFWFFETGFLCVALAVLELTL

SEQ ID NO: 213 - protein, 633 aa, Mus Musculus
MDSAETELTPAPEGRKRYSDIFQSLDNLEISLGNVTFDPLAGDPVRREDLEPDKADTATVVTEENSEASSWRDLSPEGPAPLTEEELDLRLIRTKGGVDAALEYAKAWSRYAKELLAWTDKRANYELEFAKSIMKLAEAGKVSILQQSQMPLQYIYTLFLEHDLSLGALALETVAQQKRDYYQPLAAKRMEIEKWRKEFKEQWLKEQKRMNEAVQALRRSELQYIQRREDLRARSQGSPEDPPSQASPGSNKQQERRRRSREEAQAKAHEAEALYQACVREANSRQQDLETTKRRIVSHVRKLVLQGDEVLRRVTLGLFELRGAQAERGPRSFSALAECCVPFEPGQRYQEFVRTLQPGAPPPPSPAFCFQEFTAVVHSFPQDTKKKFSGPLPPRLEEEGSPEPGPWEVASLGSQGIPGSDVDSVGGGSESRSLDSPTSSPGAGARRLVKASSTGTESSDDFEERDPDLGDGIENGVGSPFRKWTLSTAAQTHRLRRLRGPAKCRECEAFMVSGTECEECFLTCHKRCLETLLILCGHRRLPARMSLFGVDFLQLPRDFPEEVPFVITRCTAEIEHRALGLQGIYRVSGSRVRVERLCQAFENGRALVELSGNSPHDITSVLKRFLQEGGCSV

SEQ ID NO: 214 - protein, 78 aa, Mus Musculus
MDSAETELTPAPEGRKRYSDIFQSLDNLEISLGNVTFDPLAGDPVRREDLEPDKADTATVVTEERKNWICDSFGPRVV

SEQ ID NO: 215 - protein, 303 aa, Mus Musculus
MAPPALQAQPPGGSQLRFLLFLLLLLLLLSWPSQGDALAMPEQRRSGPESQLNADELRGRFQDLLSRLHANQSREDSNSEPSPDPAVRILSPEVRLGSHGQLLLRVNRASLSQGLPEAYRVHRALLLLTPTTRPWDITRPLKRALSLQGPRAPALRLRLTPPPDLAMLPSGGAQLELRLRVAAGRGRRSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVARGCHCA

SEQ ID NO: 216 - protein, 159 aa, Mus Musculus
MSNPGDVRPVPHRSKVCRCLFGPVDSEQLRRDCDALMAGCLQEARERWNFDFVTETPLEGNFVWERVRSLGLPKVYLSPGSRSRDDLGGDKRPSTSSALLQGPAPEDHVALSLSCTLVSERPEDSPGGPGTSQGRKRRQTSLTDFYHSKRRLVFCKRKP

SEQ ID NO: 217 - protein, 294 aa, Mus Musculus
MIEVLTTDSQKLLHQLNTLLEQESRCQPKVCGLKLIESAHDNGLRMTARLRDFEVKDLLSLTQFFGFDTETFSLAVNLLDRFLSKMKVQAKHLGCVGLSCFYLAVKATEEERNVPLATDLIRISQYRFTVSDLMRMEKIVLEKVCWKVKATTAFQFLQLYYSLVHDTLPFERRNDLNFERLEAQLKACHCRIIFSKAKPSVLALSILALEIQALKYVELTEGVECIQKHSKISGRDLTFWQELVSKCLTEYSSNKCSKPNGQKLKWIVSGRTARQLKHSYYRITHLPTIPETIC

SEQ ID NO: 218 - protein, 187 aa, Mus Musculus
MIEVLTTDSQKLLHQLNTLLEQESRCQPKVCGLKLIESAHDNGLRMTARLRDFEVKDLLSLTQFFGFDTETFSLAVNLLDRFLSKMKVQAKHLGCVGLSCFYLAVKATEEERNVPLATDLIRISQYRFTVSDLMRMEKIVLEKVCWKVKATTAFQFLQLYYSLVHDTLPFESLLCWRYLSLRWRSKH

SEQ ID NO: 219 - protein, 203 aa, Mus Musculus [sequence omitted]

SEQ ID NO: 220 - protein, 342 aa, Mus Musculus
MRKDTPPPLVPPAAREWNLPPNAPACMERQLEAARYRSDGSLLLGVSSLSGRCWVGSLWFFKDPSAAPNEGFCSAGVQTEAGVADLTWVGDKGILVASDSGAVELWELDENETLIVSKFCKYEHDDIVSTVTVLSSGTQAVSGSKDCCIKIWDLAQQVSLNSYRAHAGQVTCVAASPHKDSVFLSCSEDSRILLWDTRCPKPASQMACNASGYLPTALAWHPQQSEVFVFGDENGSVSLVDTKNASCTLSSAVHSQGVTRLVFSPHSVPLLTSLSEDCSLAVLDSSLSEVFRSRAHRDFVRDATWSPLNHSLLTTVGWDHQVIHHVVPLEPLPNPGPDSVVE

SEQ ID NO: 221 - protein, 285 aa, Mus Musculus
MRKDTPPPLVPPAAREWNLPPNAPACMERQLEAARYRSDGSLLLGVSSLSGRCWVGSLWFFKDPSAAPNEGFCSAGVQTEAGVADLTWVGDKGILVASDSGAVELWELDENETLIVSKFCKYEHDDIVSTVTVLSSGTQAVSGSKDCCIKIWDLAQQVSLNSYRAHAGQVTCVAASPHKDSVFLSCSEDSRILLWDTRCPKPASQMACNASGYLPTALAWHPQQSEVFVFGDENGSVSLVDTKNASCTLSSAVHSQGVTRLVFSPHRCCVSPGTWKGWVGTVVKE

SEQ ID NO: 222 - protein, 234 aa, Mus Musculus
MRKDTPPPLVPPAAREWNLPPNAPACMERQLEAARYRSDGSLLLGVSSLSGRCWVGSLWFFKDPSAAPNEGFCSAGVQTEAGVADLTWVGDKGILVASDSGAVELWELDENETLIVSKFCKYEHDDIVSTVTVLSSGTQAVSGSKDCCIKIWDLAQQVSLNSYRAHAGQVTCVAASPHKDSVFLSCSEDSRILLWDTRCPKPASQMACNASGYLPTALAWHPQQSEVFVFGKAA

SEQ ID NO: 223 - protein, 269 aa, Mus Musculus
MRKDTPPPLVPPAAREWNLPPNAPACMERQLEAARYRSDGSLLLGVSSLSGRCWVGSLWFFKDPSAAPNEGFCSAGVQTEAGVADLTWVGDKGILVASDSGAVELWELDENETLIVSKFCKYEHDDIVSTVTVLSSGTQAVSGSKDCCIKIWDLAQQVSLNSYRAHAGQVTCVAASPHKDSVFLSCSEDSRILLWDTRCPKPASQMACNASGYLPTALAWHPQQSEVFVFGDENGSVSLVDTKNASCTLSSAVHSQGVTRLCPPPDFSQ

SEQ ID NO: 224 - protein, 751 aa, Mus Musculus
MRLLRVAAALGRGPFPRVPAVLGWQGKQADWKTRRWCSSGPVPNEKIRNIGISAHIDSGKTTLTERVLYYTGRIATMHEVKGKDGVGAVMDSMELERQRGITIQSAATYTMWKDININIIDTPGHVDFTIEVERALRVLDGAVLVLCAVGGVQCQTMTVSRQMKRYNVPFLTFINKLDRMGSNPSRALQQMRSKLNHNAAFVQIPIGLEGDFKGIIDLIEERAIYFDGDFGQIVRYDEIPAGLRAAAADHRQELIECVANSDEQLGELFLEEKIPSVSDLKRAIRRATLSRSFTPVFLGSALKNKGVQPLLDAVLEYLPNPSEVQNYAILNQNDSKEKTKILMNPQRDDSHPFVGLAFKLEAGRFGQLTYVRNYQGELKKGSTIYNTRTGKKVRVQRLVRMHADMMEDVEEVYAGDICALFGIDCASGDTFTNKDNSDLSMESIHVPEPVISIAMRPSNKNDLEKFSKGIGRFTREDPTFKVHFDPESKETIVSGMGELHLEIYAQRMEREYGCPCITGKPKVAFRETIVAPVPFDFTHKKQSGGAGQFGKVIGVLEPLPPEDYTKLEFSDETFGSNVPKQFVPAVEKGFLDACEKGPLSGHKLSGLRFVLQDGAHHMVDSNEISFIRAGEGALKQALANGTLCIIEPIMSVEVIAPNEFQGTVFAGINRRHGVITGQDGIEDYFTLYADVPLNNMFGYSTELRSCTEGKGEYTMEYCRYQPCSPSTQEELINKYLEATGQLPVKKGKAKN

SEQ ID NO: 225 - protein, 560 aa, Mus Musculus
MRLLRVAAALGRGPFPRVPAVLGWQGKQADWKTRRWCSSGPVPNEKIRNIGISAHIDSGKTTLTERVLYYTGRIATMHEVKGKDGVGAVMDSMELERQRGITIQSAATYTMWKDININIIDTPGHVDFTIEVERALRVLDGAVLVLCAVGGVQCQTMTVSRQMKRYNVPFLTFINKLDRMGSNPSRALQQMRSKLNHNAAFVQIPIGLEGDFKGIIDLIEERAIYFDGDFGQIVRYDEIPAGLRAAAADHRQELIECVANSDEQLGELFLEEKIPSVSDLKRAIRRATLSRSFTPVFLGSALKNKGVQPLLDAVLEYLPNPSEVQNYAILNQNDSKEKTKILMNPQRDDSHPFVGLAFKLEAGRFGQLTYVRNYQGELKKGSTIYNTRTGKKVRVQRLVRMHADMMEDVEEVYAGDICALFGIDCASGDTFTNKDNSDLSMESIHVPEPVISIAMRPSNKNDLEKFSKGIGRFTREDPTFKVHFDPESKETIVSGMGELHLEIYAQRMEREYGCPCITGKPKVAFRETIVAPVPVASNKEKLYMLPAMQCRGVEWCNLTQ

SEQ ID NO: 226 - protein, 129 aa, Mus Musculus
MASAWPRSLPQILVLGFGLVLMRAAAGEQAPGTSPCSSGSSWSADLDKCMDCASCPARPHSDFCLGCAAAPPAHFRLLWPILGGALSLVLVLALVSSFLVWRRCRRREKFTTPIEETGGEGCPGVALIQ

SEQ ID NO: 227 - protein, 139 aa, Mus Musculus
MASAWPRSLPQILVLGFGLVLMRAAAGEQAPGSKFQVGKVSSFIRSPSFSFATCPIEAPPHALAAAPGARTSTSAWTALLVQRDHTATSAWDAPQHLLPTSGYCGPFWGALLVWSWFWRWFLVSWSGEDAAGEKSLLPP

SEQ ID NO: 228 - protein, 83 aa, Mus Musculus
MASAWPRSLPQILVLGFGLVLMRAAAGEQAPGTSPCSSGSSWSADLDKCMDCASCPARPHSDFCLGFSWSGEDAAGEKSLLPP

SEQ ID NO: 229 - protein, 120 aa, Mus Musculus
MILQQPLERGPPSRDPRATTGVTRGLNASLSPREPLHKQFLSEENMATHFSRLSLHNDHPYCSPPVTFPEALPPLRSPCPELLLWRYPGSLIPEALRLLRLGDTPSPYYPASPAGDIVEL

SEQ ID NO: 230 - protein, 202 aa, Mus Musculus
MAKAYDHLFKLLLIGDSGVGKTCLIIRFAEDNFNSTYISTIGIDFKIRTVDIEGKRIKLQVWDTAGQERFKTITTAYYRGAMGIILVYDITDEKSFENIQNWMKSIKENASAGVERLLLGNKCDMEAKRQVQREQAEKLAREHRIRFFETSAKSSVNVDEAFSSLARDILLKTGGRRSGTNSKPSSTGLKTSDKKKNKCLLG

SEQ ID NO: 231 - protein, 300 aa, Mus Musculus
MAGIPEVVAMDCEMVGLGPQRVSGLARCSIVNIHGAVLYDKYIRPEGEITDYRTQVSGVTPQHMVRATPFGEARLEILQLLKGKLVVGHDLKHDFNALKEDMSKYTIYDTSTDRLLWHEAKLQYYSRVSLRLLCKRLLHKNIQVLPGSLLGVGGCILPGTDILHLLLYVGMVRIADLRLLTPFLPPSCLACPLLPESLASARSHAVISALSSSSHLLTPLPNPSQGPQGHVDRLSGQLQDWGGSPLAPALPVSAEQLAGPLLCGRCQGHNGALQNLSATQSPARAALPWDVRLNFILIQG

SEQ ID NO: 232 - protein, 181 aa, Mus Musculus
MAGIPEVVAMDCEMVGLGPQRVSGLARCSIVNIHGAVLYDKYIRPEGEITDYRTQVSGVTPQHMVRATPFGEARLEILQLLKGKLVVGHDLKHDFNALKEDMSKYTIYDTSTDRLLWHEAKLQYYSRVSLRLLCKRLLHKNIQNNWRGHCSVEDARATMELYKISQRLRAQRGLPCPGTSD

SEQ ID NO: 233 - protein, 201 aa, Mus Musculus
MAGIPEVVAMDCEMVGLGPQRVSGLARCSIVNIHGAVLYDKYIRPEGEITDYRTQVSGVTPQHMVRATPFGEARLEILQLLKGKLVVGHDLKHDFNALKEDMSKYTIYDTSTDRLLWHEAKLQYYSRVSLRLLCKRLLHKNIQVRSLPAPMTLLVSLSSPSSLSFSLFPLRCSFSFSFLSYSSKQGVLLLSSLVLPLLPCF

SEQ ID NO: 234 - protein, 358 aa, Mus Musculus
MVWGHFPLAVFDSLGSGEMADSVKTFLQDLGRGIKDSIWGICTISKLDARIQQKREEQRRRRASSLLAQRRPQSVERKQESEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIISGCLFSILFPLFIISANEAKTPGKAYLFQLRLFSLVVFLSNRLFHKTVYLQSALSSSSSAEKFPSPHPSPAKLKAAAGH

SEQ ID NO: 235 - protein, 374 aa, Mus Musculus
MVWGHFPLAVFDSLGSGEMADSVKTFLQDLGRGIKDSIWGICTISKLDARIQQKREEQRRRRASSLLAQRRPQSVERKQESEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIISGCLFSILFPLFIISANEAKTPGKAYLFQLRLFSLVVFLSNRLFHKTVYLQPLSPAVKGVGGTGWRMWQLFSLFSSPCRGRQNPLPRALCIVPCL

SEQ ID NO: 236 - protein, 179 aa, Mus Musculus
MVWGHFPLAVFDSLGSGEMADSVKTFLQDLGRGIKDSIWGICTISKLDARIQQKREEQRRRRASSLLAQRRPQSVERKQESEPRIVSRIFQCCAWNGGVFWFSLLLFYRVFIPVLQSVTARIIGDPSLHGDVWSWLEFFLTSIFSALWVLPLFVLSKVVNAIWFQVGAGQNGTGFYMLG

SEQ ID NO: 237 - DNA, 20 bp, Artificial Sequence
  Synthetic polynucleotide
gggaaatcgtgcgtgacatt

SEQ ID NO: 238 - DNA, 17 bp, Artificial Sequence
  Synthetic polynucleotide
gcggcagtggccatctc

SEQ ID NO: 239 - DNA, 76 bp, Artificial Sequence
  Synthetic polynucleotide
gggaaatcgtgcgtgacattaaagagaagctgtgctatgttgccctagacttcgagcaagagatggccactgccgc

SEQ ID NO: 240 - DNA, 20 bp, Artificial Sequence
  Synthetic polynucleotide
taccctattgagcacggcat

SEQ ID NO: 241 - DNA, 20 bp, Artificial Sequence
  Synthetic polynucleotide
cgcagctcgttgtagaaggt

SEQ ID NO: 242 - DNA, 80 bp, Artificial Sequence
  Synthetic polynucleotide
taccccattgaacacggcatcatcacgaactgggatgacatggagaagatctggcaccactccttctacaacgagctgcg

SEQ ID NO: 243 - DNA, 21 bp, Artificial Sequence
  Synthetic polynucleotide
gcgaaagtggaaaagccaagt

SEQ ID NO: 244 - DNA, 24 bp, Artificial Sequence
  Synthetic polynucleotide
gccacatcaacaggactcttgtag

SEQ ID NO: 245 - DNA, 76 bp, Artificial Sequence
  Synthetic polynucleotide
gcgaaagtggaaaagccaagtacaaagcctaaaagacagcggcaagttgaatctacaagagtcctgttgatgtggc

SEQ ID NO: 246 - DNA, 23 bp, Artificial Sequence
  Synthetic polynucleotide
caagcataccaagaagcatttga

SEQ ID NO: 247 - DNA, 18 bp, Artificial Sequence
  Synthetic polynucleotide
gggccagacccagtctga

SEQ ID NO: 248 - DNA, 76 bp, Artificial Sequence
  Synthetic polynucleotide
caagcataccaagaagcatttgaaatcagcaaaaaggagatgcagccgacacaccccatcagactgggtctggccc

SEQ ID NO: 249 - DNA, 20 bp, Artificial Sequence
  Synthetic polynucleotide
ccaaggactgggcttagggt

SEQ ID NO: 250 - DNA, 22 bp, Artificial Sequence
  Synthetic polynucleotide
agagattcccttgtgcaaatgc

SEQ ID NO: 251 - DNA, 144 bp, Artificial Sequence
  Synthetic polynucleotide
ccaaggactgggcttagggttcaggggagccttccagggtgtctaattgccctgtctctggttctggggcagacagagagcctcaagctaggtcacaaagcgactcatactaaggatctgcagcatttgcacaagggaatctct

SEQ ID NO: 252 - DNA, 22 bp, Artificial Sequence
  Synthetic polynucleotide
gatctgggtaggtggttgttgg

SEQ ID NO: 253 - DNA, 22 bp, Artificial Sequence
  Synthetic polynucleotide
cgcacacccttataaaagtccc

SEQ ID NO: 254 - DNA, 102 bp, Artificial Sequence
  Synthetic polynucleotide
gatctgggtaggtggttgttggggcagaaaggaggtcgtagacttaggatataggaaaccaggaaaaactgactgaggaagggacttttataagggtgtgcg

SEQ ID NO: 255 - DNA, 24 bp, Artificial Sequence
  Synthetic polynucleotide
atctcgatggtaacgggtctaatc

SEQ ID NO: 256 - DNA, 20 bp, Artificial Sequence
  Synthetic polynucleotide
tttgtgctgaccttcatgcc

SEQ ID NO: 257 - DNA, 124 bp, Artificial Sequence
  Synthetic polynucleotide
atctcgatggtaacgggtctaatcagcccatgatcaacataactgtggtgatatacatttggtattttttaattttcggatgccttcctcaacatagccgtcaaggcatgaaggtcagcacaaa

SEQ ID NO: 258 - DNA, 21 bp, Artificial Sequence
  Synthetic polynucleotide
ccagcctagcctctcttttgc

SEQ ID NO: 259 - DNA, 23 bp, Artificial Sequence
  Synthetic polynucleotide
aacattcccacagggtacattca

SEQ ID NO: 260 - DNA, 121 bp, Artificial Sequence
  Synthetic polynucleotide
ccagcctagcctctcttttgcactgctggttcagcccactgggcctccgtcctttcctctggaagggacttggccttgggtgacaaattcctctttgatgaatgtaccctgtgggaatgtt

SEQ ID NO: 261 - DNA, 20 bp, Artificial Sequence
  Synthetic polynucleotide
gggccacaatggagctctac

SEQ ID NO: 262 - DNA, 26 bp, Artificial Sequence
  Synthetic polynucleotide
acaggtctcattcatggaaaactatg

SEQ ID NO: 263 - DNA, 151 bp, Artificial Sequence
  Synthetic polynucleotide
gggccacaatggagctctacaaaatctctcagcgactcagagcccagcgagggctgccctgcctgggaacatcagcctgaacttcatcctcatccaggatcagaagcagctactccttgaaggaccatagttttccatgaatgagacctgt

SEQ ID NO: 264 - DNA, 17 bp, Artificial Sequence
  Synthetic polynucleotide
ggaggagggcagagcca

SEQ ID NO: 265 - DNA, 25 bp, Artificial Sequence
  Synthetic polynucleotide
tctggttcattatcaagggaagttg

SEQ ID NO: 266 - DNA, 151 bp, Artificial Sequence
  Synthetic polynucleotide
ggaggagggcagagccagaggagggacagcctgatgcagacagccctgactcaacctgccagcccccttacctgtcagccttgaggagatggaacagcccagcctactaggcctgcccccactccccaacttcccttgataatgaaccaga

SEQ ID NO: 267 - DNA, 22 bp, Artificial Sequence
  Synthetic polynucleotide
atcctgttgggtctgcttctca

SEQ ID NO: 268 - DNA, 30 bp, Artificial Sequence
  Synthetic polynucleotide
cacaagtaaatacatagactggaatcaatg

SEQ ID NO: 269 - DNA, 155 bp, Artificial Sequence
  Synthetic polynucleotide
atcctgttgggtctgcttctcacagcgtatggggctgtgtgcttttactcatggtggcagcagcagttgttgtcacttctttggccaggcacagtgggtgatccttacagagcagcacagattcccattgattccagtctatgtatttacttgtg

SEQ ID NO: 270 - DNA, 25 bp, Artificial Sequence
  Synthetic polynucleotide
tgtttaaaatctgggctcttgattc

SEQ ID NO: 271 - DNA, 31 bp, Artificial Sequence
  Synthetic polynucleotide
gtatttccaatagtcaatgcagtaactctac

SEQ ID NO: 272 - DNA, 155 bp, Artificial Sequence
  Synthetic polynucleotide
tgtttaaaatctgggctcttgattcagaccgagttttaatactggatctcattcaatctttgtgttttttctttttcttttcttttttttccaatagtacggcagtgctaatagcagtcctttagtagagttactgcattgactattggaaatac

SEQ ID NO: 273 - DNA, 21 bp, Artificial Sequence
  Synthetic polynucleotide
gccacagtagaggctgcactt

SEQ ID NO: 274 - DNA, 24 bp, Artificial Sequence
  Synthetic polynucleotide
gaatcaactggaaatgttctaggg

SEQ ID NO: 275 - DNA, 137 bp, Artificial Sequence
  Synthetic polynucleotide
gccacagtagaggctgcacttgccactgctgtgtagcggcactctcctgactcacttaaaaattctctggaggtttaagtgaggacccagttccatctctagatatccaggctccctagaacatttccagttgattc

SEQ ID NO: 276 - DNA, 20 bp, Artificial Sequence
  Synthetic polynucleotide
ctgcaactgagcatgtgcgt

SEQ ID NO: 277 - DNA, 19 bp, Artificial Sequence
  Synthetic polynucleotide
tgcataagaaccaccgggg

SEQ ID NO: 278 - DNA, 155 bp, Artificial Sequence
  Synthetic polynucleotide
ctgcaactgagcatgtgcgtgggcgagtgcccccacctctaccggtcggccaacacgcatgcgcagatcaaagcacgcctgcatggcctgcagcccgacagagtgccggccccgtgctgtgtcccctccagctacaccccggtggttcttatgca

SEQ ID NO: 279 - DNA, 16 bp, Artificial Sequence
  Synthetic polynucleotide
ggacggccgagcacaa

SEQ ID NO: 280 - DNA, 22 bp, Artificial Sequence
  Synthetic polynucleotide
gtgtgcatgtgcataggtcaga

SEQ ID NO: 281 - DNA, 123 bp, Artificial Sequence
  Synthetic polynucleotide
ggacggccgagcacaatggtgcttgctgtacagcctcatagaccttccttggatctcccagacacacttagtggaaggagagaatccattcttgcaaattgtctgacctatgcacatgcacac

SEQ ID NO: 282 - DNA, 26 bp, Artificial Sequence
  Synthetic polynucleotide
tactgtttcctgtgaagcacatacct

SEQ ID NO: 283 - DNA, 23 bp, Artificial Sequence
  Synthetic polynucleotide
gcagagcagtaaagaccaaaacc

SEQ ID NO: 284 - DNA, 137 bp, Artificial Sequence
  Synthetic polynucleotide
tactgtttcctgtgaagcacataccttgtatgtgggaggtaaaggagcacgccagctgctccatgtcactccctctatagccatcactgtcttgtttttttgtaactcaggttaggttttggtctttactgctctgc

SEQ ID NO: 285 - DNA, 21 bp, Artificial Sequence
  Synthetic polynucleotide
aatctaggctgcctcctggag

SEQ ID NO: 286 - DNA, 28 bp, Artificial Sequence
  Synthetic polynucleotide
aggtcaattatacaaggcatgactttag

SEQ ID NO: 287 - DNA, 101 bp, Artificial Sequence
  Synthetic polynucleotide
aatctaggctgcctcctggaggaagatacttaggagttcagaagtgaagagatgaggcttataatactttttcctaaagtcatgccttgtataattgacct

SEQ ID NO: 288 - DNA, 20 bp, Artificial Sequence
  Synthetic polynucleotide
tcgtcagaattgctgcctca

SEQ ID NO: 289 - DNA, 24 bp, Artificial Sequence
  Synthetic polynucleotide
tctgtggtaaaaacctcctggagt

SEQ ID NO: 290 - DNA, 151 bp, Artificial Sequence
  Synthetic polynucleotide
tcgtcagaattgctgcctcaatctagtcccatttgagaaaatttgtttctactgtctcaataactggatgaaatatcactctgaaaacttgcctattgcactaaagctagtttaggcttgataaaacactccaggaggtttttaccacaga

SEQ ID NO: 291 - DNA, 30 bp, Artificial Sequence; identical to SEQ ID NO: 10
  Synthetic polynucleotide

SEQ ID NO: 292 - DNA, 26 bp, Artificial Sequence
  Synthetic polynucleotide
ctttgccttagaaaatatgatcctgc

SEQ ID NO: 293 - DNA, 109 bp, Artificial Sequence
  Synthetic polynucleotide
ataaaatcaatcccactttcttgttaaaaggagaaacgatctgaattttgaaagactagaagcccaacttaaggcctgccactgcaggatcatattttctaaggcaaag

SEQ ID NO: 294 - DNA, 28 bp, Artificial Sequence
  Synthetic polynucleotide
gagatccaagcactgaagtatgtagagt

SEQ ID NO: 295 - DNA, 28 bp, Artificial Sequence
  Synthetic polynucleotide
tcaggagtacagtggatacatttctctt

SEQ ID NO: 296 - DNA, 134 bp, Artificial Sequence
  Synthetic polynucleotide
gagatccaagcactgaagtatgtagagttaacagaaggagtagaatgtattcagaaacattccaaggtatgccaaggtgatagcattgatcctattagcaagctacaagagaaatgtatccactgtactcctga

SEQ ID NO: 297 - DNA, 21 bp, Artificial Sequence
  Synthetic polynucleotide
gggcactgcatggattatgtc

SEQ ID NO: 298 - DNA, 21 bp, Artificial Sequence
  Synthetic polynucleotide
ccatgcccagaataaaggagc

SEQ ID NO: 299 - DNA, 101 bp, Artificial Sequence
  Synthetic polynucleotide
gggcactgcatggattatgtcggttttacctagctttggagaccatcattttttcctaataggtttgcttcattgtttcagctcctttattctgggcatgg

SEQ ID NO: 300 - DNA, 24 bp, Artificial Sequence
  Synthetic polynucleotide
ctccgttagcaattatgggtcagt

SEQ ID NO: 301 - DNA, 21 bp, Artificial Sequence
  Synthetic polynucleotide
gaagctcatggtccttcaggg

SEQ ID NO: 302 - DNA, 102 bp, Artificial Sequence
  Synthetic polynucleotide
ctccgttagcaattatgggtcagtaactgatcttattagagctttacaactttggtttaggaaaagcacataaaatgggcgccctgaaggaccatgagcttc

SEQ ID NO: 303 - DNA, 23 bp, Artificial Sequence
  Synthetic polynucleotide
ctgtcattgtcatttttcccacc

SEQ ID NO: 304 - DNA, 22 bp, Artificial Sequence
  Synthetic polynucleotide
atgccggatcttcaatcttagg

SEQ ID NO: 305 - DNA, 118 bp, Artificial Sequence
  Synthetic polynucleotide
ctgtcattgtcatttttcccacctaaaaattccctgaggactgatctgggtactttgctctggagagctgaagtctgagcgctgtatatttggactcctaagattgaagatccggcat

SEQ ID NO: 306 - DNA, 21 bp, Artificial Sequence
  Synthetic polynucleotide
aactggctgatgaccagctgt

SEQ ID NO: 307 - DNA, 17 bp, Artificial Sequence
  Synthetic polynucleotide
tgctgtgcccgggttct

SEQ ID NO: 308 - DNA, 95 bp, Artificial Sequence
  Synthetic polynucleotide
aactggctgatgaccagctgtgctactctgtgctgaccgaggactgatgcctccttcccctgtacccactgctgaggaagaacccgggcacagca

SEQ ID NO: 309 - DNA, 17 bp, Artificial Sequence
  Synthetic polynucleotide
gtgagtcaggccgggct

SEQ ID NO: 310 - DNA, 23 bp, Artificial Sequence
  Synthetic polynucleotide
tcaaaaaatcggattctgtgctc

SEQ ID NO: 311 - DNA, 115 bp, Artificial Sequence
  Synthetic polynucleotide
gtgagtcaggccgggctgctgtggccgaatgccttgcttctgccctttatccagctctctccttcctacctcatcccctacagttggctcgagagcacagaatccgattttttga

SEQ ID NO: 312 - DNA, 21 bp, Artificial Sequence
  Synthetic polynucleotide
ttctcctgaatctggctgggt

SEQ ID NO: 313 - DNA, 23 bp, Artificial Sequence
  Synthetic polynucleotide
tggaaaaaggatgtccattcttc

SEQ ID NO: 314 - DNA, 105 bp, Artificial Sequence
  Synthetic polynucleotide
ttctcctgaatctggctgggtcccccttccttaccccaactctttaactggtgatgaaaacagcaaggagaaagggcagcctgaagaatggacatcctttttcca

SEQ ID NO: 315 - DNA, 25 bp, Artificial Sequence
  Synthetic polynucleotide
agaatccgattttttgagacaagtg

SEQ ID NO: 316 - DNA, 20 bp, Artificial Sequence
  Synthetic polynucleotide
gatctccggcctcctgtctt

SEQ ID NO: 317 - DNA, 104 bp, Artificial Sequence
  Synthetic polynucleotide
agaatccgattttttgagacaagtgccaaatccagtgtgaatgtggatgaggctttcagttccctggcccgtgacatcttgctcaagacaggaggccggagatc

SEQ ID NO: 318 - DNA, 20 bp, Artificial Sequence
  Synthetic polynucleotide
caagccctcaagcactgacc

SEQ ID NO: 319 - DNA, 21 bp, Artificial Sequence
  Synthetic polynucleotide
ggtctagcctccaggttcagg

SEQ ID NO: 320 - DNA, 113 bp, Artificial Sequence
  Synthetic polynucleotide
caagccctcaagcactgacctgaaagtatctgacaagaagaacagcaacaagtgctccttgggctgaggaacatttcttgcctcctattcaccctgaacctggaggctagacc

SEQ ID NO: 321 - DNA, 19 bp, Artificial Sequence
  Synthetic polynucleotide
gggtgggggaatctagcct

SEQ ID NO: 322 - DNA, 20 bp, Artificial Sequence
  Synthetic polynucleotide
ccccagacaaagggacatgt

SEQ ID NO: 323 - DNA, 106 bp, Artificial Sequence
  Synthetic polynucleotide
gggtgggggaatctagcctctctagagccctagccctctgacgaggaggaggtgtagtgccctgtagcttttccccaggactcgccacatgtccctttgtctgggg

SEQ ID NO: 324 - DNA, 22 bp, Artificial Sequence
  Synthetic polynucleotide
cgctgaagtcctcagtgacttg

SEQ ID NO: 325 - DNA, 22 bp, Artificial Sequence
  Synthetic polynucleotide
cactaagtgcttcgacacccac

SEQ ID NO: 326 - DNA, 161 bp, Artificial Sequence
  Synthetic polynucleotide
cgctgaagtcctcagtgacttgtcccatttcttagtagttgtacaaggagtcaggccaagatggtgcctcgggggctgagggagctcacaggaactgagcagtgactggtcctttcccagtattgaatactgagcccctgtgggtgtcgaagcacttagtg

SEQ ID NO: 327 - DNA, 19 bp, Artificial Sequence
  Synthetic polynucleotide
acagcctgatgcagacagc

SEQ ID NO: 328 - DNA, 25 bp, Artificial Sequence
  Synthetic polynucleotide
tctggttcattatcaagggaagttg

SEQ ID NO: 329 - DNA, 127 bp, Artificial Sequence
  Synthetic polynucleotide
acagcctgatgcagacagccctgactcaacctgccagcccccttacctggccagccttgaggagatggaacagcccaggctacaaggcctgcccccactcctcaacttcccttgataatgaaccaga

SEQ ID NO: 330 - DNA, 25 bp, Artificial Sequence
  Synthetic polynucleotide
cagaattgctgcctcaatctagtcc

SEQ ID NO: 331 - DNA, 24 bp, Artificial Sequence
  Synthetic polynucleotide
ggttttgcagatgtactcggttcc

SEQ ID NO: 332 - DNA, 219 bp, Artificial Sequence
  Synthetic polynucleotide
cagaattgctgcctcaatctagtcccatttgagaaaatttgtttctactgtctcaataactggatgaaatatcactctgaaaacttgcctattgcactaaagctagtttaggcttgataaaacactccaggaggtttttaccacagactgtttctattaaaactgctgcttctcatgtacaattttgttttaaaaggaaccgagtacatctgcaaaacc

(19) United States
(12) Patent Application Publication
(10) Pub. No.: US 2016/0177395 A1
(43) Pub. Date: Jun. 23, 2016

(54) BIOMARKERS FOR THE PREDICTION OF RENAL INJURY

(71) Applicant: COMPUGEN, LTD., Tel Aviv (IL)

(72) Inventors: Yaron Kinar, Te Aviv (IL); Merav Beiman, Ness Ziona (IL); Eve Montia, Rehovot (IL); Shira Wallach, Hod Hasharon (IL); Tania Pergam, Rishon Le Zion (IL); Amit Novik, Tel Mond (IL); Shirley Sameah-Greenwald, Kfar Saba (IL); Gad S. Cojocaru, Ramat-Hasharon (IL); Anat Cohen-Dayag, Rehovot (IL); Yael Furman, Ramat Hasharon (IL)

(21) Appl. No.: 14/990,762

(22) Filed: Jan. 7, 2016

Related U.S. Application Data
(63) Continuation of application No. 12/824,010, filed on Jun. 25, 2010.
Application No. 12/824,010 is a continuation-in-part of application No. PCT/IL2008/001561, filed on Dec. 1, 2008.
Application No. 12/824,010 is a continuation-in-part of application No. PCT/IL2009/000235, filed on Mar. 1, 2009.
(60) Provisional application No. 61/016,837, filed on Dec. 27, 2007.

(51) Int. Cl. C12Q 1/68

(52) U.S. Cl. CPC C12Q 1/6883; C12Q 2600/112; C12Q 2600/158; C12Q 2600/16; C12Q 2600/118

(57) ABSTRACT

The present invention relates to means and methods for predicting the onset of renal injury based on measuring the expression of polynucleotides and proteins, particularly on measuring the expression of sets of novel as well as known polynucelotides and proteins, and to kits utilizing same.

### CROSS-REFERENCE TO RELATED APPLICATIONS

The present application is a continuation-in-part of International Application No. PCT/IL2008/001561 filed Dec. 1, 2008, which claims priority to U.S. Provisional Application No. 61/016,837, filed Dec. 27, 2007, and is also a continuation-in-part of PCT/IL2009/000235 filed Mar. 1, 2009, the contents of each of which are incorporated herein by reference thereto.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 555,789 byte ASCII (text) file named “Seq_List” created on Jun. 25, 2010.

### FIELD OF THE INVENTION

The present invention relates to means and methods for predicting the onset of renal injury based on measuring the expression of polynucleotides and proteins, particularly measuring the expression of sets of novel polynucelotides and proteins as well as known genes.

### BACKGROUND OF THE INVENTION

Renal injury is a general term used to describe any damage to the kidney caused by various conditions including primary renal dysfunction, response to external substances or secondary renal pathology.

Renal injury commonly occurs after administration of pharmaceutical or toxic agents of various types. The process is typically initiated by a toxic injury to tubular epithelial cells in various nephron segments or by injury to specific cell types in the glomerulus. The initial injury is often followed by cellular proliferation and repair processes that attempts to restore normal renal function.

Early recognition of renal injury is hampered by the lack of accurate markers and the shortcoming of and over-reliance on serum markers of impaired glomerular filtration rate (i.e., serum creatinine and blood urea nitrogen, see e.g., Schrier et al, J Clin Invest, 114(1):5-14 (2004)). Drugs associated with the development of tubular nephrosis include aminoglycoside antibiotics, antifungals, antineoplastics, immunosuppresants and radiocontrast dyes, among others.

Similarly to the human clinical setting, long-term treatment of rats during preclinical drug development with relatively low doses of, for example, aminoglycoside antibiotics, heavy metal toxicants or antineoplastic drugs, leads to the development of degenerative lesions of the renal tubules. However, histopathological or clinical indications of kidney injury are not readily apparent in the early course of treatment, thus necessitating expensive and lengthy studies.

Changes in the expression of mRNA specifically expressed in the injured kidney cells are some of the earliest events that accompany renal injury. This is further accompanied by changes in the expression of other genes that contribute either to cellular repair or recovery of renal function or in those that mediate fibrosis and further pathology of the kidney (Matejka G L. et al., Exp Nephrol, 1998 6:253-264; Norman J T. et al. Proc Natl Acad Sci USA, 1988 85:6768-6772; Safirstein R. et al. Kidney Int, 1990 37:1515-1521). For example, elevation in the expression of heme oxygenase I (HO-1), kidney injury molecule-1 (KIM-1), clusterin, thymosin beta-4, osteopontin, and several growth factors have been reported in various models of renal injury (Hammerman et al, 1998 Curr Oppin Nephrol Hypertens 7:419-424; Yoshida et al, 2002 Kidney International 61: 1646-1654; Amin R P et al. 2004 Environ Health Perspect. 112(4):465-479; Thomas R. S et. al. 2001 Mol Pharmacol. 60(6): 1189-1194).

International Patent Application Publication No. WO 2006/033701 provides gene signatures as well as methods, apparatuses and reagents useful for predicting future renal tubule injury, based on the expression levels of genes in the signatures. In one particular embodiment that invention provides a method for predicting whether a compound will induce renal tubule injury using gene expression data from sub-acute treatments. However, the WO 2006/033701 application discloses that the necessary set useful for generating meaningful signatures of 186 genes. Such vast number of genes in a single signature requires cumbersome analyses, rendering the method unefficient.

International Patent Application Publication No. WO 02/095000 provides toxicity markers identified in tissues or cells exposed to a known renal toxin, based on the elucidation of the global changes in gene expression. The genes may be used as toxicity markers in drug screening and toxicity assays. That application includes a database of genes characterized by toxin-induced differential expression designed for use with microarrays and other solid-phase probes. The WO 02/095000 Application does not provide specific combination(s) of markers that can be used for toxicity prediction, and thus the methods disclosed require measuring expression of a vast number of genes.

The development of methods to predict the future onset of renal injury and gain a greater understanding of its underlying mechanism would facilitate the development of more reliable clinical diagnostics and safer therapeutic drugs. Moreover, improved preclinical markers for renal injury, particularly of well-defined gene signatures including small number of genes would dramatically reduce the time, cost, and amount of compound required for prioritizing and selecting lead candidates for progression through drug development.

International Patent Application No. PCT/IL2008/001561 to the inventors of the present invention discloses markers and marker sets for predicting the onset of renal injury. The markers are capable of detecting the expression of novel polynucleotide variants, known genes and combinations thereof, and expression of only small number of genes and/or variant suffice to obtain the required prediction.

It will be advantageous to have additional markers and marker sets useful for predicting the onset of renal injury.

### SUMMARY OF THE INVENTION

The present invention provides marker sets including novel variants, known genes and combinations thereof, the expression of which is useful in predicting the onset of renal injury, particularly an injury resulting from exposure to a toxin or pharmaceutical agent. The present invention further provides novel isolated polynucleotide and protein variants. The present invention further provides novel isolated polynucleotide and protein variants.

The present invention is based in part on the elucidation of the global changes in gene expression in tissues or cells exposed to known toxins, in particular renal toxins, as compared to unexposed, or exposed to control compounds, tissues or cells, as well as on the identification of individual genes that are differentially expressed upon exposure of the cells to a toxin. The present invention is advantageous over hitherto known methods using gene sets for predicting renal injury, because it provides small sets of only few genes necessary for accurate prediction. Moreover, the present invention provides sets based on the expression of unique polynucleotides and proteins associated with renal injury.

Thus, according to one aspect, the present invention provides an isolated polynucleotide encoding a protein having an amino acid sequence as set forth in any one of SEQ ID NOs: 62-71, 141, 143, 144, 146-153, 155, 156, 158-160, 162, 167-169, 172, 174-176, 210-214, 218, 221-223, 225, 227-228, 233, 235, 236.

According to certain embodiments, the polynucleotide has a nucleic acid sequence as set forth in any one of the SEQ. ID NOs: 1, 3, 6, 9, 12, 15, 20, 24, 27, 30, 32, 34, 36, 38, 40, 82, 84-90, 92, 94, 95-99, 101-104, 106-108, 110, 111, 113, 114, 116-119, 133, 135-137, 178, 180-184, 189, 192-194, 196, 198, 199, 203, 206, 207, or a sequence homologous thereto. According to one embodiment, the isolated polynucleotide is at least 85% homologous to any one of SEQ. ID NOs: 1, 3, 6, 9, 12, 15, 20, 24, 27, 30, 32, 34, 36, 38, 40, 82, 84-90, 92, 94, 95-99, 101-104, 106-108, 110, 111, 113, 114, 116-119, 133, 135-137, 178, 180-184, 189, 192-194, 196, 198, 199, 203, 206, 207. According to another embodiment, the isolated polynucleotide is at least 95% homologous to any one of SEQ. ID NOs: 1, 3, 6, 9, 12, 15, 20, 24, 27, 30, 32, 34, 36, 38, 40, 82, 84-90, 92, 94, 95-99, 101-104, 106-108, 110, 111, 113, 114, 116-119, 133, 135-137, 178, 180-184, 189, 192-194, 196, 198, 199, 203, 206, 207.

According to certain embodiments, the polynucleotide has a nucleic acid sequence as set forth in any one of the SEQ. ID NOs: 2, 4, 5, 7, 8, 10, 11, 13, 14, 16-19, 21-23, 25, 26, 28, 29, 31, 33, 35, 37, 39, 41.

According to other embodiments, the present invention provides an isolated protein or polypeptide having an amino acid sequence as set forth in any one of SEQ. ID NOs: 62-71, 141, 143, 144, 146-153, 155, 156, 158-160, 162, 167-169, 172, 174-176, 210-214, 218, 221-223, 225, 227-228, 233, 235, 236, or a sequence homologous thereto. According to one embodiment, the isolated protein or polypeptide is at least 85% homologous to any one of SEQ. ID NOs: 62-71, 141, 143, 144, 146-153, 155, 156, 158-160, 162, 167-169, 172, 174-176, 210-214, 218, 221-223, 225, 227-228, 233, 235, 236. According to another embodiment, the isolated polypeptide is at least 95% homologous to any one of SEQ. ID NOs: 62-71, 141, 143, 144, 146-153, 155, 156, 158-160, 162, 167-169, 172, 174-176, 210-214, 218, 221-223, 225, 227-228, 233, 235, 236.

According to certain embodiments, the isolated protein or polypeptide is encoded by an isolated polynucleotide comprising a nucleic acid sequence as set forth in any one of SEQ. ID NOs: 1, 3, 6, 9, 12, 15, 20, 24, 27, 30, 32, 34, 36, 38, 40, 82, 84-90, 92, 94, 95-99, 101-104, 106-108, 110, 111, 113, 114, 116-119, 133, 135-137, 178, 180-184, 189, 192-194, 196, 198, 199, 203, 206, 207, or a sequence homologous thereto.

According to certain embodiments of the present invention, the novel polynucleotides and proteins described herein are non-limiting examples of markers for diagnosing renal injury. The markers of the invention can be employed for various uses, including but not limited to, prognosis, prediction, screening, early diagnosis, determination of progression, staging, therapy selection and treatment monitoring of renal injury.

According to certain embodiments, the presence of at least one novel nucleic acid sequence in a biological sample predicts the onset of renal injury in the subject. According to certain embodiments, the nucleic acid sequence is as set forth in any one of SEQ ID NO: 1, 3, 6, 9, 12, 15, 20, 24, 27, 30, 32, 34, 36, 38, 40, 82, 84-90, 92, 94, 95-99, 101-104, 106-108, 110, 111, 113, 114, 116-119, 133, 135-137, 178, 180-184, 189, 192-194, 196, 198, 199, 203, 206, 207.

The present invention further provides sets of the novel polynucleotides and proteins of the present invention, sets of polynucleotides corresponding to known genes proteins encoded therefrom; and sets comprising combinations thereof, wherein expression of these nucleic acid sets is indicative of the onset of renal injury. The novel polynucleotides and proteins and sets of the invention are therefore referred to as markers of renal injury.

According to another aspect, the present invention provides a set of markers of renal injury comprising at least two markers having a nucleic acid sequence selected from the group consisting of SEQ ID NOs: 1, 3, 6, 9, 12, 15, 20, 24, 27, 30, 32, 34, 36, 38, 40, 42, 44, 46, 48, 50, 52, 54, 56, 58, 60, listed in Table 19, and the corresponding human and mouse homologues thereto as set forth in any one of SEQ ID NOs: 81-137 and 177-207, respectively, listed in Table 21.

According to certain embodiments, the at least two markers have a nucleic acid sequence selected form the group consisting of SEQ ID NOs: 1, 3, 6, 9, 12, 20, 24, 27, 30, 32, 34, 36, 38, 46, 50, 54, 56, 60 corresponding to genes listed in any one of Table 14 or 15.

According to certain embodiments, the at least two markers have a nucleic acid sequence selected form the group consisting of SEQ ID NOs: 1, 3, 6, 12, 48, 52 corresponding to genes listed in any one of Table 26 or 27, and the corresponding human and mouse homologues thereto as set forth in any one of SEQ ID NOs: 81-137 and 177-207, respectively, listed in Table 21.

According to further embodiments, the at least two markers are selected from a combination of novel polynucleotides and known genes having nucleic acid sequence selected from the group consisting of SEQ ID NOs: 1, 3, 6, 12 corresponding to genes listed in Table 13.

According to particular embodiments, the set comprises all four markers having a nucleic acid sequence set forth in SEQ ID NOs: 1, 3, 6, 12 corresponding to genes listed in Table 13, wherein expression of the four markers is indicative of the onset of renal injury.

According to additional particular embodiments, the set comprises all four markers having a nucleic acid sequence set forth in SEQ ID NOs: 1, 3, 48, 52 corresponding to genes listed in Table 26, wherein expression of the four markers is indicative of the onset of renal injury.

According to further particular embodiments, the set comprises all six markers having a nucleic acid sequence set forth in SEQ ID NOs: 1, 3, 6, 12, 48, 52 corresponding to genes listed in Table 27, wherein expression of the six markers is indicative of the onset of renal injury.

According to yet further embodiments, the at least two markers are novel polynucleotides selected from the group consisting of a nucleic acid sequence consisting of SEQ ID NOs: 1, 9, 12, 15, 24, 32, 36, 40, 44, 46, 48, 52, 54, 56, 58, 60 corresponding to genes listed in any one of Table 17 and Table 18.

According to yet additional embodiments, the at least two markers are selected from a combination of novel polynucleotides and known genes having a nucleic acid sequence selected from the group consisting of SEQ ID NOs: 1, 12, 46, 48, 56, 58, 60 corresponding to genes listed in Table 16.

According to particular embodiments, the set comprises all seven markers having a nucleic acid sequence set forth in SEQ ID NOs: 1, 12, 46, 48, 56, 58, 60 corresponding to genes listed in Table 16, wherein expression of the seven markers is indicative of the onset of renal injury.

According to another aspect, the present invention provides a set of markers of renal injury comprising at least two markers having an amino acid sequence encoded by the nucleic acid sequence selected from the group consisting of SEQ ID NOs: 1, 3, 6, 9, 12, 15, 20, 24, 27, 30, 32, 34, 36, 38, 40, 42, 44, 46, 48, 50, 52, 54, 56, 58, 60, listed in Table 12 or 19, or the corresponding human and mouse homologues, SEQ ID NOs: 81-137 and 177-207, respectively, listed in Table 21. According to one embodiment, the set of markers of renal injury comprises at least two markers having an amino acid sequence selected from the group consisting of SEQ ID NOs: 62-80, listed in Table 19, and the corresponding human and mouse homologues as set forth in SEQ ID NOs: 138-176 and 208-236, respectively, listed in Table 21.

According to certain embodiments, the at least two markers are novel polypeptides having an amino acid sequence selected form the group consisting of SEQ ID NO: 62-66, 68-70 corresponding to genes listed in Table 14.

According to other embodiments, the at least two markers are known proteins having an amino acid sequence selected from the group consisting of SEQ ID NO: 75, 77, 78, 80 corresponding to markers listed in Table 15.

According to other embodiments, the at least two markers are known proteins having an amino acid sequence selected from the group consisting of SEQ ID NO: 62, 63, 74, 76 corresponding to genes listed in Table 26.

According to other embodiments, the at least two markers are known proteins having an amino acid sequence selected from the group consisting of SEQ ID NO: 62-64, 66, 74, 76 corresponding to genes listed in Table 27.

According to further embodiments, the at least two markers are selected from a combination of novel and known proteins having an amino acid sequence selected from the group consisting of SEQ ID NO: 62-64, 66 corresponding to genes listed in Table 13.

According to particular embodiments, the set comprises all four markers having an amino acid sequence set forth in SEQ ID NO: 62-64, 66 corresponding to genes listed in Table 13, wherein expression of the four markers is indicative of the onset of renal injury.

According to particular embodiments, the set comprises all four markers having an amino acid sequence set forth in SEQ ID NO: 62, 63, 74, 76 corresponding to genes listed in Table 26, wherein expression of the four markers is indicative of the onset of renal injury.

According to particular embodiments, the set comprises all six markers having an amino acid sequence set forth in SEQ ID NO: 62-64, 66, 74, 76 corresponding to genes listed in Table 27, wherein expression of the four markers is indicative of the onset of renal injury.

According to yet further embodiments, the at least two markers are novel polypeptides having an amino acid sequence selected form the group consisting of SEQ ID NO: 62, 65-67, 69, 71 corresponding to genes listed in Table 17.

According to additional embodiments, the at least two markers are known proteins having an amino acid sequence selected from the group consisting of SEQ ID NO: 73, 74, 76-80 corresponding to genes listed in Table 18.

According to yet additional embodiments, the at least two markers are selected from a combination of novel and known proteins having an amino acid sequence selected from the group consisting of SEQ ID NO: 62, 66, 74, 78-80 corresponding to genes listed in Table 16.

According to particular embodiments, the set comprises six markers having an amino acid sequence set forth in SEQ ID NO: 62, 66, 74, 78-80 corresponding to genes listed in Table 16, wherein expression of the six markers is indicative of the onset of renal injury.

The markers of the invention described herein are information-rich with respect to classifying biological samples for onset of renal injury, even before histopathological or clinical indications are apparent. Thus, the marker sets of the present invention are highly efficient for the early detection of renal injury that may be caused by various conditions and/or treatments.

The present invention further provides a method for testing whether a compound will induce renal injury. According to yet another aspect, the present invention provides a method for predicting renal injury in a subject receiving a treatment with a compound, comprising (a) administering a dose of the compound to at least one test subject; (b) after a selected time period, obtaining a biological sample from the at least one test subject; (c) measuring in the biological sample the expression level of at least two markers selected from those listed in Table 12; and (d) determining whether the sample is in the positive class for onset of renal injury using a classifier comprising at least the two markers for which the expression level is measured.

According to certain embodiments, the subject is a mammal selected from the group consisting of a human, cat, dog, monkey, mouse, pig, rabbit, and rat. According to other embodiments, the subject is a test subject. According to typical embodiments the test subject is a rat. According to other embodiments, the test compound is administered to the subject in a form selected from the group consisting of intravenously (IV), orally (PO, per os), intraperitoneally (IP), intranasal, inhalation, eye drops and ointments. According to yet other embodiments, the biological sample is selected from the group consisting of kidney tissue, body fluid and body secretion.

The compound may be administered once or the administration may be repeated at any desired regime. It is to be understood that the selected period of time after which the sample is obtained refers to the time after the last compound administration.

According to other embodiments, the test compound is nephrotoxic agent. According to one embodiment, the nephrotoxic agent is selected from the group consisting of aminoglycosides; platinum based chemotherapy agents; heavy metals; DNA interacting drugs; antifungal agents; proximal tubule damaging agents; and vasoconstriction compounds.

According to one embodiment, the renal injury is associated with at least one kidney disease or pathology, selected from the group consisting of nephrotoxicity, renal toxicity, nephritis, kidney necrosis, kidney damage, glomerular and tubular injury, focal segmental glomerulosclerosis, kidney dysfunction, nephritic syndrome, acute renal failure, chronic renal failure, proximal tubal dysfunction, acute kidney transplant rejection and chronic kidney transplant refection.

According to other embodiments, the method is used for predicting at least one toxic effect of the compound; predicting the progression of a toxic effect of the compound; predicting the renal toxicity of the compound; or identifying an agent that modulates the onset or progression of a toxic response.

According to certain embodiments, the selected time period after which the sample is obtained from the test subject is prior to the appearance of histopathological or clinical indications of renal injury. According to one embodiment, the selected time period is any one of about 1 day, about 5 days, about 7 days, about 14 days, about 21 or about 28 days after administering the compound to the at least one test subject. According to typical embodiments, the selected time period is 1 day or less after administration. According to typical embodiments, the selected time period is 5 days after administration. According to other typical embodiments, the selected time period is 7 days after administration.

The at least two markers can be selected as to produce any one of the marker sets disclosed in the present invention. According to certain embodiments, the method is performed with a marker set comprising SEQ ID NO: 1, 3, 6, 12 corresponding to sequences within the genes listed in Table 13 or polypeptide or proteins encoded therefrom. According to other typical embodiments, the method is performed with a marker set comprising SEQ ID NO: 1, 12, 46, 48, 56, 58, 60 corresponding to sequences within the genes listed in Table 16 or polypeptide or proteins encoded therefrom. According to other typical embodiments, the method is performed with a marker set comprising SEQ ID NO: 1, 3, 6, 9, 12, 20, 24, 27, 30, 32, 34, 36, 38 corresponding to sequences within the genes listed in Table 14 or polypeptide or proteins encoded therefrom. According to other typical embodiments, the method is performed with a marker set comprising SEQ ID NO: 46, 50, 54, 56, 60 corresponding to sequences within the genes listed in Table 15 or polypeptide or proteins encoded therefrom. According to other typical embodiments, the method is performed with a marker set comprising SEQ ID NO: 1, 9, 12, 15, 24, 32, 36, 40 corresponding to sequences within the genes listed in Table 17 or polypeptide or proteins encoded therefrom. According to other typical embodiments, the method is performed with a marker set comprising SEQ ID NO: 44, 46, 48, 52, 54, 56, 58, 60 corresponding to sequences within the genes listed in Table 18 or polypeptide or proteins encoded therefrom. According to other typical embodiments, the method is performed with a marker set comprising SEQ ID NO: 1, 3, 48, 52 corresponding to sequences within the genes listed in Table 26 or polypeptide or proteins encoded therefrom. According to other typical embodiments, the method is performed with a marker set comprising SEQ ID NO: 1, 3, 6, 12, 48, 52 corresponding to sequences within the genes listed in Table 18 or polypeptide or proteins encoded therefrom.

According to certain typical embodiments, the method is performed with a marker set comprising markers listed in any one of Tables 13, 14, 15, 26 or 27, and the selected time period is 5 days after compound administration. According to other typical embodiments, the method is performed with a marker set comprising markers listed in any one of Tables 16, 17 or 18, and the selected time period is 1 day after compound administration.

According to other embodiments, the classifier is a random forest classifier. In alternative embodiments, the classifier may be another linear or non-linear classifier. According to currently typical embodiments the classifier for renal injury is capable of performing with a training log odds ratio of greater than or equal to 3.75.

Any method for detecting the marker expression as is known to a person skilled in the art may be used according to the teachings of the present invention. According to certain embodiments, the expression level of the at least two markers is detected at the polynucleotide level by an amplification or hybridization assay. According to typical embodiments, the amplification assay is selected from the group consisting of quantitative or semi-quantitative PCR, Northern blot, dot or slot blot, nuclease protection and microarray assays. According to other embodiments, the expression level of the at least two markers is detected at the polypeptide level by an immunoassay. According to typical embodiments the immunoassay is selected from the group consisting of an ELISA, an RIA, a slot blot, immunohistochemical assay, FACS, a radio-imaging assay or a Western blot.

The present invention also provides means and methods for detecting the expression of the markers disclosed herein in a sample. According to certain embodiments, the present invention provides probes and primers for detecting the polynucleotide expression of the markers disclosed herein. According to one embodiment, the probe or the primer comprises a nucleic acid sequence that specifically hybridizes to sequences within a gene selected from Table 12 or Table 21.

According to other embodiments, the present invention provides a set of at least two probes or primers, wherein each of the probes or primers comprises a sequence that specifically hybridizes to a marker selected from Table 19 or Table 21.

According to additional embodiments, the present invention provides a set of at least two probes or primers, wherein each of the probes or primers comprises a sequence that specifically hybridizes to an amplicon comprising any one of SEQ ID NOs: 251, 254, 257, 260, 263, 266, 269, 272, 275, 278, 281, 284, 287, 290, 293, 296, 299, 302, 305, 308, 311, 314, 317, 320, 323, 326.

According to further embodiments, the set comprises primers comprising a nucleic acid sequence set forth in any one of SEQ ID NO: 249-250, 252-253, 255-256, 258-259, 261-262, 264-265, 267-268, 270-271, 273-274, 276-277, 279-280, 282-283, 285-286, 288-289, 291-292, 294-295, 297-298, 300-301, 303-304, 306-307, 309-310, 312-313, 315-316, 318-319, 321-322, 324-325.

According to yet further embodiments, the set comprises probes or primers that hybridize to at least a plurality of markers selected from any one of Tables 13, Table 14, Table 15, Table 26 and Table 27. According to certain currently preferred embodiments, the plurality of markers comprises all the markers of Table 13. According to other certain currently preferred embodiments, the plurality of markers comprises all the markers of Table 26. According to other certain currently preferred embodiments, the plurality of markers comprises all the markers of Table 27.

According to yet additional embodiments, the set comprises probes or primers that hybridize to at least a plurality of markers selected from any one of Tables 16, Table 17 and Table 18. According to certain currently preferred embodiments, the plurality of markers comprises all the markers of Table 16.

The hybridization probes for detecting the polynucleotides of the present invention can be used as free polynucleotides in a solution or can be attached to a solid support as is known to a person skilled in the art. According to certain embodiments, the solid support is selected from the group consisting of a membrane, a glass support and a silicon support.

According to one embodiment, detecting the presence of the polypeptide or polynucleotide is indicative of renal injury. According to another embodiment, a change in the expression level of the polynucleotide or polypeptide compared to its expression and/or level in a sample obtained from a healthy subject is indicative of the renal injury. According to a further embodiment, a change in the expression and/or level of the polynucleotide or polypeptide compared to its level and/or expression in a sample obtained from the said subject at earlier stage is indicative of the renal injury. According to still further embodiment, detecting the presence and/or relative change in the expression and/or level of the polynucleotide or polypeptide is useful for selecting a treatment and/or monitoring a treatment of renal injury.

According to additional aspect, the present invention provides a method for selecting a treatment or monitoring a treatment for renal injury comprising (a) obtaining a first sample from a subject suffering from renal injury; (b) administering the treatment to the subject; (c) obtaining a second sample from said subject; (d) measuring in the first and second samples the expression level of at least two markers selected from those listed in Table 12 or Table 21; and (d) determining a change in the expression and/or level of the polynucleotides or polypeptides in said second sample compared to the level and/or expression in said first sample, wherein relative change in said expression and/or level of the polynucleotide or polypeptide is useful for selecting a treatment and/or monitoring a treatment of the renal injury.

The present invention also provides a kit for predicting whether renal injury will occur in a test subject comprising at least one means for detecting the expression of at least two markers as described hereinabove, further comprising reagents for performing the detection. According to certain embodiments, the kit comprises at least two primers or probes and reagents for detecting at least two genes listed in Table 12. According to other embodiments, the kit comprises at least two polypeptides and reagents for detecting at least two polypeptides encoded by the genes listed in Table 12.

In one embodiment, the kit comprises at least a plurality of polynucleotides or polypeptides corresponding to a plurality of genes selected from those listed in Table 12 as described hereinabove. In one embodiment the kit comprising a plurality of markers includes markers corresponding to at least 2 genes selected from those listed in Table 12. In another preferred embodiment the plurality of genes are variables in a classifier capable of classifying renal injury with a training log odds ratio of greater than or equal to 3.75. In one typical embodiment, the kit comprises polynucleotides or polypeptides capable of detecting a subset of genes listed in any one of tables 13, 26, 27 and 16, as described hereinabove. In one preferred embodiment, the kit comprises polynucleotide probes capable of hybridizing to a plurality of transcripts of genes selected from those listed in Table 12 as described hereinabove. In one preferred embodiment, the kit comprises polynucleotide probes capable of hybridizing to a plurality of amplicons selected from those listed in Table 20. In one preferred embodiment, the kit comprises polynucleotide probes selected from those listed in Table 20. According to further embodiments, the kit further comprises at least one solid surface, wherein a plurality of polynucleotide probes are bound the at least one solid surface. In one embodiment, the plurality of probes is bound to a single solid surface in an array. Alternatively, the plurality of probes is bound to the solid surface on a plurality of beads.

According to one embodiment, the kit comprises reagents for detecting the marker expression by employing a NAT-based technology. In one embodiment, the NAT-based assay is selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence, In Situ Hybridization or Comparative Genomic Hybridization.

According to other preferred embodiments, the kit comprises a plurality of antibodies capable of recognizing or interacting with a plurality of polypeptides encoded by genes selected from those listed in Table 12. In certain embodiments, the polypeptides are secreted proteins encoded by genes listed in Table 12. According to other embodiments, the kit further comprises at least one reagent for performing an ELISA, an RIA, a slot blot, an immunohistochemical assay, FACS, in vivo imaging, a radio-imaging assay, or a Western blot.

Other objects, features and advantages of the present invention will become clear from the following description and drawings.

### BRIEF DESCRIPTION OF THE FIGURES

FIG. 1 presents chemical structure of T1 drug

FIG. 2 presents chemical structure of T2 drug

FIGS. 3A-3B present histological section of the kidney, magnified ×200. FIG. 3A presents a control animal. The arrows represent normal aspects of cortical tubules (eosinophilic cytoplasm). FIG. 3B presents 50 mg/kg T2-treated animal. The arrows represent basophilic tubules in the cortex, indicating post-necrotic regeneration.

FIGS. 4A-4B present histological section of the kidney, magnified ×400. FIG. 4A presents a control animal. The arrows represent normal aspects of cortical tubules (eosinophilic cytoplasm). FIG. 4B presents 50 mg/kg T2-treated animal. The arrows represent basophilic tubules in the cortex, indicating post-necrotic regeneration.

FIGS. 5A-5B present histological section of the kidney, magnified ×600. FIG. 5A presents a control animal. The arrows represent normal aspects of cortical tubules (eosinophilic cytoplasm). FIG. 5B presents 50 mg/kg T2-treated animal. The arrows represent basophilic tubules in the cortex, indicating post-necrotic regeneration.

FIG. 6 presents the ROC curve of the Random-Forest classifier based on the Real Time PCR measurements from the validation stage of the labeled samples. The ROC curve is based on the out-of-the-bag method for estimating the performance of a Random-Forest classifier.

### DETAILED DESCRIPTION OF THE INVENTION

The present invention provides novel polynucleotides and polypeptides, as well as sets of these novel compounds and sets of known genes which are useful as diagnostic markers, particularly for predicting the onset of renal injury.

The present invention further provides methods for predicting whether a treatment with a compound would induce renal injury, following sub-chronic or long-term treatment. The present invention is based in part on gene expression data obtained from sub-acute or short-term treatments with a certain compound. The present invention now discloses necessary and sufficient sets of genes and specific signatures comprising these genes that allow gene expression data to be used to identify the ability of a compound treatment to induce late onset of renal injury before actual histological or clinical indication are apparent. Further, the invention provides kits comprising means for detecting the expression of the disclosed gene sets and signatures. The means and methods provided by the present invention enable the detection of a compound toxicity using short term studies, avoiding lengthy and costly long term studies.

### DEFINITIONS

“Marker”: in some embodiments, the phrase “marker” in the context of the present invention refers to a nucleic acid fragment, a peptide, or a polypeptide, which is differentially expressed in a sample taken from subjects exposed to a toxin, as compared to a comparable sample taken from subjects

As used herein, the term “expression” refers to the presence and/or level of a nucleic acid molecule, peptide or polypeptide in a sample at a certain time point, which amount may be the result of any process taking place in a cell within the sample, including, but not limited to, gene transcription and translation (gene expression) and degradation or stabilization of the gene product.

“Renal injury” refers to any damage to the kidney that can be caused by primary renal dysfunction (i.e Alport's syndrome, response to external substances (nephrotoxicants), infections, altered blood supply, malignancies, etc.) or secondary renal pathology (i.e. complications of diabetes mellitus, multiple myeloma, etc.).

Renal injury includes but is not limited to: nephritis, kidney necrosis, kidney damage, nephrotoxicity, renal toxicity, glomerular and tubular injury, focal segmental glomerulosclerosis, kidney dysfunction, nephritic syndrome, acute renal failure, chronic renal failure, proximal tubal dysfunction, acute kidney transplant rejection, chronic kidney transplant refection.

“Nephrotoxicant”: in the context of the present invention is used interchangeably with the phrase “nephrotoxic agent” and/or “renal toxin”, and refers to every substance (chemical compound and/or protein, recombinant or endogenous, including toxins or medications) that accumulates or that its clearance is via the renal system and causes renal injury. Examples of substance families that can cause such renal injury include but are not limited to: Aminoglycosides (i.e. Gentamicin Tobramycin, Amikacin, Kanamycin, Neomycin, Netilmicin, Paromomycin, Streptomycin, Tobramycin and Apramycin); Platinum based chemotherapy (i.e. Cisplatic, carboplatin); Heavy metals (i.e. Cadmium Chloride, Chromium, Arsenic, Lead, Mercury, Mangane); DNA interacting drugs (i.e. Doxorubicin, Daunorubicin, Epirubicin, Idarubicin, Mitoxantrone); Antifungal (i.e. Amphotericin B); Proximal tubule damaging agents (i.e. Acyclovir, Foscarnet, Pentamidine, Ifosfamide); and Vasoconstriction compounds (i.e. Radiocontrast agents, Cyclosporine, Tacrolimus).

As used herein, the term “test subject” refers to a subject receiving a compound or a treatment in order to evaluate the effect of the compound or treatment on the subject, including its efficacy, side effects, adverse effects and the like. According to typical embodiments, the test subject is a mammal.

“Multivariate dataset” as used herein, refers to any dataset comprising a plurality of different variables including but not limited to chemogenomic datasets comprising intensity measurements from gene expression experiments, such as those carried out on polynucleotide microarrays, or multiple protein binding affinities measured using a protein chip. Other examples of multivariate data include assemblies of data from a plurality of standard toxicological or pharmacological assays (e.g., blood analytes measured using enzymatic assays, antibody based ELISA or other detection techniques).

“Variable” as used herein, refers to any value that may vary. For example, variables may include relative or absolute amounts of biological molecules, such as mRNA or proteins, or other biological metabolites. Variables may also include dosing amounts of test compounds.

“Classifier” as used herein, refers to a function of a set of variables that is capable of answering a classification question. A “classification question” may be of any type susceptible to yielding a yes or no answer (e.g., “Is the unknown a member of the class or does it belong with everything else outside the class?”). A valid classifier is defined as a classifier capable of achieving a performance for its classification task at or above a selected threshold value. For example, a log odds ratio >3.75 represents a preferred threshold of the present invention. Higher or lower threshold values may be selected depending of the specific classification task.

“Random Forest” as used herein, refers to a type of non-linear classifier based on the majority decision of a collection of decision trees, each stating the outcome label (e.g. —the answer to the classification question), according to the values of the input variables. (Breiman, Leo “Random Forests”. Machine Learning 45 (1), 5-32, 2001). We use the random forest algorithm supplied by the R programming language.

“Signature” as used herein, refers to a combination of variables. As well as, possibly, a classification algorithm that provides a unique value or function capable of answering a classification question. A signature may include as few as one variable. Signatures include but are not limited to Random Forest classifiers.

“Log odds ratio” or “LOR” is used herein to summarize the performance of classifiers or signatures. LOR is defined generally as the natural log of the ratio of predicting a subject to be positive when it is positive, versus the odds of predicting a subject to be positive when it is negative. LOR is estimated herein using a set of training or test cross-validation partitions according to the following equation,

$$LOR = ln \frac{( {\sum }_{i = 1}^{c} {TP}_{i} + 0.5 ) * ( {\sum }_{i = 1}^{c} {TN}_{i} + 0.5 )}{( {\sum }_{i = 1}^{c} {FP}_{i} + 0.5 ) * ( {\sum }_{i = 1}^{c} {FN}_{i} + 0.5 )}$$

Where c (typically c=100 as described herein) equals the number of partitions, and TPᵢ, TNᵢ, FPᵢ and FNᵢ represent the number of true positive, true negative, false positive, and false negative occurrences in the test set of the i^{th} partition, respectively.

“Accuracy” as used herein, refers to an alternative mean of summarizing the performance of classifiers of signatures. Accuracy is the percentage of correctly labeled samples. It is estimated herein using a set of training or test cross-validation partitions according to the following equation,

$$ACC = 100 * \frac{{\sum }_{i = 1}^{c} ( {TP}_{i} + {TN}_{i} )}{{\sum }_{i = 1}^{c} {N}_{i}}$$

Where c (typically c=100 as described herein) equals the number of partitions, TPᵢ, TNᵢ, and Nᵢ represent the number of true positive, true negative, and all samples in the test set of the i^{th} partition, respectively.

“Array” as used herein, refers to a set of different biological molecules (e.g., polynucleotides, peptides, carbohydrates, etc.). An array may be immobilized in or on one or more solid substrates (e.g., glass slides, beads, or gels) or may be a collection of different molecules in solution (e.g., a set of PCR primers). An array may include a plurality of biological polymers of a single class (e.g., polynucleotides) or a mixture of different classes of biopolymers (e.g., an array including both proteins and nucleic acids immobilized on a single substrate).

“Array data” as used herein refers to any set of constants and/or variables that may be observed, measured or otherwise derived from an experiment using an array, including but not limited to: fluorescence (or other signaling moiety) intensity ratios, binding affinities, hybridization stringency, temperature, buffer concentrations.

“Proteomic data” as used herein refers to any set of constants and/or variables that may be observed, measured or otherwise derived from an experiment involving a plurality of mPvNA translation products (e.g., proteins, peptides, etc) and/or small molecular weight metabolites or exhaled gases associated with these translation products.

### General Methods of the Invention

The present invention provides novel polynucleotide and protein and gene signatures useful for detecting renal injury. The invention discloses lists of genes that may be used to create a signature that performs above a certain minimal threshold level for a specific prediction of renal injury. This set of genes also may be used to derive additional signatures with varying numbers of genes and levels of performance for particular applications (e.g., diagnostic assays and devices).

### Using Signatures for Predicting Renal Injury

A diagnostic usually consists in performing one or more assays and in assigning a sample to one or more categories based on the results of the assay(s). Desirable attributes of diagnostic assays include high sensitivity and specificity measured in terms of low false negative and false positive rates and overall accuracy. Because diagnostic assays are often used to assign large number of samples to given categories, the issues of cost per assay and throughput (number of assays per unit time or per worker hour) are of paramount importance. Typically the development of a diagnostic assay involves the following steps: (1) define the end point to diagnose, e.g., renal injury; (2) identify one or more markers whose alteration correlates with the end point, e.g., elevation of expression of a gene set; and (3) develop a specific, accurate, high-throughput and cost-effective assay for that marker. In order to increase throughput and decrease costs several diagnostics are often combined in a panel of assays, especially when the detection methodologies are compatible. For example several ELISA-based assays, each using different antibodies to ascertain different end points may be combined in a single panel and commercialized as a single kit. Even in this case, however, each of the ELISA-based assays had to be developed individually often requiring the generation of specific reagents.

The present invention provides signatures comprising as few as 2 genes, preferably as few as 4 genes, preferably as few as 5 genes, that are useful for determining a therapeutic or toxicological end-point for renal injury. These signatures (and the genes from which they are composed) may also be used in the design of improved diagnostic kits that answer the same questions as a large microarray but using a much smaller fraction of data. Generally, the reduction of information in a large chemogenomic dataset to a simple signature enables much simpler devices compatible with low cost high throughput multi-analyte measurement.

Consequently, the signatures of the present invention provide the ability to produce cheaper, higher throughput, diagnostic measurement methods or strategies. In particular, the invention provides diagnostic marker sets useful in diagnostic assays and the associated diagnostic kits. As used herein, diagnostic assays include assays that may be used for test subjects, patient prognosis and therapeutic monitoring.

Diagnostic marker sets may include markers representing a subset of genes disclosed in Table 12-18, and tables 26-27, and the genes homologous thereto, disclosed in table 21. In one preferred embodiment, the diagnostic marker set is a plurality of polynucleotides or polypeptides representing specific genes in the list contained in these tables. Such biopolymer marker sets are immediately applicable in any of the diagnostic assay methods (and the associate kits) well known for polynucleotides and polypeptides (e.g., DNA arrays, RT-PCR, immunoassays or other receptor based assays for polypeptides or proteins). Thus, a very simple diagnostic array may be designed that answers 3 or 4 specific classification questions and includes only 10-20 polynucleotides representing the approximately 5-10 genes in each of the signatures. Of course, depending on the level of accuracy required the LOR threshold for selecting a sufficient gene signature may be varied.

The diagnostic marker sets of the invention may be provided in kits, wherein the kits may or may not comprise additional reagents or components necessary for the particular diagnostic application in which the marker set is to be employed. Thus, for a polynucleotide array applications, the diagnostic marker sets may be provided in a kit which further comprises one or more of the additional requisite reagents for amplifying and/or labeling a microarray probe or target (e.g., polymerases, labeled nucleotides, and the a variety of array formats (for either polynucleotides and/or polypeptides) are well-known in the art and may be used with the methods and subsets produced by the present invention. In one embodiment, photolithographic or micromirror methods may be used to spatially direct light-induced chemical modifications of spacer units or functional groups resulting in attachment at specific localized regions on the surface of the substrate. Light-directed methods of controlling reactivity and immobilizing chemical compounds on solid substrates are well-known in the art and described in U.S. Pat. Nos. 4,562,157, 5,143,854, 5,556,961, 5,968,740, and 6,153,744, and PCT publication WO 99/42813, each of which is hereby incorporated by reference herein.

Alternatively, a plurality of molecules may be attached to a single substrate by precise deposition of chemical reagents. For example, methods for achieving high spatial resolution in depositing small volumes of a liquid reagent on a solid substrate are disclosed in U.S. Pat. Nos. 5,474,796 and 5,807,522, both of which are hereby incorporated by reference herein.

It should also be noted that in many cases a single diagnostic device may not satisfy all needs. However, even for an initial exploratory investigation {e.g., classifying drug-treated rats) DNA arrays with sufficient gene sets of varying size (number of genes), each adapted to a specific follow-up technology, can be created. In addition, in the case of drug-treated rats, different arrays may be defined for each tissue.

Alternatively, a single substrate may be produced with several different small arrays of genes in different areas on the surface of the substrate. Each of these different arrays may represent a sufficient set of genes for the same classification question but with a different optimal gene signature for each different tissue. Thus, a single array could be used for particular diagnostic question regardless of the tissue source of the sample (or even if the sample was from a mixture of tissue sources, e.g., in a forensic sample).

According to the present invention, the genes identified in Table 12 may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Table 12 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

### Nucleic Acid Assay Formats

The genes identified as being differentially expressed upon exposure to a known renal toxin (Tables 12-18 and 26-27, and Table 21) may be used in a variety of nucleic acid detection assays to detect or quantify the expression level of a gene or multiple genes in a given sample.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient.

Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface, to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 or more of such features on a single solid support. The solid support or the area within which the probes are attached may be on the order of about a square centimeter. Probes corresponding to the genes of Tables 12-18 and 26-27, or Table 21 may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al. (1996), NatBiotechnol 14: 1675-1680; McGall et al. (1996), Proc Nat Acad Sci USA 93: 13555-13460). Such probe arrays may contain at least two or more oligonucleotides that are complementary to or hybridize to two or more of the genes described in Tables 12-18 and 26-27 or Table 21. In one embodiment, such arrays contain oligonucleotides that are complementary to or hybridize to any subset of the genes in any one or all of Tables 14, 15, 17, 18, 26 and 27. In a preferred embodiment, such arrays contain oligonucleotides that are complementary to or hybridize to all or nearly all of the genes in any one of Tables 13, 16, 26 and 27. Preferred arrays contain all or nearly all of the genes listed in Tables 12-18 and 26-27 or Table 21. In a preferred embodiment, arrays are constructed that contain oligonucleotides to detect all or nearly all of the genes in any one or all of Tables 12-18, 26-27 and Table 21, in particular Tables 13, 14, 16, 27, 26 and 27 on a single solid support substrate, such as a chip.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. NatI. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55° C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes has 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Immunoassays

Assays to monitor the expression of a marker or markers as defined in Table 12 may utilize any available means of monitoring for changes in the expression level of the polypeptides and/or proteins of the present invention. As used herein, an agent is said to modulate the expression of an amino acid of the invention if it is capable of up- or down-regulating expression of the amino acid in a cell.

The genes identified as being differentially expressed upon exposure to a known renal toxin (Tables 12-18, 26-27 and Table 21) may be used in a variety of amino acid detection assays to detect or quantify the level of a polypeptide and/or protein or multiple polypeptides and/or proteins in a given sample.

Any assay format to detect polypeptide and/or protein levels may be used. For example, immunoassay, such as ELISA, an RIA, a slot blot, immunohistochemical assay, FACS, a radio-imaging assay or a Western blot, or other receptor based assays for polypeptides or proteins.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

### Kits

The invention further includes kits combining, in different combinations, high density oligonucleotide arrays, reagents for use with the arrays, protein encoded by the genes of Table 12 and homologous genes thereto, signal detection and array-processing instruments, gene expression databases and analysis and database management software described above.

The kits may be used, for example, to predict or model the toxic response of a test compound, to monitor the progression of renal disease states, to identify genes that show promise as new drug targets and to screen known and newly designed drugs as discussed above. The kits may be used in the pharmaceutical industry, where the need for receiving toxicity and other indications relating to a drug as early as possible is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting.

The general means and methods of the invention as described above are exemplified below. The following examples are offered as illustrations of specific embodiments and are not intended to limit the inventions disclosed throughout the whole of the specification.

### EXAMPLES

### Example 1

### Identification of Toxicity Markers

### Experimental Design

The renal toxins Gentamycin (antibiotic), Cisplatin (chemotherapeutic agent), Tobramycin (antibiotic), Cadmium chloride (heavy metal in the following industries: plastic, electroplating, batteries, paints, smelter), Doxorubicin (chemotherapeutic antibiotic) and Valprioc acid (antiepileptic agent that was used as a negative control), were administered to male and female Sprague-Dawley rats at various time points and routes of administration as describes below. The Sprague-Dawley Crl:CD(SD) rats were selected for the current investigation, since this strain is frequently used in preclinical investigations including investigations of microarray expression profiling during preclinical drug development.

The test items (positive and negative controls) were administered to animals in accordance with the usual route of administration in humans (i.e. IP for gentamycin, cisplatin, tobramycin and cadmium; IV for doxorubicin; and oral for valproic acid). Control animals were treated intraperitoneal with saline.

Each substance was administered to the animals once or daily for 5 days or 28 days at the appropriate dose level. The test substances and dose levels were selected according to the predicted pathological effects of each compound: the selected dose of each compound was expected to cause no histological changes after single dose administration, but to induce significant kidney toxicity after 28 days of daily treatment.

### Dose Formulation:

Gentamycin: intraperitoneal (IP) injection (80 mg/mL)

Cisplatin: injection (10 mg/20 mL)

Tobramycin (Brulamycin): injection (40 mg/1 mL and 80 mg/2 mL)

Cadmium chloride: a stock solution was prepared according to the valid SOP of IDRI (VIG/010 “Preparation of injection”).

Doxorubicin: intravenous (IV) injection (20 mg/10 mL),

Valproic acid—negative control: a suspension in concentration of 100 mg/mL for oral use (PO).

Randomization and group allocation: prior to treatment, the animals were assigned to the experimental groups based on their body weight. Mean body weight of each group at randomization was not deviate±20% of the mean population weight. Males and females were randomized separately. For the randomization, a computer program was used.

The original study groups and doses are shown in Table 1 below. Allocation to the dose group, 3 animals in each group, is shown in Table 2.

TABLE 1
Original study groups and doses

| | | | | Conc. | | | |
|---|---|---|---|---|---|---|---|
| | | | Dose | of | | | |
| | | Dose | Volume | stock | Route of | No. of animals | |
| Groups | Test items | (mg/kg) | (ml/kg) | (mg/ml) | administration | Males | Females |
| 1MF | Control/Saline | — | 5 | — | IP | 9 | 9 |
| 2MF | Gentamycin | 40 | 2 | 20 | IP | 9 | 9 |
| 3MF | Cisplatin | 0.5 | 2 | 0.25 | IP | 9 | 9 |
| 4MF | Tobramycin | 40 | 2 | 20 | IP | 9 | 9 |
| 5MF | Cadmium | 2 | 2 | 1 | IP | 9 | 9 |
| | chloride | | | | | | |
| 6MF | Doxorubicin | 4 | 2 | 2 | IV | 9 | 9 |
| 7MF | Valproic acid | 500 | 5 | 100 | PO | 9 | 9 |

TABLE 2
Allocation to the dose group, 3 animals in each group

| | Duration of treatment |
|---|---|
| Groups | (days) |
| 1MF | 1, 5, 28 |
| 2MF | 1, 5, 28 |
| 3MF | 1, 5, 28 |
| 4MF | 1, 5, 28 |
| 5MF | 1, 5, 28 |
| 6MF | 1, 5, 28 |
| 7MF | 1, 5, 28 |

### Results

The animals were treated with 6 different test items each for 3 duration of treatment (1, 5 and 28 days). The actual study design is shown in Table 3.

TABLE 3
Study design

| | | | | Conc. | | | |
|---|---|---|---|---|---|---|---|
| | | | Dose | of | | | |
| | | Dose | Volume | stock | Route of | No. of animals | |
| Groups | Test items | (mg/kg) | (ml/kg) | (mg/ml) | administration | Males | Females |
| 1MF | Control/Saline | — | 2 | — | IP | 9 | 9 |
| 2MF | Gentamycin | 40 | 2 | 20 | IP | 9 | 9 |
| 3MF | Cisplatin | 0.5 | 2 | 0.25 | IP | 9 | 9 |
| 4MF | Tobramycin | 40 | 2 | 20 | IP | 9 | 9 |
| 5MF | Cadmium cl.* | 20.75* | 2 | 10.375 | IP | 9 | 9 |
| 6MF | Doxorubicin** | 41** | 2 | 20.5 | IV | 9 | 9 |
| 7MF | Valproic acid | 500 | 5 | 100 | PO | 9 | 9 |
| 8MF*** | — | — | — | — | — | 3 | 3 |

IV—intravenous, IP—intraperitoneal, PO—oral (per os)
Modifications to the original study design:
*Animals treated with Cadmium chloride did not tolerate the treatment well, for that reason the dose was modified from 2 mg/kg to 0.75 mg/kg. Since males were in poor condition and excluded from the study, control (naïve) animals were introduced to the 5M dose group and treated with the lower dose for 24 days.
**From day 7 onwards, a sudden decay was observed in the condition of males and females treated with 4 mg/kg Doxorubicin (6MF group). This decay affected the 28-days study group of 6MF. These animals died or became moribund; therefore, naive animals were introduced into this group. The dose of the Doxorubicin treated group was lowered .from 4 mg/kg to 1 mg/kg. However, males from Day 19-20 and females from Day 22 received 0.5 mg/kg dose of Doxorubicin, due to their decrease in body weight.
***8MF control groups were introduced in the study, including 3 males and 3 females. Animals from these groups did not receive any treatment; however samples (tissue, blood, urine) were taken from them.

During the treatment period the animals were observed daily for clinical symptoms, their body weight was measured weekly and the water consumption was measured daily.

The day before autopsy, urinalysis was performed and blood samples were taken for hematology and clinical chemistry examinations.

On the day of autopsy the animals were anaesthetized with isoflurane, blood was collected for PBMC isolation then subjected to autopsy. Tissues and organs (adrenals, brain, kidneys, liver, spleen and thymus) were weighed and subjected to gross pathological and histopathological examinations.

Sterile kidney and liver samples were taken from the animals, frozen in liquid nitrogen and stored at −80° C. PBMC (Peripheral Blood Mononuclear Cell) and urine samples were stored at 80° C. as well. RNA was extracted from Kidney samples for toxicogenomic analysis for prediction of nephrotoxicity.

### Finding Summary:

### 1. Toxicants Effects

28-Day

Gentamycin: Signs of nephrotoxicity occurred in all animals of both sexes. During autopsy enlarged and discolored kidneys were observed in males. Kidney weight increased in both sexes. These changes were accompanied by clinical pathological findings: higher creatinine values in males and granular cylinders in the urine of females. The test item affected body weight gain of animals in both sexes. The nephrotoxic effect of the test item was confirmed by histopathology.

Cisplatin: The most severe nephrotoxic changes were found in Cisplatin treated groups of both sexes. Enlarged and discolored kidneys, increased kidneys and decreased thymus weight were found in both sexes. The nephrotoxic effect was detected during clinical pathology: Higher values of creatinine and urea nitrogen in both sexes & mild changes in the urine sediment. The test item affected body weight gain and water consumption in both sexes.

Tobramycin: Enlarged, discolored kidneys, with increased weights were observed in males. Clinical pathology revealed a nephrotoxic effect and higher values of creatinine and urea nitrogen in the blood as well as granular cylinders in the urine sediment of both sexes. Histopathological examinations confirmed nephrotoxic changes in both sexes.

Cadmium chloride: Mild clinical symptoms (hollow psoas, colic) were recorded in both sexes immediately after the administration. The test item affected body weight gain in both sexes. Slightly lower water consumption was recorded in males. Round bordered spleen and liver and thickening of peritoneum were recorded in both sexes, which correspond with higher liver and spleen weight. In addition increased weight of kidneys and decreased thymus were recorded in females. AST (Aspartate Aminotransferase) values were elevated in both sexes. A few transitional epithelial cells were observed in the urine of females. During histopathological examination significant nephrotoxicity was found only in a single female.

Doxorubicin: The test item caused severe clinical symptoms and lethality when administered in a 4 mg/kg dose. Two out of 3 males and 1 out of 3 females died. The lowered dose of 1 mg/kg of the test item was not lethal, but affected body weight gain in both sexes and water consumption in males. An increased weight of kidneys was found in males. The liver weight increased, while the weight of the thymus and spleen decreased significantly in both sexes. Elevated values of AST, ALT and Gamma GT were recorded in the male and decreased values of total protein, Albumin and Globulin were found in both sexes. Mild to moderate lymphoid depletion of the thymus was observed in both sexes.

Valproic acid: No histopathological signs of nephrotoxicity were found. The only toxic effect was a decreased total protein and Globulin value found in males.

Control/Saline: No histopathological signs of nephrotoxicity were found

5-Day

No lethality was recorded in any groups of both sexes.

Gentamycin: No nephrotoxicity was observed, except elevated urea nitrogen levels found in females. Body weight gain was decreased in females. No renal damage was found at histopathological examination.

Cisplatin: Mild, focal tubular epithelial cell necrosis in the medulla of kidneys was noted in a single male. No remarkable observations were found at autopsy. Decreased body weight gain and water consumption were recorded in both sexes.

Tobramycin: No renal damage was found during histopathology. Elevated urea nitrogen levels were found in females. Cadmium chloride: No renal damage was found. The test item caused mild clinical symptoms (hollow psoas, colic) immediately after the administration. Body weight gain decreased in both sexes. Slightly increased Gamma GT values were recorded in females.

Doxorubicin: No renal damage was found. Decreased body weight gain and water consumption was recorded in both sexes. Minimal to mild thymus atrophy and minimal lymphoid depletion of the spleen were found in males which was in accordance with gross pathological findings. RBC, HBG, HCT, reticulocyte and WBC counts were lower in both sexes.

Valproic acid: No renal damage was found. Slightly lower total protein and Albumin values were found in males.

Control/Saline: No histopathological signs of nephrotoxicity were found

SINGLE DAY: No renal damage was found.

Cisplatin: Decreased body weight gain was recorded in both sexes. Slightly lower lymphocytes and increased neutrophils counts were recorded in males.

Cadmium chloride: Mild clinical symptoms (hollow psoas, colic) were observed in both sexes immediately after the administration. Decreased body weight gain was recorded in both sexes. Lower lymphocytes and increased neutrophils counts were found in males.

Doxorubicin: Decreased body weight gain was recorded in both sexes. Lower relative weights of spleen were recorded in females. Higher WBC and neutrophil counts, decreasing tendency of total protein, Albumin and Globulin were found in males.: Control/Saline

No histopathological signs of nephrotoxicity were found

### 2. Histopathology

At histopathological examination after 28-day treatment, nephrotoxicity occurred in the following decreasing order: Cisplatin, Gentamycin, Tobramycin and Doxorubicin in males; Cisplatin, Cadmium chloride, Gentamycin, Doxorubicin and Tobramycin in females. Males were more sensitive to most of the substances.

Valproic acid proved to be non-nephrotoxic.

No renal damage was found after 5-day treatment in all dosed groups of both sexes, compared to the controls, except a single male treated with Cisplatin, where mild, focal tubular epithelial cell necrosis in the medulla was found.

No test item related renal damage was found after single treatment in all dosed groups of both sexes, compared to the controls.

Table 4 presents the summarized findings recorded after the 28-day treatment period in the different dosed groups of both sexes.

TABLE 4
Findings after the 28-day treatment period

| | | Findings | | | |
|---|---|---|---|---|---|
| | Dose | | Organ | Clinical | |
| Compound | (mg/kg) | Autopsy | weight | pathology | Histopathology |
| Gentamycin | 40 | ♂♀: ↑↑, pale | ♂♀: | ♂: ↑CREA | Kidneys ♂♀: lymphocytic |
| | | kidneys | ↑↑ | ♀: granular | inflammation, tubular |
| | | | kidneys | cylinders in | basophilia in the cortex. |
| | | | | urine | ♂: tubular dilatation in the |
| | | | | | medulla |
| Cisplatin | 0.5 | ♂♀: ↑↑, pale | ♂♀: | ♂♀: ↑↑BUN, | Kidneys ♂♀: lymphocytic |
| | | kidneys | ↑↑ | CREA | inflammation, tubular |
| | | | kidneys | ♂: trans. epith. | basophilia in the cortex. |
| | | | | cells in urine | Tubular dilatation, bizarre |
| | | | | ♀ (1/3): oval | forms in the medulla. |
| | | | | lipid cell in | |
| | | | | urine | |
| Tobramycin | 40 | ♂: ↑↑, pale | ♂: | ♂♀: ↑↑BUN, | Kidneys: |
| | | kidneys | ↑↑ | CREA | ♂♀: lymphocytic |
| | | | kidneys | ♂♀: granular | inflammation, tubular |
| | | | | cylinders in | basophilia in the cortex. |
| | | | | urine | ♂: tubular dilatation and |
| | | | | ♂: trans. epith. | basophilia in the medulla. |
| | | | | cells in urine | |
| Cadmium | 0.75 | ♂♀: | ♂♀: | ♂♀: ↑↑ AST | Spleen: |
| chloride | | ↑↑ liver, spleen, | ↑↑ liver, | ♀: trans. epith. | ♂: lymphoid depletion |
| | | thickening of | spleen | cells in urine | |
| | | peritoneum | | | |
| Doxorubicin | 1 | ♂♀: | ♂♀: | ♂: ↑↑AST, | Kidneys: |
| | | ↑↑ thymus | ↑↑ | ALT, γGT | ♂: tubular dilatation and |
| | | | thymus | ♂♀: ↓↓TP, | basophilia in the cortex. |
| | | | | Alb., Glob. | Tubular dilatation in the |
| | | | | | medulla. |
| | | | | | Thymus: |
| | | | | | ♂♀: lymphoid depletion |

CREA—creatinine,
BUN—Blood urea nitrogen,
AST—aspartate aminotransferase,
ALT—alanine aminotransferase,
Alb—Albumin

Table 5 presents the summarized findings recorded after the 5-day treatment period in the different dosed groups.

TABLE 5
Findings after the 5-day treatment period

| | Dose | Findings | | | |
|---|---|---|---|---|---|
| Compound | (mg/kg) | Autopsy | Organ weight | Clinical pathology | Histopathology |
| Gentamycin | 40 | No effect | No effect | ♀: ↑↑BUN | No renal damage |
| Cisplatin | 0.5 | No effect | No effect | ♂ (No. 24): ↑↑PLT, | ♂ (No. 24): tubular |
| | | | | ↓↓reticulocytes, ↑BUN | necrosis in the medulla |
| | | | | and CREA | |
| Tobramycin | 40 | No effect | No effect | ♀: ↑↑BUN | No renal damage |
| Cadmium | 2. | No effect | ♂♀: ↓thymus | ♀: ↑ γGT | No renal damage |
| chloride | | | | | |
| Doxorubicin | 4 | ♂♀: | ♂♀: | ♂♀: ↓↓RBC, HGB, | No renal damage. |
| | | ↓↓thymus | ↓↓thymus | HCT, reticulocytes, | ♂: |
| | | and spleen | and spleen | WBC | Thymus: atrophy |
| | | | | | Spleen: lymphoid |
| | | | | | depletion |

No test item related renal damage was found after single treatment in all dosed groups of both sexes, compared to the controls.

### 3. Lethality

### 28-Days Treatment

Death occurred only in Doxorubicin treated groups with the originally planned dose. No lethality was recorded after lowering the dose from 4 mg/kg to 1 mg/kg.

TABLE 6
Lethality in Doxorubicin treated groups

| | Died | Moribund |
|---|---|---|
| Males | 2/3 (Day 8) | 1/3 (Day 8) |
| Females | 1/3 (Day 9) | 2/3 (Day 9) |

Single and 5-Days Treatment

No lethality was recorded in any groups of both sexes.

### 4. Clinical Symptoms

### 28-Days Treatment

Clinical symptoms were observed only in Cadmium chloride groups and in the 4 mg/kg (originally planned dose) Doxorubicin treated groups of both sexes.

Single and 5-Day Treatment

Clinical symptoms were observed only in Cadmium chloride groups of both sexes. No clinical symptoms were observed in any other groups of both sexes.

### 5. Body Weight and Body Gain

28-Day Treatment

Effect on body weight and body weight gain was seen in groups of both sexes treated with Doxorubicin, Cadmium chloride, Cisplatin and Gentamycin.

5-Days Treatment

Decreased body weight gain was recorded in males treated with Cisplatin, Cadmium chloride, Doxorubicin, Valproic acid and in all dosed groups of females.

### 6. Water Consumption

Compared with controls the following observations were made concerning water consumption:

28-Day Treatment

Males

Sight decrease during the last week in groups dosed with Cisplatin, Cadmium chloride and Valproic acid; Significant decrease from Week 2 in the Doxorubicin treated group.

Females

Decreased during Week 4 in Cisplatin treated group.

5-Day Treatment

Decrease in Cisplatin and Doxorubicin treated groups of both sexes.

Decrease in Tobramycin and Cadmium chloride treated females

Increase in Valproic acid treated females

### 7. Autopsy

28-Day Treatment

At necropsy of animals treated with 4 mg/kg Doxorubicin who died (male No.: 52, 54 and female No.: 154) dehydration and anaemic organs were observed.

Scheduled Autopsy

Alterations were found in the kidneys in both sexes treated with Cisplatin and in males treated with Gentamycin and Tobramycin. Pale and enlarged kidneys were found in a single female (No. 145) treated with Cadmium chloride. Smaller thymuses were found in Doxorubicin treated groups of both sexes. Round bordered liver, enlarged spleen and thickening of the peritoneum were found in both sexes treated with Cadmium chloride.

5-Day Treatment

No alterations were found in the kidneys in the dosed groups of both sexes. No remarkable observations were found at autopsy in females. Smaller thymus and spleen were recorded in the Doxorubicin treated group of males.

Single Treatment

No alterations were found in the kidneys in the dosed groups of both sexes. No remarkable observations were found in females. Hemorrhagic thymus were found with different incidence in the dosed groups in males, including controls.

### 8. Organ Weights

28-Day Treatment

Compared with controls the following observations were made concerning the relative organ weights, taking into consideration the small number of groups (3/sex/group):

Increased liver weights in groups of both sexes treated with Cadmium chloride and Doxorubicin; Increased weights of kidneys in males treated with Gentamycin, Cisplatin, Tobramycin and Doxorubicin; Higher weights of kidneys in females treated with Gentamycin, Cisplatin and Cadmium chloride; Weights of spleen were higher in Gentamycin treated males and Cadmium chloride treated groups of both sexes. Lower weights were recorded in Doxorubicin treated groups of both sexes; Decreased thymus weights were found in Gentamycin and Tobramycin treated males, in Cisplatin and Doxorubicin treated groups of both sexes and in females treated with Cadmium chloride.

5-Day Treatment

No effect was observed in kidneys of the dosed groups of both sexes.

Compared with controls the following observations were made concerning the relative organ weights, taking into consideration the small number of groups (3/sex/group): Lower weights of spleen in Doxorubicin treated groups of both sexes; Lower thymus weights in Doxorubicin, Cadmium chloride and Valproic acid treated groups of both sexes.

Single Treatment

No effect was observed in kidneys of the dosed groups of both sexes. No significant alterations were recorded in males.

Compared with controls the following tendencies were observed in females concerning the relative organ weights, taking into consideration the small number of groups (3/sex/group): Lower weights of liver in Cisplatin treated group; Lower weights of thymus in Gentamycin, Cisplatin and Valproic acid treated groups; Lower weights of spleen in Cisplatin and Doxorubicin treated groups.

### Expression Analysis

RNA Isolation from Kidney Tissues

RNA was isolated from the kidney tissues using the following procedure:

Step 1: Homogenization of Tissue: Add 200 mg of tissue sample to 7 ml of cold TRI Reagent (MRC) on ice. Homogenize tissue using several short pulses (5 sec.) of the homogenizer.

Step 2: Phase separation: Add 0.2 ml of BCP (1-BROMO-3-CHLORO-PROPANE, SIGMA, Cat. No. B-9673) per 1 ml TRI Reagent. Cover the sample tightly and mix vigorously for 15 sec. Incubate the sample at RT 2-3 min. Spin the sample at 12000 g for 20 min at 4° C.

Step 3: RNA precipitation: Following centrifugation, the mixture separates into a lower red, phenol-chloroform phase, an interphase, and a colorless upper aqueous phase. Remove the top aqueous layer to a fresh tube. Add 0.5 ml Isopropanol per 1 ml TRI Reagent used in the original homogenization step. Mix the tube by vortex and incubate the sample at RT for 7-8 min. Spin the sample at 12000 g for 15 min at 4° C.

Step 4: RNA wash: Remove the sup and add a volume of 80% ethanol (made with DEPC-treated water, stored at −20° C.) equal to the original volume of TRI Reagent to rinse the pellet. Centrifuge the sample at 12000 g for 5 min at 4° C. Remove the ethanol and resuspend the RNA pellet in 400 ul DEPC water.

Step 5: RNA purification using phenol-chlorophorm extraction and MaXtract low Density tubes: Equilibrate the phenol:chlorophorm:isoamyl alcohol mix (25:24:1) (Ambion, Cat. No. 9732) to room temperature. Pellet the MaXtract tubes (QIAGEN, Cat. No. 129016) prior to use by centrifugation 1 minute at maximum speed. To the RNA dissolved add an equal volume (400 ul) of phenol:chlorophorm:isoamyl (25:24:1) alcohol mix and vortex. Transfer the entire Total RNA-phenol/chlorophorm mix to MaXtract tube. Centrifuge at 13000 rpm for 2 min. Transfer the aqueous upper phase to a fresh non-stick 1.5 ml tube (Ambion, Cat. No. AM12450). Pay attention not to touch the gel with the pipet tip as it reduces the grade of RNA purity. Repeat steps 5.3-5.5.

Step 6: RNA precipitation II: Precipitate the RNA by adding 0.1 vol 3M sodium acetate (RNAse free, Sigma Cat. No. S-7899) and 0.8 volume isopropanol (RNA vol.+sodium acetate vol.) For 400 μl RNA: 40 μl Sodium acetate and 352 μl isopropanol. Vortex 5 sec. Store at −20° C. over night. Fast cool the centrifuge. Spin the samples at full speed (14000 RPM) for 20 min at 4° C. pellet would be at the bottom.

Step 7: RNA wash II and resuspending: Pipette off the supernatant. Rinse twice the pellet with 0.5 ml 80% ethanol (made with DEPC-treated water, stored at −20° C.), centrifuge for 5 min at 9500 RPM, 4° C. Pipette out the ethanol, then short spin the samples in Eppendorf centrifuge at full speed for 10 sec., and pipette out the remainder of ethanol. Air dry the pellet. Simultaneously, preheat the DEPC treated water to 55-70° C. Check the RNA for dryness. When dry, it is almost completely transparent. Add appropriate volume of DEPC treated water to the pellet (20-150 μl) depending on the size of the pellet—do not mix! Heat the samples 10 min. at 55-70° C. Mix well by pipetation.

Final RNA product was quantified by spectrophotometric quantification. The quality of the RNA was evaluated by measuring the 260/280 and 260/230 ratios and confirmed by agarose gel. RNA was deemed of a suitable quality for microarray analysis if the 260/280 ratio was between 1.8 and 2.1, 260/230 ratio was higher than 1.5 and the gel showed no visible degradation products lower than the 18S ribosomal band.

### Microarray Hybridization

RNA samples extracted from the 1-Day and 5-Days treated animals and were sent to hybridization on Affymetrix array GeneChip® Rat Genome 230 2.0. The hybridization was performed according to the Affymetrix' following protocol:

Step 1: Target Preparation: Using protocols in Affymetrix' manual Section 2, double-stranded cDNA is synthesized from total RNA (or purified poly-A messenger RNA) isolated from tissue or cells. An in vitro transcription (NT) reaction is then done to produce biotin-labeled cRNA from the cDNA. The cRNA is fragmented before hybridization.

Step 2: Target Hybridization: A hybridization cocktail is prepared, including the fragmented target, probe array controls, BSA, and herring sperm DNA. It is then hybridized to the probe array during a 16-hour incubation. The hybridization process is describes in Affymetrix' manuals.

Step 3: Fluidics Station Setup: Specific experimental information is defined using Affymetrix® Microarray Suite or GeneChip Operating Software (GCOS) on a PC-compatible workstation. The probe array type, sample description, and comments are entered and saved with a unique experiment name. The fluidics station is then prepared for use by priming with the appropriate buffers.

Step 4: Probe Array Washing and Staining: Immediately following hybridization, the probe array undergoes an automated washing and staining protocol on the fluidics station.

Step 5: Probe Array Scan: Once the probe array has been hybridized, washed, and stained, it is scanned. Each workstation running Affymetrix Microarray Suite or GCOS can control one scanner. The software defines the probe cells and computes intensity for each cell. Each complete probe array image is stored in a separate data file identified by the experiment name and is saved with a data image file (.dat) extension.

Step 6: Data Analysis: The .dat image is analyzed for probe intensities; results are reported in tabular and graphical formats.

The expression levels were extracted using Affymetrix' MASS algorithm. Further multiplicative normalization on the data—setting the 95th percentile of the expression vector of each sample to an arbitrary constant value (1200)—was then performed.

### Classifier on Microarray Data

Finding signatures using machine learning algorithms requires two steps—selecting a limited set of features to be used for the signature, and building a classifier using these features. The feature selection stage is especially significant here for several reasons. Large number of features means a low signal to noise ratio, tampering the classifier performance, large number of features, compared to the relatively small number of samples, means that spurious features might exists. These are features that give good classification on the experiment data by chance, causing the classifier to be over-fitted to the learning set and have low prediction ability, and finally—a small number of features is important for practical application of the signature.

The initial features selection process is done by performing Mann-Whitney (Wilcoxon) rank-sum test on the data, considering only probesets whose overall normalized expression mean is over 25, to avoid working at noise-level.

Further features selection is done iteratively by building a Random Forest classifier, using the initial list of features, and estimating the importance of features as given by the algorithm's “Out of the Bag” approach. Less important features (those that have low impact on the performance of the classifier) are removed from the features list and the process is repeated.

The performance of the classifier built as just described, is estimated by calculating the LOR and Accuracy after performing a cross validation process—some of the samples are removed from the data. The whole process of features-selection and classifier building is performed on the data, and the prediction is tested on the left-out samples.

The following example illustrates the results of the process—working on the Day-5 samples of animals treated with toxic compounds, not including animals treated with Cadmium-Chloride (see pathological data, above) (“Toxic”), as compared to all controls samples (Saline and Valproic-acid treated animals at Day-1 and Day-5, as well as naïve rats) (“Control”). A list of 20 features (probesets) was initially selected, and then iteratively reduced to 6 features. The random forest classifier was used with the iterative list of features, as well as the gender of the rat used as an extra two-value feature. The random forest classifier was used with 2500 decision trees in each run. The cross validation was performed by leaving-out randomly selected samples—3 control samples and 3 toxic samples. This was repeated 350 times. The bound for deciding whether a sample is predicted as toxic or control, was chosen as to maximize the Accuracy. The “Confusion matrix” for this optimal bound is given in Table 7.

TABLE 7
“Confusion matrix”

| | Control | Toxic |
|---|---|---|
| Predicted as Control | 965 | 85 |
| Predicted as Toxic | 151 | 899 |

The Accuracy of the signatures as evaluated by this process is 89%, and the LOR is 4.2

### Selecting Genes for Validation

Following the analysis performed on the Day-5 data, 20 top-scoring probesets were selected for further validation using qRT-PCR. The probesets were mapped to the trasnscriptome produced by the LEADS clustering and assembly system (described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes) and the relevant contig and gene (whenever existing) were identified. As evident in table 8 bellow, the mapping of the probeset 1375422_at was not unique so we selected all relevant contigs.

Novel variants for the relevant genes were also identified. This was done based on the available rat ESTs and mRNAs and, when a human and mouse homologues was identified—the mouse ESTs and mRNAs and human transcripts were added the relevant rat ESTs and mRNAs to construct a combined informative contigs.

TABLE 8
Probe sets selected for qRT-PCR validation,
with their LEADS gene name and internal candidate name

| Probe set | Internal Name | Gene Name |
|---|---|---|
| 1375422_at | BE097535_DB71_T0 | BE097535 |
| 1390507_at | AI045075_DB71_T0 | ISG20 |
| 1367764_at | H31883_DB71_T0 | CCNG1 |
| 1369268_at | RATERFI_DB71_T0 | ATF3 |
| 1380692_at | BE104394_DB71_T0 | BE104394 |
| 1388674_at | RNU24174_DB71_T0 | CDKN1A |
| 1371785_at | AA686189_DB71_T0 | TNFRSF12A |
| 1393728_at | AA964541_DB71_T0 | AA964541 |
| 1369814_at | RNU90447_DB71_T0 | CCL20 |
| 1397637_at | AI502869_DB71_T0 | AI502869 |
| 1388560_at | H33998_DB71_T0 | WDR77 |
| 1392102_at | AI454051_DB71_T0 | AI454051 |
| 1370153_at | BI293562_DB71_T0 | GDF15 |
| 1388140_at | RATRAB13X_DB71_T0 | RAB13 |
| 1390833_at | AW919147_DB71_T0 | AW919147 |
| 1384934_at | BQ200887_DB71_T0 | BQ200887 |
| 1375895_at | AI549068_DB71_T0 | AI549068 |
| 1388642_at | H31799_DB71_T0 | EI24 |
| 1383162_at | AA799594_DB71_T0 | AA799594 |
| 1384070_at | H31045_DB71_T0 | H31045 |

RT Preparation and Real-Time qRT-PCR Analysis

Before RT preparation the RNA was treated with DNA-free (Ambion, cat. No. AM1906) DNase treatment and removal—according to manufacturer protocol. DNase procedure was repeated until no DNA is detected in a PCR reaction using the RNA samples.

RT preparation—Purified RNA (4 μg) is mixed with 600 ng Random Hexamer primers (Invitrogen, Cat. No. 48190-011) and 500 μM dNTP (Takara, Cat. No. 4030) in a total volume of 62.4 μl. The mixture is then incubated for 5 min at 65° C. and then quickly chilled on ice. Thereafter, 20 μl of 5× SuperscriptII first strand buffer (Invitrogen, Cat. No. Y00146), 9.6 μl 0.1M DTT (Invitrogen, Cat. No. Y00147) and 160 units RNasin (Promega, Cat. No. N251A) are added, and the mixture is incubated for 10 min at 25° C., followed by further incubation at 42° C. for 2 min. Then, 4 μl (800 units) of SuperscriptII (Invitrogen, Cat. No. 18064-022) is added and the reaction (final volume of 100 μl) is incubated for 50 min at 42° C. and then inactivated at 70° C. for 15 min. The resulting cDNA is diluted 1:20 in TE buffer (10 mM Tris pH=8, 1 mM EDTA pH=8).

cDNA (5 μl), prepared as described above, is used as a template in Real-Time PCR reactions using the SYBR Green I assay (PE Applied Biosystem) with specific primers in 100 nM concentration if not indicated otherwise and UNG Enzyme (Eurogentech or equal) Amplification is effected as follows: 50° C. for 2 min, 95° C. for 10 min, and then 40 cycles of 95° C. for 15 sec, followed by 60° C. for 1 min (if not indicated otherwise). Amplification step is followed by dissociation step. Detection is performed by using the PE Applied Biosystem SDS 7000. The cycle in which the reactions achieved a threshold level (Ct) of fluorescence is registered and is used to calculate the relative transcript quantity in the RT reactions. Non-detected samples are assigned Ct value of 41 and are calculated accordingly. The relative quantity is calculated using the equation Q=efficiencŷ-Ct. The efficiency of the PCR reaction is calculated from a standard curve, created by using serial dilutions of several reverse transcription (RT) reactions. To minimize inherent differences in the RT reaction, the resulting relative quantities are normalized to normalization factor calculated as follows:

The expression of four housekeeping (HSKP) genes from different pathways, ACTB (Entrez Gene ID: 81822), ACTG2 (Entrez Gene ID: 25365), HPRT (Entrez Gene ID: 24465) and YWHAZ (Entrez Gene ID: 25578), is found by SYBR green detection. The relative quantity (Q) of each housekeeping gene in each sample is calculated as described above. In order to calculate the “relative Q relative to MED” each Q for each HSKP gene is divided by the median quantity of this gene in all panel samples. To finally achieve the normalization factor of each sample the geometric mean of all four “relative Q relative to MED” was calculated. The normalization factor was then used for further calculations.

The sequences of the amplicons derived from the housekeeping genes measured in all the examples were as follows:

ACTB (Entrez Gene ID: 81822),

Forward Primer (SEQ ID NO: 237):

GGGAAATCGTGCGTGACATT

Reverse Primer: (SEQ ID NO: 238):

GCGGCAGTGGCCATCTC

Amplicon (SEQ ID NO: 239):

GGGAAATCGTGCGTGACATTAAAGAGAAGCTGTGCTATGTTGCCCTAGAC
TTCGAGCAAGAGATGGCCACTGCCGC

ACTG2 (Entrez Gene ID: 25365),

Forward Primer (SEQ ID NO: 240):

TACCCTATTGAGCACGGCAT

Reverse Primer (SEQ ID NO: 241):

CGCAGCTCGTTGTAGAAGGT

Amplicon (SEQ ID NO: 242):

TACCCCATTGAACACGGCATCATCACGAACTGGGATGACATGGAGAAGAT
CTGGCACCACTCCTTCTACAACGAGCTGCG

HPRT (Entrez Gene ID: 24465)

Forward Primer (SEQ ID NO: 243):

GCGAAAGTGGAAAAGCCAAGT

Reverse Primer (SEQ ID NO: 244):

GCCACATCAACAGGACTCTTGTAG

Amplicon (SEQ ID NO: 245):

GCGAAAGTGGAAAAGCCAAGTACAAAGCCTAAAAGACAGCGGCAAGTTGA
ATCTACAAGAGTCCTGTTGATGTGGC

YWHAZ (Entrez Gene ID: 25578)

Forward Primer (SEQ ID NO: 246):

CAAGCATACCAAGAAGCATTTGA

Reverse Primer (SEQ ID NO: 247):

GGGCCAGACCCAGTCTGA

Amplicon (SEQ ID NO: 248):

CAAGCATACCAAGAAGCATTTGAAATCAGCAAAAAGGAGATGCAGCCGAC
ACACCCCATCAGACTGGGTCTGGCCC

The markers of the present invention were tested with regard to their expression in a panel of kidney tissues samples from treated and control rats. Unless otherwise noted, all experimental data relates to the novel polynucleotides and proteins of the present invention, named according to the segment being tested (as expression was tested through RT-PCR as described). A description of the tissue samples used in the kidney testing panel is provided in Table 9 below. Tests were then performed as described in the “Materials and Experimental Procedures” section above.

The name comprises of the rat's group, as was described in table 2, the rats ID number, day of treatment and the nephrotoxicant it was exposed to:

Gent—Gentamycin, Cis—Cisplatin, Tob—Tobramycin, CadCl—Cadmium chloride, Dox—Doxorubicin, ValpA—Valproic Acid.

TABLE 9
Definitions of the tissue samples included in the panel based on their origin and treatment

| # | Toxin used | Day | | Name |
|---|---|---|---|---|
| 1 | Gentamycin | 1 | day 1 toxicants M | 1_2M10_day1_Gent |
| 2 | Gentamycin | 1 | | 2_2M11_day1_Gent |
| 3 | Gentamycin | 1 | | 3_2M12_day1_Gent |
| 4 | Cisplatin | 1 | | 4_3M19_day1_Cis |
| 5 | Cisplatin | 1 | | 5_3M20_day1_Cis |
| 6 | Cisplatin | 1 | | 6_3M21_day1_Cis |
| 7 | Tobramycin | 1 | | 7_4M28_day1_Tob |
| 8 | Tobramycin | 1 | | 8_4M29_day1_Tob |
| 9 | Tobramycin | 1 | | 9_4M30_day1_Tob |
| 10 | Cadmium chloride | 1 | | 10_5M37_day1_CadCl |
| 11 | Cadmium chloride | 1 | | 11_5M38_day1_CadCl |
| 12 | Cadmium chloride | 1 | | 12_5M39_day1_CadCl |
| 13 | Doxorubicin | 1 | | 13_6M46_day1_Dox |
| 14 | Doxorubicin | 1 | | 14_6M47_day1_Dox |
| 15 | Doxorubicin | 1 | | 15_6M48_day1_Dox |
| 16 | Gentamycin | 1 | day 1 toxicants F | 16_2F110_day1_Gent |
| 17 | Gentamycin | 1 | | 17_2F111_day1_Gent |
| 18 | Gentamycin | 1 | | 18_2F112_day1_Gent |
| 19 | Cisplatin | 1 | | 19_3F119_day1_Cis |
| 20 | Cisplatin | 1 | | 20_3F120_day1_Cis |
| 21 | Cisplatin | 1 | | 21_3F121_day1_Cis |
| 22 | Tobramycin | 1 | | 22_4F128_day1_Tob |
| 23 | Tobramycin | 1 | | 23_4F129_day1_Tob |
| 24 | Tobramycin | 1 | | 24_4F130_day1_Tob |
| 25 | Cadmium chloride | 1 | | 25_5F137_day1_CadCl |
| 26 | Cadmium chloride | 1 | | 26_5F138_day1_CadCl |
| 27 | Cadmium chloride | 1 | | 27_5F139_day1_CadCl |
| 28 | Doxorubicin | 1 | | 28_6F146_day1_Dox |
| 29 | Doxorubicin | 1 | | 29_6F147_day1_Dox |
| 30 | Doxorubicin | 1 | | 30_6F148_day1_Dox |
| 31 | Gentamycin | 5 | day 5 toxicants M | 31_2M13_day5_Gent |
| 32 | Gentamycin | 5 | | 32_2M14_day5_Gent |
| 33 | Gentamycin | 5 | | 33_2M15_day5_Gent |
| 34 | Cisplatin | 5 | | 34_3M22_day5_Cis |
| 35 | Cisplatin | 5 | | 35_3M23_day5_Cis |
| 36 | Cisplatin | 5 | | 36_3M24_day5_Cis |
| 37 | Tobramycin | 5 | | 37_4M31_day5_Tob |
| 38 | Tobramycin | 5 | | 38_4M32_day5_Tob |
| 39 | Tobramycin | 5 | | 39_4M33_day5_Tob |
| 40 | Cadmium chloride | 5 | | 40_5M40_day5_CadCl |
| 41 | Cadmium chloride | 5 | | 41_5M41_day5_CadCl |
| 42 | Cadmium chloride | 5 | | 42_5M42_day5_CadCl |
| 43 | Doxorubicin | 5 | | 43_6M49_day5_Dox |
| 44 | Doxorubicin | 5 | | 44_6M50_day5_Dox |
| 45 | Gentamycin | 5 | day 5 toxicants F | 45_2F113_day5_Gent |
| 46 | Gentamycin | 5 | | 46_2F114_day5_Gent |
| 47 | Gentamycin | 5 | | 47_2F115_day5_Gent |
| 48 | Cisplatin | 5 | | 48_3F122_day5_Cis |
| 49 | Cisplatin | 5 | | 49_3F123_day5_Cis |
| 50 | Cisplatin | 5 | | 50_3F124_day5_Cis |
| 51 | Tobramycin | 5 | | 51_4F131_day5_Tob |
| 52 | Tobramycin | 5 | | 52_4F132_day5_Tob |
| 53 | Tobramycin | 5 | | 53_4F133_day5_Tob |
| 54 | Cadmium chloride | 5 | | 54_5F141_day5_CadCl |
| 55 | Cadmium chloride | 5 | | 55_5F142_day5_CadCl |
| 56 | Doxorubicin | 5 | | 56_6F149_day5_Dox |
| 57 | Doxorubicin | 5 | | 57_6F150_day5_Dox |
| 58 | Doxorubicin | 5 | | 58_6F151_day5_Dox |
| 59 | Naive | 28 | Controls All days | 59_8M81_day28_Naive |
| 60 | Naive | 28 | | 60_8M82_day28_Naive |
| 61 | Naive | 28 | | 61_8M83_day28_Naive |
| 62 | Naive | 28 | | 62_8F181_day28_Naive |
| 63 | Naive | 28 | | 63_8F182_day28_Naive |
| 64 | Naive | 28 | | 64_8F183_day28_Naive |
| 65 | Control/Saline | 1 | | 65_1M1_day1_Saline |
| 66 | Control/Saline | 1 | | 66_1M2_day1_Saline |
| 67 | Control/Saline | 1 | | 67_1M3_day1_Saline |
| 68 | Control/Saline | 1 | | 68_1F101_day1_Saline |
| 69 | Control/Saline | 1 | | 69_1F102_day1_Saline |
| 70 | Control/Saline | 1 | | 70_1F103_day1_Saline |
| 71 | Control/Saline | 5 | | 71_1M4_day5_Saline |
| 72 | Control/Saline | 5 | | 72_1M5_day5_Saline |
| 73 | Control/Saline | 5 | | 73_1M6_day5_Saline |
| 74 | Control/Saline | 5 | | 74_1F104_day5_Saline |
| 75 | Control/Saline | 5 | | 75_1F105_day5_Saline |
| 76 | Gentamycin | 28 | day 28 toxicants M | 76_2M16_day28_Gent |
| 77 | Gentamycin | 28 | | 77_2M17_day28_Gent |
| 78 | Gentamycin | 28 | | 78_2M18_day28_Gent |
| 79 | Cisplatin | 28 | | 79_3M25_day28_Cis |
| 80 | Cisplatin | 28 | | 80_3M26_day28_Cis |
| 81 | Cisplatin | 28 | | 81_3M27_day28_Cis |
| 82 | Tobramycin | 28 | | 82_4M34_day28_Tob |
| 83 | Tobramycin | 28 | | 83_4M35_day28_Tob |
| 84 | Tobramycin | 28 | | 84_4M36_day28_Tob |
| 85 | Cadmium chloride | 28 | | 85_5M43_day28_CadCl |
| 86 | Cadmium chloride | 28 | | 86_5M44_day28_CadCl |
| 87 | Cadmium chloride | 28 | | 87_5M45_day28_CadCl |
| 88 | Doxorubicin | 28 | | 88_6M71_day28_Dox |
| 89 | Doxorubicin | 28 | | 89_6M72_day28_Dox |
| 90 | Doxorubicin | 28 | | 90_6M73_day28_Dox |
| 91 | Gentamycin | 28 | day 28 toxicants F | 91_2F116_day28_Gent |
| 92 | Gentamycin | 28 | | 92_2F117_day28_Gent |
| 93 | Gentamycin | 28 | | 93_2F118_day28_Gent |
| 94 | Cisplatin | 28 | | 94_3F125_day28_Cis |
| 95 | Cisplatin | 28 | | 95_3F126_day28_Cis |
| 96 | Cisplatin | 28 | | 96_3F127_day28_Cis |
| 97 | Tobramycin | 28 | | 97_4F134_day28_Tob |
| 98 | Tobramycin | 28 | | 98_4F135_day28_Tob |
| 99 | Tobramycin | 28 | | 99_4F136_day28_Tob |
| 100 | Cadmium chloride | 28 | | 100_5F143_day28_CadCl |
| 101 | Cadmium chloride | 28 | | 101_5F144_day28_CadCl |
| 102 | Cadmium chloride | 28 | | 102_5F145_day28_CadCl |
| 103 | Doxorubicin | 28 | | 103_6F171_day28_Dox |
| 104 | Doxorubicin | 28 | | 104_6F172_day28_Dox |
| 105 | Doxorubicin | 28 | | 105_6F173_day28_Dox |
| 106 | Control/Saline | 28 | Day 28 control | 106_1M7_day28_Saline |
| 107 | Control/Saline | 28 | | 107_1M8_day28_Saline |
| 108 | Control/Saline | 28 | | 108_1M9_day28_Saline |
| 109 | Control/Saline | 28 | | 109_1F107_day28_Saline |
| 110 | Control/Saline | 28 | | 110_1F108_day28_Saline |
| 111 | Control/Saline | 28 | | 111_1F109_day28_Saline |
| 112 | Valproic acid | 1 | Valproic acid - all days | 112_7M55_day1_ValpA |
| 113 | Valproic acid | 1 | | 113_7M56_day1_ValpA |
| 114 | Valproic acid | 1 | | 114_7M57_day1_ValpA |
| 115 | Valproic acid | 1 | | 115_7F155_day1_ValpA |
| 116 | Valproic acid | 1 | | 116_7F156_day1_ValpA |
| 117 | Valproic acid | 1 | | 117_7F157_day1_ValpA |
| 118 | Valproic acid | 5 | | 118_7M58_day5_ValpA |
| 119 | Valproic acid | 5 | | 119_7M59_day5_ValpA |
| 120 | Valproic acid | 5 | | 120_7M60_day5_ValpA |
| 121 | Valproic acid | 5 | | 121_7F158_day5_ValpA |
| 122 | Valproic acid | 5 | | 122_7F159_day5_ValpA |
| 123 | Valproic acid | 5 | | 123_7F160_day5_ValpA |
| 124 | Valproic acid | 28 | | 124_7M61_day28_ValpA |
| 125 | Valproic acid | 28 | | 125_7M62_day28_ValpA |
| 126 | Valproic acid | 28 | | 126_7M63_day28_ValpA |
| 127 | Valproic acid | 28 | | 127_7F161_day28_ValpA |
| 128 | Valproic acid | 28 | | 128_7F162_day28_ValpA |
| 129 | Valproic acid | 28 | | 129_7F163_day28_ValpA |

Classifier on qRT-PCR Data

The following tables summarize the microarray and qRT-PCR data for the markers of the present invention. The mean normalized expression level as measured by the microarray, as well as the mean normalized qRT-PCR expression levels, and the relevant Mann-Whitney scores p-values are given for each candidate.

TABLE 10
Mean expression levels and Mann-Whitney rank sum score p-values for microarray data of Day-1,
Day-5 and control samples

| | | Microarray Data | | | | |
|---|---|---|---|---|---|---|
| Candidate | | Normal | Tox Day-1 | Tox Day-5 | Day-1 | Day-5 |
| Probeset | Gene name | mean | mean | mean | p-value | p-value |
| 1371785_at | TNFRSF12A | 212 | 312 | 392 | 0.002 | 3.00E⁻⁰⁵ |
| 1383162_at | AA799594 | 4055 | 3521 | 3385 | 0.002 | 6.00E⁻⁰³ |
| 1393728_at | AA964541 | 35 | 46 | 56 | 0.01 | 6.00E⁻⁰⁵ |
| 1390507_at | ISG20 | 32 | 47 | 48 | 0.0006 | 6.00E⁻⁰⁶ |
| 1392102_at | AI454051 | 85 | 87 | 98 | 0.95 | 6.00E⁻⁰⁴ |
| 1397637_at | AI502869 | 65 | 59 | 56 | 0.03 | 3.00E⁻⁰⁴ |
| 1390833_at | AW919147 | 32 | 28 | 25 | 0.04 | 2.00E⁻⁰⁴ |
| 1370153_at | GDF15 | 38 | 48 | 73 | 0.01 | 1.00E⁻⁰⁴ |
| 1384070_at | H31045 | 88 | 99 | 108 | 0.02 | 3.50E⁻⁰⁵ |
| 1388642_at | EI24 | 867 | 860 | 917 | 0.38 | 8.00E⁻⁰⁴ |
| 1367764_at | CCNG1 | 757 | 864 | 1229 | 0.03 | 4.50E⁻⁰⁵ |
| 1388560_at | WDR77 | 532 | 529 | 572 | 0.82 | 4.00E⁻⁰⁴ |
| 1369268_at | ATF3 | 30 | 69 | 86 | 0.007 | 4.00E⁻⁰⁶ |
| 1388140_at | RAB13 | 171 | 180 | 193 | 0.004 | 8.00E⁻⁰⁴ |
| 1388674_at | CDKN1A | 97 | 123 | 192 | 0.003 | 5.00E⁻⁰⁶ |

TABLE 11
Mean expression levels and Mann-Whitney rank sum score p-values for qRT-PCR
data of Day-1, Day-5 and control samples

| | | qRT-PCR Data | | | | |
|---|---|---|---|---|---|---|
| | | | Tox | Tox | | |
| Candidate | | Normal | Day-1 | Day-5 | Day-1 | Day 5 |
| Internal name | Gene name | mean | mean | mean | p-value | p-value |
| W41270_DB81_seg11 | TNFRSF12A | 1.08 | 1.42 | 2.27 | 4.00E⁻⁰⁴ | 7.50E⁻¹⁰ |
| AA686189_DB71_seg6 | TNFRSF12A | 0.99 | 1.27 | 1.44 | 8.00E⁻⁰⁴ | 7.50E⁻⁰⁶ |
| AA799594_DB71_seg0 | AA799594 | 1.66 | 1.45 | 1.48 | 8.00E⁻⁰³ | 0.01 |
| AA964541_DB71_seg0 | AA964541 | 0.87 | 1.24 | 1.43 | 2.00E⁻⁰⁶ | 3.00E⁻⁰⁸ |
| W64472_DB81_seg2 | ISG20 | 1.06 | 1.14 | 2.32 | 0.35 | 2.00E⁻¹⁰ |
| AI045075_DB71_seg6 | ISG20 | 1.14 | 1.58 | 1.46 | 2.50E⁻⁰⁴ | 6.50E⁻⁰⁴ |
| AI454051_DB71_seg0 | AI454051 | 1.53 | 1.46 | 1.48 | 0.4 | 0.7 |
| AI502869_DB71_seg0 | AI502869 | 1.62 | 1.34 | 1.21 | 7.00E⁻⁰³ | 2.50E⁻⁰⁵ |
| AW919147_DB71_seg0 | AW919147 | 1.60 | 1.52 | 1.43 | 0.4 | 0.01 |
| BI293562_DB71_seg2 | GDF15 | 0.97 | 1.11 | 1.64 | 0.03 | 7.50E⁻⁰⁷ |
| H31045_DB71_seg5 | H31045 | 1.51 | 0.99 | 1.51 | 0.015 | 0.5 |
| W83813_DB81_seg27 | EI24 | 1.30 | 1.42 | 2.18 | 0.55 | 1.50E⁻⁰⁸ |
| H31799_DB71_seg23 | EI24 | 1.48 | 1.56 | 1.43 | 0.25 | 0.01 |
| MUSCYCG1R_DB81_seg15-17 | CCNG1 | 1.20 | 1.17 | 2.18 | 0.25 | 1.00E⁻⁰⁷ |
| MUSCYCG1R_DB81_seg19-20 | CCNG1 | 1.16 | 1.35 | 2.2 | 6.00E⁻⁰³ | 3.00E⁻⁰⁹ |
| H31883_DB71_seg13 | CCNG1 | 1.25 | 1.41 | 1.51 | 5.00E⁻⁰³ | 0.04 |
| W33294_DB81_seg23 | WDR77 | 1.44 | 1.27 | 1.9 | 0.05 | 7.00E⁻⁰⁴ |
| W33294_DB81_seg44 | WDR77 | 1.37 | 1.42 | 2.02 | 0.6 | 8.00E⁻⁰⁶ |
| H33998_DB71_seg19 | WDR77 | 1.31 | 1.58 | 2.13 | 0.04 | 2.00E⁻⁰⁷ |
| RATLRFI_DB71_seg9 | ATF3 | 0.84 | 1.47 | 1.39 | 0.025 | 5.00E⁻⁰⁴ |
| RATRAB13X_DB81_seg15-17 | RAB13 | 1.32 | 1.37 | 1.89 | 0.05 | 3.00E⁻⁰⁷ |
| RATRAB13X_DB81_seg22 | RAB13 | 1.36 | 1.37 | 2.12 | 0.04 | 3.00E⁻⁰⁷ |
| RATRAB13X_s DB71_eg11-13 | RAB13 | 1.40 | 1.59 | 1.42 | 1.00E⁻⁰³ | 0.6 |
| MMU09507_DB81_seg15 | CDKN1A | 0.88 | 0.94 | 1.74 | 0.08 | 2.00E⁻⁰⁶ |
| RNU24174_DB71_seg8 | CDKN1A | 0.76 | 0.91 | 1.21 | 2.00E⁻⁰⁵ | 3.00E⁻⁰⁵ |

The qRT-PCR data was used to construct the optimal Random-Forest classifier for Day-5 (Table 13) and Day-1 (Table 16). The optimal performance of the Day-5 signatures, as measured by cross validation is—Accuracy of 94% and LOR of 5.6. We further tested the performance of the optimal classifier on the Day-5 animals treated with Cadmium Chloride and found that 5 out of 6 samples were classified correctly as toxic. The optimal performance of the Day-1 signature is—Accuracy of 89% and LOR of 4.2

Table 14 and Table 15 further list genes that contribute to signatures for classification of Day-5 data. Table 17 and Table 18 further list genes that contribute to signatures of classification of Day-1. These genes can be used to replace the genes in Table 13 and Table 16 to yield classifiers with LOR of at least 3.75.

TABLE 12
All genes

| Candidate | | | | NV*/ | |
|---|---|---|---|---|---|
| ID | Contig | Internal name | Gene name | Genbank ID | Reference |
| 1 | AA686189 | W41270_DB81_seg11 | TNFRSF12A | NV of NM181086 | |
| 2 | AA686189 | AA686189_DB71_seg6 | TNFRSF12A | NM181086 | Peter et al., Physiol. |
| | | | | | Genomics 25: 375-386, |
| | | | | | 2006. |
| 3 | AA799594 | AA799594_DB71_seg0 | AA799594 | AI008646; | |
| | | | | DY471185 (ESTS, | |
| | | | | no RNA) | |
| 4 | AA964541 | AA964541_DB71_seg0 | AA964541 | AA964541 (one | WO02095000 |
| | | | | sequence - EST) | |
| 5 | AI045075 | W64472_DB81_seg2 | ISG20 | NV of NM001008510 | |
| 6 | AI045075 | AI045075_DB71_seg6 | ISG20 | NM001008510 | WO02095000 |
| 7 | AI454051 | AI454051_DB71_seg0 | AI454051 | BE118959 (EST, no | |
| | | | | RNA) | |
| 8 | AI502869 | AI502869_DB71_seg0 | AI502869 | AI502869; DV713733; | |
| | | | | AI145914; CB796640 | |
| | | | | (ESTs, no RNA) | |
| 9 | AW919147 | AW919147_DB71_seg0 | AW919147 | AW91914; BF392593 | |
| | | | | (ESTS, no RNA) | |
| 10 | BI293562 | BI293562_DB71_seg2 | GDF15 | NM01921; BI293562 | Peter et al., |
| | | | | (BI293562 is an EST | Physiol. |
| | | | | that extends the | Genomics 25: |
| | | | | transcript) | 375-386, 2006. |
| 11 | H31045 | H31045_DB71_seg5 | H31045 | CO569017; DY312216; | |
| | | | | CF115262; BM392249; | |
| | | | | AI556451 (ESTS, no | |
| | | | | RNA.) | |
| 12 | H31799 | W83813_DB81_seg27 | EI24 | NV of NM001025660 | |
| 13 | H31799 | H31799_DB71_seg23 | EI24 | NM001025660 | |
| 14 | H31883 | MUSCYCG1R_DB81_seg15-17 | CCNG1 | NV of NM012923 | |
| 15 | H31883 | MUSCYCG1R_DB81_seg19-20 | CCNG1 | NV of NM012923 | |
| 16 | H31883 | H31883_DB71_seg13 | CCNG1 | NM012923 | Thompson et |
| | | | | | al., Environ |
| | | | | | Health Perspect. |
| | | | | | 2004 |
| | | | | | Mar; 112(4): 488-94; |
| | | | | | WO02095000 |
| 17 | H33998 | W33294_DB81_seg23 | WDR77 | NV of NM001008771 | |
| 18 | H33998 | W33294_DB81_seg44 | WDR77 | NV of NM001008771 | |
| 19 | H33998 | H33998_DB71_seg19 | WDR77 | NM001008771 | |
| 20 | RATLRFI | RATLRFI_DB71_seg9 | ATF3 | NM012912 | Peter et al., |
| | | | | | Physiol. |
| | | | | | Genomics 25: |
| | | | | | 375-386, 2006. |
| | | | | | WO2004048598; |
| | | | | | WO200295000 |
| 21 | RATRAB13X | RATRAB13X_DB81_seg15-17 | RAB13 | NV of NM031092 | |
| 22 | RATRAB13X | RATRAB13X_DB81_seg22 | RAB13 | NV of NM031092 | |
| 23 | RATRAB13X | RATRAB13X_DB71_seg11-13 | RAB13 | NM031092 | WO02095000 |
| 24 | RNU24174 | MMU09507_DB81_seg15 | CDKN1A | NV of NM080782 | |
| 25 | RNU24174 | RNU24174_DB71_seg8 | CDKN1A | NM080782 | Peter et al., |
| | | | | | Physiol. |
| | | | | | Genomics 25: |
| | | | | | 375-386, 2006. |
| | | | | | WO2004048598; |
| | | | | | WO0295000 |

*NV identifies novel polynucleotides and proteins, which may or may be not variants of known proteins.

TABLE 13
Optimal signature for Day-5 data

| Candi- | | | |
|---|---|---|---|
| date ID | Contig | Internal name | Gene name |
| 1 | AA686189 | W41270_DB81_seg11 | TNFRSF12A |
| 5 | AI045075 | W64472_DB81_seg2 | ISG20 |
| 12 | H31799 | W83813_DB81_seg27 | EI24 |
| 15 | H31883 | MUSCYCG1R_DB81_seg19-20 | CCNG1 |

TABLE 14
Novel-Variants for Day-5 signatures

| Can- | | | |
|---|---|---|---|
| di- | | | |
| date | | | |
| ID | Contig | Internal name | Gene name |
| 1 | AA686189 | W41270_DB81_seg11 | TNFRSF12A |
| 3 | AA799594 | AA799594_DB71_seg0 | AA799594 |
| 5 | AI045075 | W64472_DB81_seg2 | ISG20 |
| 7 | AI454051 | AI454051_DB71_seg0 | AI454051 |
| 8 | AI502869 | AI502869_DB71_seg0 | AI502869 |
| 9 | AW919147 | AW919147_DB71_seg0 | AW919147 |
| 12 | H31799 | W83813_DB81_seg27 | EI24 |
| 14 | H31883 | MUSCYCG1R_DB81_seg15-17 | CCNG1 |
| 15 | H31883 | MUSCYCG1R_DB81_seg19-20 | CCNG1 |
| 18 | H33998 | W33294_DB81_seg44 | WDR77 |
| 21 | RATRAB13X | RATRAB13X_DB81_seg15-17 | RAB13 |
| 22 | RATRAB13X | RATRAB13X_DB81_seg22 | RAB13 |
| 24 | RNU24174 | MMU09507_DB81_seg15 | CDKN1A |

TABLE 15
Wild-Types for Day-5 signatures

| Candidate ID | Contig | Internal name | Gene name |
|---|---|---|---|
| 4 | AA964541 | AA964541_DB71_seg0 | AA964541 |
| 10 | BI293562 | BI293562_DB71_seg2 | GDF15 |
| 19 | H33998 | H33998_DB71_seg19 | WDR77 |
| 20 | RATLRFI | RATLRFI_DB71_seg9 | ATF3 |
| 25 | RNU24174 | RNU24174_DB71_seg8 | CDKN1A |

TABLE 16
Optimal signature for Day-1 data

| Can- | | | |
|---|---|---|---|
| di- | | | |
| date | | | |
| ID | Contig | Internal name | Gene name |
| 1 | AA686189 | W41270_DB81_seg11 | TNFRSF12A |
| 4 | AA964541 | AA964541_DB71_seg0 | AA964541 |
| 6 | AI045075 | AI045075_DB71_seg6 | ISG20 |
| 15 | H31883 | MUSCYCG1R_DB81_seg19-20 | CCNG1 |
| 20 | RATLRFI | RATLRFI_DB71_seg9 | ATF3 |
| 23 | RATRAB13X | RATRAB13X_DB71_seg11-13 | RAB13 |
| 25 | RNU24174 | RNU24174_DB71_seg8 | CDKN1A |

TABLE 17
Novel-Variants for Day-1 signatures

| Can- | | | |
|---|---|---|---|
| di- | | | |
| date | | | |
| ID | Contig | Internal name | Gene name |
| 1 | AA686189 | W41270_DB81_seg11 | TNFRSF12A |
| 3 | AA799594 | AA799594_DB71_seg0 | AA799594 |
| 8 | AI502869 | AI502869_DB71_seg0 | AI502869 |
| 11 | H31045 | H31045_DB71_seg5 | H31045 |
| 14 | H31883 | MUSCYCG1R_DB81_seg15-17 | CCNG1 |
| 15 | H31883 | MUSCYCG1R_DB81_seg19-20 | CCNG1 |
| 17 | H33998 | W33294_DB81_seg23 | WDR77 |
| 21 | RATRAB13X | RATRAB13X_DB81_seg15-17 | RAB13 |

TABLE 18
Wild-Types for Day-1 signatures

| Candi- | | | |
|---|---|---|---|
| date ID | Contig | Internal name | Gene name |
| 2 | AA686189 | AA686189_DB71_seg6 | TNFRSF12A |
| 4 | AA964541 | AA964541_DB71_seg0 | AA964541 |
| 6 | AI045075 | AI045075_DB71_seg6 | ISG20 |
| 16 | H31883 | H31883_DB71_seg13 | CCNG1 |
| 19 | H33998 | H33998_DB71_seg19 | WDR77 |
| 20 | RATLRFI | RATLRFI_DB71_seg9 | ATF3 |
| 23 | RATRAB13X | RATRAB13X_DB71_seg11-13 | RAB13 |
| 25 | RNU24174 | RNU24174_DB71_seg8 | CDKN1A |

Table 19 below summarizes the SEQ ID NOs for nodes, detector nodes, other unique nodes, transcripts and proteins for each marker of the invention. Nodes are segments within a transcript that might, according to the predictions made by the LEADS platform, represent a single exon or an alternative extension of an exon. Unique nodes are the ones that appear only in a novel variant or polynucleotide and not in the wild-type. The nodes listed for new variants and polynucleotides are unique nodes. When other unique nodes exist for new-variants and polynucleotides of the same genes, they are also given. The detector nodes are the set of nodes within which the primers were designed. When possible it is the same as the unique node (and not given separately), but when the unique node is too short the detector node might include other neighboring nodes as well.

TABLE 19
Candidate SEQ IDs

| | | | | | Other | | |
|---|---|---|---|---|---|---|---|
| | | | | Detector | unique | | |
| | | | Node | nodes | nodes | Transcript | Protein |
| | | | SEQ. | SEQ. | SEQ. | SEQ. | SEQ. |
| | | Candidate | ID | ID | ID | ID | ID |
| Contig | Internal name | ID | No. | No. | No. | No. | No. |
| AA686189 | W41270_DB81_seg11 | 1 | 2 | | | 1 | 62 |
| AA686189 | AA686189_DB71_seg6 | 2 | 45 | | | 44 | 73 |
| AA799594 | AA799594_DB71_seg0 | 3 | 33 | | | 32 | |
| AA964541 | AA964541_DB71_seg0 | 4 | 47 | | | 46 | |
| AI045075 | W64472_DB81_seg2 | 5 | 4 | | 5 | 3 | 63 |
| AI045075 | AI045075_DB71_seg6 | 6 | 49 | | | 48 | 74 |
| AI454051 | AI454051_DB71_seg0 | 7 | 35 | | | 34 | |
| AI502869 | AI502869_DB71_seg0 | 8 | 37 | | | 36 | |
| AW919147 | AW919147_DB71_seg0 | 9 | 39 | | | 38 | |
| BI293562 | BI293562_DB71_seg2 | 10 | 51 | | | 50 | 75 |
| H31045 | H31045_DB71_seg5 | 11 | 41 | | | 40 | 71 |
| H31799 | W83813_DB81_seg27 | 12 | 7 | | 8 | 6 | 64 |
| H31799 | H31799_DB71_seg23 | 13 | 43 | | | 42 | 72 |
| H31883 | MUSCYCG1R_DB81_seg15-17 | 14 | 10 | 11 | | 9 | 65 |
| H31883 | MUSCYCG1R_DB81_seg19-20 | 15 | 13 | 14 | | 12 | 66 |
| H31883 | H31883_DB71_seg13 | 16 | 53 | | | 52 | 76 |
| H33998 | W33294_DB81_seg23 | 17 | 16 | | 17 18 19 | 15 | 67 |
| H33998 | W33294_DB81_seg44 | 18 | 21 | | 22 23 | 20 | 68 |
| H33998 | H33998_DB71_seg19 | 19 | 55 | | | 54 | 77 |
| RATLRFI | RATLRFI_DB71_seg9 | 20 | 57 | | | 56 | 78 |
| RATRAB13X | RATRAB13X_DB81_seg15-17 | 21 | 25 | 26 | | 24 | 69 |
| RATRAB13X | RATRAB13X_DB81_seg22 | 22 | 28 | | 29 | 27 | 70 |
| RATRAB13X | RATRAB13X_DB71_seg11-13 | 23 | 59 | | | 58 | 79 |
| RNU24174 | MMU09507_DB81_seg15 | 24 | 31 | | | 30 | |
| RNU24174 | RNU24174_DB71_seg8 | 25 | 61 | | | 60 | 80 |

Table 20 below summarizes the SEQ ID NOs for primers used to amplify specific amplicons for each marker of the invention.

TABLE 20
Candidate Primers and Amplicons

| | | | Forward | Reverse | |
|---|---|---|---|---|---|
| | | | primer | primer | Amplicon |
| | | Candidate | SEQ. ID | SEQ. ID | SEQ. ID |
| Contig | Internal name | ID | No. | No. | No. |
| AA686189 | W41270_DB81_seg11 | 1 | 252 | 253 | 254 |
| AA686189 | AA686189_DB71_seg6 | 2 | 249 | 250 | 251 |
| AA799594 | AA799594_DB71_seg0 | 3 | 255 | 256 | 257 |
| AA964541 | AA964541_DB71_seg0 | 4 | 258 | 259 | 260 |
| AI045075 | W64472_DB81_seg2 | 5 | 264 | 265 | 266 |
| AI045075 | W64472_DB81_seg2_F6R1 | 5 | 327 | 328 | 329 |
| AI045075 | AI045075_DB71_seg6 | 6 | 261 | 262 | 263 |
| AI454051 | AI454051_DB71_seg0 | 7 | 267 | 268 | 269 |
| AI502869 | AI502869_DB71_seg0 | 8 | 270 | 271 | 272 |
| AW919147 | AW919147_DB71_seg0 | 9 | 273 | 274 | 275 |
| BI293562 | BI293562_DB71_seg2 | 10 | 276 | 277 | 278 |
| H31045 | H31045_DB71_seg5 | 11 | 279 | 280 | 281 |
| H31799 | W83813_DB81_seg27 | 12 | 285 | 286 | 287 |
| H31799 | H31799_DB71_seg23 | 13 | 282 | 283 | 284 |
| H31883 | MUSCYCG1R_DB81_seg15-17 | 14 | 291 | 292 | 293 |
| H31883 | MUSCYCG1R_DB81_seg19-20 | 15 | 294 | 295 | 296 |
| H31883 | H31883_DB71_seg13 | 16 | 288 | 289 | 290 |
| H31883 | H31883_DB71_seg13_F5R5 | 16 | 330 | 331 | 332 |
| H33998 | W33294_DB81_seg23 | 17 | 297 | 298 | 299 |
| H33998 | W33294_DB81_seg44 | 18 | 300 | 301 | 302 |
| H33998 | H33998_DB71_seg19 | 19 | 303 | 304 | 305 |
| RATLRFI | RATLRFI_DB71_seg9 | 20 | 306 | 307 | 308 |
| RATRAB13X | RATRAB13X_DB81_seg15-17 | 21 | 309 | 310 | 311 |
| RATRAB13X | RATRAB13X_DB81_seg22 | 22 | 312 | 313 | 314 |
| RATRAB13X | RATRAB13X_DB71_seg11-13 | 23 | 315 | 316 | 317 |
| RNU24174 | MMU09507_DB81_seg15 | 24 | 321 | 322 | 323 |
| RNU24174 | RNU24174_DB71_seg8 | 25 | 324 | 325 | 326 |

Table 21 below provides human and mouse orthologous sequences for each rat marker of the invention.

TABLE 21
Candidates Homologues

| | | | Human | Human | Mouse | Mouse |
|---|---|---|---|---|---|---|
| | | | homologue | homologue | homologue | homologue |
| | | Candidate | transcripts | protein SEQ. | transcripts | proteins |
| Contig | Internal name | ID | SEQ. ID No. | ID No. | SEQ. ID No. | SEQ. ID No. |
| AA686189 | W41270_DB81_ | 1 | 105 106 107 | 157 158 159 | 197 198 199 | 226 227 228 |
| | seg11 | | 108 | 160 | 200 | 229 |
| AA686189 | AA686189_DB71_ | 2 | 105 106 107 | 157 158 159 | 197 198 199 | 226 227 228 |
| | seg6 | | 108 | 160 | 200 | 229 |
| AA799594 | AA799594_DB71_ | 3 | | | | |
| | seg0 | | | | | |
| AA964541 | AA964541_DB71_ | 4 | | | 190 | 219 |
| | seg0 | | | | | |
| AI045075 | W64472_DB81_ | 5 | 112 113 114 | 163 164 | 202 203 204 | 231 232 233 |
| | seg2 | | | | | |
| AI045075 | AI045075_DB71_ | 6 | 112 113 114 | 163 164 | 202 203 204 | 231 232 233 |
| | seg6 | | | | | |
| AI454051 | AI454051_DB71_ | 7 | | | 185 | |
| | seg0 | | | | | |
| AI502869 | AI502869_DB71_ | 8 | | | | |
| | seg0 | | | | | |
| AW919147 | AW919147_DB71_ | 9 | | | 195 196 | 224 225 |
| | seg0 | | | | | |
| BI293562 | BI293562_DB71_ | 10 | 100 101 102 | 154 155 156 | 186 | 215 |
| | seg2 | | 103 104 | | | |
| H31045 | H31045_DB71_ | 11 | 134 135 136 | 173 174 175 | 179 180 181 | 209 210 211 |
| | seg5 | | 137 | 176 | 182 183 184 | 212 213 214 |
| H31799 | W83813_DB81_ | 12 | 83 84 85 86 87 | 140 141 142 | 205 206 207 | 234 235 236 |
| | seg27 | | 88 89 | 143 144 | | |
| H31799 | H31799_DB71_ | 13 | 83 84 85 86 87 | 140 141 142 | 205 206 207 | 234 235 236 |
| | seg23 | | 88 89 | 143 144 | | |
| H31883 | MUSCYCG1R_ | 14 | 109 110 111 | 161 162 | 188 189 | 217 218 |
| | DB81_seg15-17 | | | | | |
| H31883 | MUSCYCG1R_ | 15 | 109 110 111 | 161 162 | 188 189 | 217 218 |
| | DB81_seg19-20 | | | | | |
| H31883 | H31883_DB71_ | 16 | 109 110 111 | 161 162 | 188 189 | 217 218 |
| | seg13 | | | | | |
| H33998 | W33294_DB81_ | 17 | 90 91 92 93 94 | 145 146 147 | 191 192 193 | 220 221 222 |
| | seg23 | | 95 96 97 98 99 | 148 149 150 | 194 | 223 |
| | | | | 151 152 153 | | |
| H33998 | W33294_DB81_ | 18 | 90 91 92 93 94 | 145 146 147 | 191 192 193 | 220 221 222 |
| | seg44 | | 95 96 97 98 99 | 148 149 150 | 194 | 223 |
| | | | | 151 152 153 | | |
| H33998 | H33998_DB71_ | 19 | 90 91 92 93 94 | 145 146 147 | 191 192 193 | 220 221 |
| | seg19 | | 95 96 97 98 99 | 148 149 150 | 194 | 222 223 |
| | | | | 151 152 153 | | |
| RATLRFI | RATLRFI_DB71_ | 20 | 81 82 | 138 139 | 177 178 | 208 |
| | seg9 | | | | | |
| RATRAB13X | RATRAB13X_ | 21 | 115 116 117 | 165 166 167 | 201 | 230 |
| | DB81_seg15-17 | | 118 119 | 168 169 | | |
| RATRAB13X | RATRAB13X_ | 22 | 115 116 117 | 165 166 167 | 201 | 230 |
| | DB81_seg22 | | 118 119 | 168 169 | | |
| RATRAB13X | RATRAB13X_ | 23 | 115 116 117 | 165 166 167 | 201 | 230 |
| | DB71_seg11-13 | | 118 119 | 168 169 | | |
| RNU24174 | MMU09507_ | 24 | 120 121 122 | 170 171 172 | 187 | 216 |
| | DB81_seg15 | | 123 124 125 | | | |
| | | | 126 127 128 | | | |
| | | | 129 130 131 | | | |
| | | | 132 133 | | | |
| RNU24174 | RNU24174_DB71_ | 25 | 120 121 122 | 170 171 172 | 187 | 216 |
| | seg8 | | 123 124 125 | | | |
| | | | 126 127 128 | | | |
| | | | 129 130 131 | | | |
| | | | 132 133 | | | |

### Example 2

### Real Time qPCR Analysis of the Selected Markers

### Example 2.1

Expression of tumor necrosis factor receptor superfamily, member 12a, AA686189, transcripts which are detectable by amplicon as depicted in sequence name AA686189_DB71_seg6 in kidney tissues of treated or untreated rats

Expression of tumor necrosis factor receptor superfamily, member 12a transcripts detectable by or according to seg6—AA686189_DB71_seg6_F1R1 (SEQ ID NO: 251) amplicon and primers AA686189_DB71_seg6_F1 (SEQ ID NO: 249) and AA686189_DB71_seg6_R1 (SEQ ID NO: 250) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AA686189_DB71_seg6 in Table 22 contains the normalized expression values of the above-indicated tumor necrosis factor receptor superfamily, member 12a transcript in treated or untreated kidney samples.

As is evident from the column entitled AA686189_DB71_seg6 in Table 22, the level of expression of the tumor necrosis factor receptor superfamily, member 12a transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 8.00E⁻⁰⁴ and P-value for day 5: 7.5E⁻⁰⁶.

Primer pairs are also optionally and preferably encompassed within the present invention; for example, for the above experiment, the following primer pair was used as a non-limiting illustrative example only of a suitable primer pair AA686189_DB71_seg6_F1 (SEQ ID NO: 249) forward primer; and AA686189_DB71_seg6_R1 (SEQ ID NO: 250) reverse primer.

The present invention also preferably encompasses any amplicon obtained through the use of any suitable primer pair; for example, for the above experiment, the following amplicon was obtained as a non-limiting illustrative example only of a suitable amplicon: AA686189_DB71_seg6_F1R1 (SEQ ID NO: 251).

Forward primer (AA686189_DB71_seg6_F1 (SEQ ID NO: 249)):

CCAAGGACTGGGCTTAGGGT

Reverse primer (AA686189_DB71_seg6_R1 (SEQ ID NO: 250)):

AGAGATTCCCTTGTGCAAATGC

Amplicon (AA686189_DB71_seg6_F1R1 (SEQ ID NO: 251))

CCAAGGACTGGGCTTAGGGTTCAGGGGAGCCTTCCAGGGTGTCTAATTGC
CCTGTCTCTGGTTCTGGGGCAGACAGAGAGCCTCAAGCTAGGTCACAAAG
CGACTCATACTAAGGATCTGCAGCATTTGCACAAGGGAATCTCT

### Example 2.2

Expression of tumor necrosis factor receptor superfamily, member 12a, AA686189, transcripts which are detectable by amplicon as depicted in sequence name W41270_DB81_seg11 in kidney tissues of treated or untreated rats

Expression of tumor necrosis factor receptor superfamily, member 12a transcripts detectable by or according to seg11—W41270_DB81_seg11_F2R2 (SEQ ID NO: 254) amplicon and primers W41270_DB81_seg11_F2 (SEQ ID NO: 252) and W41270_DB81_seg11R2 (SEQ ID NO: 253) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled W41270_DB81_seg11 in Table 22 contains the normalized expression values of the above-indicated tumor necrosis factor receptor superfamily, member 12a transcript in treated or untreated kidney samples.

As is evident from the column entitled W41270_DB81_seg11 in Table 22, the level of expression of the tumor necrosis factor receptor superfamily, member 12a transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 4.00E⁻⁰⁴, and P-value fro day 5: 7.50E⁻¹⁰.

Primer pairs are also optionally and preferably encompassed within the present invention; for example, for the above experiment, the following primer pair was used as a non-limiting illustrative example only of a suitable primer pair: W41270_DB81_seg11_F2 (SEQ ID NO: 252) forward primer; and W41270_DB81_seg11_R2 (SEQ ID NO: 253) reverse primer.

The present invention also preferably encompasses any amplicon obtained through the use of any suitable primer pair; for example, for the above experiment, the following amplicon was obtained as a non-limiting illustrative example only of a suitable amplicon: W41270_DB81_seg11_F2R2 (SEQ ID NO: 254).

Forward primer (W41270_DB81_seg11_F2 (SEQ ID NO: 252)):

GATCTGGGTAGGTGGTTGTTGG

Reverse primer (W41270_DB81_seg11_R2 (SEQ ID NO: 253)):

CGCACACCCTTATAAAAGTCCC

Amplicon (W41270_DB81_seg11_F2R2 (SEQ ID NO: 254)):

GATCTGGGTAGGTGGTTGTTGGGGCAGAAAGGAGGTCGTAGACTTAGGAT
ATAGGAAACCAGGAAAAACTGACTGAGGAAGGGACTTTTATAAGGGTGTGC
G

### Example 2.3

Expression of AA799594, transcripts which are detectable by amplicon as depicted in sequence name AA799594_DB71_seg0 in kidney tissues of treated or untreated rats

Expression of AA799594 detectable by or according to seg0—AA799594_DB71_seg0_F1R1 (SEQ ID NO: 257) amplicon and primers AA799594_DB71_seg0_F1 (SEQ ID NO: 255) and AA799594_DB71_seg0_R1 (SEQ ID NO: 256) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AA799594_DB71_seg0 in Table 22 contains the normalized expression values of the above-indicated AA799594 transcript in treated or untreated kidney samples.

As is evident from the column entitled AA799594_DB71_seg0 in Table 22, the level of expression of AA799594 transcript detectable by the above amplicon was significantly higher in the samples treated with toxic compounds (samples 1-58 and 76-105, in kidney tissue panel described in hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 8.00E⁻⁰³ and P-value for day 5: 0.01.

Primer pairs are also optionally and preferably encompassed within the present invention; for example, for the above experiment, the following primer pair was used as a non-limiting illustrative example only of a suitable primer pair:

AA799594_DB71_seg0_F1 (SEQ ID NO: 255) forward primer; and AA799594_DB71_seg0_R1 (SEQ ID NO: 256) reverse primer.

The present invention also preferably encompasses any amplicon obtained through the use of any suitable primer pair; for example, for the above experiment, the following amplicon was obtained as a non-limiting illustrative example only of a suitable amplicon: AA799594_DB71_seg0_F1R1 (SEQ ID NO: 257).

Forward primer (AA799594_DB71_seg0_F1 (SEQ ID NO: 255)):

ATCTCGATGGTAACGGGTCTAATC

Reverse primer (AA799594_DB71_seg0_R1 (SEQ ID NO: 256)):

TTTGTGCTGACCTTCATGCC

Amplicon (AA799594_DB71_seg0_F1R1 (SEQ ID NO: 257)):

ATCTCGATGGTAACGGGTCTAATCAGCCCATGATCAACATAACTGTGGTG
ATATACATTTGGTATTTTTTAATTTTCGGATGCCTTCCTCAACATAGCCG
TCAAGGCATGAAGGTCAGCACAAA

### Example 2.4

Expression of AA964541, transcripts which are detectable by amplicon as depicted in sequence name AA964541_DB71_seg0 in kidney tissues of treated or untreated rats

Expression of AA964541 transcripts detectable by or according to seg0—AA964541_DB71_seg0_F2R2 (SEQ ID NO: 260) amplicon and primers AA964541_DB71_seg0_F2 (SEQ ID NO: 258) and AA964541_DB71_seg0_R2 (SEQ ID NO: 259) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AA964541_DB71_seg0 in Table 22 contains the normalized expression values of the above-indicated AA964541 transcript in treated or untreated kidney samples.

As is evident from the column entitled AA964541_DB71_seg0 in Table 22, the level of expression of

AA964541 transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 2.00E⁻⁰⁶, and P-Value for day 5: 3.00E⁻⁰⁸.

Primer pairs are also optionally and preferably encompassed within the present invention; for example, for the above experiment, the following primer pair was used as a non-limiting illustrative example only of a suitable primer pair: AA964541_DB71_seg0_F2 (SEQ ID NO: 258) forward primer; and AA964541_DB71_seg0_R2 (SEQ ID NO: 259) reverse primer.

The present invention also preferably encompasses any amplicon obtained through the use of any suitable primer pair; for example, for the above experiment, the following amplicon was obtained as a non-limiting illustrative example only of a suitable amplicon:

AA964541_DB71_seg0_F2R2 (SEQ ID NO: 260).

Forward primer (AA964541_DB71_seg0_F2 (SEQ ID NO: 258)):

CCAGCCTAGCCTCTCTTTTGC

Reverse primer (AA964541_DB71_seg0_R2 (SEQ ID NO: 259)):

AACATTCCCACAGGGTACATTCA

Amplicon (AA964541_DB71_seg0_F2R2 (SEQ ID NO: 260)):

CCAGCCTAGCCTCTCTTTTGCACTGCTGGTTCAGCCCACTGGGCCTCCGT
CCTTTCCTCTGGAAGGGACTTGGCCTTGGGTGACAAATTCCTCTTTGATG
AATGTACCCTGTGGGAATGTT

### Example 2.5

Expression of Interferon stimulated exonuclease 20 (ISG20), AI045075, transcripts which are detectable by amplicon as depicted in sequence name AI045075_DB71_seg6 in kidney tissues of treated or untreated rats

Expression of Interferon stimulated exonuclease 20 (ISG20) transcripts detectable by or according to seg6—AI045075_DB71_seg6_F2R2 (SEQ ID NO: 263) amplicon and primers AI045075_DB71_seg6_F2 (SEQ ID NO: 261) and AI045075_DB71_seg6_R2 (SEQ ID NO: 262) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AI045075_DB71_seg6 in Table 22 contains the normalized expression values of the above-indicated Interferon stimulated exonuclease 20 (ISG20) transcript in treated or untreated kidney samples.

As is evident from the column entitled AI045075_DB71_seg6 in Table 22, the level of expression of the Interferon stimulated exonuclease 20 (ISG20) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-Value for day 1: 2.50E⁻⁰⁴, and P-Value for day 5: 6.50E⁻⁰⁴.

Primer pairs are also optionally and preferably encompassed within the present invention; for example, for the above experiment, the following primer pair was used as a non-limiting illustrative example only of a suitable primer pair: AI045075_DB71_seg6_F2 (SEQ ID NO: 261) forward primer; and AI045075_DB71_seg6_R2 (SEQ ID NO: 262) reverse primer.

The present invention also preferably encompasses any amplicon obtained through the use of any suitable primer pair; for example, for the above experiment, the following amplicon was obtained as a non-limiting illustrative example only of a suitable amplicon: AI045075_DB71_seg6_F2R2 (SEQ ID NO: 263).

Forward primer (AI045075_DB71_seg6_F2 (SEQ ID NO: 261)):

GGGCCACAATGGAGCTCTAC

Reverse primer (AI045075_DB71_seg6_R2 (SEQ ID NO: 262)):

ACAGGTCTCATTCATGGAAAACTATG

Amplicon (AI045075_DB71_seg6_F2R2 (SEQ ID NO: 263)):

GGGCCACAATGGAGCTCTACAAAATCTCTCAGCGACTCAGAGCCCAGCGA
GGGCTGCCCTGCCTGGGAACATCAGCCTGAACTTCATCCTCATCCAGGAT
CAGAAGCAGCTACTCCTTGAAGGACCATAGTTTTCCATGAATGAGACCTG
T

### Example 2.6

Expression of Interferon stimulated exonuclease 20 (ISG20), AI045075, transcripts which are detectable by amplicon as depicted in sequence name W64472_DB81_seg2_{—} in kidney tissues of treated or untreated rats

Expression of Interferon stimulated exonuclease 20 (ISG20), AI045075, transcripts detectable by or according to seg2—W64472_DB81_seg2_F1R1 (SEQ ID NO: 266) amplicon and primers W64472_DB81_seg2_F1 (SEQ ID NO: 264) and W64472_DB81_seg2_R1 (SEQ ID NO: 265) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled W64472_DB81_seg2 in Table 22 contains the normalized expression values of the above-indicated Interferon stimulated exonuclease 20 (ISG20), AI045075, transcript in treated or untreated kidney samples.

As is evident from the column entitled W64472_DB81_seg2 in Table 22, the level of expression of the Interferon stimulated exonuclease 20 (ISG20), AI045075, transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 2.00E⁻¹⁰.

Primer pairs are also optionally and preferably encompassed within the present invention; for example, for the above experiment, the following primer pair was used as a non-limiting illustrative example only of a suitable primer pair: W64472_DB81_seg2_F1 (SEQ ID NO: 264) forward primer; and W64472_DB81_seg2_R1 (SEQ ID NO: 265) reverse primer.

The present invention also preferably encompasses any amplicon obtained through the use of any suitable primer pair; for example, for the above experiment, the following amplicon was obtained as a non-limiting illustrative example only of a suitable amplicon:

W64472_DB81_seg2_F1R1 (SEQ ID NO: 266).

Forward primer (W64472_DB81_seg2_F1 (SEQ ID NO: 264)):

GGAGGAGGGCAGAGCCA

Reverse primer (W64472_DB81_seg2_R1 (SEQ ID NO: 265)):

TCTGGTTCATTATCAAGGGAAGTTG

Amplicon (W64472_DB81_seg2_F1R1 (SEQ ID NO: 266)):

GGAGGAGGGCAGAGCCAGAGGAGGGACAGCCTGATGCAGACAGCCCTGAC
TCAACCTGCCAGCCCCCTTACCTGTCAGCCTTGAGGAGATGGAACAGCCC
AGCCTACTAGGCCTGCCCCCACTCCCCAACTTCCCTTGATAATGAACCAG
A

### Example 2.7

Expression of AI454051 transcripts which are detectable by amplicon as depicted in sequence name AI454051_DB71_seg0 in kidney tissues of treated or untreated rats

Expression of AI454051 transcripts detectable by or according to seg0—AI454051_DB71_seg0_F1R1 (SEQ ID NO: 269) amplicon and primers AI454051_DB71_seg0_F1 (SEQ ID NO: 267) and AI454051_DB71_seg0_R1 (SEQ ID NO: 268) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AI454051_DB71_seg0 in Table 22 contains the normalized expression values of the above-indicated AI454051 transcript in treated or untreated kidney samples.

As is evident from the column entitled AI454051_DB71_seg0 in Table 22, the level of expression of the AI454051 transcript detectable by the above amplicon was lower in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove).

Primer pairs are also optionally and preferably encompassed within the present invention; for example, for the above experiment, the following primer pair was used as a non-limiting illustrative example only of a suitable primer pair: AI454051_DB71_seg0_F1 (SEQ ID NO: 267) forward primer; and AI454051_DB71_seg0_R1 (SEQ ID NO: 268) reverse primer.

The present invention also preferably encompasses any amplicon obtained through the use of any suitable primer pair; for example, for the above experiment, the following amplicon was obtained as a non-limiting illustrative example only of a suitable amplicon: AI454051_DB71_seg0_F1R1 (SEQ ID NO: 269).

Forward primer (AI454051_DB71_seg0_F1 (SEQ ID NO: 267)):

ATCCTGTTGGGTCTGCTTCTCA

Reverse primer (AI454051_DB71_seg0_R1 (SEQ ID NO: 268)):

CACAAGTAAATACATAGACTGGAATCAATG

Amplicon (AI454051_DB71_seg0_F1R1 (SEQ ID NO: 269)):

ATCCTGTTGGGTCTGCTTCTCACAGCGTATGGGGCTGTGTGCTTTTACTC
ATGGTGGCAGCAGCAGTTGTTGTCACTTCTTTGGCCAGGCACAGTGGGTG
ATCCTTACAGAGCAGCACAGATTCCCATTGATTCCAGTCTATGTATTTAC
TTGTG

### Example 2.8

### Expression of AI502869 Transcripts which are Detectable by Amplicon as Depicted in Sequence Name AI502869_DB71_Seg0 in Kidney Tissues of Treated or Untreated Rats

Expression of AI502869 transcripts detectable by or according to seg0—AI502869_DB71_seg0_F1R1 (SEQ ID NO: 272) amplicon and primers AI502869_DB71_seg0_F1 (SEQ ID NO: 270) and AI502869_DB71_seg0_R1 (SEQ ID NO: 271) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AI502869_DB71_seg0 in Table 22 contains the normalized expression values of the above-indicated AI502869 transcript in treated or untreated kidney samples.

As is evident from the column entitled AI502869_DB71_seg0_F1R1 (SEQ ID NO: 272) in Table 22, the level of expression of the AI502869 transcript detectable by the above amplicon was significantly lower in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 7.00E⁻⁰³ and P-value for day 5: 2.50E⁻⁰⁵.

Primer pairs are also optionally and preferably encompassed within the present invention; for example, for the above experiment, the following primer pair was used as a non-limiting illustrative example only of a suitable primer pair: AI502869_DB71_seg0_F1 (SEQ ID NO: 270) forward primer; and AI502869_DB71_seg0_R1 (SEQ ID NO: 271) reverse primer.

The present invention also preferably encompasses any amplicon obtained through the use of any suitable primer pair; for example, for the above experiment, the following amplicon was obtained as a non-limiting illustrative example only of a suitable amplicon:

Forward primer (AI502869_DB71_seg0_F1 (SEQ ID NO: 270)):

TGTTTAAAATCTGGGCTCTTGATTC

Reverse primer (AI502869_DB71_seg0_R1 (SEQ ID NO: 271)):

GTATTTCCAATAGTCAATGCAGTAACTCTAC

Amplicon (AI502869_DB71_seg0_F1R1 (SEQ ID NO: 272)):

TGTTTAAAATCTGGGCTCTTGATTCAGACCGAGTTTTAATACTGGATCTC
ATTCAATCTTTGTGTTTTTTCTTTTTCTTTTCTTTTTTTTCCAATAGTAC
GGCAGTGCTAATAGCAGTCCTTTAGTAGAGTTACTGCATTGACTATTGGA
AATAC

### Example 2.9

### Expression of AW919147 Transcripts which are Detectable by Amplicon as Depicted in Sequence Name AW919147_DB71_Seg0 in Kidney Tissues of Treated or Untreated Rats

Expression of AW919147 transcripts detectable by or according to seg0—AW919147_DB71_seg0_F2R2 (SEQ ID NO: 275) amplicon and primers AW919147_DB71_seg0_F2 (SEQ ID NO: 273) and AW919147_DB71_seg0_R2 (SEQ ID NO: 274) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled AW919147_DB71_seg0 in Table 22 contains the normalized expression values of the above-indicated AW919147 transcript in treated or untreated kidney samples.

As is evident from the column entitled AW919147_DB71_seg0 in Table 22, the level of expression of the AW919147 transcript detectable by the above amplicon was lower in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 0.01.

Primer pairs are also optionally and preferably encompassed within the present invention; for example, for the above experiment, the following primer pair was used as a non-limiting illustrative example only of a suitable primer pair: AW919147_DB71_seg0_F2 (SEQ ID NO: 273) forward primer; and AW919147_DB71_seg0_R2 (SEQ ID NO: 274) reverse primer.

The present invention also preferably encompasses any amplicon obtained through the use of any suitable primer pair; for example, for the above experiment, the following amplicon was obtained as a non-limiting illustrative example only of a suitable amplicon AW919147_DB71_seg0_F2R2 (SEQ ID NO: 275):

Forward primer (AW919147_DB71_seg0_F2 (SEQ ID NO: 273)):

GCCACAGTAGAGGCTGCACTT

Reverse primer (AW919147_DB71_seg0_R2 (SEQ ID NO: 274)):

GAATCAACTGGAAATGTTCTAGGG

Amplicon (AW919147_DB71_seg0_F2R2 (SEQ ID NO: 275)):

GCCACAGTAGAGGCTGCACTTGCCACTGCTGTGTAGCGGCACTCTCCTGA
CTCACTTAAAAATTCTCTGGAGGTTTAAGTGAGGACCCAGTTCCATCTCT
AGATATCCAGGCTCCCTAGAACATTTCCAGTTGATTC

### Example 2.10

### Expression of Growth Differentiation Factor 15 (GDF15), BI293562, Transcripts which are Detectable by Amplicon as Depicted in Sequence Name BI293562_DB71_Seg2 in Kidney Tissues of Treated or Untreated Rats

Expression of growth differentiation factor 15 (GDF15) transcripts detectable by or according to seg2—BI293562_DB71_seg2_F2R2 (SEQ ID NO: 278) amplicon and primers BI293562_DB71_seg2_F2 (SEQ ID NO: 276) and BI293562_DB71_seg2_R2 (SEQ ID NO: 277) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled BI293562_DB71_seg2 in Table 22 contains the normalized expression values of the above-indicated growth differentiation factor 15 (GDF15) transcript in treated or untreated kidney samples.

As is evident from the column entitled BI293562_DB71_seg2 in Table 22, the level of expression of the growth differentiation factor 15 (GDF15) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 0.03 and P-value for day 5: 7.50E⁻⁰⁷.

Primer pairs are also optionally and preferably encompassed within the present invention; for example, for the above experiment, the following primer pair was used as a non-limiting illustrative example only of a suitable primer pair: BI293562_DB71_seg2_F2 (SEQ ID NO: 276) forward primer; and BI293562_DB71_seg2_R2 (SEQ ID NO: 277) reverse primer.

The present invention also preferably encompasses any amplicon obtained through the use of any suitable primer pair; for example, for the above experiment, the following amplicon was obtained as a non-limiting illustrative example only of a suitable amplicon BI293562_DB71_seg2_F2R2 (SEQ ID NO: 278):

Forward primer (BI293562_DB71_seg2_F2 (SEQ ID NO: 276)):

CTGCAACTGAGCATGTGCGT

Reverse primer (BI293562_DB71_seg2_R2 (SEQ ID NO: 277)):

TGCATAAGAACCACCGGGG

Amplicon (BI293562_DB71_seg2_F2R2 (SEQ ID NO: 278)):

CTGCAACTGAGCATGTGCGTGGGCGAGTGCCCCCACCTCTACCGGTCGGC
CAACACGCATGCGCAGATCAAAGCACGCCTGCATGGCCTGCAGCCCGACA
GAGTGCCGGCCCCGTGCTGTGTCCCCTCCAGCTACACCCCGGTGGTTCTT
ATGCA

### Example 2.11

### Expression H31045 Transcripts which are Detectable by Amplicon as Depicted in Sequence Name

H31045_DB71_Seg5 in Kidney Tissues of Treated or Untreated Rats

Expression of H31045 transcripts detectable by or according to seg5—H31045_DB71_seg5_F3R3 (SEQ ID NO: 281) amplicon and primers H31045_DB71_seg5_F3 (SEQ ID NO: 279) and H31045_DB71_seg5_R3 (SEQ ID NO: 280) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled H31045_DB71_seg5 in Table 22 contains the normalized expression values of the above-indicated H31045 transcript in treated or untreated kidney samples.

As is evident from the column entitled H31045_DB71_seg5 in Table 22, the level of expression of the H31045 transcript detectable by the above amplicon was significantly lower in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 0.015.

Primer pairs are also optionally and preferably encompassed within the present invention; for example, for the above experiment, the following primer pair was used as a non-limiting illustrative example only of a suitable primer pair: H31045_DB71_seg5_F3 (SEQ ID NO: 279) forward primer; and H31045_DB71_seg5_R3 (SEQ ID NO: 280) reverse primer.

The present invention also preferably encompasses any amplicon obtained through the use of any suitable primer pair; for example, for the above experiment, the following amplicon was obtained as a non-limiting illustrative example only of a suitable amplicon H31045_DB71_seg5_F3R3 (SEQ ID NO: 281):

Forward primer (H31045_DB71_seg5_F3 (SEQ ID NO: 279)):

GGACGGCCGAGCACAA

Reverse primer (H31045_DB71_seg5_R3 (SEQ ID NO: 280)):

GTGTGCATGTGCATAGGTCAGA

Amplicon (H31045_DB71_seg5_F3R3 (SEQ ID NO: 281)):

GGACGGCCGAGCACAATGGTGCTTGCTGTACAGCCTCATAGACCTTCCTT
GGATCTCCCAGACACACTTAGTGGAAGGAGAGAATCCATTCTTGCAAATT
GTCTGACCTATGCACATGCACAC

### Example 2.12

### Expression Etoposide Induced 2.4 mRNA (EI24) H31799 Transcripts which are Detectable by Amplicon as Depicted in Sequence Name H31799_DB71_Seg23 in Kidney Tissues of Treated or Untreated Rats

Expression of Etoposide induced 2.4 mRNA (EI24) transcripts detectable by or according to seg23—H31799_DB71_seg23_F1R1 (SEQ ID NO: 284) amplicon and primers H31799_DB71_seg23_F1 (SEQ ID NO: 282) and H31799_DB71_seg23_R1 (SEQ ID NO: 283) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled H31799_DB71_seg23 in Table 22 contains the normalized expression values of the above-indicated Etoposide induced 2.4 mRNA (EI24) transcript in treated or untreated kidney samples.

As is evident from the column entitled H31799_DB71_seg23 in Table 22, the level of expression of the Etoposide induced 2.4 mRNA (EI24) transcript detectable by the above amplicon was higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 0.01.

Primer pairs are also optionally and preferably encompassed within the present invention; for example, for the above experiment, the following primer pair was used as a non-limiting illustrative example only of a suitable primer pair: H31799_DB71_seg23_F1 (SEQ ID NO: 282) forward primer; and H31799_DB71_seg23_R1 (SEQ ID NO: 283) reverse primer.

The present invention also preferably encompasses any amplicon obtained through the use of any suitable primer pair; for example, for the above experiment, the following amplicon was obtained as a non-limiting illustrative example only of a suitable amplicon H31799_DB71_seg23_F1R1 (SEQ ID NO: 284):

Forward primer (H31799_DB71_seg23_F1 (SEQ ID NO: 282)):

TACTGTTTCCTGTGAAGCACATACCT

Reverse primer (H31799_DB71_seg23_R1 (SEQ ID NO: 283)):

GCAGAGCAGTAAAGACCAAAACC

Amplicon (H31799_DB71_seg23_F1R1 (SEQ ID NO: 284)):

TACTGTTTCCTGTGAAGCACATACCTTGTATGTGGGAGGTAAAGGAGCAC
GCCAGCTGCTCCATGTCACTCCCTCTATAGCCATCACTGTCTTGTTTTTT
TGTAACTCAGGTTAGGTTTTGGTCTTTACTGCTCTGC

### Example 2.13

### Expression Etoposide Induced 2.4 (EI24) mRNA, H31799, Transcripts which are Detectable by Amplicon as Depicted in Sequence Name W83813_DB81_Seg27 in Kidney Tissues of Treated or Untreated Rats

Expression of Etoposide induced 2.4 mRNA (EI24) transcripts detectable by or according to seg27—W83813_DB81_seg27_F1R1 (SEQ ID NO: 287) amplicon and primers W83813_DB81_seg27_F1 (SEQ ID NO: 285) and W83813_DB81_seg27_R1 (SEQ ID NO: 286) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled W83813_DB81_seg27 in Table 22 contains the normalized expression values of the above-indicated Etoposide induced 2.4 mRNA (EI24) transcript in treated or untreated kidney samples.

As is evident from the column entitled W83813_DB81_seg27 in Table 22, the level of expression of the Etoposide induced 2.4 mRNA (EI24) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 1.50E⁻⁰⁸.

Primer pairs are also optionally and preferably encompassed within the present invention; for example, for the above experiment, the following primer pair was used as a non-limiting illustrative example only of a suitable primer pair: W83813_DB81_seg27_F1 (SEQ ID NO: 285) forward primer; and W83813_DB81_seg27_R1 (SEQ ID NO: 286) reverse primer.

The present invention also preferably encompasses any amplicon obtained through the use of any suitable primer pair; for example, for the above experiment, the following amplicon was obtained as a non-limiting illustrative example only of a suitable amplicon W83813_DB81_seg27_F1R1 (SEQ ID NO: 287):

Forward primer (W83813_DB81_seg27_F1 (SEQ ID NO: 285)):

AATCTAGGCTGCCTCCTGGAG

Reverse primer (W83813_DB81_seg27_R1 (SEQ ID NO: 286)):

AGGTCAATTATACAAGGCATGACTTTAG

Amplicon (W83813_DB81_seg27_F1R1 (SEQ ID NO: 287)):

AATCTAGGCTGCCTCCTGGAGGAAGATACTTAGGAGTTCAGAAGTGAAGA
GATGAGGCTTATAATACTTTTTCCTAAAGTCATGCCTTGTATAATTGACC
T

### Example 2.14

### Expression of Cyclin-G1 (CCNG1), H31883, Transcripts which are Detectable by Amplicon as Depicted in Sequence Name H31883_DB71_Seg13 in Kidney Tissues of Treated or Untreated Rats

Expression of Cyclin-G1 (CCNG1) transcripts detectable by or according to seg13—H31883_DB71_seg13_F1R1 (SEQ ID NO: 290) amplicon and primers H31883_DB71_seg13_F1 (SEQ ID NO: 288) and H31883_DB71_seg13_R1 (SEQ ID NO: 289) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled H31883_DB71_seg13 in Table 22 contains the normalized expression values of the above-indicated Cyclin-G1 (CCNG1) transcript in treated or untreated kidney samples.

As is evident from the column entitled H31883_DB71_seg13 in Table 22, the level of expression of the Cyclin-G1 (CCNG1) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 5.00E⁻⁰³ and P-value for day 5: 0.04.

Primer pairs are also optionally and preferably encompassed within the present invention; for example, for the above experiment, the following primer pair was used as a non-limiting illustrative example only of a suitable primer pair: H31883_DB71_seg13_F1 (SEQ ID NO: 288) forward primer; and H31883_DB71_seg13_R1 (SEQ ID NO: 289) reverse primer.

The present invention also preferably encompasses any amplicon obtained through the use of any suitable primer pair; for example, for the above experiment, the following amplicon was obtained as a non-limiting illustrative example only of a suitable amplicon: H31883_DB71_seg13_F1R1 (SEQ ID NO: 290).

Forward primer (H31883_DB71_seg13_F1 (SEQ ID NO: 288)):

TCGTCAGAATTGCTGCCTCA

Reverse primer (H31883_DB71_seg13_R1 (SEQ ID NO: 289)):

TCTGTGGTAAAAACCTCCTGGAGT

Amplicon (H31883_DB71_seg13_F1R1 (SEQ ID NO: 290)):

TCGTCAGAATTGCTGCCTCAATCTAGTCCCATTTGAGAAAATTTGTTTCT
ACTGTCTCAATAACTGGATGAAATATCACTCTGAAAACTTGCCTATTGCA
CTAAAGCTAGTTTAGGCTTGATAAAACACTCCAGGAGGTTTTTACCACAG
A

### Example 2.15

### Expression of Cyclin-G1 (CCNG1), 1131883, Transcripts which are Detectable by Amplicon as Depicted in Sequence Name MUSCYCG1R_DB81_Seg15-17 in Kidney Tissues of Treated or Untreated Rats

Expression of Cyclin-G1 (CCNG1) detectable by or according to seg15-17—MUSCYCG1R_DB81_seg15-17_F1R1 (SEQ ID NO: 293) amplicon and primers MUSCYCG1R_DB81_seg15-17_F1 (SEQ ID NO: 291) and MUSCYCG1R_DB81_seg15-17_R1 (SEQ ID NO: 292) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled MUSCYCG1R_DB81_seg15-17 in Table 22 contains the normalized expression values of the above-indicated Cyclin-G1 (CCNG1) transcript in treated or untreated kidney samples.

As is evident from the column entitled MUSCYCG1R_DB81_seg15-17 in Table 22, the level of expression of the Cyclin-G1 (CCNG1) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 1.00E⁻⁰⁷.

Primer pairs are also optionally and preferably encompassed within the present invention; for example, for the above experiment, the following primer pair was used as a non-limiting illustrative example only of a suitable primer pair: MUSCYCG1R_DB81_seg15-17_F1 (SEQ ID NO: 291) forward primer; and MUSCYCG1R_DB81_seg15-17_R1 (SEQ ID NO: 292) reverse primer.

The present invention also preferably encompasses any amplicon obtained through the use of any suitable primer pair; for example, for the above experiment, the following amplicon was obtained as a non-limiting illustrative example only of a suitable amplicon: MUSCYCG1R_DB81_seg15-17_F1R1 (SEQ ID NO: 293).

Forward primer (MUSCYCG1R_DB81_seg15-17_F1 (SEQ ID NO: 291)):

ATAAAATCAATCCCACTTTCTTGTTAAAAG

Reverse primer (MUSCYCG1R_DB81_seg15-17_R1 (SEQ ID NO: 292)):

CTTTGCCTTAGAAAATATGATCCTGC

Amplicon (MUSCYCG1R_DB81_seg15-17_F1R1 (SEQ ID NO: 293)):

ATAAAATCAATCCCACTTTCTTGTTAAAAGGAGAAACGATCTGAATTTTG
AAAGACTAGAAGCCCAACTTAAGGCCTGCCACTGCAGGATCATATTTTCT
AAGGCAAAG

### Example 2.16

### Expression of Cyclin-G1 (CCNG1), 1131883, Transcripts which are Detectable by Amplicon as Depicted in Sequence Name MUSCYCG1R_DB81_Seg19-20 in Kidney Tissues of Treated or Untreated Rats

Expression of Cyclin-G1 (CCNG1) transcripts detectable by or according to seg19-20—MUSCYCG1R_DB81_seg19-20_F1R1 (SEQ ID NO: 296) amplicon and primers MUSCYCG1R_DB81_seg19-20_F1 (SEQ ID NO: 294) and MUSCYCG1R_DB81_seg19-20_R1 (SEQ ID NO: 295) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled MUSCYCG1R_DB81_seg19-20 in Table 22 contains the normalized expression values of the above-indicated Cyclin-G1 (CCNG1) transcript in treated or untreated kidney samples.

As is evident from the column entitled MUSCYCG1R_DB81_seg19-20 in Table 22, the level of expression of the Cyclin-G1 (CCNG1) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 6.00E⁻⁰³ and P-value for day 5: 3.00E⁻⁰⁹.

Primer pairs are also optionally and preferably encompassed within the present invention; for example, for the above experiment, the following primer pair was used as a non-limiting illustrative example only of a suitable primer pair: MUSCYCG1R_DB81_seg19-20_F1 (SEQ ID NO: 294) forward primer; and MUSCYCG1R_DB81_seg19-20_R1 (SEQ ID NO: 295) reverse primer.

The present invention also preferably encompasses any amplicon obtained through the use of any suitable primer pair; for example, for the above experiment, the following amplicon was obtained as a non-limiting illustrative example only of a suitable amplicon: MUSCYCG1R_DB81_seg19-20_F1R1 (SEQ ID NO: 296).

Forward primer (MUSCYCG1R_DB81_seg19-20_F1 (SEQ ID NO: 294)):

GAGATCCAAGCACTGAAGTATGTAGAGT

Reverse primer (MUSCYCG1R_DB81_seg19-20_R1 (SEQ ID NO: 295)):

TCAGGAGTACAGTGGATACATTTCTCTT

Amplicon (MUSCYCG1R_DB81_seg19-20_F1R1 (SEQ ID NO: 296)):

GAGATCCAAGCACTGAAGTATGTAGAGTTAACAGAAGGAGTAGAATGTAT
TCAGAAACATTCCAAGGTATGCCAAGGTGATAGCATTGATCCTATTAGCA
AGCTACAAGAGAAATGTATCCACTGTACTCCTGA

### Example 2.17

### Expression of Repeat Domain 77 (WDR77), 1133998, Transcripts which are Detectable by Amplicon as Depicted in Sequence Name W33294_DB81_Seg23 in Kidney Tissues of Treated or Untreated Rats

Expression of repeat domain 77 (WDR77) transcripts detectable by or according to seg23—W33294_DB81_seg23_F1R1 (SEQ ID NO: 299) amplicon and primers W33294_DB81_seg23_F1 (SEQ ID NO: 297) and W33294_DB81_seg23_R1 (SEQ ID NO: 298) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled W33294_DB81_seg23 in Table 22 contains the normalized expression values of the above-indicated repeat domain 77 (WDR77) transcript in treated or untreated kidney samples.

As is evident from the column entitled W33294_DB81_seg23 in Table 22, the level of expression of the repeat domain 77 (WDR77) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 7.00E⁻⁰⁴.

Primer pairs are also optionally and preferably encompassed within the present invention; for example, for the above experiment, the following primer pair was used as a non-limiting illustrative example only of a suitable primer pair: W33294_DB81_seg23_F1 (SEQ ID NO: 297) forward primer; and W33294_DB81_seg23_R1 (SEQ ID NO: 298) reverse primer.

The present invention also preferably encompasses any amplicon obtained through the use of any suitable primer pair; for example, for the above experiment, the following amplicon was obtained as a non-limiting illustrative example only of a suitable amplicon: W33294_DB81_seg23_F1R1 (SEQ ID NO: 299).

Forward primer (W33294_DB81_seg23_F1 (SEQ ID NO: 297)):

GGGCACTGCATGGATTATGTC

Reverse primer (W33294_DB81_seg23_R1 (SEQ ID NO: 298)):

CCATGCCCAGAATAAAGGAGC

Amplicon (W33294_DB81_seg23_F1R1 (SEQ ID NO: 299)):

GGGCACTGCATGGATTATGTCGGTTTTACCTAGCTTTGGAGACCATCATT
TTTTCCTAATAGGTTTGCTTCATTGTTTCAGCTCCTTTATTCTGGGCA
TGG

### Example 2.18

### Expression of Repeat Domain 77 (WDR77), 1133998, Transcripts which are Detectable by Amplicon as Depicted in Sequence Name W33294_DB81_Seg44 in Kidney Tissues of Treated or Untreated Rats

Expression of repeat domain 77 (WDR77) transcripts detectable by or according to seg44—W33294_DB81_seg44_F1R1 (SEQ ID NO: 302) amplicon and primers W33294_DB81_seg44_F1 (SEQ ID NO: 300) and W33294_DB81_seg44_R1 (SEQ ID NO: 301) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled W33294_DB81_seg44 in Table 22 contains the normalized expression values of the above-indicated repeat domain 77 (WDR77) transcript in treated or untreated kidney samples.

As is evident from the column entitled W33294_DB81_seg44 in Table 22, the level of expression of the repeat domain 77 (WDR77) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 8.00E⁻⁰⁶.

Primer pairs are also optionally and preferably encompassed within the present invention; for example, for the above experiment, the following primer pair was used as a non-limiting illustrative example only of a suitable primer pair: W33294_DB81_seg44_F1 (SEQ ID NO: 300) forward primer; and W33294_DB81_seg44_R1 (SEQ ID NO: 301) reverse primer.

The present invention also preferably encompasses any amplicon obtained through the use of any suitable primer pair; for example, for the above experiment, the following amplicon was obtained as a non-limiting illustrative example only of a suitable amplicon: W33294_DB81_seg44_F1R1 (SEQ ID NO: 302).

Forward primer (W33294_DB81_seg44_F1 (SEQ ID NO: 300)):

CTCCGTTAGCAATTATGGGTCAGT

Reverse primer (W33294_DB81_seg44_R1 (SEQ ID NO: 301)):

GAAGCTCATGGTCCTTCAGGG

Amplicon (W33294_DB81_seg44_F1R1 (SEQ ID NO: 302)):

CTCCGTTAGCAATTATGGGTCAGTAACTGATCTTATTAGAGCTTTACAAC
TTTGGTTTAGGAAAAGCACATAAAATGGGCGCCCTGAAGGACCATGAGCT
TC

### Example 2.19

### Expression of Repeat Domain 77 (WDR77), 1133998, Transcripts which are Detectable by Amplicon as Depicted in Sequence Name H33998_DB71_Seg19 in Kidney Tissues of Treated or Untreated Rats

Expression of repeat domain 77 (WDR77) transcripts detectable by or according to seg19—H33998_DB71_seg19_F3R3 (SEQ ID NO: 305) amplicon and primers H33998_DB71_seg19_F3 (SEQ ID NO: 303) and H33998_DB71_seg19_R3 (SEQ ID NO: 304) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled H33998_DB71_seg19 in Table 22 contains the normalized expression values of the above-indicated repeat domain 77 (WDR77) transcript in treated or untreated kidney samples.

As is evident from the column entitled H33998_DB71_seg19 in Table 22, the level of expression of the repeat domain 77 (WDR77) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers-59-75 and 106-129, in kidney tissue panel described in Table 9 hereinabove). Statistical analysis was applied to verify the significance of these results, P-value for day 1: 0.04 and P-value fro day 5: 2.00E⁻⁰⁷.

Primer pairs are also optionally and preferably encompassed within the present invention; for example, for the above experiment, the following primer pair was used as a non-limiting illustrative example only of a suitable primer pair: H33998_DB71_seg19_F3 (SEQ ID NO: 303) forward primer; and H33998_DB71_seg19_R3 (SEQ ID NO: 304) reverse primer.

The present invention also preferably encompasses any amplicon obtained through the use of any suitable primer pair; for example, for the above experiment, the following amplicon was obtained as a non-limiting illustrative example only of a suitable amplicon: H33998_DB71_seg19_F3R3 (SEQ ID NO: 305).

Forward primer (H33998_DB71_seg19_F3R3 (SEQ ID NO: 303)):

CTGTCATTGTCATTTTTCCCACC

Reverse primer (H33998_DB71_seg19_F3R3 (SEQ ID NO: 304)):

ATGCCGGATCTTCAATCTTAGG

Amplicon (H33998_DB71_seg19_F3R3 (SEQ ID NO: 305)):

CTGTCATTGTCATTTTTCCCACCTAAAAATTCCCTGAGGACTGATCTGGG
TACTTTGCTCTGGAGAGCTGAAGTCTGAGCGCTGTATATTTGGACTCCTA
AGATTGAAGATCCGGCAT

### Example 2.20

### Expression of Activating Transcription Factor 3 (ATF3), RATLRFI, Transcripts which are Detectable by Amplicon as Depicted in Sequence Name RATLRFI_DB71_Seg9 in Kidney Tissues of Treated or Untreated Rats

Expression of Activating transcription factor 3 (ATF3) transcripts detectable by or according to seg9—RATLRFI_DB71_seg9_F1R1 (SEQ ID NO: 308) amplicon and primers RATLRFI_DB71_seg9_F1 (SEQ ID NO: 306) and RATLRFI_DB71_seg9_R1 (SEQ ID NO: 307) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled RATLRFI_DB71_seg9 in Table 22 contains the normalized expression values of the above-indicated Activating transcription factor 3 (ATF3) transcript in treated or untreated kidney samples.

As is evident from the column entitled RATLRFI_DB71_seg9 in Table 22, the level of expression of the Activating transcription factor 3 (ATF3) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 0.025 and P-value fro day 5: 5.00E⁻⁰⁴.

Primer pairs are also optionally and preferably encompassed within the present invention; for example, for the above experiment, the following primer pair was used as a non-limiting illustrative example only of a suitable primer pair: RATLRFI_DB71_seg9_F1 (SEQ ID NO: 306) forward primer; and RATLRFI_DB71_seg9_R1 (SEQ ID NO: 307) reverse primer.

The present invention also preferably encompasses any amplicon obtained through the use of any suitable primer pair; for example, for the above experiment, the following amplicon was obtained as a non-limiting illustrative example only of a suitable amplicon: RATLRFI_DB71_seg9_F1R1 (SEQ ID NO: 308).

Forward primer (RATLRFI_DB71_seg9_F1 (SEQ ID NO: 306)):

AACTGGCTGATGACCAGCTGT

Reverse primer (RATLRFI_DB71_seg9_R1 (SEQ ID NO: 307)):

TGCTGTGCCCGGGTTCT

Amplicon (RATLRFI_DB71_seg9_F1R1 (SEQ ID NO: 308)):

AACTGGCTGATGACCAGCTGTGCTACTCTGTGCTGACCGAGGACTGATGC
CTCCTTCCCCTGTACCCACTGCTGAGGAAGAACCCGGGCACAGCA

### Example 2.21

### Expression of Ras-Related Protein Rab-13 (RAB13), RATRAB13X, Transcripts which are Detectable by Amplicon as Depicted in Sequence Name RATRAB13X_DB81_Seg15-17 in Kidney Tissues of Treated or Untreated Rats

Expression of Ras-related protein Rab-13 (RAB13) transcripts detectable by or according to seg15-17—RATRAB13X_DB81_seg15-17_F1R1 (SEQ ID NO: 311) amplicon and primers RATRAB13X_DB81_seg15-17_F1 (SEQ ID NO: 309) and RATRAB13X_DB81_seg15-17_R1 (SEQ ID NO: 310) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled RATRAB13X_DB81_seg15-17 in Table 22 contains the normalized expression values of the above-indicated Ras-related protein Rab-13 (RAB13) transcript in treated or untreated kidney samples.

As is evident from the column entitled RATRAB13X_DB81_seg15-17 in Table 22, the level of expression of the Ras-related protein Rab-13 (RAB13) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 0.05 and P-value for day 5: 3.00E⁻⁰⁷.

Primer pairs are also optionally and preferably encompassed within the present invention; for example, for the above experiment, the following primer pair was used as a non-limiting illustrative example only of a suitable primer pair: RATRAB13X_DB81_seg15-17_F1 (SEQ ID NO: 309) forward primer; and RATRAB13X_DB81_seg15-17_R1 (SEQ ID NO: 310) reverse primer.

The present invention also preferably encompasses any amplicon obtained through the use of any suitable primer pair; for example, for the above experiment, the following amplicon was obtained as a non-limiting illustrative example only of a suitable amplicon: RATRAB13X_DB81_seg15-17_F1R1 (SEQ ID NO: 311).

Forward primer (RATRAB13X_DB81_seg15-17_F1 (SEQ ID NO: 309)):

GTGAGTCAGGCCGGGCT

Reverse primer (RATRAB13X_DB81_seg15-17_R1 (SEQ ID NO: 310)):

TCAAAAAATCGGATTCTGTGCTC

Amplicon (RATRAB13X_DB81_seg15-17_F1R1 (SEQ ID NO: 311)):

GTGAGTCAGGCCGGGCTGCTGTGGCCGAATGCCTTGCTTCTGCCCTTTAT
CCAGCTCTCTCCTTCCTACCTCATCCCCTACAGTTGGCTCGAGAGCACAG
AATCCGATTTTTTGA

### Example 2.22

### Expression of Ras-Related Protein Rab-13 (RAB13), RATRAB13X, Transcripts which are Detectable by Amplicon as Depicted in Sequence Name RATRAB13X_DB81_Seg22 in Kidney Tissues of Treated or Untreated Rats

Expression of Ras-related protein Rab-13 (RAB13) transcripts detectable by or according to seg22—RATRAB13X_DB81_seg22_F2R2 (SEQ ID NO: 314) amplicon and primers RATRAB13X_DB81_seg22_F2 (SEQ ID NO: 312) and RATRAB13X_DB81_seg22_R2 (SEQ ID NO: 313) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled RATRAB13X_DB81_seg22 in Table 22 contains the normalized expression values of the above-indicated Ras-related protein Rab-13 (RAB13) transcript in treated or untreated kidney samples.

As is evident from the column entitled RATRAB13X_DB81_seg22 in Table 22, the level of expression of the Ras-related protein Rab-13 (RAB13) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 0.04 and P-value for day 5: 3.00E⁻⁰⁷.

Primer pairs are also optionally and preferably encompassed within the present invention; for example, for the above experiment, the following primer pair was used as a non-limiting illustrative example only of a suitable primer pair: RATRAB13X_DB81_seg22_F2 (SEQ ID NO: 312) forward primer; and RATRAB13X_DB81_seg22_R2 (SEQ ID NO: 313) reverse primer.

The present invention also preferably encompasses any amplicon obtained through the use of any suitable primer pair; for example, for the above experiment, the following amplicon was obtained as a non-limiting illustrative example only of a suitable amplicon: RATRAB13X_DB81_seg22_F2R2 (SEQ ID NO: 314).

Forward primer (RATRAB13X_DB81_seg22_F2 (SEQ ID NO: 312)):

TTCTCCTGAATCTGGCTGGGT

Reverse primer (RATRAB13X_DB81_seg22_R2 (SEQ ID NO: 313)):

TGGAAAAAGGATGTCCATTCTTC

Amplicon (RATRAB13X_DB81_seg22_F2R2 (SEQ ID NO: 314)):

TTCTCCTGAATCTGGCTGGGTCCCCCTTCCTTACCCCAACTCTTTAACTG
GTGATGAAAACAGCAAGGAGAAAGGGCAGCCTGAAGAATGGACATCCTTT
TTCCA

### Example 2.23

### Expression of Ras-Related Protein Rab-13 (RAB13), RATRAB13X, Transcripts which are Detectable by Amplicon as Depicted in Sequence Name RATRAB13X_DB71_Seg11-13 in Kidney Tissues of Treated or Untreated Rats

Expression of Ras-related protein Rab-13 (RAB13) transcripts detectable by or according to seg11-13—RATRAB13X_DB71_seg11-13_F1R1 (SEQ ID NO: 317) amplicon and primers RATRAB13X_DB71_seg11-13_F1 (SEQ ID NO: 315) and RATRAB13X_DB71_seg11-13_R1 (SEQ ID NO: 316) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled RATRAB13X_DB71_seg11-13 in Table 22 contains the normalized expression values of the above-indicated Ras-related protein Rab-13 (RAB13) transcript in treated or untreated kidney samples.

As is evident from the column entitled RATRAB13X_DB71_seg11-13 in Table 22, the level of expression of the Ras-related protein Rab-13 (RAB13) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 1.00E⁻⁰³.

Primer pairs are also optionally and preferably encompassed within the present invention; for example, for the above experiment, the following primer pair was used as a non-limiting illustrative example only of a suitable primer pair: RATRAB13X_DB71_seg11-13_F1 (SEQ ID NO: 315) forward primer; and RATRAB13X_DB71_seg11-13_R1 (SEQ ID NO: 316) reverse primer.

The present invention also preferably encompasses any amplicon obtained through the use of any suitable primer pair; for example, for the above experiment, the following amplicon was obtained as a non-limiting illustrative example only of a suitable amplicon: RATRAB13X_DB71_seg11-13_F1R1 (SEQ ID NO: 317).

Forward primer (RATRAB13X_DB71_seg11-13_F1 (SEQ ID NO: 315)):

AGAATCCGATTTTTTGAGACAAGTG

Reverse primer (RATRAB13X_DB71_seg11-13_R1 (SEQ ID NO: 316)):

GATCTCCGGCCTCCTGTCTT

Amplicon (RATRAB13X_DB71_seg11-13_F1R1 (SEQ ID NO: 317)):

AGAATCCGATTTTTTGAGACAAGTGCCAAATCCAGTGTGAATGTGGATGA
GGCTTTCAGTTCCCTGGCCCGTGACATCTTGCTCAAGACAGGAGGCCGGA
GATC

### Example 2.24

### Expression of Cyclin-Dependent Kinase Inhibitor 1a (CDKN1A), RNU24174, Transcripts which are Detectable by Amplicon as Depicted in Sequence Name MMU09507_DB81_Seg15 in Kidney Tissues of Treated or Untreated Rats

Expression of Cyclin-dependent kinase inhibitor 1A (CDKN1A) transcripts detectable by or according to seg15—MMU09507_DB81_seg15_F1R1 (SEQ ID NO: 323) amplicon and primers MMU09507_DB81_seg15_F1 (SEQ ID NO: 321) and MMU09507_DB81_seg15_R1 (SEQ ID NO: 322) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled MMU09507_DB81_seg15 in Table 22 contains the normalized expression values of the above-indicated Cyclin-dependent kinase inhibitor 1A (CDKN1A) transcript in treated or untreated kidney samples.

As is evident from the column entitled MMU09507_DB81_seg15 in Table 22, the level of expression of the Cyclin-dependent kinase inhibitor 1A (CDKN1A) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 3.00E⁻⁰³.

Primer pairs are also optionally and preferably encompassed within the present invention; for example, for the above experiment, the following primer pair was used as a non-limiting illustrative example only of a suitable primer pair: MMU09507_DB81_seg15_F1 (SEQ ID NO: 321) forward primer; and MMU09507_DB81_seg15_R1 (SEQ ID NO: 322) reverse primer.

The present invention also preferably encompasses any amplicon obtained through the use of any suitable primer pair; for example, for the above experiment, the following amplicon was obtained as a non-limiting illustrative example only of a suitable amplicon: MMU09507_DB81_seg15_F1R1 (SEQ ID NO: 323).

Forward primer (MMU09507_DB81_seg15_F1 (SEQ ID NO: 321)):

GGGTGGGGGAATCTAGCCT

Reverse primer (MMU09507_DB81_seg15_R1 (SEQ ID NO: 322)):

CCCCAGACAAAGGGACATGT

Amplicon (MMU09507_DB81_seg15_F1R1 (SEQ ID NO: 323)):

GGGTGGGGGAATCTAGCCTCTCTAGAGCCCTAGCCCTCTGACGAGGAGGA
GGTGTAGTGCCCTGTAGCTTTTCCCCAGGACTCGCCACATGTCCCTTTGT
CTGGGG

### Example 2.25

### Expression of Cyclin-Dependent Kinase Inhibitor 1a (CDKN1A), RNU24174, Transcripts which are Detectable by Amplicon as Depicted in Sequence Name RNU24174_DB71_Seg8 in Kidney Tissues of Treated or Untreated Rats

Expression of Cyclin-dependent kinase inhibitor 1A (CDKN1A) transcripts detectable by or according to seg8—RNU24174_DB71_seg8_F1R1 (SEQ ID NO: 326) amplicon and primers RNU24174_DB71_seg8_F1 (SEQ ID NO: 324) and RNU24174_DB71_seg8_R1 (SEQ ID NO: 325) was measured by real time PCR. The value of the expression was measured by Real-Time PCR and normalized relative to the expression of the house keeping genes, as described in section “RT PREPARATION and Real-TIME RT-PCR ANALYSIS” hereinabove.

The column entitled RNU24174_DB71_seg8 in Table 22 contains the normalized expression values of the above-indicated Cyclin-dependent kinase inhibitor 1A (CDKN1A) transcript in treated or untreated kidney samples.

As is evident from the column entitled RNU24174_DB71_seg8 in Table 22, the level of expression of the Cyclin-dependent kinase inhibitor 1A (CDKN1A) transcript detectable by the above amplicon was significantly higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 1: 2.00E⁻⁰⁵ and P-value for day 5: 3.00E⁻⁰⁵.

Primer pairs are also optionally and preferably encompassed within the present invention; for example, for the above experiment, the following primer pair was used as a non-limiting illustrative example only of a suitable primer pair: RNU24174_DB71_seg8_F1 (SEQ ID NO: 324) forward primer; and RNU24174_DB71_seg8_R1 (SEQ ID NO: 325) reverse primer.

The present invention also preferably encompasses any amplicon obtained through the use of any suitable primer pair; for example, for the above experiment, the following amplicon was obtained as a non-limiting illustrative example only of a suitable amplicon: RNU24174_DB71_seg8_F1R1 (SEQ ID NO: 326).

Forward primer (RNU24174_DB71_seg8_F1 (SEQ ID NO: 324)):

CGCTGAAGTCCTCAGTGACTTG

Reverse primer (RNU24174_DB71_seg8_R1 (SEQ ID NO: 325)):

CACTAAGTGCTTCGACACCCAC

Amplicon (RNU24174_DB71_seg8_F1R1 (SEQ ID NO: 326)):

CGCTGAAGTCCTCAGTGACTTGTCCCATTTCTTAGTAGTTGTACAAGGAG
TCAGGCCAAGATGGTGCCTCGGGGGCTGAGGGAGCTCACAGGAACTGAGC
AGTGACTGGTCCTTTCCCAGTATTGAATACTGAGCCCCTGTGGGTGTCGA
AGCACTTAGTG

Table 22 presents the normalized Real Time PCR results for all 25 amplicons detailed in examples 2.1-2.25 for all samples checked. The qRT-PCR measurements were normalized according to each samples normalization factor as described in “RT Preparation and Real-Time qRT-PCR Analysis” and further multiplied by a constant factor for ease of viewing.

TABLE 22
normalized Real Time PCR

| Sample | AA686189_DB71_seg6 | AA799594_DB71_seg0 | AA964541_DB71_seg0 | AI045075_DB71_seg6 |
|---|---|---|---|---|
| 68_1F101_day1_Saline | 0.44 | 1.17 | 0.25 | 0.51 |
| 69_1F102_day1_Saline | 0.38 | 1.16 | 0.28 | 0.44 |
| 70_1F103_day1_Saline | 0.64 | 0.92 | 0.77 | 0.54 |
| 74_1F104_day5_Saline | 0.63 | 1.69 | 0.41 | 0.42 |
| 75_1F105_day5_Saline | 0.54 | 1.56 | 0.42 | 0.59 |
| 109_1F107_day28_Saline | 0.78 | 1.6 | 0.49 | 0.79 |
| 110_1F108_day28_Saline | 0.62 | 1.08 | 0.46 | 0.65 |
| 111_1F109_day28_Saline | 0.68 | 0.9 | 0.45 | 0.9 |
| 65_1M1_day1_Saline | 0.72 | 1.02 | 0.54 | 0.79 |
| 66_1M2_day1_Saline | 0.65 | 0.87 | 0.65 | 0.36 |
| 67_1M3_day1_Saline | 0.55 | 1.17 | 0.4 | 0.84 |
| 71_1M4_day5_Saline | 0.45 | 1.28 | 0.49 | 0.6 |
| 72_1M5_day5_Saline | 0.54 | 0.67 | 0.38 | 0.56 |
| 73_1M6_day5_Saline | 0.41 | 0.86 | 0.41 | 0.45 |
| 106_1M7_day28_Saline | 0.81 | 1.06 | 0.48 | 0.64 |
| 107_1M8_day28_Saline | 0.36 | 1.24 | 0.25 | 0.42 |
| 108_1M9_day28_Saline | 0.73 | 1.36 | 0.3 | 0.55 |
| 16_2F110_day1_Gent | 1.19 | 1.25 | 1.35 | 1.5 |
| 17_2F111_day1_Gent | 0.65 | 0.72 | 0.74 | 0.93 |
| 18_2F112_day1_Gent | 1.07 | 0.93 | 1.29 | 0 |
| 45_2F113_day5_Gent | 0.64 | 0.99 | 0.86 | 0.76 |
| 46_2F114_day5_Gent | 0.72 | 0.89 | 0.58 | 1.04 |
| 47_2F115_day5_Gent | 1.21 | 1.52 | 1.04 | 0.62 |
| 91_2F116_day28_Gent | 2.7 | 0.84 | 3.19 | 2.09 |
| 92_2F117_day28_Gent | 2.09 | 0.8 | 1.77 | 3.14 |
| 93_2F118_day28_Gent | 2.48 | 0.89 | 2.16 | 1.74 |
| 1_2M10_day1_Gent | 0.94 | 0.57 | 0.9 | 1.07 |
| 2_2M11_day1_Gent | 1.04 | 0.95 | 1.19 | 1.03 |
| 3_2M12_day1_Gent | 0.6 | 0.92 | 0.44 | 0.78 |
| 31_2M13_day5_Gent | 0.81 | 0.89 | 0.91 | 1.1 |
| 32_2M14_day5_Gent | 0.83 | 0.56 | 0.76 | 0 |
| 33_2M15_day5_Gent | 0.75 | 1.04 | 0.87 | 1.04 |
| 76_2M16_day28_Gent | 1.98 | 0.54 | 3.33 | 1.25 |
| 77_2M17_day28_Gent | 1.23 | 0.5 | 1.45 | 1.99 |
| 78_2M18_day28_Gent | 2.51 | 0.67 | 3.18 | 1.29 |
| 19_3F119_day1_Cis | 0.7 | 1.17 | 0.88 | 1.01 |
| 20_3F120_day1_Cis | 0.44 | 0.39 | 0.58 | 0.68 |
| 21_3F121_day1_Cis | 0.66 | 0.98 | 0.41 | 0.79 |
| 48_3F122_day5_Cis | 1.08 | 1.16 | 0.99 | 0.86 |
| 49_3F123_day5_Cis | 0.93 | 1.34 | 0.74 | 0.53 |
| 50_3F124_day5_Cis | 0.76 | 1.11 | 0.76 | 0.76 |
| 94_3F125_day28_Cis | 0 | 0.74 | 2.63 | 2.17 |
| 95_3F126_day28_Cis | 3.57 | 0.64 | 5.28 | 3.09 |
| 96_3F127_day28_Cis | 6.06 | 0.52 | 6.85 | 3.37 |
| 4_3M19_day1_Cis | 0.48 | 0.59 | 0.4 | 0.64 |
| 5_3M20_day1_Cis | 0.42 | 0.99 | 0.29 | 0.62 |
| 6_3M21_day1_Cis | 0.68 | 0.87 | 0.56 | 1.09 |
| 34_3M22_day5_Cis | 1.5 | 0.93 | 1.94 | 1.93 |
| 35_3M23_day5_Cis | 0.69 | 0.71 | 0.67 | 0.81 |
| 36_3M24_day5_Cis | 4.48 | 0.75 | 4.8 | 1.33 |
| 79_3M25_day28_Cis | 3.15 | 0.36 | 3.72 | 3.18 |
| 80_3M26_day28_Cis | 4.29 | 0.97 | 4.71 | 2.72 |
| 81_3M27_day28_Cis | 3.86 | 0.62 | 3.97 | 1.57 |
| 22_4F128_day1_Tob | 1.19 | 0.87 | 0.56 | 1.66 |
| 23_4F129_day1_Tob | 0.77 | 0.83 | 0.82 | 0.88 |
| 24_4F130_day1_Tob | 2.14 | 1.07 | 2.02 | 3.16 |
| 51_4F131_day5_Tob | 0.85 | 1.38 | 0.82 | 0.92 |
| 52_4F132_day5_Tob | 1.04 | 0.88 | 0.77 | 1.61 |
| 53_4F133_day5_Tob | 0.78 | 0.88 | 0.65 | 1.56 |
| 97_4F134_day28_Tob | 2.1 | 0.71 | 1.68 | 0.94 |
| 98_4F135_day28_Tob | 1.65 | 0.75 | 1.37 | 0.79 |
| 99_4F136_day28_Tob | 2.23 | 0.84 | 1.59 | 1.19 |
| 7_4M28_day1_Tob | 0.65 | 0.94 | 0.47 | 0.56 |
| 8_4M29_day1_Tob | 0.85 | 1 | 0.75 | 1.03 |
| 9_4M30_day1_Tob | 1.11 | 0.61 | 1.28 | 1.07 |
| 37_4M31_day5_Tob | 0.53 | 0.81 | 0.53 | 0.74 |
| 38_4M32_day5_Tob | 0.6 | 0.74 | 0.59 | 0.63 |
| 39_4M33_day5_Tob | 0.44 | 1.03 | 0.34 | 0.69 |
| 82_4M34_day28_Tob | 2.37 | 0.63 | 2.42 | 1.04 |
| 83_4M35_day28_Tob | 3.35 | 0.66 | 3.33 | 1.76 |
| 84_4M36_day28_Tob | 2.4 | 0.62 | 2.07 | 0.99 |
| 25_5F137_day1_CadCl | 0.38 | 1.25 | 0.41 | 0.6 |
| 26_5F138_day1_CadCl | 0.52 | 1.43 | 0.55 | 0.54 |
| 27_5F139_day1_CadCl | 0.36 | 1.27 | 0.3 | 0.5 |
| 54_5F141_day5_CadCl | 0.47 | 1.12 | 0.29 | 0.98 |
| 55_5F142_day5_CadCl | 0.29 | 0.9 | 0.27 | 0.49 |
| 100_5F143_day28_CadCl | 0.64 | 1.14 | 0.32 | 1.72 |
| 101_5F144_day28_CadCl | 0.58 | 0.88 | 0.42 | 1.15 |
| 102_5F145_day28_CadCl | 1.94 | 0.37 | 1.72 | 0.86 |
| 10_5M37_day1_CadCl | 0.38 | 1.09 | 0.28 | 0.66 |
| 11_5M38_day1_CadCl | 0.59 | 1.15 | 0.6 | 0.87 |
| 12_5M39_day1_CadCl | 0.64 | 1.05 | 0.56 | 0.84 |
| 40_5M40_day5_CadCl | 0.39 | 1.23 | 0.42 | 0.85 |
| 41_5M41_day5_CadCl | 0.5 | 0.98 | 0.41 | 0.81 |
| 42_5M42_day5_CadCl | 0.38 | 1.01 | 0.42 | 0.83 |
| 85_5M43_day28_CadCl | 0.4 | 0.66 | 0.32 | 0.75 |
| 86_5M44_day28_CadCl | 0.51 | 0.77 | 0.55 | 0.63 |
| 87_5M45_day28_CadCl | 0.82 | 0.83 | 0.72 | 0.71 |
| 28_6F146_day1_Dox | 0.78 | 1.1 | 0.62 | 0.61 |
| 29_6F147_day1_Dox | 0.4 | 1.52 | 0.46 | 0.64 |
| 30_6F148_day1_Dox | 0.26 | 1.52 | 0.33 | 3.12 |
| 56_6F149_day5_Dox | 0.51 | 0.91 | 0.5 | 0.76 |
| 57_6F150_day5_Dox | 0.48 | 0.84 | 0.46 | 0.93 |
| 58_6F151_day5_Dox | 0.38 | 1.24 | 0.4 | 0.57 |
| 103_6F171_day28_Dox | 1.5 | 1.24 | 1.04 | 0.66 |
| 104_6F172_day28_Dox | 0.62 | 1.39 | 0.55 | 0.82 |
| 105_6F173_day28_Dox | 0.04 | 1.29 | 1.3 | 1.6 |
| 13_6M46_day1_Dox | 0.59 | 1.26 | 0.54 | 1.12 |
| 14_6M47_day1_Dox | 0.49 | 0.92 | 0.55 | 1.21 |
| 15_6M48_day1_Dox | 0.39 | 0.99 | 0.38 | 0.78 |
| 43_6M49_day5_Dox | 1.2 | 0.84 | 1.14 | 0.93 |
| 44_6M50_day5_Dox | 0.6 | 1.22 | 0.48 | 2.05 |
| 88_6M71_day28_Dox | 6.37 | 0.45 | 5.73 | 1.49 |
| 89_6M72_day28_Dox | 0.92 | 1.06 | 0.96 | 0.9 |
| 90_6M73_day28_Dox | 1.28 | 0.92 | 1.43 | 0.87 |
| 115_7F155_day1_ValpA | 0.45 | 1.42 | 0.4 | 1.22 |
| 116_7F156_day1_ValpA | 0.42 | 1.1 | 0.31 | 0.01 |
| 117_7F157_day1_ValpA | 0.37 | 1.1 | 0.32 | 0.57 |
| 121_7F158_day5_ValpA | 0.36 | 1.33 | 0.38 | 0.79 |
| 122_7F159_day5_ValpA | 0.02 | 1.21 | 0.36 | 0.66 |
| 123_7F160_day5_ValpA | 0.53 | 1.37 | 0.28 | 0.66 |
| 127_7F161_day28_ValpA | 0.45 | 1.17 | 0.28 | 0.68 |
| 128_7F162_day28_ValpA | 0.58 | 1.55 | 0.32 | 1.12 |
| 129_7F163_day28_ValpA | 0.42 | 1.32 | 0.28 | 1.23 |
| 112_7M55_day1_ValpA | 0.4 | 0.92 | 0.39 | 0.88 |
| 113_7M56_day1_ValpA | 0.5 | 1.28 | 0.33 | 0.83 |
| 114_7M57_day1_ValpA | 0.48 | 0.84 | 0.42 | 0.72 |
| 118_7M58_day5_ValpA | 0.37 | 0.99 | 0.15 | 0.92 |
| 119_7M59_day5_ValpA | 0.36 | 0.86 | 0.25 | 0.47 |
| 120_7M60_day5_ValpA | 0.29 | 0.91 | 0.18 | 0.42 |
| 124_7M61_day28_ValpA | 0.21 | 1.05 | 0.15 | 0.62 |
| 125_7M62_day28_ValpA | 0.45 | 0.81 | 0.36 | 0.86 |
| 126_7M63_day28_ValpA | 0.4 | 1.23 | 0.25 | 0.59 |
| 62_8F181_day28_Naive | 0.47 | 1.59 | 0.39 | 0.55 |
| 63_8F182_day28_Naive | 0.6 | 1.23 | 0.37 | 0.51 |
| 64_8F183_day28_Naive | 0.42 | 0.84 | 0.38 | 0.52 |
| 59_8M81_day28_Naive | 0.74 | 1.46 | 0.68 | 0.7 |
| 60_8M82_day28_Naive | 0.46 | 1.26 | 0.3 | 0.33 |
| 61_8M83_day28_Naive | 0.45 | 1.18 | 0.34 | 0.53 |

| Sample | AI454051_DB71_seg0 | AW919147_DB71_seg0 | BI293562_DB71_seg2 |
|---|---|---|---|
| 68_1F101_day1_Saline | 0.96 | 0.9 | 0.61 |
| 69_1F102_day1_Saline | 0.99 | 0.96 | 0.67 |
| 70_1F103_day1_Saline | 0.88 | 1.03 | 0.68 |
| 74_1F104_day5_Saline | 1.08 | 0.92 | 0.2 |
| 75_1F105_day5_Saline | 0.93 | 0.9 | 0.41 |
| 109_1F107_day28_Saline | 1.3 | 1.09 | 0.29 |
| 110_1F108_day28_Saline | 1.33 | 1.12 | 0.37 |
| 111_1F109_day28_Saline | 0.7 | 0.84 | 0.16 |
| 65_1M1_day1_Saline | 0.91 | 0.9 | 1.45 |
| 66_1M2_day1_Saline | 0.11 | 0.73 | 0.42 |
| 67_1M3_day1_Saline | 1.13 | 1.05 | 0.7 |
| 71_1M4_day5_Saline | 1.01 | 0.86 | 0.83 |
| 72_1M5_day5_Saline | 0.73 | 1.17 | 0.4 |
| 73_1M6_day5_Saline | 0.49 | 0.92 | 0.33 |
| 106_1M7_day28_Saline | 0.86 | 1.3 | 0.63 |
| 107_1M8_day28_Saline | 0.88 | 1.28 | 0.32 |
| 108_1M9_day28_Saline | 1.16 | 1.7 | 0.87 |
| 16_2F110_day1_Gent | 0.9 | 1.72 | 0.59 |
| 17_2F111_day1_Gent | 0.75 | 0.72 | 0.94 |
| 18_2F112_day1_Gent | 0.57 | 0.76 | 1.04 |
| 45_2F113_day5_Gent | 0.63 | 0.86 | 0.95 |
| 46_2F114_day5_Gent | 1.04 | 0.88 | 0.76 |
| 47_2F115_day5_Gent | 1.19 | 0.95 | 2.8 |
| 91_2F116_day28_Gent | 0.67 | 0.73 | 2.01 |
| 92_2F117_day28_Gent | 1 | 1.12 | 1.19 |
| 93_2F118_day28_Gent | 0.95 | 0.89 | 0.81 |
| 1_2M10_day1_Gent | 0.4 | 0.72 | 0.93 |
| 2_2M11_day1_Gent | 0.54 | 1.1 | 0.55 |
| 3_2M12_day1_Gent | 0.79 | 0.99 | 0.41 |
| 31_2M13_day5_Gent | 0.8 | 0.71 | 2.32 |
| 32_2M14_day5_Gent | 0.63 | 0.79 | 1.61 |
| 33_2M15_day5_Gent | 0.82 | 0.78 | 0.93 |
| 76_2M16_day28_Gent | 1.14 | 0.75 | 0.63 |
| 77_2M17_day28_Gent | 0.64 | 0.57 | 0.72 |
| 78_2M18_day28_Gent | 0.71 | 0.93 | 1.16 |
| 19_3F119_day1_Cis | 0.94 | 0.8 | 0.74 |
| 20_3F120_day1_Cis | 1.22 | 1.09 | 0.5 |
| 21_3F121_day1_Cis | 1 | 0.87 | 0.38 |
| 48_3F122_day5_Cis | 1.05 | 1.39 | 0.74 |
| 49_3F123_day5_Cis | 1.43 | 1.05 | 0.67 |
| 50_3F124_day5_Cis | 1.39 | 0.83 | 0.7 |
| 94_3F125_day28_Cis | 0.68 | 1.08 | 5.98 |
| 95_3F126_day28_Cis | 0.8 | 0.54 | 12.27 |
| 96_3F127_day28_Cis | 0.87 | 0.72 | 8.01 |
| 4_3M19_day1_Cis | 0.65 | 0.87 | 0.7 |
| 5_3M20_day1_Cis | 0.96 | 1.21 | 0.39 |
| 6_3M21_day1_Cis | 1.45 | 0.88 | 0.71 |
| 34_3M22_day5_Cis | 0.92 | 1.08 | 3.45 |
| 35_3M23_day5_Cis | 1.16 | 0.8 | 0.81 |
| 36_3M24_day5_Cis | 0.86 | 0.88 | 1.76 |
| 79_3M25_day28_Cis | 0.73 | 0.66 | 6.24 |
| 80_3M26_day28_Cis | 0.82 | 0.84 | 2.93 |
| 81_3M27_day28_Cis | 0.78 | 0.54 | 3.33 |
| 22_4F128_day1_Tob | 1.16 | 0.8 | 0.29 |
| 23_4F129_day1_Tob | 1.36 | 1.06 | 0.42 |
| 24_4F130_day1_Tob | 0.8 | 0.98 | 0.73 |
| 51_4F131_day5_Tob | 1.26 | 0.88 | 0.71 |
| 52_4F132_day5_Tob | 1.23 | 0.99 | 1.62 |
| 53_4F133_day5_Tob | 1.32 | 1.05 | 0.84 |
| 97_4F134_day28_Tob | 0.94 | 0.8 | 1.32 |
| 98_4F135_day28_Tob | 1.29 | 0.86 | 1.02 |
| 99_4F136_day28_Tob | 0.72 | 0.72 | 0.35 |
| 7_4M28_day1_Tob | 0.86 | 0.82 | 0.58 |
| 8_4M29_day1_Tob | 1.29 | 1.33 | 1.38 |
| 9_4M30_day1_Tob | 1.17 | 1.22 | 1.04 |
| 37_4M31_day5_Tob | 0.61 | 0.58 | 0.3 |
| 38_4M32_day5_Tob | 0.57 | 0.63 | 0.44 |
| 39_4M33_day5_Tob | 0.63 | 1.14 | 0.59 |
| 82_4M34_day28_Tob | 1 | 0.76 | 0.84 |
| 83_4M35_day28_Tob | 0.94 | 1.23 | 1.78 |
| 84_4M36_day28_Tob | 1 | 0.63 | 1.07 |
| 25_5F137_day1_CadCl | 1.47 | 1 | 0.36 |
| 26_5F138_day1_CadCl | 1.46 | 1.21 | 0.4 |
| 27_5F139_day1_CadCl | 1.08 | 0.76 | 0.61 |
| 54_5F141_day5_CadCl | 1.54 | 1.06 | 0.43 |
| 55_5F142_day5_CadCl | 1.35 | 1.86 | 0.63 |
| 100_5F143_day28_CadCl | 0.79 | 0.77 | 0.4 |
| 101_5F144_day28_CadCl | 1.05 | 0.91 | 0.81 |
| 102_5F145_day28_CadCl | 0.54 | 0.42 | 2.59 |
| 10_5M37_day1_CadCl | 1.48 | 1 | 0.5 |
| 11_5M38_day1_CadCl | 2.13 | 1.1 | 0.51 |
| 12_5M39_day1_CadCl | 0.77 | 1.02 | 0.39 |
| 40_5M40_day5_CadCl | 0.84 | 1.3 | 0.48 |
| 41_5M41_day5_CadCl | 0.68 | 0.94 | 0.88 |
| 42_5M42_day5_CadCl | 0.92 | 1 | 0.78 |
| 85_5M43_day28_CadCl | 1.29 | 1.31 | 0.72 |
| 86_5M44_day28_CadCl | 1.31 | 1.18 | 1.35 |
| 87_5M45_day28_CadCl | 0.75 | 0.68 | 0.31 |
| 28_6F146_day1_Dox | 1.2 | 1.35 | 0.38 |
| 29_6F147_day1_Dox | 1.05 | 1.2 | 0.39 |
| 30_6F148_day1_Dox | 1.54 | 1.09 | 0.42 |
| 56_6F149_day5_Dox | 1.17 | 0.99 | 0.68 |
| 57_6F150_day5_Dox | 1.35 | 0.89 | 0.51 |
| 58_6F151_day5_Dox | 1.44 | 1.1 | 0.7 |
| 103_6F171_day28_Dox | 0.85 | 0.88 | 0.99 |
| 104_6F172_day28_Dox | 0.92 | 1.05 | 0.58 |
| 105_6F173_day28_Dox | 0.87 | 0.82 | 0 |
| 13_6M46_day1_Dox | 0.8 | 1.1 | 0.53 |
| 14_6M47_day1_Dox | 0.8 | 1.11 | 0.42 |
| 15_6M48_day1_Dox | 1.03 | 0.91 | 0.32 |
| 43_6M49_day5_Dox | 0.3 | 1.01 | 1.39 |
| 44_6M50_day5_Dox | 0.95 | 1.16 | 1.04 |
| 88_6M71_day28_Dox | 0.96 | 0.48 | 1.81 |
| 89_6M72_day28_Dox | 1.11 | 1.42 | 0.55 |
| 90_6M73_day28_Dox | 0.72 | 1.2 | 0.71 |
| 115_7F155_day1_ValpA | 1.56 | 1.43 | 0.45 |
| 116_7F156_day1_ValpA | 1.08 | 0.84 | 0.38 |
| 117_7F157_day1_ValpA | 1.17 | 1.84 | 0.4 |
| 121_7F158_day5_ValpA | 1.49 | 1.69 | 0.36 |
| 122_7F159_day5_ValpA | 1.52 | 1.83 | 0.39 |
| 123_7F160_day5_ValpA | 1.72 | 0.94 | 0.12 |
| 127_7F161_day28_ValpA | 1.16 | 0.92 | 0.18 |
| 128_7F162_day28_ValpA | 1.31 | 1.31 | 0.59 |
| 129_7F163_day28_ValpA | 1.32 | 1.06 | 0.5 |
| 112_7M55_day1_ValpA | 1.08 | 0.83 | 0.46 |
| 113_7M56_day1_ValpA | 0.97 | 0.96 | 0.52 |
| 114_7M57_day1_ValpA | 0.86 | 1.02 | 0.5 |
| 118_7M58_day5_ValpA | 1 | 0.97 | 0.59 |
| 119_7M59_day5_ValpA | 0.99 | 1.27 | 0.45 |
| 120_7M60_day5_ValpA | 0.81 | 0.79 | 0.62 |
| 124_7M61_day28_ValpA | 0.91 | 1.12 | 0.21 |
| 125_7M62_day28_ValpA | 1.16 | 1.16 | 0.31 |
| 126_7M63_day28_ValpA | 0.95 | 1.11 | 0.35 |
| 62_8F181_day28_Naive | 0.92 | 1.02 | 0.41 |
| 63_8F182_day28_Naive | 1.07 | 1.08 | 0.49 |
| 64_8F183_day28_Naive | 0.81 | 1.23 | 0.38 |
| 59_8M81_day28_Naive | 1.75 | 1.27 | 0.72 |
| 60_8M82_day28_Naive | 0.75 | 0.8 | 0.3 |
| 61_8M83_day28_Naive | 0.66 | 0.94 | 0.39 |

| Sample | H31045_DB71_seg5 | H31799_DB71_seg23 | H31883_DB71_seg13 | AI502869_DB71_seg0 |
|---|---|---|---|---|
| 68_1F101_day1_Saline | 0.16 | 1.05 | 0.88 | 0.98 |
| 69_1F102_day1_Saline | 1.03 | 0.96 | 0.36 | 0.68 |
| 70_1F103_day1_Saline | 0.24 | 1.06 | 0.75 | 0.73 |
| 74_1F104_day5_Saline | 0.15 | 1.16 | 0.71 | 0.99 |
| 75_1F105_day5_Saline | 0.67 | 0.7 | 0.59 | 0.67 |
| 109_1F107_day28_Saline | 0.81 | 1.21 | 0.83 | 1.25 |
| 110_1F108_day28_Saline | 1 | 1.13 | 0.89 | 2.1 |
| 111_1F109_day28_Saline | 2.96 | 1.06 | 0.83 | 1.32 |
| 65_1M1_day1_Saline | 0.93 | 1.12 | 0.68 | 0.62 |
| 66_1M2_day1_Saline | 0.69 | 0.87 | 0.69 | 0.6 |
| 67_1M3_day1_Saline | 0.19 | 1.06 | 0.78 | 0.62 |
| 71_1M4_day5_Saline | 0.28 | 0.98 | 0.63 | 0.82 |
| 72_1M5_day5_Saline | 0.18 | 1.11 | 0.56 | 0.86 |
| 73_1M6_day5_Saline | 0.25 | 0.88 | 0.68 | 0.52 |
| 106_1M7_day28_Saline | 0.42 | 0.82 | 0.88 | 1.24 |
| 107_1M8_day28_Saline | 2.19 | 1.13 | 0.73 | 1.14 |
| 108_1M9_day28_Saline | 0.36 | 1.06 | 0.84 | 1.31 |
| 16_2F110_day1_Gent | 0.27 | 1.19 | 0.99 | 0.83 |
| 17_2F111_day1_Gent | 0.22 | 0.88 | 0.92 | 0.61 |
| 18_2F112_day1_Gent | 0.73 | 0.93 | 0.82 | 0.53 |
| 45_2F113_day5_Gent | 0.52 | 0.92 | 0.73 | 0.73 |
| 46_2F114_day5_Gent | 1.01 | 0.91 | 0.55 | 0.62 |
| 47_2F115_day5_Gent | 0.4 | 1.36 | 0.91 | 0.96 |
| 91_2F116_day28_Gent | 0.79 | 0.88 | 0.88 | 0.66 |
| 92_2F117_day28_Gent | 0.84 | 1.05 | 0.98 | 0.74 |
| 93_2F118_day28_Gent | 0.67 | 1.17 | 0.96 | 0.73 |
| 1_2M10_day1_Gent | 0.16 | 1 | 0.82 | 1.53 |
| 2_2M11_day1_Gent | 0.19 | 1.04 | 0.61 | 0.99 |
| 3_2M12_day1_Gent | 0.15 | 1.2 | 0.73 | 0.76 |
| 31_2M13_day5_Gent | 1.57 | 1.08 | 1.01 | 0.51 |
| 32_2M14_day5_Gent | 1.47 | 0.76 | 0.7 | 0.43 |
| 33_2M15_day5_Gent | 0.33 | 0.88 | 0.65 | 0.53 |
| 76_2M16_day28_Gent | 1.11 | 1.17 | 1.05 | 1.36 |
| 77_2M17_day28_Gent | 4.65 | 0.37 | 0.64 | 0.92 |
| 78_2M18_day28_Gent | 0.45 | 1.06 | 0.8 | 0.77 |
| 19_3F119_day1_Cis | 0.24 | 1.03 | 2.03 | 0.67 |
| 20_3F120_day1_Cis | 0.28 | 1.09 | 1.06 | 0.78 |
| 21_3F121_day1_Cis | 0.22 | 1.04 | 1.07 | 0.74 |
| 48_3F122_day5_Cis | 1.8 | 1.1 | 1.17 | 0.8 |
| 49_3F123_day5_Cis | 0.34 | 0.74 | 1.44 | 0.93 |
| 50_3F124_day5_Cis | 0.51 | 1.23 | 1.2 | 0.74 |
| 94_3F125_day28_Cis | 0.73 | 1.05 | 2.72 | 1.03 |
| 95_3F126_day28_Cis | 0.73 | 1.15 | 4.07 | 1.03 |
| 96_3F127_day28_Cis | 0.81 | 0.98 | 3.83 | 1.25 |
| 4_3M19_day1_Cis | 0.82 | 1.22 | 0.74 | 0.87 |
| 5_3M20_day1_Cis | 0.21 | 1.27 | 0.71 | 0.87 |
| 6_3M21_day1_Cis | 1.22 | 1.12 | 1.52 | 0.84 |
| 34_3M22_day5_Cis | 3.25 | 0.92 | 1.73 | 0.52 |
| 35_3M23_day5_Cis | 0.23 | 1.05 | 1.3 | 1.13 |
| 36_3M24_day5_Cis | 1.66 | 1.06 | 2.43 | 0.61 |
| 79_3M25_day28_Cis | 0.88 | 0.88 | 2.41 | 0.74 |
| 80_3M26_day28_Cis | 2.92 | 1.21 | 3.37 | 1.35 |
| 81_3M27_day28_Cis | 2.96 | 0.9 | 2.23 | 0.95 |
| 22_4F128_day1_Tob | 0.2 | 0.91 | 0.87 | 0.52 |
| 23_4F129_day1_Tob | 0.18 | 0.87 | 0.68 | 1.32 |
| 24_4F130_day1_Tob | 0.74 | 1.2 | 0.87 | 0.79 |
| 51_4F131_day5_Tob | 1.19 | 1.1 | 0.82 | 0.81 |
| 52_4F132_day5_Tob | 0.42 | 0.92 | 0.69 | 0.74 |
| 53_4F133_day5_Tob | 0.38 | 0.83 | 0.52 | 0.74 |
| 97_4F134_day28_Tob | 0.76 | 1.03 | 0.84 | 1.08 |
| 98_4F135_day28_Tob | 1 | 0.71 | 0.63 | 2.38 |
| 99_4F136_day28_Tob | 0.63 | 1.15 | 0.84 | 1.04 |
| 7_4M28_day1_Tob | 0.2 | 1.09 | 0.83 | 0.87 |
| 8_4M29_day1_Tob | 1.03 | 1.14 | 0.69 | 1.02 |
| 9_4M30_day1_Tob | 0.22 | 1.17 | 0.61 | 1.28 |
| 37_4M31_day5_Tob | 0.9 | 0.71 | 0.48 | 0.42 |
| 38_4M32_day5_Tob | 0.29 | 0.72 | 0.47 | 0.5 |
| 39_4M33_day5_Tob | 1.65 | 0.96 | 0.82 | 0.72 |
| 82_4M34_day28_Tob | 2.08 | 0.94 | 0.6 | 1.22 |
| 83_4M35_day28_Tob | 2.68 | 0.88 | 0.73 | 0.99 |
| 84_4M36_day28_Tob | 2.92 | 1.12 | 0.73 | 1.22 |
| 25_5F137_day1_CadCl | 1.97 | 1 | 0.73 | 0.54 |
| 26_5F138_day1_CadCl | 0.39 | 1.24 | 0.63 | 1.37 |
| 27_5F139_day1_CadCl | 0.39 | 1 | 0.87 | 0.56 |
| 54_5F141_day5_CadCl | 0.56 | 0.83 | 0.87 | 0.94 |
| 55_5F142_day5_CadCl | 0.32 | 0.99 | 0.86 | 0.73 |
| 100_5F143_day28_CadCl | 2.88 | 1.59 | 0.97 | 0.73 |
| 101_5F144_day28_CadCl | 0.83 | 0.94 | 1.02 | 1.22 |
| 102_5F145_day28_CadCl | 0.57 | 0.67 | 0.86 | 0.44 |
| 10_5M37_day1_CadCl | 0.19 | 0.91 | 0.65 | 1.14 |
| 11_5M38_day1_CadCl | 0.19 | 0.9 | 0.71 | 2.59 |
| 12_5M39_day1_CadCl | 0.18 | 1.15 | 0.78 | 0.86 |
| 40_5M40_day5_CadCl | 0.42 | 1.17 | 0.83 | 0.69 |
| 41_5M41_day5_CadCl | 0.4 | 0.98 | 0.78 | 0.68 |
| 42_5M42_day5_CadCl | 0.46 | 0.94 | 0.64 | 0.85 |
| 85_5M43_day28_CadCl | 2.23 | 1.05 | 0.95 | 2.47 |
| 86_5M44_day28_CadCl | 2.41 | 1.02 | 0.8 | 2.93 |
| 87_5M45_day28_CadCl | 2 | 0.76 | 0.86 | 1.18 |
| 28_6F146_day1_Dox | 1.15 | 1.04 | 0.93 | 0.71 |
| 29_6F147_day1_Dox | 0.3 | 0.99 | 0.89 | 0.81 |
| 30_6F148_day1_Dox | 1.95 | 0.87 | 0.67 | 0.67 |
| 56_6F149_day5_Dox | 1.86 | 0.95 | 1.16 | 0.51 |
| 57_6F150_day5_Dox | 1.19 | 1.08 | 1.03 | 1 |
| 58_6F151_day5_Dox | 0.52 | 0.66 | 0.9 | 1.07 |
| 103_6F171_day28_Dox | 2.16 | 1.16 | 1.48 | 1.33 |
| 104_6F172_day28_Dox | 2.58 | 1.25 | 1.77 | 1.48 |
| 105_6F173_day28_Dox | 0.51 | 1.57 | 2.29 | 0.98 |
| 13_6M46_day1_Dox | 0.23 | 1.07 | 1.02 | 0.77 |
| 14_6M47_day1_Dox | 0.32 | 1.26 | 1.07 | 0.68 |
| 15_6M48_day1_Dox | 0.75 | 0.92 | 0.9 | 0.78 |
| 43_6M49_day5_Dox | 0.35 | 0.56 | 1.21 | 0.73 |
| 44_6M50_day5_Dox | 1.41 | 0.9 | 1.38 | 0.71 |
| 88_6M71_day28_Dox | 1.57 | 0.64 | 1.45 | 0.42 |
| 89_6M72_day28_Dox | 2.33 | 1.11 | 1.59 | 1.49 |
| 90_6M73_day28_Dox | 2.12 | 1.06 | 1.93 | 1.28 |
| 115_7F155_day1_ValpA | 0.68 | 1.29 | 1.02 | 0.86 |
| 116_7F156_day1_ValpA | 1.71 | 0.9 | 0.01 | 1.09 |
| 117_7F157_day1_ValpA | 0.53 | 0 | 0.66 | 1.13 |
| 121_7F158_day5_ValpA | 0.56 | 0.94 | 1.54 | 1.81 |
| 122_7F159_day5_ValpA | 1 | 1.19 | 1.1 | 2.53 |
| 123_7F160_day5_ValpA | 0.57 | 1.15 | 1.05 | 1.94 |
| 127_7F161_day28_ValpA | 4.35 | 0.98 | 0.92 | 1.24 |
| 128_7F162_day28_ValpA | 0.58 | 0.67 | 0.9 | 1.19 |
| 129_7F163_day28_ValpA | 1.41 | 0.99 | 0.81 | 1.18 |
| 112_7M55_day1_ValpA | 3.33 | 1.11 | 0.75 | 1.33 |
| 113_7M56_day1_ValpA | 2.68 | 1.23 | 0.9 | 1 |
| 114_7M57_day1_ValpA | 0.73 | 1.16 | 0.95 | 1.43 |
| 118_7M58_day5_ValpA | 2.42 | 0.51 | 1 | 1.37 |
| 119_7M59_day5_ValpA | 0.83 | 1.06 | 0.48 | 1.06 |
| 120_7M60_day5_ValpA | 2.53 | 1.02 | 0.63 | 1.29 |
| 124_7M61_day28_ValpA | 1.58 | 0.98 | 0.71 | 0.97 |
| 125_7M62_day28_ValpA | 0.2 | 1.03 | 1.11 | 1.46 |
| 126_7M63_day28_ValpA | 0.47 | 0.75 | 0.72 | 1.43 |
| 62_8F181_day28_Naive | 1.21 | 1.05 | 0.79 | 1.24 |
| 63_8F182_day28_Naive | 0.25 | 0.94 | 0.73 | 0.78 |
| 64_8F183_day28_Naive | 0.25 | 1 | 0.66 | 0.77 |
| 59_8M81_day28_Naive | 0.54 | 1.26 | 0 | 1 |
| 60_8M82_day28_Naive | 0.26 | 1.05 | 0.58 | 0.79 |
| 61_8M83_day28_Naive | 0.29 | 0.82 | 0.68 | 0.64 |

| Sample | H33998_DB71_seg19 | RATLRFI_DB71_seg9 | RATRAB13X_DB71_seg11-13 |
|---|---|---|---|
| 68_1F101_day1_Saline | 0.73 | 0.37 | 0.97 |
| 69_1F102_day1_Saline | 1.01 | 0.28 | 0.78 |
| 70_1F103_day1_Saline | 0.88 | 0.09 | 0.98 |
| 74_1F104_day5_Saline | 0.93 | 0.36 | 0.97 |
| 75_1F105_day5_Saline | 1.1 | 0.49 | 0.94 |
| 109_1F107_day28_Saline | 0.41 | 0.44 | 1 |
| 110_1F108_day28_Saline | 0.59 | 0.37 | 1.11 |
| 111_1F109_day28_Saline | 0.57 | 0.58 | 0.92 |
| 65_1M1_day1_Saline | 0.51 | 0.01 | 0.83 |
| 66_1M2_day1_Saline | 0.98 | 0.32 | 1.06 |
| 67_1M3_day1_Saline | 0.51 | 0.36 | 0.87 |
| 71_1M4_day5_Saline | 1.26 | 0.21 | 0.76 |
| 72_1M5_day5_Saline | 1.13 | 0.25 | 0.76 |
| 73_1M6_day5_Saline | 0.98 | 0.22 | 0.64 |
| 106_1M7_day28_Saline | 0.6 | 0.32 | 0.82 |
| 107_1M8_day28_Saline | 0.36 | 0.12 | 0.77 |
| 108_1M9_day28_Saline | 0.73 | 0.43 | 0.91 |
| 16_2F110_day1_Gent | 1.31 | 0.98 | 1.16 |
| 17_2F111_day1_Gent | 0.83 | 1.54 | 0.7 |
| 18_2F112_day1_Gent | 0.56 | 1.09 | 0.77 |
| 45_2F113_day5_Gent | 1.9 | 0.48 | 0.9 |
| 46_2F114_day5_Gent | 1.5 | 0.43 | 0.82 |
| 47_2F115_day5_Gent | 2.19 | 4.15 | 1.12 |
| 91_2F116_day28_Gent | 0.4 | 5.63 | 0.83 |
| 92_2F117_day28_Gent | 0.7 | 4.17 | 1.12 |
| 93_2F118_day28_Gent | 0.56 | 2.43 | 0.88 |
| 1_2M10_day1_Gent | 1.51 | 2.74 | 2.79 |
| 2_2M11_day1_Gent | 1.35 | 1.72 | 1.14 |
| 3_2M12_day1_Gent | 0.99 | 0.27 | 1.03 |
| 31_2M13_day5_Gent | 1.91 | 1.35 | 0.9 |
| 32_2M14_day5_Gent | 0.83 | 1.13 | 0.61 |
| 33_2M15_day5_Gent | 1.16 | 0.59 | 0.8 |
| 76_2M16_day28_Gent | 0.4 | 2.56 | 1.17 |
| 77_2M17_day28_Gent | 0.49 | 1.59 | 0.76 |
| 78_2M18_day28_Gent | 0.42 | 2.51 | 1.22 |
| 19_3F119_day1_Cis | 0.85 | 1.49 | 0.91 |
| 20_3F120_day1_Cis | 1 | 0.3 | 1.12 |
| 21_3F121_day1_Cis | 0.87 | 0.57 | 1.09 |
| 48_3F122_day5_Cis | 1.79 | 0.79 | 1.06 |
| 49_3F123_day5_Cis | 1.84 | 0.44 | 1.01 |
| 50_3F124_day5_Cis | 1.95 | 0.32 | 1.02 |
| 94_3F125_day28_Cis | 0.55 | 2.33 | 1.29 |
| 95_3F126_day28_Cis | 0.69 | 3.05 | 1.62 |
| 96_3F127_day28_Cis | 0.85 | 3.8 | 1.78 |
| 4_3M19_day1_Cis | 0.65 | 0.24 | 0.87 |
| 5_3M20_day1_Cis | 0.75 | 0.2 | 1.15 |
| 6_3M21_day1_Cis | 1.14 | 0.37 | 1.16 |
| 34_3M22_day5_Cis | 1.49 | 0.65 | 1.13 |
| 35_3M23_day5_Cis | 0.46 | 0.34 | 0.74 |
| 36_3M24_day5_Cis | 2.14 | 1.97 | 1.27 |
| 79_3M25_day28_Cis | 0.61 | 3.03 | 1.57 |
| 80_3M26_day28_Cis | 0.62 | 2.4 | 1.24 |
| 81_3M27_day28_Cis | 0.41 | 2.26 | 1.31 |
| 22_4F128_day1_Tob | 0.75 | 1.03 | 1.11 |
| 23_4F129_day1_Tob | 0.76 | 0.68 | 1.06 |
| 24_4F130_day1_Tob | 0.77 | 3.08 | 1.22 |
| 51_4F131_day5_Tob | 1.65 | 0.75 | 1.21 |
| 52_4F132_day5_Tob | 1.88 | 3.08 | 0.98 |
| 53_4F133_day5_Tob | 1.61 | 0.79 | 0.87 |
| 97_4F134_day28_Tob | 0.99 | 1.94 | 1.25 |
| 98_4F135_day28_Tob | 0.44 | 2.39 | 0.9 |
| 99_4F136_day28_Tob | 0.74 | 1.98 | 1.29 |
| 7_4M28_day1_Tob | 0.7 | 0.22 | 1.12 |
| 8_4M29_day1_Tob | 0.98 | 1.63 | 1.01 |
| 9_4M30_day1_Tob | 0.71 | 3.36 | 1.08 |
| 37_4M31_day5_Tob | 1.1 | 0.88 | 0.59 |
| 38_4M32_day5_Tob | 0.88 | 0.72 | 0.71 |
| 39_4M33_day5_Tob | 1.3 | 0.17 | 0.81 |
| 82_4M34_day28_Tob | 0.61 | 2.01 | 1.12 |
| 83_4M35_day28_Tob | 0.54 | 3.78 | 1.34 |
| 84_4M36_day28_Tob | 0.69 | 2.37 | 1.31 |
| 25_5F137_day1_CadCl | 2.29 | 0.27 | 0.95 |
| 26_5F138_day1_CadCl | 2.51 | 0.33 | 1.2 |
| 27_5F139_day1_CadCl | 0.01 | 0.32 | 0.95 |
| 54_5F141_day5_CadCl | 2 | 0.35 | 0.95 |
| 55_5F142_day5_CadCl | 0.82 | 0.31 | 0.9 |
| 100_5F143_day28_CadCl | 1.26 | 0.66 | 1.37 |
| 101_5F144_day28_CadCl | 0.59 | 0.85 | 0.92 |
| 102_5F145_day28_CadCl | 0.43 | 1.78 | 0.96 |
| 10_5M37_day1_CadCl | 0.76 | 0.27 | 1.05 |
| 11_5M38_day1_CadCl | 0.66 | 0.36 | 1.22 |
| 12_5M39_day1_CadCl | 0.69 | 0.23 | 1.14 |
| 40_5M40_day5_CadCl | 1.39 | 0.17 | 0.75 |
| 41_5M41_day5_CadCl | 1.36 | 0.18 | 0.75 |
| 42_5M42_day5_CadCl | 1.64 | 0.29 | 0.62 |
| 85_5M43_day28_CadCl | 0.45 | 0.28 | 1.11 |
| 86_5M44_day28_CadCl | 0.26 | 0.67 | 1.08 |
| 87_5M45_day28_CadCl | 0.41 | 0.56 | 0.88 |
| 28_6F146_day1_Dox | 1.25 | 0.25 | 1.1 |
| 29_6F147_day1_Dox | 1.34 | 0.21 | 0.9 |
| 30_6F148_day1_Dox | 2.14 | 0.31 | 1 |
| 56_6F149_day5_Dox | 2.39 | 0.46 | 1.17 |
| 57_6F150_day5_Dox | 1.29 | 0.29 | 1.16 |
| 58_6F151_day5_Dox | 3.68 | 0.18 | 0.94 |
| 103_6F171_day28_Dox | 0.48 | 0.72 | 0.74 |
| 104_6F172_day28_Dox | 0.58 | 0.4 | 0.95 |
| 105_6F173_day28_Dox | 0.96 | 1.47 | 1.05 |
| 13_6M46_day1_Dox | 0.97 | 0.46 | 0.88 |
| 14_6M47_day1_Dox | 1.07 | 0.48 | 1.09 |
| 15_6M48_day1_Dox | 2.75 | 0.24 | 0.86 |
| 43_6M49_day5_Dox | 1.19 | 0.47 | 0.7 |
| 44_6M50_day5_Dox | 1.37 | 0.25 | 0.83 |
| 88_6M71_day28_Dox | 0.64 | 8.34 | 0.91 |
| 89_6M72_day28_Dox | 0.44 | 0.53 | 0.77 |
| 90_6M73_day28_Dox | 0.54 | 1.08 | 0.84 |
| 115_7F155_day1_ValpA | 1.48 | 0.55 | 1.32 |
| 116_7F156_day1_ValpA | 0.82 | 0.32 | 0.96 |
| 117_7F157_day1_ValpA | 1.15 | 0.45 | 1.01 |
| 121_7F158_day5_ValpA | 0.76 | 0.39 | 0.79 |
| 122_7F159_day5_ValpA | 0.88 | 0.41 | 1.26 |
| 123_7F160_day5_ValpA | 1.19 | 0.39 | 1.41 |
| 127_7F161_day28_ValpA | 0.71 | 0.36 | 0.91 |
| 128_7F162_day28_ValpA | 0.56 | 0.54 | 0.91 |
| 129_7F163_day28_ValpA | 0.58 | 0.53 | 0.96 |
| 112_7M55_day1_ValpA | 0.37 | 0.39 | 0.79 |
| 113_7M56_day1_ValpA | 0.77 | 0.36 | 0.93 |
| 114_7M57_day1_ValpA | 0.53 | 0.44 | 0.83 |
| 118_7M58_day5_ValpA | 1.36 | 0.22 | 0.67 |
| 119_7M59_day5_ValpA | 1.11 | 0.21 | 0.8 |
| 120_7M60_day5_ValpA | 1.03 | 0.3 | 0.65 |
| 124_7M61_day28_ValpA | 0.69 | 0.23 | 0.88 |
| 125_7M62_day28_ValpA | 0.78 | 0.48 | 0.99 |
| 126_7M63_day28_ValpA | 0.52 | 0.33 | 0.69 |
| 62_8F181_day28_Naive | 0.69 | 0.39 | 1.07 |
| 63_8F182_day28_Naive | 1.23 | 0.45 | 1.07 |
| 64_8F183_day28_Naive | 0.85 | 0.34 | 0.79 |
| 59_8M81_day28_Naive | 1.25 | 0.42 | 1.08 |
| 60_8M82_day28_Naive | 0.99 | 0.25 | 0.66 |
| 61_8M83_day28_Naive | 0 | 0.26 | 0.77 |

| Sample | RNU24174_DB71_seg8 | W41270_DB81_seg11 |
|---|---|---|
| 68_1F101_day1_Saline | 0.57 | 0.6 |
| 69_1F102_day1_Saline | 0.41 | 0.86 |
| 70_1F103_day1_Saline | 0.32 | 1.31 |
| 74_1F104_day5_Saline | 0.26 | 0.59 |
| 75_1F105_day5_Saline | 0.28 | 0.83 |
| 109_1F107_day28_Saline | 0.33 | 0.36 |
| 110_1F108_day28_Saline | 0.24 | 0.41 |
| 111_1F109_day28_Saline | 0.33 | 0.39 |
| 65_1M1_day1_Saline | 0.24 | 0.57 |
| 66_1M2_day1_Saline | 0.15 | 0.99 |
| 67_1M3_day1_Saline | 0.29 | 0.54 |
| 71_1M4_day5_Saline | 0.15 | 0.68 |
| 72_1M5_day5_Saline | 0.33 | 1 |
| 73_1M6_day5_Saline | 0.12 | 0.46 |
| 106_1M7_day28_Saline | 0.24 | 0.33 |
| 107_1M8_day28_Saline | 0.15 | 0.26 |
| 108_1M9_day28_Saline | 0.27 | 0.33 |
| 16_2F110_day1_Gent | 0.47 | 1.24 |
| 17_2F111_day1_Gent | 0.5 | 0.67 |
| 18_2F112_day1_Gent | 0.47 | 1.19 |
| 45_2F113_day5_Gent | 0.31 | 2.04 |
| 46_2F114_day5_Gent | 0.22 | 1.34 |
| 47_2F115_day5_Gent | 0.81 | 2.42 |
| 91_2F116_day28_Gent | 1.53 | 0.94 |
| 92_2F117_day28_Gent | 1.2 | 0.81 |
| 93_2F118_day28_Gent | 0.59 | 0.81 |
| 1_2M10_day1_Gent | 0.74 | 0.46 |
| 2_2M11_day1_Gent | 0.37 | 0.68 |
| 3_2M12_day1_Gent | 0.26 | 0.57 |
| 31_2M13_day5_Gent | 0.65 | 2.03 |
| 32_2M14_day5_Gent | 0.36 | 1.08 |
| 33_2M15_day5_Gent | 0.4 | 1.2 |
| 76_2M16_day28_Gent | 1.91 | 1.42 |
| 77_2M17_day28_Gent | 0.69 | 0.75 |
| 78_2M18_day28_Gent | 0.65 | 0.84 |
| 19_3F119_day1_Cis | 0.57 | 1.11 |
| 20_3F120_day1_Cis | 0.32 | 0.86 |
| 21_3F121_day1_Cis | 0.36 | 0.76 |
| 48_3F122_day5_Cis | 0.97 | 1.97 |
| 49_3F123_day5_Cis | 0.7 | 1.9 |
| 50_3F124_day5_Cis | 0.6 | 1.79 |
| 94_3F125_day28_Cis | 8.38 | 1.45 |
| 95_3F126_day28_Cis | 12.63 | 1.8 |
| 96_3F127_day28_Cis | 12.14 | 1.48 |
| 4_3M19_day1_Cis | 0.2 | 0.33 |
| 5_3M20_day1_Cis | 0.27 | 0.63 |
| 6_3M21_day1_Cis | 0.69 | 0.71 |
| 34_3M22_day5_Cis | 1.3 | 1.51 |
| 35_3M23_day5_Cis | 0.31 | 1.07 |
| 36_3M24_day5_Cis | 3.6 | 3.12 |
| 79_3M25_day28_Cis | 10.21 | 1.94 |
| 80_3M26_day28_Cis | 7.3 | 1.11 |
| 81_3M27_day28_Cis | 8.08 | 1.45 |
| 22_4F128_day1_Tob | 0.29 | 1.01 |
| 23_4F129_day1_Tob | 0.32 | 0.44 |
| 24_4F130_day1_Tob | 0.53 | 0.83 |
| 51_4F131_day5_Tob | 0.46 | 2.6 |
| 52_4F132_day5_Tob | 0.76 | 3.25 |
| 53_4F133_day5_Tob | 0.47 | 2.67 |
| 97_4F134_day28_Tob | 0.55 | 1.01 |
| 98_4F135_day28_Tob | 0.65 | 0.73 |
| 99_4F136_day28_Tob | 0.69 | 0.9 |
| 7_4M28_day1_Tob | 0.29 | 0.57 |
| 8_4M29_day1_Tob | 0.49 | 0.79 |
| 9_4M30_day1_Tob | 0.51 | 0.76 |
| 37_4M31_day5_Tob | 0.09 | 0.62 |
| 38_4M32_day5_Tob | 0.2 | 0.81 |
| 39_4M33_day5_Tob | 0.14 | 1.21 |
| 82_4M34_day28_Tob | 0.54 | 0.92 |
| 83_4M35_day28_Tob | 0.93 | 1.03 |
| 84_4M36_day28_Tob | 0.62 | 0.79 |
| 25_5F137_day1_CadCl | 0.3 | 1.44 |
| 26_5F138_day1_CadCl | 0.29 | 1.78 |
| 27_5F139_day1_CadCl | 0.49 | 1.48 |
| 54_5F141_day5_CadCl | 0.33 | 2.49 |
| 55_5F142_day5_CadCl | 0.27 | 1.17 |
| 100_5F143_day28_CadCl | 0.21 | 0.86 |
| 101_5F144_day28_CadCl | 0.45 | 0.55 |
| 102_5F145_day28_CadCl | 1.21 | 0.83 |
| 10_5M37_day1_CadCl | 0.2 | 0.5 |
| 11_5M38_day1_CadCl | 0.4 | 0.62 |
| 12_5M39_day1_CadCl | 0.22 | 0.43 |
| 40_5M40_day5_CadCl | 0.12 | 1.02 |
| 41_5M41_day5_CadCl | 0.13 | 1.43 |
| 42_5M42_day5_CadCl | 0.12 | 1.52 |
| 85_5M43_day28_CadCl | 0.25 | 0.33 |
| 86_5M44_day28_CadCl | 0.39 | 0.27 |
| 87_5M45_day28_CadCl | 0.25 | 0.39 |
| 28_6F146_day1_Dox | 0.29 | 2.24 |
| 29_6F147_day1_Dox | 0.35 | 1.2 |
| 30_6F148_day1_Dox | 0.31 | 1.56 |
| 56_6F149_day5_Dox | 0.83 | 1.71 |
| 57_6F150_day5_Dox | 0.59 | 1.37 |
| 58_6F151_day5_Dox | 0.63 | 2.13 |
| 103_6F171_day28_Dox | 1.99 | 0.84 |
| 104_6F172_day28_Dox | 2.06 | 0.44 |
| 105_6F173_day28_Dox | 4.39 | 0.77 |
| 13_6M46_day1_Dox | 0.48 | 1.74 |
| 14_6M47_day1_Dox | 0.49 | 0.95 |
| 15_6M48_day1_Dox | 0.36 | 0.91 |
| 43_6M49_day5_Dox | 1.08 | 1.64 |
| 44_6M50_day5_Dox | 0.87 | 1.35 |
| 88_6M71_day28_Dox | 3.5 | 1.64 |
| 89_6M72_day28_Dox | 2.2 | 0.54 |
| 90_6M73_day28_Dox | 2.91 | 0.5 |
| 115_7F155_day1_ValpA | 0.45 | 0.87 |
| 116_7F156_day1_ValpA | 0.28 | 0.42 |
| 117_7F157_day1_ValpA | 0.28 | 0.64 |
| 121_7F158_day5_ValpA | 0.35 | 0.54 |
| 122_7F159_day5_ValpA | 0.51 | 0.55 |
| 123_7F160_day5_ValpA | 0.36 | 0.37 |
| 127_7F161_day28_ValpA | 0.23 | 0.65 |
| 128_7F162_day28_ValpA | 0.28 | 0.3 |
| 129_7F163_day28_ValpA | 0.29 | 0.4 |
| 112_7M55_day1_ValpA | 0.16 | 0.57 |
| 113_7M56_day1_ValpA | 0.19 | 0.49 |
| 114_7M57_day1_ValpA | 0.25 | 0.57 |
| 118_7M58_day5_ValpA | 0.2 | 0.7 |
| 119_7M59_day5_ValpA | 0.14 | 0.86 |
| 120_7M60_day5_ValpA | 0.17 | 0.62 |
| 124_7M61_day28_ValpA | 0.09 | 0.39 |
| 125_7M62_day28_ValpA | 0.19 | 0.42 |
| 126_7M63_day28_ValpA | 0.09 | 0.36 |
| 62_8F181_day28_Naive | 0.26 | 0.62 |
| 63_8F182_day28_Naive | 0.36 | 0.01 |
| 64_8F183_day28_Naive | 0.37 | 0.83 |
| 59_8M81_day28_Naive | 0.2 | 1.24 |
| 60_8M82_day28_Naive | 0.15 | 0.59 |
| 61_8M83_day28_Naive | 0.24 | 0.37 |

| Sample | W64472_DB81_seg2 | W83813_DB81_seg27 | MUSCYCG1R_DB81_seg15-17 |
|---|---|---|---|
| 68_1F101_day1_Saline | 0.45 | 0.94 | 0.67 |
| 69_1F102_day1_Saline | 0.35 | 0.77 | 0.94 |
| 70_1F103_day1_Saline | 0.43 | 1.03 | 0.6 |
| 74_1F104_day5_Saline | 0.35 | 1.03 | 0.76 |
| 75_1F105_day5_Saline | 0.56 | 0.99 | 0.75 |
| 109_1F107_day28_Saline | 0.24 | 0.48 | 0.57 |
| 110_1F108_day28_Saline | 0.38 | 0.66 | 0.76 |
| 111_1F109_day28_Saline | 0.61 | 0.67 | 0.7 |
| 65_1M1_day1_Saline | 0.65 | 0.68 | 0.47 |
| 66_1M2_day1_Saline | 0.56 | 0.87 | 0.41 |
| 67_1M3_day1_Saline | 1.02 | 0.94 | 0.4 |
| 71_1M4_day5_Saline | 1.02 | 1.29 | 0.86 |
| 72_1M5_day5_Saline | 0.75 | 1.2 | 0.92 |
| 73_1M6_day5_Saline | 0.62 | 1.07 | 0.54 |
| 106_1M7_day28_Saline | 0.4 | 0.5 | 0.41 |
| 107_1M8_day28_Saline | 0.23 | 0.38 | 0.45 |
| 108_1M9_day28_Saline | 0.53 | 0.43 | 0.43 |
| 16_2F110_day1_Gent | 0.63 | 0.84 | 0.63 |
| 17_2F111_day1_Gent | 0.45 | 0.79 | 0.53 |
| 18_2F112_day1_Gent | 0.61 | 0.78 | 0.66 |
| 45_2F113_day5_Gent | 1.5 | 2.07 | 1.51 |
| 46_2F114_day5_Gent | 1.2 | 1.82 | 1.61 |
| 47_2F115_day5_Gent | 0.94 | 1.7 | 1.51 |
| 91_2F116_day28_Gent | 0.92 | 0.45 | 0.69 |
| 92_2F117_day28_Gent | 0.66 | 0.34 | 0.62 |
| 93_2F118_day28_Gent | 0.54 | 0.62 | 0.57 |
| 1_2M10_day1_Gent | 0.4 | 0.53 | 0.5 |
| 2_2M11_day1_Gent | 0.54 | 0.58 | 0.49 |
| 3_2M12_day1_Gent | 0.54 | 0.71 | 0.41 |
| 31_2M13_day5_Gent | 1.95 | 1.35 | 0.95 |
| 32_2M14_day5_Gent | 1.62 | 0.88 | 0.79 |
| 33_2M15_day5_Gent | 1.61 | 1.4 | 1.02 |
| 76_2M16_day28_Gent | 1.49 | 0.78 | 0.65 |
| 77_2M17_day28_Gent | 0.84 | 0.54 | 0.56 |
| 78_2M18_day28_Gent | 0.81 | 0.38 | 0.54 |
| 19_3F119_day1_Cis | 0.8 | 1.1 | 0.77 |
| 20_3F120_day1_Cis | 0.54 | 0.97 | 0.85 |
| 21_3F121_day1_Cis | 0.55 | 0.99 | 0.72 |
| 48_3F122_day5_Cis | 2.23 | 2.12 | 3.12 |
| 49_3F123_day5_Cis | 2.1 | 2.25 | 3.59 |
| 50_3F124_day5_Cis | 1.99 | 2.12 | 2.07 |
| 94_3F125_day28_Cis | 1.81 | 0.71 | 1.96 |
| 95_3F126_day28_Cis | 3.16 | 0.7 | 2.57 |
| 96_3F127_day28_Cis | 2.87 | 0.78 | 2.7 |
| 4_3M19_day1_Cis | 0.3 | 0.5 | 0.54 |
| 5_3M20_day1_Cis | 0.51 | 0.66 | 0.35 |
| 6_3M21_day1_Cis | 0.92 | 0.94 | 0.67 |
| 34_3M22_day5_Cis | 2.64 | 1.39 | 2.31 |
| 35_3M23_day5_Cis | 0.95 | 0.94 | 0.92 |
| 36_3M24_day5_Cis | 4.55 | 1.41 | 2.34 |
| 79_3M25_day28_Cis | 2.8 | 0.51 | 2.09 |
| 80_3M26_day28_Cis | 3.04 | 0.51 | 2.31 |
| 81_3M27_day28_Cis | 1.65 | 0.57 | 1.93 |
| 22_4F128_day1_Tob | 0.35 | 0.86 | 0.72 |
| 23_4F129_day1_Tob | 0.26 | 0.61 | 0.46 |
| 24_4F130_day1_Tob | 0.49 | 1 | 0.61 |
| 51_4F131_day5_Tob | 1.4 | 2.61 | 1.95 |
| 52_4F132_day5_Tob | 1.55 | 1.93 | 1.44 |
| 53_4F133_day5_Tob | 1.27 | 1.76 | 1.15 |
| 97_4F134_day28_Tob | 0.48 | 0.75 | 0.77 |
| 98_4F135_day28_Tob | 0.43 | 0.49 | 0.59 |
| 99_4F136_day28_Tob | 0.62 | 0.63 | 0.73 |
| 7_4M28_day1_Tob | 0.45 | 0.65 | 0.43 |
| 8_4M29_day1_Tob | 0.66 | 0.73 | 0.49 |
| 9_4M30_day1_Tob | 0.44 | 0.48 | 0.47 |
| 37_4M31_day5_Tob | 0.93 | 0.7 | 0.4 |
| 38_4M32_day5_Tob | 0.96 | 1.13 | 0.69 |
| 39_4M33_day5_Tob | 1.3 | 1.4 | 1.07 |
| 82_4M34_day28_Tob | 0.44 | 0.48 | 0.72 |
| 83_4M35_day28_Tob | 0.85 | 0.56 | 0.59 |
| 84_4M36_day28_Tob | 0.79 | 0.55 | 0.71 |
| 25_5F137_day1_CadCl | 1.07 | 2.07 | 1.91 |
| 26_5F138_day1_CadCl | 1.07 | 2.19 | 1.36 |
| 27_5F139_day1_CadCl | 1.1 | 1.66 | 1.68 |
| 54_5F141_day5_CadCl | 1.65 | 3.79 | 1.72 |
| 55_5F142_day5_CadCl | 0.78 | 1.59 | 1.13 |
| 100_5F143_day28_CadCl | 0.89 | 0.95 | 0.88 |
| 101_5F144_day28_CadCl | 0.55 | 0.64 | 0.71 |
| 102_5F145_day28_CadCl | 0.43 | 0.46 | 0.65 |
| 10_5M37_day1_CadCl | 0.55 | 0.47 | 0.61 |
| 11_5M38_day1_CadCl | 1.45 | 0.51 | 0.73 |
| 12_5M39_day1_CadCl | 0.41 | 0.65 | 0.36 |
| 40_5M40_day5_CadCl | 2.23 | 1.48 | 1.2 |
| 41_5M41_day5_CadCl | 1.89 | 1.67 | 0.97 |
| 42_5M42_day5_CadCl | 1.63 | 1.76 | 1.15 |
| 85_5M43_day28_CadCl | 0.67 | 0.46 | 0.72 |
| 86_5M44_day28_CadCl | 0.47 | 0.34 | 0.44 |
| 87_5M45_day28_CadCl | 0.57 | 0.53 | 0.58 |
| 28_6F146_day1_Dox | 1.58 | 2.9 | 1.66 |
| 29_6F147_day1_Dox | 1.09 | 1.56 | 1.12 |
| 30_6F148_day1_Dox | 1.15 | 2 | 1.36 |
| 56_6F149_day5_Dox | 1.97 | 2.28 | 1.51 |
| 57_6F150_day5_Dox | 1.36 | 1.99 | 1.97 |
| 58_6F151_day5_Dox | 2.36 | 2.9 | 3.17 |
| 103_6F171_day28_Dox | 0.8 | 0.58 | 1.07 |
| 104_6F172_day28_Dox | 0.69 | 0.61 | 1.52 |
| 105_6F173_day28_Dox | 1.73 | 0.63 | 1.26 |
| 13_6M46_day1_Dox | 0.86 | 0.68 | 0.56 |
| 14_6M47_day1_Dox | 0.77 | 0.78 | 0.64 |
| 15_6M48_day1_Dox | 0.56 | 0.64 | 0.66 |
| 43_6M49_day5_Dox | 3 | 1.3 | 1.65 |
| 44_6M50_day5_Dox | 2.54 | 1.41 | 2 |
| 88_6M71_day28_Dox | 1.71 | 0.51 | 0.99 |
| 89_6M72_day28_Dox | 0.83 | 0.55 | 0.98 |
| 90_6M73_day28_Dox | 1.15 | 0.51 | 1.28 |
| 115_7F155_day1_ValpA | 0.77 | 0.98 | 0.86 |
| 116_7F156_day1_ValpA | 0.36 | 0.52 | 0.61 |
| 117_7F157_day1_ValpA | 0.47 | 0.74 | 0.8 |
| 121_7F158_day5_ValpA | 0.49 | 0.59 | 0.94 |
| 122_7F159_day5_ValpA | 0.57 | 0.55 | 0.77 |
| 123_7F160_day5_ValpA | 0.33 | 0.74 | 1.07 |
| 127_7F161_day28_ValpA | 0.47 | 0.69 | 0.89 |
| 128_7F162_day28_ValpA | 0.2 | 0.41 | 0.61 |
| 129_7F163_day28_ValpA | 0.24 | 0.58 | 0.68 |
| 112_7M55_day1_ValpA | 0.85 | 0.82 | 0.67 |
| 113_7M56_day1_ValpA | 0.73 | 0.64 | 0.42 |
| 114_7M57_day1_ValpA | 0.44 | 0.53 | 0.51 |
| 118_7M58_day5_ValpA | 0.99 | 0.68 | 0.8 |
| 119_7M59_day5_ValpA | 0.83 | 1.11 | 1.1 |
| 120_7M60_day5_ValpA | 0.93 | 0.93 | 1.03 |
| 124_7M61_day28_ValpA | 0.66 | 0.52 | 0.77 |
| 125_7M62_day28_ValpA | 0.55 | 0.65 | 0.71 |
| 126_7M63_day28_ValpA | 0.24 | 0.39 | 0.39 |
| 62_8F181_day28_Naive | 0.47 | 1.03 | 0.68 |
| 63_8F182_day28_Naive | 0.57 | 1.24 | 0.65 |
| 64_8F183_day28_Naive | 0.46 | 1.03 | 0.58 |
| 59_8M81_day28_Naive | 1.26 | 2 | 1.31 |
| 60_8M82_day28_Naive | 0.69 | 0.94 | 0.83 |
| 61_8M83_day28_Naive | 0.39 | 0.58 | 0.45 |

| Sample | MUSCYCG1R_DB81_seg19-20 | W33294_DB81_seg23 | W33294_DB81_seg44 |
|---|---|---|---|
| 68_1F101_day1_Saline | 0.74 | 0.78 | 0.8 |
| 69_1F102_day1_Saline | 0.84 | 0.65 | 0.96 |
| 70_1F103_day1_Saline | 0.64 | 0.8 | 0.72 |
| 74_1F104_day5_Saline | 1.28 | 0.86 | 0.65 |
| 75_1F105_day5_Saline | 0.75 | 0.97 | 0.88 |
| 109_1F107_day28_Saline | 0.57 | 1 | 0.88 |
| 110_1F108_day28_Saline | 0.5 | 0.79 | 0.59 |
| 111_1F109_day28_Saline | 0.79 | 1.24 | 1 |
| 65_1M1_day1_Saline | 0.44 | 0.69 | 0.64 |
| 66_1M2_day1_Saline | 0.43 | 0.3 | 0.69 |
| 67_1M3_day1_Saline | 0.36 | 0.46 | 0.53 |
| 71_1M4_day5_Saline | 0.87 | 0.78 | 1.69 |
| 72_1M5_day5_Saline | 0.85 | 1.18 | 1 |
| 73_1M6_day5_Saline | 0.78 | 0.9 | 0.86 |
| 106_1M7_day28_Saline | 0.44 | 0.61 | 0.63 |
| 107_1M8_day28_Saline | 0.34 | 0.62 | 0.48 |
| 108_1M9_day28_Saline | 0.51 | 0.84 | 0.55 |
| 16_2F110_day1_Gent | 0.67 | 1 | 1.02 |
| 17_2F111_day1_Gent | 0.55 | 0.51 | 0.8 |
| 18_2F112_day1_Gent | 0.44 | 0.94 | 0.77 |
| 45_2F113_day5_Gent | 1.23 | 1.64 | 1.64 |
| 46_2F114_day5_Gent | 1.41 | 1.96 | 1.72 |
| 47_2F115_day5_Gent | 1.42 | 1.51 | 2.13 |
| 91_2F116_day28_Gent | 0.56 | 0.64 | 0.66 |
| 92_2F117_day28_Gent | 0.74 | 1.3 | 0.71 |
| 93_2F118_day28_Gent | 0.42 | 0.85 | 1.04 |
| 1_2M10_day1_Gent | 1.7 | 0.57 | 0.77 |
| 2_2M11_day1_Gent | 0.48 | 0.74 | 0.5 |
| 3_2M12_day1_Gent | 0.92 | 0.61 | 0.64 |
| 31_2M13_day5_Gent | 1.43 | 1.25 | 1.12 |
| 32_2M14_day5_Gent | 1.04 | 0.82 | 0.93 |
| 33_2M15_day5_Gent | 1 | 0.95 | 0.94 |
| 76_2M16_day28_Gent | 0.79 | 0.57 | 0.42 |
| 77_2M17_day28_Gent | 0.4 | 0.8 | 0.85 |
| 78_2M18_day28_Gent | 0.55 | 0.67 | 0.69 |
| 19_3F119_day1_Cis | 0.86 | 0.36 | 1 |
| 20_3F120_day1_Cis | 0.81 | 0.28 | 0.95 |
| 21_3F121_day1_Cis | 0.81 | 1.11 | 1.04 |
| 48_3F122_day5_Cis | 2.77 | 2.03 | 2.31 |
| 49_3F123_day5_Cis | 3.44 | 1.99 | 2.19 |
| 50_3F124_day5_Cis | 2.45 | 1.58 | 2.15 |
| 94_3F125_day28_Cis | 1.64 | 0.95 | 0.35 |
| 95_3F126_day28_Cis | 2.47 | 0.97 | 0.67 |
| 96_3F127_day28_Cis | 1.67 | 0.82 | 0.73 |
| 4_3M19_day1_Cis | 0.85 | 0.59 | 0.6 |
| 5_3M20_day1_Cis | 0.71 | 0.49 | 0.84 |
| 6_3M21_day1_Cis | 1.18 | 0.64 | 0.96 |
| 34_3M22_day5_Cis | 1.83 | 0.84 | 1.47 |
| 35_3M23_day5_Cis | 1.47 | 0.59 | 1.16 |
| 36_3M24_day5_Cis | 2.33 | 1.34 | 1.51 |
| 79_3M25_day28_Cis | 2.13 | 0.95 | 0.83 |
| 80_3M26_day28_Cis | 1.97 | 0.82 | 0.89 |
| 81_3M27_day28_Cis | 2.21 | 0.88 | 0.83 |
| 22_4F128_day1_Tob | 0.73 | 0.85 | 0.98 |
| 23_4F129_day1_Tob | 0.63 | 0.56 | 0.57 |
| 24_4F130_day1_Tob | 0.58 | 0.59 | 0.62 |
| 51_4F131_day5_Tob | 1.42 | 2.16 | 2.01 |
| 52_4F132_day5_Tob | 1.31 | 2.37 | 2.06 |
| 53_4F133_day5_Tob | 1.2 | 1.2 | 1.72 |
| 97_4F134_day28_Tob | 0.45 | 0.91 | 0.95 |
| 98_4F135_day28_Tob | 0.62 | 1.16 | 0.75 |
| 99_4F136_day28_Tob | 0.58 | 0.91 | 0.7 |
| 7_4M28_day1_Tob | 0.72 | 0.52 | 0.78 |
| 8_4M29_day1_Tob | 0.84 | 0.91 | 0.88 |
| 9_4M30_day1_Tob | 0.63 | 0.35 | 0.46 |
| 37_4M31_day5_Tob | 0.72 | 0.61 | 0.48 |
| 38_4M32_day5_Tob | 0.72 | 0.81 | 0.69 |
| 39_4M33_day5_Tob | 1.53 | 1.25 | 1.27 |
| 82_4M34_day28_Tob | 0.49 | 1.02 | 0.75 |
| 83_4M35_day28_Tob | 0.51 | 0.78 | 0.86 |
| 84_4M36_day28_Tob | 0.6 | 0.73 | 0.86 |
| 25_5F137_day1_CadCl | 2.22 | 1.9 | 1.79 |
| 26_5F138_day1_CadCl | 1 | 1.74 | 1.39 |
| 27_5F139_day1_CadCl | 1.68 | 1.36 | 1.57 |
| 54_5F141_day5_CadCl | 1.21 | 1.76 | 2.05 |
| 55_5F142_day5_CadCl | 1.01 | 1.7 | 1.37 |
| 100_5F143_day28_CadCl | 0.54 | 0.94 | 0.88 |
| 101_5F144_day28_CadCl | 0.62 | 0.97 | 0.69 |
| 102_5F145_day28_CadCl | 0.56 | 0.85 | 0.7 |
| 10_5M37_day1_CadCl | 0.58 | 0.62 | 0.63 |
| 11_5M38_day1_CadCl | 1.03 | 0.31 | 0.26 |
| 12_5M39_day1_CadCl | 0.78 | 0.37 | 0.63 |
| 40_5M40_day5_CadCl | 1.22 | 1.41 | 1.32 |
| 41_5M41_day5_CadCl | 1.12 | 1.45 | 1.43 |
| 42_5M42_day5_CadCl | 1.04 | 1.03 | 1.42 |
| 85_5M43_day28_CadCl | 0.31 | 0.7 | 0.73 |
| 86_5M44_day28_CadCl | 0.75 | 0.65 | 0.53 |
| 87_5M45_day28_CadCl | 0.44 | 0.65 | 0.53 |
| 28_6F146_day1_Dox | 1.17 | 2.47 | 2.1 |
| 29_6F147_day1_Dox | 1.12 | 1.04 | 1.3 |
| 30_6F148_day1_Dox | 1.16 | 1.36 | 2.02 |
| 56_6F149_day5_Dox | 1.94 | 1.06 | 1.19 |
| 57_6F150_day5_Dox | 1.79 | 2.06 | 1.76 |
| 58_6F151_day5_Dox | 3.43 | 2.03 | 2.76 |
| 103_6F171_day28_Dox | 1.24 | 0.82 | 0.87 |
| 104_6F172_day28_Dox | 1.29 | 0.97 | 1.09 |
| 105_6F173_day28_Dox | 0.93 | 0.62 | 0.65 |
| 13_6M46_day1_Dox | 0.98 | 0.74 | 0.74 |
| 14_6M47_day1_Dox | 1.04 | 0.61 | 1.01 |
| 15_6M48_day1_Dox | 0.95 | 0.82 | 0.83 |
| 43_6M49_day5_Dox | 1.7 | 1.25 | 1 |
| 44_6M50_day5_Dox | 1.66 | 1.06 | 0.95 |
| 88_6M71_day28_Dox | 0.88 | 0.96 | 0.45 |
| 89_6M72_day28_Dox | 1.33 | 1 | 0.77 |
| 90_6M73_day28_Dox | 1.26 | 0.82 | 0.66 |
| 115_7F155_day1_ValpA | 0.68 | 1.67 | 0.86 |
| 116_7F156_day1_ValpA | 0.43 | 0.84 | 0.72 |
| 117_7F157_day1_ValpA | 0.77 | 1.21 | 1.19 |
| 121_7F158_day5_ValpA | 0.6 | 1.14 | 1.06 |
| 122_7F159_day5_ValpA | 0.45 | 1.13 | 0.85 |
| 123_7F160_day5_ValpA | 1.11 | 1.24 | 0.79 |
| 127_7F161_day28_ValpA | 0.55 | 0.98 | 0.61 |
| 128_7F162_day28_ValpA | 0.62 | 0.89 | 0.74 |
| 129_7F163_day28_ValpA | 0.5 | 1.16 | 0.95 |
| 112_7M55_day1_ValpA | 0.63 | 1.32 | 1.01 |
| 113_7M56_day1_ValpA | 0.53 | 0.94 | 0.7 |
| 114_7M57_day1_ValpA | 0.44 | 0.92 | 0.69 |
| 118_7M58_day5_ValpA | 0.88 | 1.09 | 1.66 |
| 119_7M59_day5_ValpA | 1.1 | 1.32 | 1.33 |
| 120_7M60_day5_ValpA | 1.08 | 1.01 | 1.31 |
| 124_7M61_day28_ValpA | 0.56 | 0.52 | 0.75 |
| 125_7M62_day28_ValpA | 0.44 | 0.85 | 0.85 |
| 126_7M63_day28_ValpA | 0.33 | 0.88 | 0.34 |
| 62_8F181_day28_Naive | 0.55 | 0.94 | 0.73 |
| 63_8F182_day28_Naive | 0.7 | 1.02 | 0.76 |
| 64_8F183_day28_Naive | 0.79 | 0.82 | 0.77 |
| 59_8M81_day28_Naive | 1.24 | 1.65 | 2.01 |
| 60_8M82_day28_Naive | 0.86 | 0.85 | 1.12 |
| 61_8M83_day28_Naive | 0.44 | 0.79 | 0.39 |

| Sample | RATRAB13X_DB81_seg15-17 | RATRAB13X_DB81_seg22 | MMU09507_DB81_seg15 |
|---|---|---|---|
| 68_1F101_day1_Saline | 0.7 | 1.29 | 1.03 |
| 69_1F102_day1_Saline | 0.59 | 0.81 | 1.13 |
| 70_1F103_day1_Saline | 0.68 | 0.66 | 0.51 |
| 74_1F104_day5_Saline | 0.64 | 0.82 | 0.42 |
| 75_1F105_day5_Saline | 0.77 | 1.04 | 0.01 |
| 109_1F107_day28_Saline | 1.08 | 0.69 | 0.67 |
| 110_1F108_day28_Saline | 0.95 | 0.61 | 0.2 |
| 111_1F109_day28_Saline | 1.03 | 0.97 | 0.33 |
| 65_1M1_day1_Saline | 0.48 | 1 | 0.14 |
| 66_1M2_day1_Saline | 0.55 | 0.65 | 0.24 |
| 67_1M3_day1_Saline | 0.48 | 0.44 | 0.24 |
| 71_1M4_day5_Saline | 1.11 | 0.86 | 1 |
| 72_1M5_day5_Saline | 1.02 | 1.06 | 0.37 |
| 73_1M6_day5_Saline | 0.78 | 0.75 | 0.25 |
| 106_1M7_day28_Saline | 0.58 | 0.68 | 0.23 |
| 107_1M8_day28_Saline | 0.5 | 0.51 | 0.17 |
| 108_1M9_day28_Saline | 0.7 | 0.8 | 0.19 |
| 16_2F110_day1_Gent | 0.9 | 0.98 | 0.41 |
| 17_2F111_day1_Gent | 0.63 | 0.71 | 0.43 |
| 18_2F112_day1_Gent | 0.55 | 0.51 | 0.23 |
| 45_2F113_day5_Gent | 1.68 | 2.16 | 0.78 |
| 46_2F114_day5_Gent | 1.5 | 2.11 | 0.62 |
| 47_2F115_day5_Gent | 1.59 | 1.91 | 1.13 |
| 91_2F116_day28_Gent | 0.84 | 0.68 | 1.38 |
| 92_2F117_day28_Gent | 0.9 | 0.56 | 0.96 |
| 93_2F118_day28_Gent | 0.88 | 0.57 | 0.7 |
| 1_2M10_day1_Gent | 0 | 0.8 | 0.56 |
| 2_2M11_day1_Gent | 1.16 | 0.99 | 0.32 |
| 3_2M12_day1_Gent | 0.82 | 0.6 | 0.37 |
| 31_2M13_day5_Gent | 1.25 | 1.25 | 0.71 |
| 32_2M14_day5_Gent | 0.76 | 0.77 | 0.41 |
| 33_2M15_day5_Gent | 1.35 | 1.16 | 0.68 |
| 76_2M16_day28_Gent | 0.77 | 0.91 | 0.82 |
| 77_2M17_day28_Gent | 0.76 | 0.88 | 0.59 |
| 78_2M18_day28_Gent | 1.22 | 0.58 | 0.84 |
| 19_3F119_day1_Cis | 0.82 | 0.88 | 0 |
| 20_3F120_day1_Cis | 1.11 | 0.87 | 0.44 |
| 21_3F121_day1_Cis | 1.1 | 1.02 | 0.52 |
| 48_3F122_day5_Cis | 1.37 | 1.71 | 1.96 |
| 49_3F123_day5_Cis | 1.53 | 1.92 | 2.32 |
| 50_3F124_day5_Cis | 1.53 | 1.99 | 1.14 |
| 94_3F125_day28_Cis | 1.33 | 0.61 | 8.1 |
| 95_3F126_day28_Cis | 1.27 | 0.65 | 6.58 |
| 96_3F127_day28_Cis | 1.21 | 0.89 | 5.53 |
| 4_3M19_day1_Cis | 0.73 | 0.65 | 0.35 |
| 5_3M20_day1_Cis | 0.84 | 0.66 | 0.19 |
| 6_3M21_day1_Cis | 0.98 | 1.11 | 0.5 |
| 34_3M22_day5_Cis | 1.8 | 1.53 | 2.07 |
| 35_3M23_day5_Cis | 1.03 | 1.2 | 0.57 |
| 36_3M24_day5_Cis | 1.77 | 1.28 | 3.87 |
| 79_3M25_day28_Cis | 1.08 | 0.66 | 8.05 |
| 80_3M26_day28_Cis | 0.95 | 0.76 | 2.96 |
| 81_3M27_day28_Cis | 1.28 | 0.65 | 5.45 |
| 22_4F128_day1_Tob | 1.02 | 0.41 | 0.35 |
| 23_4F129_day1_Tob | 0.77 | 0.71 | 0.3 |
| 24_4F130_day1_Tob | 0.87 | 0.78 | 0.22 |
| 51_4F131_day5_Tob | 1.71 | 2.49 | 1.04 |
| 52_4F132_day5_Tob | 1.44 | 2.11 | 0.99 |
| 53_4F133_day5_Tob | 1.23 | 1.57 | 0.92 |
| 97_4F134_day28_Tob | 1.28 | 0.88 | 0.84 |
| 98_4F135_day28_Tob | 1.01 | 0.63 | 0.52 |
| 99_4F136_day28_Tob | 0.73 | 0.88 | 0.85 |
| 7_4M28_day1_Tob | 0.78 | 0.82 | 0.34 |
| 8_4M29_day1_Tob | 0.98 | 0.95 | 0.7 |
| 9_4M30_day1_Tob | 0.91 | 0.82 | 0.56 |
| 37_4M31_day5_Tob | 0.75 | 0.68 | 0.17 |
| 38_4M32_day5_Tob | 1.04 | 0.81 | 0.28 |
| 39_4M33_day5_Tob | 1.32 | 1.26 | 0.27 |
| 82_4M34_day28_Tob | 0.93 | 0.67 | 0.66 |
| 83_4M35_day28_Tob | 1.05 | 0.8 | 0.97 |
| 84_4M36_day28_Tob | 0.94 | 0.62 | 0.68 |
| 25_5F137_day1_CadCl | 1.63 | 1.73 | 0.81 |
| 26_5F138_day1_CadCl | 1.8 | 1.83 | 0.42 |
| 27_5F139_day1_CadCl | 1.55 | 1.16 | 1.1 |
| 54_5F141_day5_CadCl | 1.81 | 1.56 | 0.87 |
| 55_5F142_day5_CadCl | 0.97 | 1.1 | 0.56 |
| 100_5F143_day28_CadCl | 0.83 | 0.7 | 0.33 |
| 101_5F144_day28_CadCl | 0.89 | 0.9 | 0.66 |
| 102_5F145_day28_CadCl | 0.82 | 0.46 | 1.58 |
| 10_5M37_day1_CadCl | 0.73 | 0.92 | 0.15 |
| 11_5M38_day1_CadCl | 0.92 | 1.29 | 0.32 |
| 12_5M39_day1_CadCl | 1.01 | 0.67 | 0.33 |
| 40_5M40_day5_CadCl | 1.18 | 1.65 | 0.33 |
| 41_5M41_day5_CadCl | 0.88 | 1.58 | 0.49 |
| 42_5M42_day5_CadCl | 1.07 | 1.21 | 0.38 |
| 85_5M43_day28_CadCl | 1.43 | 0.63 | 0.42 |
| 86_5M44_day28_CadCl | 0.8 | 0.87 | 0.45 |
| 87_5M45_day28_CadCl | 0.64 | 0.49 | 0.42 |
| 28_6F146_day1_Dox | 0.99 | 2.07 | 0.48 |
| 29_6F147_day1_Dox | 1.04 | 0.89 | 0.52 |
| 30_6F148_day1_Dox | 1.32 | 1.66 | 0.93 |
| 56_6F149_day5_Dox | 1.85 | 1.95 | 2.07 |
| 57_6F150_day5_Dox | 1.51 | 1.89 | 1.46 |
| 58_6F151_day5_Dox | 1.9 | 3.3 | 1.82 |
| 103_6F171_day28_Dox | 0.66 | 0.54 | 4.47 |
| 104_6F172_day28_Dox | 0.69 | 1.05 | 2.99 |
| 105_6F173_day28_Dox | 0.91 | 0.63 | 2.89 |
| 13_6M46_day1_Dox | 0.88 | 0.69 | 0.74 |
| 14_6M47_day1_Dox | 1.15 | 0.58 | 0.71 |
| 15_6M48_day1_Dox | 0.75 | 0.77 | 0.59 |
| 43_6M49_day5_Dox | 0.96 | 1.06 | 1.74 |
| 44_6M50_day5_Dox | 1.2 | 1.32 | 1.67 |
| 88_6M71_day28_Dox | 0.54 | 0.37 | 3.04 |
| 89_6M72_day28_Dox | 0.78 | 0.64 | 2.93 |
| 90_6M73_day28_Dox | 1.02 | 0.45 | 2.13 |
| 115_7F155_day1_ValpA | 1.28 | 1.05 | 0.48 |
| 116_7F156_day1_ValpA | 0.7 | 0.56 | 0.34 |
| 117_7F157_day1_ValpA | 0.82 | 0.87 | 0.27 |
| 121_7F158_day5_ValpA | 0.7 | 1.07 | 0.62 |
| 122_7F159_day5_ValpA | 0.91 | 0.78 | 0.31 |
| 123_7F160_day5_ValpA | 1.47 | 1.13 | 0.42 |
| 127_7F161_day28_ValpA | 0.73 | 0.71 | 0.23 |
| 128_7F162_day28_ValpA | 0.82 | 0.85 | 0.45 |
| 129_7F163_day28_ValpA | 0.44 | 0.74 | 0.24 |
| 112_7M55_day1_ValpA | 0.79 | 1.45 | 0.2 |
| 113_7M56_day1_ValpA | 0.7 | 0.86 | 0.2 |
| 114_7M57_day1_ValpA | 0.75 | 0.63 | 0.29 |
| 118_7M58_day5_ValpA | 1.38 | 0.95 | 0.26 |
| 119_7M59_day5_ValpA | 1.14 | 1.14 | 0.3 |
| 120_7M60_day5_ValpA | 1.45 | 1.1 | 0.24 |
| 124_7M61_day28_ValpA | 0.59 | 0.96 | 0.12 |
| 125_7M62_day28_ValpA | 0.77 | 0.75 | 0.11 |
| 126_7M63_day28_ValpA | 0.49 | 0.62 | 0.21 |
| 62_8F181_day28_Naive | 0.68 | 0.9 | 0.5 |
| 63_8F182_day28_Naive | 0.77 | 0.84 | 0.6 |
| 64_8F183_day28_Naive | 0.68 | 0.88 | 0.73 |
| 59_8M81_day28_Naive | 1.77 | 1.46 | 0.47 |
| 60_8M82_day28_Naive | 0.83 | 0.8 | 0.8 |
| 61_8M83_day28_Naive | 0.54 | 0.53 | 0.43 |

### Example 3

### Validation Analysis of the Selected Markers

To validate the optimal signature for classification of renal toxicity of a compound after five days of application a test of the signatory polynucleotide/gene expression in rat kidney samples consisting of tissues from rats exposed to three drug compounds and a single control group was carried out (Teva Pharmaceutical Industries, IL). The purpose of this analysis was to test the ability of the optimized signature to correctly predict the level of toxicity of the three drug compounds prior to the ability to demonstrate renal damage using histopathological examination of the rat kidney samples.

In this Example, the samples with known renal toxic effect (detailed in plates 1-3 described in Table 25 and in Table 9) are referred to as the “labeled samples”, and the samples of the blind test (detailed in plates 4-7 described in Table 25), where no information on the renal toxic effect was available, are referred to as the “un-labeled samples”. The experimental details and the analysis performed initially on the labeled samples, as described in Example 2, are referred to as the “discovery stage” of the study, while the details and analysis performed at the second stage of the study are referred to as the “validation stage”.

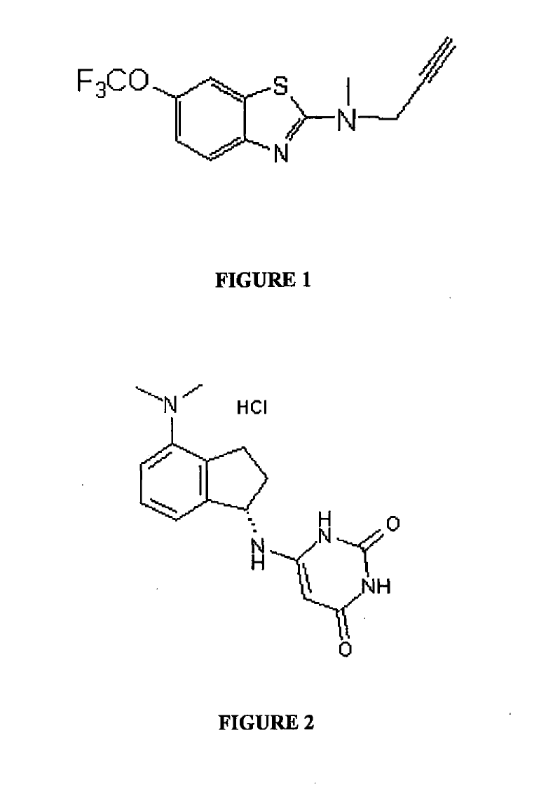

The signature disclosed in Table 13 herein was further revised and refined, and then validated by testing the un-labeled rat kidney samples, consisting of tissues from rats exposed to three drug compounds (T1 and T2 presented in FIGS. 1-2 and compound T3, Riluzole (6-(trifluoromethoxy)benzothiazol-2-amine) and from a single control group.

qRT-PCR was performed on the labeled and un-labeled samples as described in section “RT Preparation and Real-Time qRT-PCR Analysis” hereinabove, using primers for 8 amplicons of 4 genes (a wild-type and a splice-variant amplicon for each gene disclosed in Table 13), as detailed in Example 2 hereinabove. New primers were designed for part of the transcripts, and new conditions for several qRT-PCR reactions were used, as described below.

Two equivalent modified signatures were constructed based on new qRT-PCR results obtained from the test conducted with the labeled samples, which are listed in Tables 26 and 27 below. The modified signatures were then applied to the un-labeled samples and successfully predicted the level of toxicity of the three test compounds as well as of the control.

Table 23 provides the normalized qRT-PCR results for the amplicons used for both classifiers (shown in Tables 26 and 27) on the labeled and un-labeled samples. The measurements were normalized in two steps. First—according to the house-keeping-genes normalization factor, and second—each plate was normalized according to ratio of the intensity measurements from samples appearing in it and in the shuffled (normalization) plate (Table 25 provides the plates details and the section “Inter-Plate Normalization” provides details of the normalization process). The normalized values were further scaled by a factor, for ease of viewing. This data, as well as the additional gender column was used by the classifiers to reach the toxicity calls. Table 23 also provides the true label (Normal/Toxic) for the labeled samples.

TABLE 23
Normalized qRT-PCR results for amplicons used with labeled and un-labeled samples

| Sample | W41270_DB81_seg11 | H31883_DB71_seg13 | MUSCYCG1R_DB81_seg19-20 |
|---|---|---|---|
| 56_6F149_day5_Dox | 0.851 | 1.377 | 1.438 |
| 48_3F122_day5_Cis | 0.705 | 2.695 | 1.643 |
| 47_2F115_day5_Gent | 2.64 | 1.515 | 1.644 |
| 110_1F108_day28_Saline | 0.775 | 0.741 | 0.431 |
| 34_3M22_day5_Cis | 1.262 | 3.816 | 2.034 |
| 44_6M50_day5_Dox | 1.017 | 2.129 | 1.321 |
| 106_1M7_day28_Saline | 0.489 | 0.728 | 0.804 |
| 116_7F156_day1_ValpA | 1.134 | 1.026 | 2.289 |
| 59_8M81_day28_Naive | 1.117 | 0.965 | 1.232 |
| 124_7M61_day28_ValpA | 0.543 | 0.732 | 0.588 |
| 65_1M1_day1_Saline | 1.029 | 0.933 | 1.176 |
| 74_1F104_day5_Saline | 0.801 | 0.911 | 1.516 |
| 57_6F150_day5_Dox | 1.053 | 0.905 | 1.723 |
| 43_6M49_day5_Dox | 1.551 | 2.586 | 1.536 |
| 128_7F162_day28_ValpA | 0.95 | 0.774 | 1.731 |
| 120_7M60_day5_ValpA | 0.631 | 0.846 | 1.019 |
| 114_7M57_day1_ValpA | 0.939 | 1.488 | 1.581 |
| 37_4M31_day5_Tob | 0.559 | 0.863 | 0.99 |
| 126_7M63_day28_ValpA | 0.459 | 0.713 | 0.589 |
| 112_7M55_day1_ValpA | 1.405 | 1.506 | 1.842 |
| 73_1M6_day5_Saline | 0.413 | 0.966 | 0.768 |
| 62_8F181_day28_Naive | 0.931 | 1.262 | 0.933 |
| 123_7F160_day5_ValpA | 1.428 | 0.85 | 1.509 |
| 109_1F107_day28_Saline | 0.743 | 0.775 | 0.942 |
| 118_7M58_day5_ValpA | 1.002 | 0.975 | 1.372 |
| 68_1F101_day1_Saline | 1.725 | 1.074 | 1.424 |
| 117_7F157_day1_ValpA | 1.225 | 0.999 | 1.929 |
| 36_3M24_day5_Cis | 3.597 | 5.526 | 4.156 |
| 33_2M15_day5_Gent | 0.983 | 0.948 | 1.085 |
| 127_7F161_day28_ValpA | 1.044 | 0.872 | 1.171 |
| 75_1F105_day5_Saline | 0.044 | 0.838 | 1.262 |
| 52_4F132_day5_Tob | 2.483 | 0.855 | 1.589 |
| 50_3F124_day5_Cis | 1.404 | 3.771 | 4.947 |
| 63_8F182_day28_Naive | 0.809 | 0.95 | 0.772 |
| 38_4M32_day5_Tob | 0.802 | 1.237 | 0.968 |
| 72_1M5_day5_Saline | 0.654 | 0.963 | 1.245 |
| 67_1M3_day1_Saline | 0.805 | 1.2 | 0.903 |
| 35_3M23_day5_Cis | 0.843 | 1.509 | 0.981 |
| 51_4F131_day5_Tob | 1.529 | 0.988 | 1.6 |
| 32_2M14_day5_Gent | 0.954 | 1.09 | 0.993 |
| 122_7F159_day5_ValpA | 0.617 | 0.791 | 0.94 |
| 121_7F158_day5_ValpA | 1.258 | 0.694 | 1.425 |
| 119_7M59_day5_ValpA | 0.997 | 1.082 | 1.585 |
| 71_1M4_day5_Saline | 0.605 | 0.731 | 0.975 |
| 111_1F109_day28_Saline | 0.996 | 0.904 | 1.014 |
| 60_8M82_day28_Naive | 0.631 | 1.255 | 1.006 |
| 129_7F163_day28_ValpA | 0.587 | 0.57 | 1.037 |
| 69_1F102_day1_Saline | 0.573 | 0.793 | 1.449 |
| 108_1M9_day28_Saline | 0.831 | 0.812 | 0.634 |
| 46_2F114_day5_Gent | 1.104 | 0.78 | 0.955 |
| 125_7M62_day28_ValpA | 1.011 | 0.979 | 0.52 |
| 113_7M56_day1_ValpA | 0.547 | 0.721 | 0.637 |
| 45_2F113_day5_Gent | 1.699 | 1.142 | 1.442 |
| 61_8M83_day28_Naive | 0.411 | 0.637 | 1.209 |
| 53_4F133_day5_Tob | 2.317 | 0.858 | 1.424 |
| 107_1M8_day28_Saline | 0.562 | 0.719 | 0.642 |
| 115_7F155_day1_ValpA | 1.544 | 1.041 | 0.9 |
| 58_6F151_day5_Dox | 1.791 | 1.227 | 2.913 |
| 31_2M13_day5_Gent | 0.82 | 1.402 | 1.842 |
| 49_3F123_day5_Cis | 2.113 | 1.642 | 3.172 |
| 39_4M33_day5_Tob | 0.946 | 1.646 | 2.062 |
| 70_1F103_day1_Saline | 1.021 | 1.173 | 1.629 |
| 66_1M2_day1_Saline | 0.753 | 0.883 | 0.705 |
| 64_8F183_day28_Naive | 0.843 | 0.943 | 1.282 |
| 54_UK5F126_T1_Ds2_Day1 | 1.265 | 1.153 | 0.609 |
| 123_UK4F119_T1_Ds1_Day5 | 0.657 | 1.01 | 0.816 |
| 60_UK7F139_T2_Ds1_Day1 | 3.38 | 0.7 | 0.126 |
| 17_UK5M29_T1_Ds2_Day1 | 0.653 | 1.19 | 0.835 |
| 117_UK2F107_control_Day5 | 1.229 | 2.274 | 0.68 |
| 109_UK12M69_T3_Ds1_Day5 | 0.482 | 1.3 | 0.62 |
| 18_UK5M30_T1_Ds2_Day1 | 0.383 | 0.623 | 0.696 |
| 57_UK5F129_T1_Ds2_Day1 | 0.472 | 0.798 | 0.804 |
| 151_UK14F180_T3_Ds2_Day5 | 0.363 | 0.705 | 1.004 |
| 7_UK3M13_T1_Ds1_Day1 | 0.734 | 0.923 | 0.299 |
| 113_UK14M80_T3_Ds2_Day5 | 0.605 | 0.846 | 0.658 |
| 72_UK11F164_T3_Ds1_Day1 | 0.282 | 0.582 | 0.114 |
| 104_UK10M56_T2_Ds2_Day5 | 0.668 | 1.256 | 0.655 |
| 91_UK6M31_T1_Ds2_Day5 | 1.164 | 1.663 | 1.018 |
| 124_UK4F120_T1_Ds1_Day5 | 0.703 | 1.013 | 1.499 |
| 114_UK14M81_T3_Ds2_Day5 | 0.43 | 0.949 | 1.003 |
| 69_UK9F154_T2_Ds2_Day1 | 0.92 | 0.762 | 0.111 |
| 46_UK1F105_control_Day1 | 0.349 | 0.692 | 0.164 |
| 34_UK11M64_T3_Ds1_Day1 | 0.649 | 1.074 | 0.726 |
| 130_UK6F132_T1_Ds2_Day5 | 1 | 1.554 | 0.919 |
| 3_UK1M3_control_Day1 | 0.398 | 0.756 | 0.171 |
| 75_UK13F173_T3_Ds2_Day1 | 0.897 | 0.662 | 0.798 |
| 101_UK8M47_T2_Ds1_Day5 | 0.397 | 1.149 | 0.172 |
| 102_UK8M48_T2_Ds1_Day5 | 0.337 | 2.147 | 0.179 |
| 96_UK6M36_T1_Ds2_Day5 | 0.71 | 0.996 | 0.756 |
| 39_UK13M76_T3_Ds2_Day1 | 0.359 | 0.643 | 0.158 |
| 19_UK7M37_T2_Ds1_Day1 | 0.623 | 0.7 | 0.51 |
| 144_UK12F167_T3_Ds1_Day5 | 1.957 | 1.301 | 0.638 |
| 2_UK1M2_control_Day1 | 0.342 | 0.812 | 0.222 |
| 52_UK3F118_T1_Ds1_Day1 | 0.566 | 0.706 | 2.31 |
| 31_UK11M61_T3_Ds1_Day1 | 0.64 | 0.919 | 0.96 |
| 129_UK6F131_T1_Ds2_Day5 | 0.009 | 1.36 | 0.359 |
| 23_UK7M41_T2_Ds1_Day1 | 0.481 | 0.88 | 0.938 |
| 43_UK1F102_control_Day1 | 0.406 | 2.051 | 0.328 |
| 81_UK2M8_control_Day5 | 0.686 | 1.318 | 0.252 |
| 80_UK2M7_control_Day5 | 0.788 | 1.981 | 0.273 |
| 70_UK11F161_T3_Ds1_Day1 | 0.457 | 0.764 | 0.232 |
| 16_UK5M28_T1_Ds2_Day1 | 0.78 | 0.517 | 0.385 |
| 79_UK13F178_T3_Ds2_Day1 | 0.49 | 0.802 | 0.92 |
| 50_UK3F116_T1_Ds1_Day1 | 0.66 | 0.913 | 1.49 |
| 112_UK12M72_T3_Ds1_Day5 | 0.722 | 1.65 | 0.649 |
| 146_UK12F169_T3_Ds1_Day5 | 1.807 | 1.003 | 0.716 |
| 140_UK10F156_T2_Ds2_Day5 | 1.186 | 1.386 | 0.345 |
| 143_UK10F160_T2_Ds2_Day5 | 0.308 | 0.676 | 0.169 |
| 10_UK3M16_T1_Ds1_Day1 | 0.181 | 0.757 | 0.291 |
| 94_UK6M34_T1_Ds2_Day5 | 0.391 | 1.037 | 0.871 |
| 147_UK12F170_T3_Ds1_Day5 | 0.483 | 1.338 | 1.035 |
| 71_UK11F163_T3_Ds1_Day1 | 0.575 | 0.611 | 0.183 |
| 122_UK2F112_control_Day5 | 0.592 | 1.127 | 0.159 |
| 47_UK1F106_control_Day1 | 0.405 | 0.823 | 0.196 |
| 15_UK5M27_T1_Ds2_Day1 | 0.755 | 0.906 | 0.76 |
| 97_UK8M43_T2_Ds1_Day5 | 0.233 | 1.884 | 0.107 |
| 106_UK10M59_T2_Ds2_Day5 | 0.497 | 1.01 | 0.513 |
| 118_UK2F108_control_Day5 | 0.911 | 2.171 | 0.279 |
| 148_UK12F171_T3_Ds1_Day5 | 0.899 | 0.576 | 1.226 |
| 35_UK11M65_T3_Ds1_Day1 | 0.374 | 0.663 | 1.044 |
| 21_UK7M39_T2_Ds1_Day1 | 0.662 | 0.67 | 0.555 |
| 38_UK13M75_T3_Ds2_Day1 | 0.347 | 0.969 | 0.203 |
| 55_UK5F127_T1_Ds2_Day1 | 0.885 | 0.849 | 0.739 |
| 100_UK8M46_T2_Ds1_Day5 | 0.493 | 1.539 | 0.113 |
| 67_UK9F152_T2_Ds2_Day1 | 1.747 | 0.832 | 0.211 |
| 131_UK6F133_T1_Ds2_Day5 | 0.964 | 1.84 | 0.852 |
| 142_UK10F159_T2_Ds2_Day5 | 0.711 | 1.128 | 0.148 |
| 128_UK4F124_T1_Ds1_Day5 | 1.527 | 1.373 | 1.598 |
| 95_UK6M35_T1_Ds2_Day5 | 0.713 | 1.348 | 0.761 |
| 8_UK3M14_T1_Ds1_Day1 | 0.396 | 0.615 | 0.271 |
| 138_UK8F147_T2_Ds1_Day5 | 0.626 | 1.163 | 1.399 |
| 41_UK13M78_T3_Ds2_Day1 | 0.174 | 0.632 | 0.275 |
| 76_UK13F175_T3_Ds2_Day1 | 0.912 | 1.119 | 2.153 |
| 152_UK14F181_T3_Ds2_Day5 | 0.762 | 0.942 | 1.567 |
| 86_UK4M20_T1_Ds1_Day5 | 0.473 | 1.353 | 0.183 |
| 59_UK7F137_T2_Ds1_Day1 | 0.473 | 0.613 | 0.115 |
| 111_UK12M71_T3_Ds1_Day5 | 0.832 | 1.082 | 0.857 |
| 145_UK12F168_T3_Ds1_Day5 | 1.015 | 1.358 | 0.625 |
| 28_UK9M52_T2_Ds2_Day1 | 1.016 | 0.773 | 0.531 |
| 108_UK12M67_T3_Ds1_Day5 | 0.699 | 1.585 | 0.525 |
| 11_UK3M17_T1_Ds1_Day1 | 0.198 | 0.73 | 0.376 |
| 24_UK7M42_T2_Ds1_Day1 | 0.76 | 0.695 | 0.859 |
| 42_UK1F101_control_Day1 | 0.415 | 0.976 | 0.276 |
| 29_UK9M53_T2_Ds2_Day1 | 0.424 | 0.635 | 0.505 |
| 61_UK7F140_T2_Ds1_Day1 | 0.474 | 0.464 | 0.124 |
| 20_UK7M38_T2_Ds1_Day1 | 0.876 | 0.951 | 0.888 |
| 141_UK10F158_T2_Ds2_Day5 | 1.237 | 1.161 | 0.185 |
| 87_UK4M21_T1_Ds1_Day5 | 0.702 | 1.266 | 0.149 |
| 150_UK14F179_T3_Ds2_Day5 | 1.066 | 0.718 | 0.875 |
| 136_UK8F144_T2_Ds1_Day5 | 1.149 | 1.329 | 1.006 |
| 93_UK6M33_T1_Ds2_Day5 | 1.825 | 2.423 | 1.197 |
| 85_UK4M19_T1_Ds1_Day5 | 0.356 | 1.783 | 0.214 |
| 4_UK1M4_control_Day1 | 0.617 | 0.668 | 0.192 |
| 126_UK4F122_T1_Ds1_Day5 | 1.325 | 1.477 | 3.532 |
| 36_UK11M66_T3_Ds1_Day1 | 0.468 | 0.569 | 1.137 |
| 74_UK11F166_T3_Ds1_Day1 | 0.465 | 1.366 | 0.132 |
| 14_UK5M26_T1_Ds2_Day1 | 0.462 | 0.696 | 0.478 |
| 119_UK2F109_control_Day5 | 0.994 | 1.083 | 0.162 |
| 25_UK9M49_T2_Ds2_Day1 | 0.589 | 0.722 | 0.585 |
| 103_UK10M55_T2_Ds2_Day5 | 0.857 | 1.146 | 0.997 |
| 27_UK9M51_T2_Ds2_Day1 | 0.545 | 0.618 | 0.475 |
| 26_UK9M50_T2_Ds2_Day1 | 0.528 | 0.999 | 0.661 |
| 44_UK1F103_control_Day1 | 0.563 | 0.692 | 0.161 |
| 62_UK7F141_T2_Ds1_Day1 | 0.959 | 0.536 | 0.267 |
| 92_UK6M32_T1_Ds2_Day5 | 0.453 | 1.13 | 0.631 |
| 12_UK3M18_T1_Ds1_Day1 | 0.231 | 0.524 | 0.135 |
| 5_UK1M5_control_Day1 | 0.3 | 1.146 | 0.184 |
| 153_UK14F182_T3_Ds2_Day5 | 1.119 | 1.058 | 1.708 |
| 56_UK5F128_T1_Ds2_Day1 | 0.413 | 0.611 | 0.738 |
| 58_UK5F130_T1_Ds2_Day1 | 0.762 | 0.707 | 0.652 |
| 132_UK6F134_T1_Ds2_Day5 | 1.254 | 2.468 | 1.238 |
| 49_UK3F114_T1_Ds1_Day1 | 0.758 | 0.783 | 1.436 |
| 90_UK4M24_T1_Ds1_Day5 | 0.657 | 1.097 | 0.296 |
| 64_UK9F149_T2_Ds2_Day1 | 0.384 | 0.602 | 0.197 |
| 110_UK12M70_T3_Ds1_Day5 | 0.495 | 0.946 | 0.398 |
| 127_UK4F123_T1_Ds1_Day5 | 1.014 | 2.104 | 3.397 |
| 121_UK2F111_control_Day5 | 0.394 | 0.882 | 0.222 |
| 83_UK2M10_control_Day5 | 0.719 | 1.033 | 0.103 |
| 98_UK8M44_T2_Ds1_Day5 | 0.321 | 1.66 | 0.148 |
| 99_UK8M45_T2_Ds1_Day5 | 0.477 | 1.079 | 0.127 |
| 107_UK10M60_T2_Ds2_Day5 | 0.788 | 1.057 | 0.746 |
| 73_UK11F165_T3_Ds1_Day1 | 0.344 | 0.637 | 0.134 |
| 40_UK13M77_T3_Ds2_Day1 | 0.418 | 0.923 | 0.29 |
| 89_UK4M23_T1_Ds1_Day5 | 1.11 | 1.249 | 0.186 |
| 134_UK6F136_T1_Ds2_Day5 | 1.111 | 1.684 | 0.528 |
| 133_UK6F135_T1_Ds2_Day5 | 1.126 | 1.672 | 1.583 |
| 1_UK1M1_control_Day1 | 0.578 | 0.554 | 0.148 |
| 30_UK9M54_T2_Ds2_Day1 | 0.577 | 0.522 | 0.458 |
| 82_UK2M9_control_Day5 | 0.269 | 1.398 | 0.157 |
| 37_UK13M73_T3_Ds2_Day1 | 0.359 | 0.767 | 0.152 |
| 45_UK1F104_control_Day1 | 0.754 | 0.709 | 0.197 |
| 105_UK10M57_T2_Ds2_Day5 | 0.457 | 0.942 | 0.686 |
| 135_UK8F143_T2_Ds1_Day5 | 0.83 | 0.936 | 0.736 |
| 33_UK11M63_T3_Ds1_Day1 | 0.568 | 0.949 | 0.767 |
| 77_UK13F176_T3_Ds2_Day1 | 0.667 | 1.13 | 1.45 |
| 63_UK7F142_T2_Ds1_Day1 | 0.518 | 0.646 | 0.157 |
| 48_UK3F113_T1_Ds1_Day1 | 0.646 | 0.912 | 1.195 |
| 65_UK9F150_T2_Ds2_Day1 | 1.729 | 1.006 | 0.167 |
| 84_UK2M11_control_Day5 | 0.152 | 0.819 | 0.093 |
| 125_UK4F121_T1_Ds1_Day5 | 1.337 | 1.458 | 1.317 |
| 139_UK8F148_T2_Ds1_Day5 | 1.524 | 0.978 | 1.386 |
| 9_UK3M15_T1_Ds1_Day1 | 0.47 | 0.87 | 0.249 |
| 115_UK14M82_T3_Ds2_Day5 | 0.472 | 1.087 | 1.024 |
| 137_UK8F145_T2_Ds1_Day5 | 1.111 | 1.439 | 1.293 |
| 13_UK5M25_T1_Ds2_Day1 | 0.644 | 0.517 | 0.654 |
| 66_UK9F151_T2_Ds2_Day1 | 1.125 | 1.079 | 0.192 |
| 88_UK4M22_T1_Ds1_Day5 | 0.638 | 1.23 | 0.189 |
| 149_UK12F172_T3_Ds1_Day5 | 1.093 | 1.094 | 1.413 |
| 32_UK11M62_T3_Ds1_Day1 | 0.551 | 0.7 | 0.441 |
| 68_UK9F153_T2_Ds2_Day1 | 0.594 | 0.84 | 0.14 |
| 22_UK7M40_T2_Ds1_Day1 | 0.823 | 0.629 | 0.47 |
| 6_UK1M6_control_Day1 | 0.245 | 0.66 | 0.402 |
| 120_UK2F110_control_Day5 | 0.456 | 1.399 | 0.233 |
| 78_UK13F177_T3_Ds2_Day1 | 0.956 | 1.47 | 2.313 |
| 53_UK5F125_T1_Ds2_Day1 | 1.034 | 0.867 | 1.261 |
| 116_UK14M83_T3_Ds2_Day5 | 0.766 | 2.34 | 1.119 |
| 51_UK3F117_T1_Ds1_Day1 | 1.082 | 0.957 | 1.891 |
| 154_UK14F183_T3_Ds2_Day5 | 0.944 | 1.259 | 1.475 |
| 16_2F110_day1_Gent | 1.361 | 0.772 | 0.842 |
| 42_5M42_day5_CadCl | 1.067 | 1.057 | 1.036 |
| 102_5F145_day28_CadCl | 1.138 | 0.785 | 0.769 |
| 9_4M30_day1_Tob | 1.601 | 1.137 | 1.582 |
| 97_4F134_day28_Tob | 1.189 | 0.624 | 0.713 |
| 105_6F173_day28_Dox | 1.263 | 1.328 | 0.985 |
| 79_3M25_day28_Cis | 4.741 | 4.41 | 3.509 |
| 78_2M18_day28_Gent | 1.614 | 0.853 | 0.863 |
| 54_5F141_day5_CadCl | 2.101 | 1.154 | 2.018 |
| 5_3M20_day1_Cis | 0.679 | 0.668 | 0.806 |
| 77_2M17_day28_Gent | 1.342 | 0.861 | 1.072 |
| 82_4M34_day28_Tob | 1.217 | 0.324 | 0.76 |
| 12_5M39_day1_CadCl | 0.727 | 0.919 | 1.224 |
| 26_5F138_day1_CadCl | 1.183 | 1.005 | 1.097 |
| 94_3F125_day28_Cis | 4.104 | 2.839 | 3.443 |
| 93_2F118_day28_Gent | 1.315 | 0.553 | 0.617 |
| 17_2F111_day1_Gent | 1.037 | 1.118 | 1.155 |
| 92_2F117_day28_Gent | 1.194 | 0.817 | 0.865 |
| 89_6M72_day28_Dox | 0.73 | 1.54 | 2.342 |
| 55_5F142_day5_CadCl | 1.109 | 0.786 | 1.292 |
| 15_6M48_day1_Dox | 0.824 | 0.993 | 1.387 |
| 3_2M12_day1_Gent | 0.904 | 0.987 | 1.361 |
| 84_4M36_day28_Tob | 1.099 | 0.69 | 1.087 |
| 87_5M45_day28_CadCl | 0.529 | 0.652 | 1.356 |
| 25_5F137_day1_CadCl | 1.265 | 1.446 | 2.129 |
| 98_4F135_day28_Tob | 1.267 | 0.528 | 0.889 |
| 41_5M41_day5_CadCl | 0.769 | 1.132 | 3.613 |
| 83_4M35_day28_Tob | 1.373 | 0.551 | 1.268 |
| 2_2M11_day1_Gent | 1.08 | 1.115 | 2.011 |
| 13_6M46_day1_Dox | 1.286 | 1.283 | 1.761 |
| 24_4F130_day1_Tob | 1.084 | 1.104 | 0.967 |
| 101_5F144_day28_CadCl | 0.772 | 0.712 | 1.068 |
| 21_3F121_day1_Cis | 0.759 | 0.853 | 1.514 |
| 88_6M71_day28_Dox | 2.381 | 1.434 | 1.437 |
| 100_5F143_day28_CadCl | 0.578 | 0.681 | 0.68 |
| 10_5M37_day1_CadCl | 0.993 | 1.239 | 2.276 |
| 7_4M28_day1_Tob | 0.577 | 1.132 | 1.506 |
| 18_2F112_day1_Gent | 2.078 | 0.84 | 1.387 |
| 28_6F146_day1_Dox | 1.43 | 1.41 | 2.256 |
| 8_4M29_day1_Tob | 0.949 | 0.912 | 1.173 |
| 91_2F116_day28_Gent | 1.188 | 0.554 | 0.729 |
| 22_4F128_day1_Tob | 0.809 | 0.563 | 1.025 |
| 86_5M44_day28_CadCl | 0.654 | 0.849 | 2.128 |
| 14_6M47_day1_Dox | 1.148 | 1.042 | 1.516 |
| 40_5M40_day5_CadCl | 1.211 | 1.051 | 2.866 |
| 96_3F127_day28_Cis | 2.053 | 2.398 | 1.813 |
| 30_6F148_day1_Dox | 1.501 | 1.46 | 1.627 |
| 103_6F171_day28_Dox | 1.44 | 1.642 | 1.991 |
| 19_3F119_day1_Cis | 1.331 | 1.416 | 1.492 |
| 85_5M43_day28_CadCl | 0.522 | 0.578 | 0.932 |
| 80_3M26_day28_Cis | 1.904 | 2.653 | 3.094 |
| 27_5F139_day1_CadCl | 0.983 | 1.632 | 1.856 |
| 76_2M16_day28_Gent | 1.941 | 0.842 | 1.661 |
| 90_6M73_day28_Dox | 0.652 | 1.611 | 2.105 |
| 29_6F147_day1_Dox | 0.935 | 1.116 | 1.551 |
| 23_4F129_day1_Tob | 0.983 | 0.661 | 0.987 |
| 1_2M10_day1_Gent | 0.85 | 1.053 | 2.861 |
| 20_3F120_day1_Cis | 1.029 | 2.092 | 3.259 |
| 104_6F172_day28_Dox | 0.787 | 1.408 | 1.779 |
| 6_3M21_day1_Cis | 1.224 | 2.559 | 2.003 |
| 95_3F126_day28_Cis | 4.516 | 5.592 | 7.02 |
| 81_3M27_day28_Cis | 3.866 | 3.563 | 3.473 |
| 99_4F136_day28_Tob | 1.798 | 0.807 | 0.903 |
| 4_3M19_day1_Cis | 0.496 | 1.134 | 1.277 |
| 11_5M38_day1_CadCl | 1 | 1.143 | 3.981 |

| Sample | W64472_DB81_seg2 | W83813_DB81_seg27 | AI045075_DB71_seg6 | Gender | Label |
|---|---|---|---|---|---|
| 56_6F149_day5_Dox | 2.03 | 1.442 | 1.248 | F | Toxic |
| 48_3F122_day5_Cis | 1.795 | 1.716 | 1.804 | F | Toxic |
| 47_2F115_day5_Gent | 1.733 | 1.688 | 1.337 | F | Toxic |
| 110_1F108_day28_Saline | 0.865 | 1.019 | 0.724 | F | Normal |
| 34_3M22_day5_Cis | 3.64 | 0.857 | 1.683 | M | Toxic |
| 44_6M50_day5_Dox | 2.924 | 0.986 | 1.348 | M | Toxic |
| 106_1M7_day28_Saline | 0.775 | 0.535 | 0.544 | M | Normal |
| 116_7F156_day1_ValpA | 1.082 | 1.179 | 1.006 | F | Normal |
| 59_8M81_day28_Naive | 2.184 | 1.679 | 1.27 | M | Normal |
| 124_7M61_day28_ValpA | 1.085 | 0.493 | 0.696 | M | Normal |
| 65_1M1_day1_Saline | 1.57 | 1.166 | 1.202 | M | Normal |
| 74_1F104_day5_Saline | 1.013 | 0.889 | 0.535 | F | Normal |
| 57_6F150_day5_Dox | 1.441 | 1.177 | 1.319 | F | Toxic |
| 43_6M49_day5_Dox | 3.4 | 1.095 | 1.844 | M | Toxic |
| 128_7F162_day28_ValpA | 0.788 | 1.254 | 0.906 | F | Normal |
| 120_7M60_day5_ValpA | 0.92 | 0.778 | 1.017 | M | Normal |
| 114_7M57_day1_ValpA | 1.123 | 0.912 | 1.436 | M | Normal |
| 37_4M31_day5_Tob | 1.193 | 0.589 | 1.327 | M | Toxic |
| 126_7M63_day28_ValpA | 0.965 | 0.46 | 0.669 | M | Normal |
| 112_7M55_day1_ValpA | 1.887 | 1.495 | 2.189 | M | Normal |
| 73_1M6_day5_Saline | 0.801 | 0.629 | 0.518 | M | Normal |
| 62_8F181_day28_Naive | 1.493 | 1.594 | 1.044 | F | Normal |
| 123_7F160_day5_ValpA | 1.226 | 1.17 | 1.145 | F | Normal |
| 109_1F107_day28_Saline | 0.718 | 0.642 | 0.982 | F | Normal |
| 118_7M58_day5_ValpA | 1.169 | 0.876 | 0.997 | M | Normal |
| 68_1F101_day1_Saline | 1.041 | 1.508 | 0.905 | F | Normal |
| 117_7F157_day1_ValpA | 0.995 | 1.13 | 0.983 | F | Normal |
| 36_3M24_day5_Cis | 6.414 | 1.461 | 4.298 | M | Toxic |
| 33_2M15_day5_Gent | 2.048 | 1.042 | 1.374 | M | Toxic |
| 127_7F161_day28_ValpA | 1.51 | 0.875 | 0.822 | F | Normal |
| 75_1F105_day5_Saline | 1.154 | 1.395 | 0.845 | F | Normal |
| 52_4F132_day5_Tob | 1.871 | 1.262 | 1.644 | F | Toxic |
| 50_3F124_day5_Cis | 1.976 | 1.366 | 1.503 | F | Toxic |
| 63_8F182_day28_Naive | 0.899 | 1.367 | 0.915 | F | Normal |
| 38_4M32_day5_Tob | 1.449 | 0.892 | 1.614 | M | Toxic |
| 72_1M5_day5_Saline | 0.943 | 1.059 | 0.763 | M | Normal |
| 67_1M3_day1_Saline | 3.263 | 1.107 | 1.751 | M | Normal |
| 35_3M23_day5_Cis | 1.448 | 0.758 | 0.93 | M | Toxic |
| 51_4F131_day5_Tob | 1.063 | 1.058 | 1.367 | F | Toxic |
| 32_2M14_day5_Gent | 1.962 | 0.702 | 1.402 | M | Toxic |
| 122_7F159_day5_ValpA | 0.702 | 0.926 | 0.883 | F | Normal |
| 121_7F158_day5_ValpA | 0.812 | 1.146 | 1.052 | F | Normal |
| 119_7M59_day5_ValpA | 0.894 | 0.992 | 0.877 | M | Normal |
| 71_1M4_day5_Saline | 1.141 | 0.796 | 0.887 | M | Normal |
| 111_1F109_day28_Saline | 0.864 | 1.01 | 1.061 | F | Normal |
| 60_8M82_day28_Naive | 2.082 | 1.127 | 1.021 | M | Normal |
| 129_7F163_day28_ValpA | 0.478 | 0.761 | 0.77 | F | Normal |
| 69_1F102_day1_Saline | 0.5 | 0.819 | 0.476 | F | Normal |
| 108_1M9_day28_Saline | 1.037 | 0.889 | 0.829 | M | Normal |
| 46_2F114_day5_Gent | 1.427 | 1.441 | 1.778 | F | Toxic |
| 125_7M62_day28_ValpA | 0.991 | 1.208 | 0.719 | M | Normal |
| 113_7M56_day1_ValpA | 1.092 | 0.818 | 1.098 | M | Normal |
| 45_2F113_day5_Gent | 1.403 | 1.395 | 1.636 | F | Toxic |
| 61_8M83_day28_Naive | 0.965 | 0.736 | 0.548 | M | Normal |
| 53_4F133_day5_Tob | 1.785 | 0.942 | 2.402 | F | Toxic |
| 107_1M8_day28_Saline | 0.596 | 0.284 | 0.393 | M | Normal |
| 115_7F155_day1_ValpA | 1.596 | 1.366 | 1.522 | F | Normal |
| 58_6F151_day5_Dox | 2.561 | 1.399 | 1.18 | F | Toxic |
| 31_2M13_day5_Gent | 1.53 | 0.76 | 1.256 | M | Toxic |
| 49_3F123_day5_Cis | 1.265 | 1.265 | 1.09 | F | Toxic |
| 39_4M33_day5_Tob | 1.447 | 1.117 | 1.224 | M | Toxic |
| 70_1F103_day1_Saline | 0.876 | 1.169 | 0.753 | F | Normal |
| 66_1M2_day1_Saline | 0.897 | 0.674 | 0.582 | M | Normal |
| 64_8F183_day28_Naive | 0.683 | 1.224 | 0.868 | F | Normal |
| 54_UK5F126_T1_Ds2_Day1 | 0.944 | 1.186 | 0.483 | F | |
| 123_UK4F119_T1_Ds1_Day5 | 0.571 | 0.795 | 0.692 | F | |
| 60_UK7F139_T2_Ds1_Day1 | 1.863 | 0.305 | 1.308 | F | |
| 17_UK5M29_T1_Ds2_Day1 | 1.213 | 0.984 | 0.759 | M | |
| 117_UK2F107_control_Day5 | 4.213 | 0.933 | 3.974 | F | |
| 109_UK12M69_T3_Ds1_Day5 | 0.852 | 0.754 | 0.615 | M | |
| 18_UK5M30_T1_Ds2_Day1 | 0.661 | 0.34 | 0.448 | M | |
| 57_UK5F129_T1_Ds2_Day1 | 0.559 | 0.689 | 0.547 | F | |
| 151_UK14F180_T3_Ds2_Day5 | 0.368 | 0.729 | 0.37 | F | |
| 7_UK3M13_T1_Ds1_Day1 | 1.118 | 0.216 | 0.843 | M | |
| 113_UK14M80_T3_Ds2_Day5 | 0.779 | 0.728 | 1.451 | M | |
| 72_UK11F164_T3_Ds1_Day1 | 0.483 | 0.322 | 0.354 | F | |
| 104_UK10M56_T2_Ds2_Day5 | 0.731 | 0.601 | 0.609 | M | |
| 91_UK6M31_T1_Ds2_Day5 | 0.803 | 1.092 | 0.797 | M | |
| 124_UK4F120_T1_Ds1_Day5 | 0.528 | 0.885 | 0.627 | F | |
| 114_UK14M81_T3_Ds2_Day5 | 0.656 | 0.933 | 0.53 | M | |
| 69_UK9F154_T2_Ds2_Day1 | 0.804 | 0.201 | 1.43 | F | |
| 46_UK1F105_control_Day1 | 0.734 | 0.542 | 0.825 | F | |
| 34_UK11M64_T3_Ds1_Day1 | 1.31 | 0.663 | 1.103 | M | |
| 130_UK6F132_T1_Ds2_Day5 | 0.863 | 1.125 | 0.766 | F | |
| 3_UK1M3_control_Day1 | 0.883 | 0.267 | 1.049 | M | |
| 75_UK13F173_T3_Ds2_Day1 | 0.659 | 0.67 | 0.444 | F | |
| 101_UK8M47_T2_Ds1_Day5 | 0.929 | 0.248 | 1.016 | M | |
| 102_UK8M48_T2_Ds1_Day5 | 0.996 | 0.203 | 1.11 | M | |
| 96_UK6M36_T1_Ds2_Day5 | 0.982 | 0.885 | 1.01 | M | |
| 39_UK13M76_T3_Ds2_Day1 | 1.566 | 0.137 | 1.146 | M | |
| 19_UK7M37_T2_Ds1_Day1 | 0.632 | 0.666 | 0.744 | M | |
| 144_UK12F167_T3_Ds1_Day5 | 1.176 | 1.146 | 1.082 | F | |
| 2_UK1M2_control_Day1 | 0.91 | 0.216 | 0.885 | M | |
| 52_UK3F118_T1_Ds1_Day1 | 0.683 | 0.843 | 0.636 | F | |
| 31_UK11M61_T3_Ds1_Day1 | 1.296 | 0.497 | 0.678 | M | |
| 129_UK6F131_T1_Ds2_Day5 | 0.554 | 0.55 | 0.629 | F | |
| 23_UK7M41_T2_Ds1_Day1 | 1.305 | 0.84 | 1.159 | M | |
| 43_UK1F102_control_Day1 | 1.43 | 0.612 | 0.851 | F | |
| 81_UK2M8_control_Day5 | 1.317 | 0.25 | 2.054 | M | |
| 80_UK2M7_control_Day5 | 2.363 | 0.112 | 1.078 | M | |
| 70_UK11F161_T3_Ds1_Day1 | 0.52 | 0.402 | 0.596 | F | |
| 16_UK5M28_T1_Ds2_Day1 | 0.462 | 0.56 | 0.396 | M | |
| 79_UK13F178_T3_Ds2_Day1 | 0.602 | 0.761 | 0.527 | F | |
| 50_UK3F116_T1_Ds1_Day1 | 0.49 | 0.926 | 0.572 | F | |
| 112_UK12M72_T3_Ds1_Day5 | 1.034 | 1.388 | 1.102 | M | |
| 146_UK12F169_T3_Ds1_Day5 | 0.822 | 0.659 | 1.032 | F | |
| 140_UK10F156_T2_Ds2_Day5 | 1.178 | 0.555 | 1.842 | F | |
| 143_UK10F160_T2_Ds2_Day5 | 0.485 | 0.354 | 0.637 | F | |
| 10_UK3M16_T1_Ds1_Day1 | 0.778 | 0.155 | 0.574 | M | |
| 94_UK6M34_T1_Ds2_Day5 | 0.527 | 0.654 | 0.539 | M | |
| 147_UK12F170_T3_Ds1_Day5 | 0.479 | 0.701 | 0.501 | F | |
| 71_UK11F163_T3_Ds1_Day1 | 0.859 | 0.394 | 0.85 | F | |
| 122_UK2F112_control_Day5 | 1.05 | 0.522 | 1.214 | F | |
| 47_UK1F106_control_Day1 | 0.495 | 0.276 | 0.605 | F | |
| 15_UK5M27_T1_Ds2_Day1 | 1.326 | 0.889 | 0.932 | M | |
| 97_UK8M43_T2_Ds1_Day5 | 1.445 | 0.11 | 1.566 | M | |
| 106_UK10M59_T2_Ds2_Day5 | 0.899 | 0.615 | 1.338 | M | |
| 118_UK2F108_control_Day5 | 1.325 | 0.362 | 1.597 | F | |
| 148_UK12F171_T3_Ds1_Day5 | 0.546 | 1.155 | 0.727 | F | |
| 35_UK11M65_T3_Ds1_Day1 | 0.567 | 0.898 | 0.918 | M | |
| 21_UK7M39_T2_Ds1_Day1 | 0.801 | 0.955 | 0.98 | M | |
| 38_UK13M75_T3_Ds2_Day1 | 2.291 | 0.276 | 1.479 | M | |
| 55_UK5F127_T1_Ds2_Day1 | 0.898 | 0.861 | 1.067 | F | |
| 100_UK8M46_T2_Ds1_Day5 | 1.42 | 0.209 | 1.449 | M | |
| 67_UK9F152_T2_Ds2_Day1 | 1.104 | 0.289 | 2.008 | F | |
| 131_UK6F133_T1_Ds2_Day5 | 0.989 | 1.025 | 0.935 | F | |
| 142_UK10F159_T2_Ds2_Day5 | 0.554 | 0.269 | 0.799 | F | |
| 128_UK4F124_T1_Ds1_Day5 | 0.745 | 1.511 | 1.162 | F | |
| 95_UK6M35_T1_Ds2_Day5 | 1.181 | 1.077 | 1.161 | M | |
| 8_UK3M14_T1_Ds1_Day1 | 1.501 | 0.178 | 1.13 | M | |
| 138_UK8F147_T2_Ds1_Day5 | 0.464 | 0.865 | 0.771 | F | |
| 41_UK13M78_T3_Ds2_Day1 | 1.042 | 0.164 | 0.566 | M | |
| 76_UK13F175_T3_Ds2_Day1 | 0.847 | 0.874 | 1.132 | F | |
| 152_UK14F181_T3_Ds2_Day5 | 0.587 | 0.81 | 0.666 | F | |
| 86_UK4M20_T1_Ds1_Day5 | 1.835 | 0.125 | 2.031 | M | |
| 59_UK7F137_T2_Ds1_Day1 | 0.492 | 0.183 | 0.54 | F | |
| 111_UK12M71_T3_Ds1_Day5 | 0.88 | 1.054 | 1.285 | M | |
| 145_UK12F168_T3_Ds1_Day5 | 0.601 | 0.791 | 0.928 | F | |
| 28_UK9M52_T2_Ds2_Day1 | 1.462 | 0.773 | 1.006 | M | |
| 108_UK12M67_T3_Ds1_Day5 | 0.8 | 0.818 | 1.133 | M | |
| 11_UK3M17_T1_Ds1_Day1 | 0.981 | 0.143 | 0.805 | M | |
| 24_UK7M42_T2_Ds1_Day1 | 0.791 | 0.798 | 0.709 | M | |
| 42_UK1F101_control_Day1 | 0.569 | 0.323 | 0.674 | F | |
| 29_UK9M53_T2_Ds2_Day1 | 0.692 | 0.595 | 0.729 | M | |
| 61_UK7F140_T2_Ds1_Day1 | 0.468 | 0.213 | 0.419 | F | |
| 20_UK7M38_T2_Ds1_Day1 | 1.076 | 0.931 | 1.059 | M | |
| 141_UK10F158_T2_Ds2_Day5 | 1.029 | 0.354 | 3.003 | F | |
| 87_UK4M21_T1_Ds1_Day5 | 0.991 | 0.208 | 0.904 | M | |
| 150_UK14F179_T3_Ds2_Day5 | 0.623 | 0.711 | 0.977 | F | |
| 136_UK8F144_T2_Ds1_Day5 | 0.532 | 0.922 | 1.02 | F | |
| 93_UK6M33_T1_Ds2_Day5 | 2.203 | 1.236 | 1.793 | M | |
| 85_UK4M19_T1_Ds1_Day5 | 0.687 | 0.165 | 0.937 | M | |
| 4_UK1M4_control_Day1 | 1.352 | 0.235 | 1.318 | M | |
| 126_UK4F122_T1_Ds1_Day5 | 0.945 | 1.328 | 1.367 | F | |
| 36_UK11M66_T3_Ds1_Day1 | 1.106 | 0.512 | 0.845 | M | |
| 74_UK11F166_T3_Ds1_Day1 | 1.064 | 0.356 | 1.252 | F | |
| 14_UK5M26_T1_Ds2_Day1 | 0.785 | 0.656 | 0.544 | M | |
| 119_UK2F109_control_Day5 | 0.907 | 0.515 | 1.067 | F | |
| 25_UK9M49_T2_Ds2_Day1 | 0.656 | 0.741 | 0.541 | M | |
| 103_UK10M55_T2_Ds2_Day5 | 1.446 | 1.022 | 0.831 | M | |
| 27_UK9M51_T2_Ds2_Day1 | 1.23 | 0.777 | 1.015 | M | |
| 26_UK9M50_T2_Ds2_Day1 | 0.866 | 0.725 | 0.588 | M | |
| 44_UK1F103_control_Day1 | 0.524 | 0.2 | 0.503 | F | |
| 62_UK7F141_T2_Ds1_Day1 | 1.072 | 0.29 | 0.836 | F | |
| 92_UK6M32_T1_Ds2_Day5 | 0.784 | 0.596 | 0.73 | M | |
| 12_UK3M18_T1_Ds1_Day1 | 0.439 | 0.169 | 0.4 | M | |
| 5_UK1M5_control_Day1 | 1.411 | 0.299 | 1.289 | M | |
| 153_UK14F182_T3_Ds2_Day5 | 1.24 | 1.599 | 1.436 | F | |
| 56_UK5F128_T1_Ds2_Day1 | 0.415 | 0.549 | 0.514 | F | |
| 58_UK5F130_T1_Ds2_Day1 | 0.502 | 0.725 | 0.639 | F | |
| 132_UK6F134_T1_Ds2_Day5 | 1.223 | 0.454 | 1.478 | F | |
| 49_UK3F114_T1_Ds1_Day1 | 0.752 | 0.825 | 0.49 | F | |
| 90_UK4M24_T1_Ds1_Day5 | 0.902 | 0.207 | 0.783 | M | |
| 64_UK9F149_T2_Ds2_Day1 | 0.741 | 0.365 | 0.857 | F | |
| 110_UK12M70_T3_Ds1_Day5 | 0.499 | 0.72 | 1.175 | M | |
| 127_UK4F123_T1_Ds1_Day5 | 0.938 | 1.593 | 1.089 | F | |
| 121_UK2F111_control_Day5 | 0.609 | 0.276 | 0.702 | F | |
| 83_UK2M10_control_Day5 | 1.034 | 0.412 | 1.069 | M | |
| 98_UK8M44_T2_Ds1_Day5 | 1.258 | 0.27 | 1.516 | M | |
| 99_UK8M45_T2_Ds1_Day5 | 1.447 | 0.299 | 1.237 | M | |
| 107_UK10M60_T2_Ds2_Day5 | 0.623 | 0.962 | 0.83 | M | |
| 73_UK11F165_T3_Ds1_Day1 | 0.507 | 0.205 | 0.37 | F | |
| 40_UK13M77_T3_Ds2_Day1 | 1.597 | 0.218 | 1.063 | M | |
| 89_UK4M23_T1_Ds1_Day5 | 1.35 | 0.2 | 1.364 | M | |
| 134_UK6F136_T1_Ds2_Day5 | 0.652 | 1.074 | 0.868 | F | |
| 133_UK6F135_T1_Ds2_Day5 | 1.056 | 1.674 | 1.487 | F | |
| 1_UK1M1_control_Day1 | 0.783 | 0.17 | 0.633 | M | |
| 30_UK9M54_T2_Ds2_Day1 | 0.837 | 0.614 | 0.471 | M | |
| 82_UK2M9_control_Day5 | 2.621 | 0.25 | 1.879 | M | |
| 37_UK13M73_T3_Ds2_Day1 | 1.35 | 0.211 | 1.117 | M | |
| 45_UK1F104_control_Day1 | 0.897 | 0.381 | 1.062 | F | |
| 105_UK10M57_T2_Ds2_Day5 | 1.049 | 0.626 | 0.878 | M | |
| 135_UK8F143_T2_Ds1_Day5 | 0.616 | 0.961 | 1.923 | F | |
| 33_UK11M63_T3_Ds1_Day1 | 1.844 | 0.989 | 1.025 | M | |
| 77_UK13F176_T3_Ds2_Day1 | 1.043 | 0.929 | 1.211 | F | |
| 63_UK7F142_T2_Ds1_Day1 | 0.969 | 0.387 | 0.49 | F | |
| 48_UK3F113_T1_Ds1_Day1 | 0.548 | 0.917 | 0.762 | F | |
| 65_UK9F150_T2_Ds2_Day1 | 1.73 | 0.32 | 3.044 | F | |
| 84_UK2M11_control_Day5 | 0.629 | 0.162 | 0.645 | M | |
| 125_UK4F121_T1_Ds1_Day5 | 0.606 | 0.893 | 0.831 | F | |
| 139_UK8F148_T2_Ds1_Day5 | 0.572 | 0.824 | 1.232 | F | |
| 9_UK3M15_T1_Ds1_Day1 | 1.502 | 0.245 | 1.416 | M | |
| 115_UK14M82_T3_Ds2_Day5 | 0.494 | 1.103 | 0.765 | M | |
| 137_UK8F145_T2_Ds1_Day5 | 0.773 | 1.303 | 0.94 | F | |
| 13_UK5M25_T1_Ds2_Day1 | 0.592 | 0.473 | 0.541 | M | |
| 66_UK9F151_T2_Ds2_Day1 | 1.211 | 0.444 | 2.152 | F | |
| 88_UK4M22_T1_Ds1_Day5 | 0.684 | 0.323 | 0.717 | M | |
| 149_UK12F172_T3_Ds1_Day5 | 0.677 | 1.096 | 1.021 | F | |
| 32_UK11M62_T3_Ds1_Day1 | 0.808 | 0.789 | 0.713 | M | |
| 68_UK9F153_T2_Ds2_Day1 | 0.666 | 0.254 | 0.71 | F | |
| 22_UK7M40_T2_Ds1_Day1 | 1.477 | 1.182 | 1.253 | M | |
| 6_UK1M6_control_Day1 | 0.749 | 0.127 | 0.476 | M | |
| 120_UK2F110_control_Day5 | 0.898 | 0.34 | 0.976 | F | |
| 78_UK13F177_T3_Ds2_Day1 | 1.227 | 1.454 | 0.575 | F | |
| 53_UK5F125_T1_Ds2_Day1 | 1.028 | 1.045 | 0.965 | F | |
| 116_UK14M83_T3_Ds2_Day5 | 3.181 | 0.911 | 1.037 | M | |
| 51_UK3F117_T1_Ds1_Day1 | 1.047 | 1.186 | 0.981 | F | |
| 154_UK14F183_T3_Ds2_Day5 | 0.704 | 1.018 | 0.58 | F | |
| 16_2F110_day1_Gent | 0.763 | 0.963 | 1.251 | F | |
| 42_5M42_day5_CadCl | 1.337 | 1.104 | 1.083 | M | |
| 102_5F145_day28_CadCl | 0.414 | 0.259 | 0.728 | F | |
| 9_4M30_day1_Tob | 1.43 | 1.126 | 2.04 | M | |
| 97_4F134_day28_Tob | 1.073 | 0.876 | 0.959 | F | |
| 105_6F173_day28_Dox | 2.157 | 0.734 | 0.964 | F | |
| 79_3M25_day28_Cis | 5.917 | 1.587 | 4.504 | M | |
| 78_2M18_day28_Gent | 1.55 | 0.852 | 1.734 | M | |
| 54_5F141_day5_CadCl | 2.282 | 1.782 | 1.866 | F | |
| 5_3M20_day1_Cis | 0.815 | 0.799 | 0.495 | M | |
| 77_2M17_day28_Gent | 1.353 | 0.857 | 3.06 | M | |
| 82_4M34_day28_Tob | 0.61 | 0.39 | 0.679 | M | |
| 12_5M39_day1_CadCl | 1.499 | 1.034 | 0.951 | M | |
| 26_5F138_day1_CadCl | 1.059 | 1.194 | 1.133 | F | |
| 94_3F125_day28_Cis | 2.504 | 1.749 | 2.301 | F | |
| 93_2F118_day28_Gent | 0.806 | 0.421 | 0.967 | F | |
| 17_2F111_day1_Gent | 0.963 | 0.98 | 0.889 | F | |
| 92_2F117_day28_Gent | 1.079 | 0.441 | 2.632 | F | |
| 89_6M72_day28_Dox | 1.613 | 0.686 | 0.669 | M | |
| 55_5F142_day5_CadCl | 0.854 | 0.906 | 0.805 | F | |
| 15_6M48_day1_Dox | 1.26 | 0.956 | 1.257 | M | |
| 3_2M12_day1_Gent | 1.567 | 1.02 | 1.072 | M | |
| 84_4M36_day28_Tob | 1.379 | 0.832 | 1.273 | M | |
| 87_5M45_day28_CadCl | 0.781 | 0.531 | 0.672 | M | |
| 25_5F137_day1_CadCl | 1.155 | 1.772 | 1.64 | F | |
| 98_4F135_day28_Tob | 1.144 | 0.621 | 1.021 | F | |
| 41_5M41_day5_CadCl | 1.662 | 1.157 | 1.409 | M | |
| 83_4M35_day28_Tob | 1.281 | 0.803 | 1.397 | M | |
| 2_2M11_day1_Gent | 1.869 | 1.409 | 1.848 | M | |
| 13_6M46_day1_Dox | 2.478 | 0.936 | 1.622 | M | |
| 24_4F130_day1_Tob | 1.5 | 1.153 | 5.307 | F | |
| 101_5F144_day28_CadCl | 0.827 | 0.783 | 0.975 | F | |
| 21_3F121_day1_Cis | 0.81 | 0.844 | 0.631 | F | |
| 88_6M71_day28_Dox | 3.226 | 0.442 | 1.434 | M | |
| 100_5F143_day28_CadCl | 1.098 | 1.033 | 1.58 | F | |
| 10_5M37_day1_CadCl | 1.622 | 1.027 | 1.108 | M | |
| 7_4M28_day1_Tob | 1.083 | 0.999 | 0.825 | M | |
| 18_2F112_day1_Gent | 1.408 | 0.898 | 2.053 | F | |
| 28_6F146_day1_Dox | 1.548 | 2.054 | 1.631 | F | |
| 8_4M29_day1_Tob | 1.619 | 1.049 | 1.223 | M | |
| 91_2F116_day28_Gent | 1.164 | 0.434 | 1.78 | F | |
| 22_4F128_day1_Tob | 0.73 | 0.799 | 1.195 | F | |
| 86_5M44_day28_CadCl | 1.107 | 0.817 | 0.836 | M | |
| 14_6M47_day1_Dox | 1.45 | 0.964 | 1.454 | M | |
| 40_5M40_day5_CadCl | 1.675 | 1.671 | 2.393 | M | |
| 96_3F127_day28_Cis | 2.643 | 0.786 | 1.555 | F | |
| 30_6F148_day1_Dox | 1.367 | 1.752 | 1.268 | F | |
| 103_6F171_day28_Dox | 1.462 | 0.719 | 0.653 | F | |
| 19_3F119_day1_Cis | 1.261 | 1.209 | 0.788 | F | |
| 85_5M43_day28_CadCl | 0.807 | 0.633 | 0.333 | M | |
| 80_3M26_day28_Cis | 3.371 | 0.786 | 1.428 | M | |
| 27_5F139_day1_CadCl | 1.372 | 1.894 | 1.263 | F | |
| 76_2M16_day28_Gent | 1.245 | 0.756 | 1.469 | M | |
| 90_6M73_day28_Dox | 1.604 | 0.586 | 0.785 | M | |
| 29_6F147_day1_Dox | 0.977 | 1.003 | 0.741 | F | |
| 23_4F129_day1_Tob | 1.166 | 0.982 | 1.623 | F | |
| 1_2M10_day1_Gent | 1.48 | 1.161 | 1.221 | M | |
| 20_3F120_day1_Cis | 1.096 | 1.345 | 0.899 | F | |
| 104_6F172_day28_Dox | 1.023 | 0.955 | 0.54 | F | |
| 6_3M21_day1_Cis | 2.455 | 1.09 | 1.64 | M | |
| 95_3F126_day28_Cis | 5.783 | 1.864 | 3.336 | F | |
| 81_3M27_day28_Cis | 3.008 | 1.371 | 1.753 | M | |
| 99_4F136_day28_Tob | 1.118 | 0.987 | 1.478 | F | |
| 4_3M19_day1_Cis | 1.176 | 0.902 | 0.811 | M | |
| 11_5M38_day1_CadCl | 1.512 | 0.957 | 1.069 | M | |

Experimental Design

Three compounds (T1, T2, T3) and a control were administered to male and female rats at various time points by oral gavage: 3 treatments (T1, T2, T3) were given in 2 doses (Ds1, Ds2) for two periods (one day and 5 days). Each group consisted of 6 male and 6 female rats. The additional groups consisted of 6 males and 6 female rats treated with control compound for a single day and for five days, as described in Table 24.

TABLE 24
Un-labeled samples groups and doses

| | | | | | Animals |
|---|---|---|---|---|---|
| | | Dose | Route of | Days of | per |
| Groups | Test Items | (mg/kg) | administration | treatment | group |
| 1 M, F | Control - | — | Oral gavage | 1 | 6 |
| 2 M, F | 1% methyl- | — | Oral gavage | 5 | 6 |
| | cellulose | | | | |
| 3 M, F | T1 | 10 | Oral gavage | 1 | 6 |
| 4 M, F | | 10 | Oral gavage | 5 | 6 |
| 5 M, F | | 40 | Oral gavage | 1 | 6 |
| 6 M, F | | 40 | Oral gavage | 5 | 6 |
| 7 M, F | T2 | 25 | Oral gavage | 1 | 6 |
| 8 M, F | | 25 | Oral gavage | 5 | 6 |
| 9 M, F | | 50 | Oral gavage | 1 | 6 |
| 10 M, F | | 50 | Oral gavage | 5 | 6 |
| 11 M, F | T3 | 5 | Oral gavage | 1 | 6 |
| 12 M, F | | 5 | Oral gavage | 5 | 6 |
| 13 M, F | | 10 | Oral gavage | 1 | 6 |
| 14 M, F | | 10 | Oral gavage | 5 | 6 |

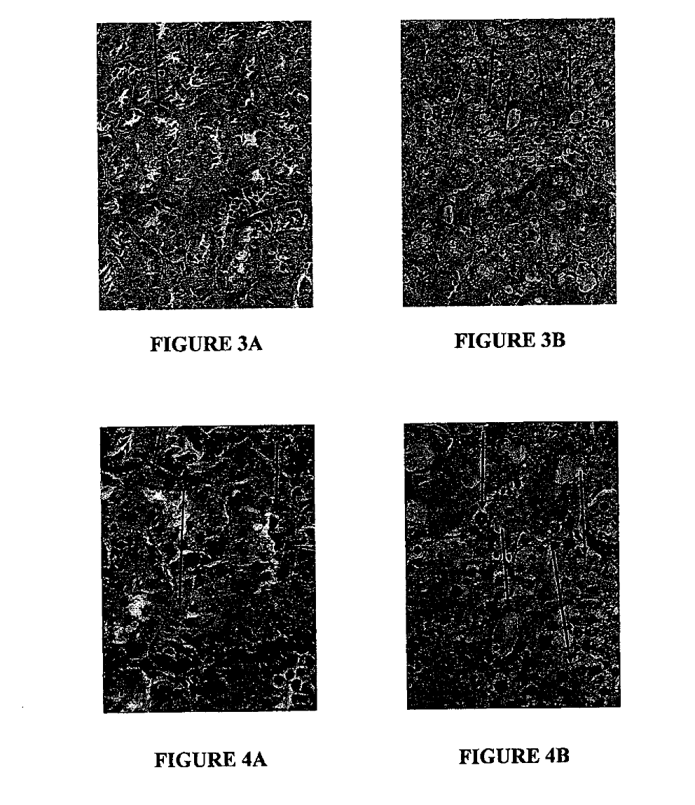

T2 is a drug known to cause renal damage after 7 days of treatment at the 50 mg/kg dose. The chemical structure of T2 is shown in FIG. 2. T2 “no observed effect level” (NOEL, i.e., the maximal dose at which no toxic effect is visible) is 15 mg/kg. Kidneys obtained from rats receiving 50 mg/kg of T2, demonstrated direct cortical tubular toxicity. FIGS. 3-5 demonstrate the results of histopathological examination of the treated kidneys after 7 days (FIGS. 3B, 4B and 5B), or of the control animals (FIGS. 3A, 4A and 5A). As demonstrated in FIGS. 3B, 4B and 5B the changes were multifocal, consisting of tubular basophilia, indicating regeneration following necrosis.

FIG. 3 demonstrates histological section of the kidney, magnified ×200. FIG. 3A represents a histological section of the kidney of a control animal. Normal aspects of cortical tubules (eosinophilic cytoplasm) are marked by arrows. FIG. 3B represents a histological section of the kidney of a 50 mg/kg treated animal. Basophilic tubules in the Cortex, indicating post-necrotic regeneration are marked by arrows.

FIG. 4 demonstrates histological section of the kidney, magnified ×400. FIG. 4A represents a histological section of the kidney of a control animal. Normal aspects of cortical tubules (eosinophilic cytoplasm) are marked by arrows. FIG. 4B represents a histological section of the kidney of a 50 mg/kg treated animal. Basophilic tubules in the Cortex, indicating post-necrotic regeneration are marked by arrows.

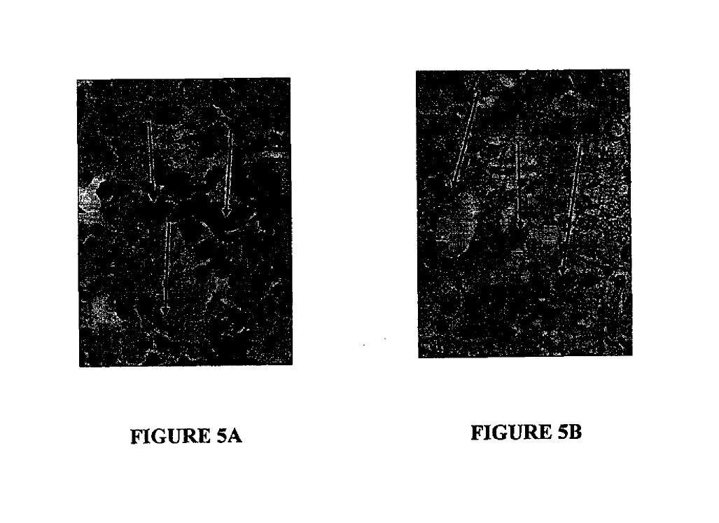

FIG. 5 demonstrates histological section of the kidney, magnified ×600. FIG. 5A represents a histological section of the kidney of a control animal. Normal aspects of cortical tubules (eosinophilic cytoplasm) are marked by arrows. FIG. 5B represents a histological section of the kidney of a 50 mg/kg treated animal. Basophilic tubules in the Cortex, indicating post-necrotic regeneration are marked by arrows.

T1 is another drug which shows renal damage, mainly in females, after 28 days of treatment at the 30 mg/kg dose. The chemical structure of T1 is shown in FIG. 1.

T3 is Riluzole (6-(trifluoromethoxy)benzothiazol-2-amine), which shows no renal damage after up to 35 days of treatment, and therefore was used as a negative control.

At autopsy, both kidneys of each animal were removed and each kidney was cross sectioned. Half of each kidney was sent to RNA extraction and analysis.

RNA Isolation from Kidney Tissues

RNA was isolated as described previously. RNA samples which did not meet the conventional RNA quality criteria including A260: A280 ratio, A260:A230 ratio; and RNA integrity as detected in agarose gel, were omitted from further processing.

RT Preparation and Real-Time qRT-PCR Analysis

RT was prepared and qRT-PCR performed and analyzed as described hereinabove. qRT-PCR was performed for the four genes comprising the signature of the discovery stage described in Table 13. Two amplicons were tested for each gene—one representing the wild-type transcript, and another representing an alternative splice variant. Each amplicon was measured using the labeled samples described in Table 9 herein, as well as the un-labeled samples. Samples from both groups (labeled and un-labeled) were tested in seven 96 well plates as described in Table 25. In order to correct possible run to run variations, the following controls were added: 1. several samples were repeated in all 96 wells plates; 2. control shuffled plate was designed which combined samples from all seven test plates.

Table 25 presents plates set up in a validation stage. The names of the un-labeled samples comprise of the rat's group, the rats ID number starts with UK (stand for unknown), the toxicant it was exposed to (T1, T2 or T3), the dose given Ds1 or Ds2 and day of treatment.

TABLE 25
Plates set up in the validation stage
Plate 1

| | | | | |
|---|---|---|---|---|
| 1 | Gentamycin | 1 | day 1 toxicants M | 1_2M10_day1_Gent |
| 2 | Gentamycin | 1 | | 2_2M11_day1_Gent |
| 3 | Gentamycin | 1 | | 3_2M12_day1_Gent |
| 4 | Cisplatin | 1 | | 4_3M19_day1_Cis |
| 5 | Cisplatin | 1 | | 5_3M20_day1_Cis |
| 6 | Cisplatin | 1 | | 6_3M21_day1_Cis |
| 7 | Tobramycin | 1 | | 7_4M28_day1_Tob |
| 8 | Tobramycin | 1 | | 8_4M29_day1_Tob |
| 9 | Tobramycin | 1 | | 9_4M30_day1_Tob |
| 10 | Cadmium | 1 | | 10_5M37_day1_CadCl |
| | chloride | | | |
| 11 | Cadmium | 1 | | 11_5M38_day1_CadCl |
| | chloride | | | |
| 12 | Cadmium | 1 | | 12_5M39_day1_CadCl |
| | chloride | | | |
| 13 | Doxorubicin | 1 | | 13_6M46_day1_Dox |
| 14 | Doxorubicin | 1 | | 14_6M47_day1_Dox |
| 15 | Doxorubicin | 1 | | 15_6M48_day1_Dox |
| 16 | Valproic acid | 1 | | 112_7M55_day1_ValpA |
| 17 | Valproic acid | 1 | | 113_7M56_day1_ValpA |
| 18 | Valproic acid | 1 | | 114_7M57_day1_ValpA |
| 19 | Gentamycin | 1 | day 1 toxicants F | 16_2F110_day1_Gent |
| 20 | Gentamycin | 1 | | 17_2F111_day1_Gent |
| 21 | Gentamycin | 1 | | 18_2F112_day1_Gent |
| 22 | Cisplatin | 1 | | 19_3F119_day1_Cis |
| 23 | Cisplatin | 1 | | 20_3F120_day1_Cis |
| 24 | Cisplatin | 1 | | 21_3F121_day1_Cis |
| 25 | Tobramycin | 1 | | 22_4F128_day1_Tob |
| 26 | Tobramycin | 1 | | 23_4F129_day1_Tob |
| 27 | Tobramycin | 1 | | 24_4F130_day1_Tob |
| 28 | Cadmium | 1 | | 25_5F137_day1_CadCl |
| | chloride | | | |
| 29 | Cadmium | 1 | | 26_5F138_day1_CadCl |
| | chloride | | | |
| 30 | Cadmium | 1 | | 27_5F139_day1_CadCl |
| | chloride | | | |
| 31 | Doxorubicin | 1 | | 28_6F146_day1_Dox |
| 32 | Doxorubicin | 1 | | 29_6F147_day1_Dox |
| 33 | Doxorubicin | 1 | | 30_6F148_day1_Dox |
| 34 | Valproic acid | 1 | | 115_7F155_day1_ValpA |
| 35 | Valproic acid | 1 | | 116_7F156_day1_ValpA |
| 36 | Valproic acid | 1 | | 117_7F157_day1_ValpA |
| 37 | Naïve | 28 | Controls All days | 59_8M81_day28_Naïve |
| 38 | Naïve | 28 | | 60_8M82_day28_Naïve |
| 39 | Naïve | 28 | | 61_8M83_day28_Naïve |
| 40 | Naïve | 28 | | 62_8F181_day28_Naïve |
| 41 | Naïve | 28 | | 63_8F182_day28_Naïve |
| 42 | Naïve | 28 | | 64_8F183_day28_Naïve |
| 43 | Control/Saline | 1 | | 65_1M1_day1_Saline |
| 44 | Control/Saline | 1 | | 66_1M2_day1_Saline |
| 45 | Control/Saline | 1 | | 67_1M3_day1_Saline |
| 46 | Control/Saline | 1 | | 68_1F101_day1_Saline |
| 47 | Control/Saline | 1 | | 69_1F102_day1_Saline |
| 48 | Control/Saline | 1 | | 70_1F103_day1_Saline |

Plate 2

| | | | | |
|---|---|---|---|---|
| 1 | Gentamycin | 5 | day 5 toxicants M | 31_2M13_day5_Gent |
| 2 | Gentamycin | 5 | | 32_2M14_day5_Gent |
| 3 | Gentamycin | 5 | | 33_2M15_day5_Gent |
| 4 | Cisplatin | 5 | | 34_3M22_day5_Cis |
| 5 | Cisplatin | 5 | | 35_3M23_day5_Cis |
| 6 | Cisplatin | 5 | | 36_3M24_day5_Cis |
| 7 | Tobramycin | 5 | | 37_4M31_day5_Tob |
| 8 | Tobramycin | 5 | | 38_4M32_day5_Tob |
| 9 | Tobramycin | 5 | | 39_4M33_day5_Tob |
| 10 | Cadmium | 5 | | 40_5M40_day5_CadCl |
| | chloride | | | |
| 11 | Cadmium | 5 | | 41_5M41_day5_CadCl |
| | chloride | | | |
| 12 | Cadmium | 5 | | 42_5M42_day5_CadCl |
| | chloride | | | |
| 13 | Doxorubicin | 5 | | 43_6M49_day5_Dox |
| 14 | Doxorubicin | 5 | | 44_6M50_day5_Dox |
| 15 | Valproic acid | 5 | | 118_7M58_day5_ValpA |
| 16 | Valproic acid | 5 | | 119_7M59_day5_ValpA |
| 17 | Valproic acid | 5 | | 120_7M60_day5_ValpA |
| 18 | Gentamycin | 5 | day 5 toxicants F | 45_2F113_day5_Gent |
| 19 | Gentamycin | 5 | | 46_2F114_day5_Gent |
| 20 | Gentamycin | 5 | | 47_2F115_day5_Gent |
| 21 | Cisplatin | 5 | | 48_3F122_day5_Cis |
| 22 | Cisplatin | 5 | | 49_3F123_day5_Cis |
| 23 | Cisplatin | 5 | | 50_3F124_day5_Cis |
| 24 | Tobramycin | 5 | | 51_4F131_day5_Tob |
| 25 | Tobramycin | 5 | | 52_4F132_day5_Tob |
| 26 | Tobramycin | 5 | | 53_4F133_day5_Tob |
| 27 | Cadmium | 5 | | 54_5F141_day5_CadCl |
| | chloride | | | |
| 28 | Cadmium | 5 | | 55_5F142_day5_CadCl |
| | chloride | | | |
| 29 | Doxorubicin | 5 | | 56_6F149_day5_Dox |
| 30 | Doxorubicin | 5 | | 57_6F150_day5_Dox |
| 31 | Doxorubicin | 5 | | 58_6F151_day5_Dox |
| 32 | Valproic acid | 5 | | 121_7F158_day5_ValpA |
| 33 | Valproic acid | 5 | | 122_7F159_day5_ValpA |
| 34 | Valproic acid | 5 | | 123_7F160_day5_ValpA |
| 35 | Control/Saline | 5 | | 71_1M4_day5_Saline |
| 36 | Control/Saline | 5 | | 72_1M5_day5_Saline |
| 37 | Control/Saline | 5 | | 73_1M6_day5_Saline |
| 38 | Control/Saline | 5 | | 74_1F104_day5_Saline |
| 39 | Control/Saline | 5 | | 75_1F105_day5_Saline |
| 40 | Amph. Control | 1 | Control | 130_1M6a_day1_Ctrl |
| 41 | Amph. Control | 5 | | 131_1M13a_day5_Ctrl |
| 42 | Amph. Control | 28 | | 132_1M20a_day28_Ctrl |
| 43 | Amph. Control | 1 | | 133_1F106a_day1_Ctrl |
| 44 | Amph. Control | 5 | | 134_1F113a_day5_Ctrl |
| 45 | Amph. Control | 28 | | 135_1F120a_day28_Ctrl |
| 46 | Amphotericin | 1 | Amphotericin | 136_6M61a_day1_Amp |
| 47 | Amphotericin | 1 | | 137_6M62a_day1_Amp |
| 48 | Amphotericin | 1 | | 138_6M63a_day1_Amp |
| 49 | Amphotericin | 5 | | 139_6M67a_day5_Amp |
| 50 | Amphotericin | 5 | | 140_6M68a_day5_Amp |
| 51 | Amphotericin | 5 | | 141_6M69a_day5_Amp |
| 52 | Amphotericin | 28 | | 142_6M73a_day28_Amp |
| 53 | Amphotericin | 28 | | 143_6M74a_day28_Amp |
| 54 | Amphotericin | 28 | | 144_6M75a_day28_Amp |
| 55 | Amphotericin | 1 | | 145_6F161a_day1_Amp |
| 56 | Amphotericin | 1 | | 146_6F162a_day1_Amp |
| 57 | Amphotericin | 1 | | 147_6F163a_day1_Amp |
| 58 | Amphotericin | 5 | | 148_6F167a_day5_Amp |
| 59 | Amphotericin | 5 | | 149_6F168a_day5_Amp |
| 60 | Amphotericin | 5 | | 150_6F169a_day5_Amp |
| 61 | Amphotericin | 28 | | 151_6F173a_day28_Amp |
| 62 | Amphotericin | 28 | | 152_6F174a_day28_Amp |
| 63 | Amphotericin | 28 | | 153_6F175a_day28_Amp |

Plate 3

| | | | | |
|---|---|---|---|---|
| 1 | Gentamycin | 28 | day 28 toxicants M | 76_2M16_day28_Gent |
| 2 | Gentamycin | 28 | | 77_2M17_day28_Gent |
| 3 | Gentamycin | 28 | | 78_2M18_day28_Gent |
| 4 | Cisplatin | 28 | | 79_3M25_day28_Cis |
| 5 | Cisplatin | 28 | | 80_3M26_day28_Cis |
| 6 | Cisplatin | 28 | | 81_3M27_day28_Cis |
| 7 | Tobramycin | 28 | | 82_4M34_day28_Tob |
| 8 | Tobramycin | 28 | | 83_4M35_day28_Tob |
| 9 | Tobramycin | 28 | | 84_4M36_day28_Tob |
| 10 | Cadmium | 28 | | 85_5M43_day28_CadCl |
| | chloride | | | |
| 11 | Cadmium | 28 | | 86_5M44_day28_CadCl |
| | chloride | | | |
| 12 | Cadmium | 28 | | 87_5M45_day28_CadCl |
| | chloride | | | |
| 13 | Doxorubicin | 28 | | 88_6M71_day28_Dox |
| 14 | Doxorubicin | 28 | | 89_6M72_day28_Dox |
| 15 | Doxorubicin | 28 | | 90_6M73_day28_Dox |
| 16 | Valproic acid | 28 | | 124_7M61_day28_ValpA |
| 17 | Valproic acid | 28 | | 125_7M62_day28_ValpA |
| 18 | Valproic acid | 28 | | 126_7M63_day28_ValpA |
| 19 | Gentamycin | 28 | day 28 toxicants F | 91_2F116_day28_Gent |
| 20 | Gentamycin | 28 | | 92_2F117_day28_Gent |
| 21 | Gentamycin | 28 | | 93_2F118_day28_Gent |
| 22 | Cisplatin | 28 | | 94_3F125_day28_Cis |
| 23 | Cisplatin | 28 | | 95_3F126_day28_Cis |
| 24 | Cisplatin | 28 | | 96_3F127_day28_Cis |
| 25 | Tobramycin | 28 | | 97_4F134_day28_Tob |
| 26 | Tobramycin | 28 | | 98_4F135_day28_Tob |
| 27 | Tobramycin | 28 | | 99_4F136_day28_Tob |
| 28 | Cadmium | 28 | | 100_5F143_day28_CadCl |
| | chloride | | | |
| 29 | Cadmium | 28 | | 101_5F144_day28_CadCl |
| | chloride | | | |
| 30 | Cadmium | 28 | | 102_5F145_day28_CadCl |
| | chloride | | | |
| 31 | Doxorubicin | 28 | | 103_6F171_day28_Dox |
| 32 | Doxorubicin | 28 | | 104_6F172_day28_Dox |
| 33 | Doxorubicin | 28 | | 105_6F173_day28_Dox |
| 34 | Valproic acid | 28 | | 127_7F161_day28_ValpA |
| 35 | Valproic acid | 28 | | 128_7F162_day28_ValpA |
| 36 | Valproic acid | 28 | | 129_7F163_day28_ValpA |
| 37 | Control/Saline | 28 | Day 28 control | 106_1M7_day28_Saline |
| 38 | Control/Saline | 28 | | 107_1M8_day28_Saline |
| 39 | Control/Saline | 28 | | 108_1M9_day28_Saline |
| 40 | Control/Saline | 28 | | 109_1F107_day28_Saline |
| 41 | Control/Saline | 28 | | 110_1F108_day28_Saline |
| 42 | Control/Saline | 28 | | 111_1F109_day28_Saline |

Plate 4

| | | | | |
|---|---|---|---|---|
| 1 | control | 1 | 1_UK1M1_control_Day1 | TT4: Male Day 1 |
| 2 | control | 1 | 2_UK1M2_control_Day1 | |
| 3 | control | 1 | 3_UK1M3_control_Day1 | |
| 4 | control | 1 | 4_UK1M4_control_Day1 | |
| 5 | control | 1 | 5_UK1M5_control_Day1 | |
| 6 | control | 1 | 6_UK1M6_control_Day1 | |
| 7 | T1 Ds1 | 1 | 7_UK3M13_T1 Ds1_Day1 | |
| 8 | T1 Ds1 | 1 | 8_UK3M14_T1 Ds1_Day1 | |
| 9 | T1 Ds1 | 1 | 9_UK3M15_T1 Ds1_Day1 | |
| 10 | T1 Ds1 | 1 | 10_UK3M16_T1 Ds1_Day1 | |
| 11 | T1 Ds1 | 1 | 11_UK3M17_T1 Ds1_Day1 | |
| 12 | T1 Ds1 | 1 | 12_UK3M18_T1 Ds1_Day1 | |
| 13 | T1 Ds2 | 1 | 13_UK5M25_T1 Ds2_Day1 | |
| 14 | T1 Ds2 | 1 | 14_UK5M26_T1 Ds2_Day1 | |
| 15 | T1 Ds2 | 1 | 15_UK5M27_T1 Ds2_Day1 | |
| 16 | T1 Ds2 | 1 | 16_UK5M28_T1 Ds2_Day1 | |
| 17 | T1 Ds2 | 1 | 17_UK5M29_T1 Ds2_Day1 | |
| 18 | T1 Ds2 | 1 | 18_UK5M30_T1 Ds2_Day1 | |
| 19 | T2 Ds1 | 1 | 19_UK7M37_T2 Ds1_Day1 | |
| 20 | T2 Ds1 | 1 | 20_UK7M38_T2 Ds1_Day1 | |
| 21 | T2 Ds1 | 1 | 21_UK7M39_T2 Ds1_Day1 | |
| 22 | T2 Ds1 | 1 | 22_UK7M40_T2 Ds1_Day1 | |
| 23 | T2 Ds1 | 1 | 23_UK7M41_T2 Ds1_Day1 | |
| 24 | T2 Ds1 | 1 | 24_UK7M42_T2 Ds1_Day1 | |
| 25 | T2 Ds2 | 1 | 25_UK9M49_T2 Ds2_Day1 | |
| 26 | T2 Ds2 | 1 | 26_UK9M50_T2 Ds2_Day1 | |
| 27 | T2 Ds2 | 1 | 27_UK9M51_T2 Ds2_Day1 | |
| 28 | T2 Ds2 | 1 | 28_UK9M52_T2 Ds2_Day1 | |
| 29 | T2 Ds2 | 1 | 29_UK9M53_T2 Ds2_Day1 | |
| 30 | T2 Ds2 | 1 | 30_UK9M54_T2 Ds2_Day1 | |
| 31 | T3 Ds1 | 1 | 31_UK11M61_T3 Ds1_Day1 | |
| 32 | T3 Ds1 | 1 | 32_UK11M62_T3 Ds1_Day1 | |
| 33 | T3 Ds1 | 1 | 33_UK11M63_T3 Ds1_Day1 | |
| 34 | T3 Ds1 | 1 | 34_UK11M64_T3 Ds1_Day1 | |
| 35 | T3 Ds1 | 1 | 35_UK11M65_T3 Ds1_Day1 | |
| 36 | T3 Ds1 | 1 | 36_UK11M66_T3 Ds1_Day1 | |
| 37 | T3 Ds2 | 1 | 37_UK13M73_T3 Ds2_Day1 | |
| 38 | T3 Ds2 | 1 | 38_UK13M75_T3 Ds2_Day1 | |
| 39 | T3 Ds2 | 1 | 39_UK13M76_T3 Ds2_Day1 | |
| 40 | T3 Ds2 | 1 | 40_UK13M77_T3 Ds2_Day1 | |
| 41 | T3 Ds2 | 1 | 41_UK13M78_T3 Ds2_Day1 | |

Plate 5

| | | | | |
|---|---|---|---|---|
| 42 | control | 1 | 42_UK1F101_control_Day1 | TT5: [symbol] |
| 43 | control | 1 | 43_UK1F102_control_Day1 | |
| 44 | control | 1 | 44_UK1F103_control_Day1 | |
| 45 | control | 1 | 45_UK1F104_control_Day1 | |
| 46 | control | 1 | 46_UK1F105_control_Day1 | |
| 47 | control | 1 | 47_UK1F106_control_Day1 | |
| 48 | T1 Ds1 | 1 | 48_UK3F113_T1 Ds1_Day1 | |
| 49 | T1 Ds1 | 1 | 49_UK3F114_T1 Ds1_Day1 | |
| 50 | T1 Ds1 | 1 | 50_UK3F116_T1 Ds1_Day1 | |
| 51 | T1 Ds1 | 1 | 51_UK3F117_T1 Ds1_Day1 | |
| 52 | T1 Ds1 | 1 | 52_UK3F118_T1 Ds1_Day1 | |
| 53 | T1 Ds2 | 1 | 53_UK5F125_T1 Ds2_Day1 | |
| 54 | T1 Ds2 | 1 | 54_UK5F126_T1 Ds2_Day1 | |
| 55 | T1 Ds2 | 1 | 55_UK5F127_T1 Ds2_Day1 | |
| 56 | T1 Ds2 | 1 | 56_UK5F128_T1 Ds2_Day1 | |
| 57 | T1 Ds2 | 1 | 57_UK5F129_T1 Ds2_Day1 | |
| 58 | T1 Ds2 | 1 | 58_UK5F130_T1 Ds2_Day1 | |
| 59 | T2 Ds1 | 1 | 59_UK7F137_T2 Ds1_Day1 | |
| 60 | T2 Ds1 | 1 | 60_UK7F139_T2 Ds1_Day1 | |
| 61 | T2 Ds1 | 1 | 61_UK7F140_T2 Ds1_Day1 | |
| 62 | T2 Ds1 | 1 | 62_UK7F141_T2 Ds1_Day1 | |
| 63 | T2 Ds1 | 1 | 63_UK7F142_T2 Ds1_Day1 | |
| 64 | T2 Ds2 | 1 | 64_UK9F149_T2 Ds2_Day1 | |
| 65 | T2 Ds2 | 1 | 65_UK9F150_T2 Ds2_Day1 | |
| 66 | T2 Ds2 | 1 | 66_UK9F151_T2 Ds2_Day1 | |
| 67 | T2 Ds2 | 1 | 67_UK9F152_T2 Ds2_Day1 | |
| 68 | T2 Ds2 | 1 | 68_UK9F153_T2 Ds2_Day1 | |
| 69 | T2 Ds2 | 1 | 69_UK9F154_T2 Ds2_Day1 | |
| 70 | T3 Ds1 | 1 | 70_UK11F161_T3 Ds1_Day1 | |
| 71 | T3 Ds1 | 1 | 71_UK11F163_T3 Ds1_Day1 | |
| 72 | T3 Ds1 | 1 | 72_UK11F164_T3 Ds1_Day1 | |
| 73 | T3 Ds1 | 1 | 73_UK11F165_T3 Ds1_Day1 | |
| 74 | T3 Ds1 | 1 | 74_UK11F166_T3 Ds1_Day1 | |
| 75 | T3 Ds2 | 1 | 75_UK13F173_T3 Ds2_Day1 | |
| 76 | T3 Ds2 | 1 | 76_UK13F175_T3 Ds2_Day1 | |
| 77 | T3 Ds2 | 1 | 77_UK13F176_T3 Ds2_Day1 | |
| 78 | T3 Ds2 | 1 | 78_UK13F177_T3 Ds2_Day1 | |
| 79 | T3 Ds2 | 1 | 79_UK13F178_T3 Ds2_Day1 | |

Plate 6

| | | | | |
|---|---|---|---|---|
| 80 | control | 5 | 80_UK2M7_control_Day5 | TT6 Male Day 5 |
| 81 | control | 5 | 81_UK2M8_control_Day5 | |
| 82 | control | 5 | 82_UK2M9_control_Day5 | |
| 83 | control | 5 | 83_UK2M10_control_Day5 | |
| 84 | control | 5 | 84_UK2M11_control_Day5 | |
| 85 | T1 Ds1 | 5 | 85_UK4M19_T1 Ds1_Day5 | |
| 86 | T1 Ds1 | 5 | 86_UK4M20_T1 Ds1_Day5 | |
| 87 | T1 Ds1 | 5 | 87_UK4M21_T1 Ds1_Day5 | |
| 88 | T1 Ds1 | 5 | 88_UK4M22_T1 Ds1_Day5 | |
| 89 | T1 Ds1 | 5 | 89_UK4M23_T1 Ds1_Day5 | |
| 90 | T1 Ds1 | 5 | 90_UK4M24_T1 Ds1_Day5 | |
| 91 | T1 Ds2 | 5 | 91_UK6M31_T1 Ds2_Day5 | |
| 92 | T1 Ds2 | 5 | 92_UK6M32_T1 Ds2_Day5 | |
| 93 | T1 Ds2 | 5 | 93_UK6M33_T1 Ds2_Day5 | |
| 94 | T1 Ds2 | 5 | 94_UK6M34_T1 Ds2_Day5 | |
| 95 | T1 Ds2 | 5 | 95_UK6M35_T1 Ds2_Day5 | |
| 96 | T1 Ds2 | 5 | 96_UK6M36_T1 Ds2_Day5 | |
| 97 | T2 Ds1 | 5 | 97_UK8M43_T2 Ds1_Day5 | |
| 98 | T2 Ds1 | 5 | 98_UK8M44_T2 Ds1_Day5 | |
| 99 | T2 Ds1 | 5 | 99_UK8M45_T2 Ds1_Day5 | |
| 100 | T2 Ds1 | 5 | 100_UK8M46_T2 Ds1_Day5 | |
| 101 | T2 Ds1 | 5 | 101_UK8M47_T2 Ds1_Day5 | |
| 102 | T2 Ds1 | 5 | 102_UK8M48_T2 Ds1_Day5 | |
| 103 | T2 Ds2 | 5 | 103_UK10M55_T2 Ds2_Day5 | |
| 104 | T2 Ds2 | 5 | 104_UK10M56_T2 Ds2_Day5 | |
| 105 | T2 Ds2 | 5 | 105_UK10M57_T2 Ds2_Day5 | |
| 106 | T2 Ds2 | 5 | 106_UK10M59_T2 Ds2_Day5 | |
| 107 | T2 Ds2 | 5 | 107_UK10M60_T2 Ds2_Day5 | |
| 108 | T3 Ds1 | 5 | 108_UK12M67_T3 Ds1_Day5 | |
| 109 | T3 Ds1 | 5 | 109_UK12M69_T3 Ds1_Day5 | |
| 110 | T3 Ds1 | 5 | 110_UK12M70_T3 Ds1_Day5 | |
| 111 | T3 Ds1 | 5 | 111_UK12M71_T3 Ds1_Day5 | |
| 112 | T3 Ds1 | 5 | 112_UK12M72_T3 Ds1_Day5 | |
| 113 | T3 Ds2 | 5 | 113_UK14M80_T3 Ds2_Day5 | |
| 114 | T3 Ds2 | 5 | 114_UK14M81_T3 Ds2_Day5 | |
| 115 | T3 Ds2 | 5 | 115_UK14M82_T3 Ds2_Day5 | |
| 116 | T3 Ds2 | 5 | 116_UK14M83_T3 Ds2_Day5 | |

Plate 7

| | | | | |
|---|---|---|---|---|
| 117 | control | 5 | 117_UK2F107_control_Day5 | TT7: Female Day 5 |
| 118 | control | 5 | 118_UK2F108_control_Day5 | |
| 119 | control | 5 | 119_UK2F109_control_Day5 | |
| 120 | control | 5 | 120_UK2F110_control_Day5 | |
| 121 | control | 5 | 121_UK2F111_control_Day5 | |
| 122 | control | 5 | 122_UK2F112_control_Day5 | |
| 123 | T1 Ds1 | 5 | 123_UK4F119_T1 Ds1_Day5 | |
| 124 | T1 Ds1 | 5 | 124_UK4F120_T1 Ds1_Day5 | |
| 125 | T1 Ds1 | 5 | 125_UK4F121_T1 Ds1_Day5 | |
| 126 | T1 Ds1 | 5 | 126_UK4F122_T1 Ds1_Day5 | |
| 127 | T1 Ds1 | 5 | 127_UK4F123_T1 Ds1_Day5 | |
| 128 | T1 Ds1 | 5 | 128_UK4F124_T1 Ds1_Day5 | |
| 129 | T1 Ds2 | 5 | 129_UK6F131_T1 Ds2_Day5 | |
| 130 | T1 Ds2 | 5 | 130_UK6F132_T1 Ds2_Day5 | |
| 131 | T1 Ds2 | 5 | 131_UK6F133_T1 Ds2_Day5 | |
| 132 | T1 Ds2 | 5 | 132_UK6F134_T1 Ds2_Day5 | |
| 133 | T1 Ds2 | 5 | 133_UK6F135_T1 Ds2_Day5 | |
| 134 | T1 Ds2 | 5 | 134_UK6F136_T1 Ds2_Day5 | |
| 135 | T2 Ds1 | 5 | 135_UK8F143_T2 Ds1_Day5 | |
| 136 | T2 Ds1 | 5 | 136_UK8F144_T2 Ds1_Day5 | |
| 137 | T2 Ds1 | 5 | 137_UK8F145_T2 Ds1_Day5 | |
| 138 | T2 Ds1 | 5 | 138_UK8F147_T2 Ds1_Day5 | |
| 139 | T2 Ds1 | 5 | 139_UK8F148_T2 Ds1_Day5 | |
| 140 | T2 Ds2 | 5 | 140_UK10F156_T2 Ds2_Day5 | |
| 141 | T2 Ds2 | 5 | 141_UK10F158_T2 Ds2_Day5 | |
| 142 | T2 Ds2 | 5 | 142_UK10F159_T2 Ds2_Day5 | |
| 143 | T2 Ds2 | 5 | 143_UK10F160_T2 Ds2_Day5 | |
| 144 | T3 Ds1 | 5 | 144_UK12F167_T3 Ds1_Day5 | |
| 145 | T3 Ds1 | 5 | 145_UK12F168_T3 Ds1_Day5 | |
| 146 | T3 Ds1 | 5 | 146_UK12F169_T3 Ds1_Day5 | |
| 147 | T3 Ds1 | 5 | 147_UK12F170_T3 Ds1_Day5 | |
| 148 | T3 Ds1 | 5 | 148_UK12F171_T3 Ds1_Day5 | |
| 149 | T3 Ds1 | 5 | 149_UK12F172_T3 Ds1_Day5 | |
| 150 | T3 Ds2 | 5 | 150_UK14F179_T3 Ds2_Day5 | |
| 151 | T3 Ds2 | 5 | 151_UK14F180_T3 Ds2_Day5 | |
| 152 | T3 Ds2 | 5 | 152_UK14F181_T3 Ds2_Day5 | |
| 153 | T3 Ds2 | 5 | 153_UK14F182_T3 Ds2_Day5 | |
| 154 | T3 Ds2 | 5 | 154_UK14F183_T3 Ds2_Day5 | |

Shuffled plate

| Toxin used | Day | Final name |
|---|---|---|
| Cadmium | 1 | 26_5F138_day1_CadCl |
| chloride | | |
| Cisplatin | 1 | 6_3M21_day1_Cis |
| Cisplatin | 1 | 5_3M20_day1_Cis |
| Control/Saline | 1 | 66_1M2_day1_Saline |
| Doxorubicin | 1 | 30_6F148_day1_Dox |
| Doxorubicin | 1 | 28_6F146_day1_Dox |
| Naïve | 28 | 61_8M83_day28_Naïve |
| Naïve | 28 | 62_8F181_day28_Naïve |
| Tobramycin | 1 | 8_4M29_day1_Tob |
| Valproic acid | 1 | 112_7M55_day1_ValpA |
| Valproic acid | 1 | 117_7F157_day1_ValpA |
| Cadmium | 5 | 54_5F141_day5_CadCl |
| chloride | | |
| Cadmium | 5 | 41_5M41_day5_CadCl |
| chloride | | |
| Cisplatin | 5 | 50_3F124_day5_Cis |
| Cisplatin | 5 | 36_3M24_day5_Cis |
| Control/Saline | 5 | 73_1M6_day5_Saline |
| Control/Saline | 5 | 71_1M4_day5_Saline |
| Doxorubicin | 5 | 43_6M49_day5_Dox |
| Tobramycin | 5 | 51_4F131_day5_Tob |
| Valproic acid | 5 | 123_7F160_day5_ValpA |
| Valproic acid | 5 | 122_7F159_day5_ValpA |
| Cadmium | 28 | 85_5M43_day28_CadCl |
| chloride | | |
| Cadmium | 28 | 86_5M44_day28_CadCl |
| chloride | | |
| Cisplatin | 28 | 79_3M25_day28_Cis |
| Cisplatin | 28 | 94_3F125_day28_Cis |
| Cisplatin | 28 | 81_3M27_day28_Cis |
| Control/Saline | 28 | 106_1M7_day28_Saline |
| Control/Saline | 28 | 107_1M8_day28_Saline |
| Control/Saline | 28 | 111_1F109_day28_Saline |
| Doxorubicin | 28 | 104_6F172_day28_Dox |
| Tobramycin | 28 | 84_4M36_day28_Tob |
| Valproic acid | 28 | 126_7M63_day28_ValpA |
| control | 1 | 2_UK1M2_control_Day1 |
| T1 Ds1 | 1 | 7_UK3M13_T1 Ds1_Day1 |
| T1 Ds1 | 1 | 12_UK3M18_T1 Ds1_Day1 |
| T1 Ds2 | 1 | 16_UK5M28_T1 Ds2_Day1 |
| T2 Ds1 | 1 | 24_UK7M42_T2 Ds1_Day1 |
| T2 Ds1 | 1 | 20_UK7M38_T2 Ds1_Day1 |
| T3 Ds1 | 1 | 31_UK11M61_T3 Ds1_Day1 |
| T3 Ds2 | 1 | 38_UK13M75_T3 Ds2_Day1 |
| T3 Ds2 | 1 | 41_UK13M78_T3 Ds2_Day1 |
| control | 1 | 43_UK1F102_control_Day1 |
| control | 1 | 46_UK1F105_control_Day1 |
| T1 Ds1 | 1 | 51_UK3F117_T1 Ds1_Day1 |
| T1 Ds1 | 1 | 52_UK3F118_T1 Ds1_Day1 |
| T1 Ds2 | 1 | 56_UK5F128_T1 Ds2_Day1 |
| T2 Ds1 | 1 | 63_UK7F142_T2 Ds1_Day1 |
| T2 Ds1 | 1 | 61_UK7F140_T2 Ds1_Day1 |
| T2 Ds2 | 1 | 68_UK9F153_T2 Ds2_Day1 |
| T2 Ds2 | 1 | 66_UK9F151_T2 Ds2_Day1 |
| T3 Ds1 | 1 | 72_UK11F164_T3 Ds1_Day1 |
| T3 Ds2 | 1 | 78_UK13F177_T3 Ds2_Day1 |
| control | 5 | 80_UK2M7_control_Day5 |
| control | 5 | 83_UK2M10_control_Day5 |
| control | 5 | 84_UK2M11_control_Day5 |
| control | 5 | 82_UK2M9_control_Day5 |
| T1 Ds1 | 5 | 86_UK4M20_T1 Ds1_Day5 |
| T1 Ds1 | 5 | 88_UK4M22_T1 Ds1_Day5 |
| T1 Ds2 | 5 | 93_UK6M33_T1 Ds2_Day5 |
| T1 Ds2 | 5 | 94_UK6M34_T1 Ds2_Day5 |
| T2 Ds1 | 5 | 97_UK8M43_T2 Ds1_Day5 |
| T3 Ds1 | 5 | 108_UK12M67_T3 Ds1_Day5 |
| T3 Ds1 | 5 | 110_UK12M70_T3 Ds1_Day5 |
| control | 5 | 118_UK2F108_control_Day5 |
| T1 Ds1 | 5 | 125_UK4F121_T1 Ds1_Day5 |
| T1 Ds2 | 5 | 129_UK6F131_T1 Ds2_Day5 |
| T1 Ds2 | 5 | 131_UK6F133_T1 Ds2_Day5 |
| T2 Ds1 | 5 | 139_UK8F148_T2 Ds1_Day5 |
| T2 Ds2 | 5 | 140_UK10F156_T2 Ds2_Day5 |
| T2 Ds2 | 5 | 143_UK10F160_T2 Ds2_Day5 |
| T3 Ds1 | 5 | 147_UK12F170_T3 Ds1_Day5 |
| T3 Ds1 | 5 | 146_UK12F169_T3 Ds1_Day5 |
| T3 Ds2 | 5 | 151_UK14F180_T3 Ds2_Day5 |
| T3 Ds2 | 5 | 153_UK14F182_T3 Ds2_Day5 |

[symbol]

indicates data missing or illegible when filed

### Plate 6

### Plate 7

### Inter-Plate Normalization

To account for possible differences of qRT-PCR measurements between the different plates due to experimental artifacts, a normalization plate was planned. This plate, designated hereinabove ‘shuffled plate’, contained samples from all other plates. Real Time PCR was performed on this plate using the primers appropriate for all candidates in tables 26 and 27. For each other plate (plates 1-7 above) and each amplicon, the samples appearing also in the shuffled plate were examined—the ratio between the transcript abundance as measured in the plate to the abundance as measured in the shuffled plate was calculated, outliers were manually removed, and the geometric mean of the left ratios was taken as an additional multiplicative normalization factor for the plate and amplicon.

### Real Time qPCR Analysis of the Selected Markers

### Example 3.1

### Expression of Tumor Necrosis Factor Receptor Superfamily, Member 12a, AA686189, Transcripts which are Detectable by Amplicon as Depicted in Sequence Name W41270_DB81_Seg11 in Kidney Tissues of Treated or Untreated Rats

Expression of tumor necrosis factor receptor superfamily, member 12a transcripts detectable by or according to seg11—W41270_DB81_seg11_F2R2 (SEQ ID NO: 254) amplicon and primers W41270_DB81_seg11_F2 (SEQ ID NO: 252) and W41270_DB81_seg11_R2 (SEQ ID NO: 253) was measured by real time PCR. The value of the expression was measured by Real-Time PCR and normalized relative to the expression of the house keeping genes, as described in section “RT Preparation and Real-Time RT-PCR Analysis” hereinabove and by the shuffled plate values, as described in section “Inter-Plate Normalization”. Following optimization, described in Example 3 herein, the experiments were done using primers concentration of 50 nM.

The column entitled W41270_DB81_seg11 in Table 23 contains the normalized expression values of the above-indicated tumor necrosis factor receptor superfamily, member 12a transcript in treated or untreated kidney samples.

As is evident from the column entitled W41270_DB81_seg11 in Table 23, the level of expression of the tumor necrosis factor receptor superfamily, member 12a transcript detectable by the above amplicon was higher in the samples treated with toxic compounds (samples 1-58 and 76-105, in kidney tissue panel described in Table 9 hereinabove) than in the control samples (naïve, saline and valproic acid treated samples; samples numbers 59-75 and 106-129, in kidney tissue panel described in Table 9 hereinabove). Statistical analysis was applied to verify the significance of these results, with a P-value for day 5 of 3.4E⁻⁰⁵.

Primer pairs are also optionally and preferably encompassed within the present invention; for example, for the above experiment, the following primer pair was used as a non-limiting illustrative example only of a suitable primer pair: W41270_DB81_seg11_F2 (SEQ ID NO: 252) forward primer; and W41270_DB81_seg11_R2 (SEQ ID NO: 253) reverse primer.

The present invention also preferably encompasses any amplicon obtained through the use of any suitable primer pair; for example, for the above experiment, the following amplicon was obtained as a non-limiting illustrative example only of a suitable amplicon:

W41270_DB81_seg11_F2R2 (SEQ ID NO: 254).

Forward primer (W41270_DB81_seg11_F2 (SEQ ID NO: 252)):

GATCTGGGTAGGTGGTTGTTGG

Reverse primer (W41270_DB81_seg11_R2 (SEQ ID NO: 253)):

CGCACACCCTTATAAAAGTCCC

Amplicon (W41270_DB81_seg11_F2R2 (SEQ ID NO: 254)):

GATCTGGGTAGGTGGTTGTTGGGGCAGAAAGGAGGTCGTAGACTTAGGAT
ATAGGAAACCAGGAAAAACTGACTGAGGAAGGGACTTTTATAAGGGTGTG
CG

### Example 3.2

### Expression of Interferon Stimulated Exonuclease 20 (ISG20), AI045075, Transcripts which are Detectable by Amplicon as Depicted in Sequence Name AI045075_DB71_Seg6 in Kidney Tissues of Treated or Untreated Rats

Expression of Interferon stimulated exonuclease 20 (ISG20) transcripts detectable by or according to seg6—AI045075_DB71_seg6_F2R2 (SEQ ID NO: 263) amplicon and primers AI045075_DB71_seg6_F2 (SEQ ID NO: 261) and AI045075_DB71_seg6_R2 (SEQ ID NO: 262) was measured by real time PCR. The value of the expression was measured by Real-Time PCR and normalized relative to the expression of the house keeping genes, as described in section “RT Preparation and Real-Time RT-PCR Analysis” hereinabove and by the shuffled plate values, as described in section “Inter-Plate Normalization”.

The column entitled AI045075_DB71_seg6 in Table 23 contains the normalized expression values of the above-indicated Interferon stimulated exonuclease 20 (ISG20) transcript in treated or untreated kidney samples.

As is evident from the column entitled AI045075_DB71_seg6 in Table 23, the level of expression of the Interferon stimulated exonuclease 20 (ISG20) transcript detectable by the above amplicon was higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 1.1E⁻⁰⁶.

Primer pairs are also optionally and preferably encompassed within the present invention; for example, for the above experiment, the following primer pair was used as a non-limiting illustrative example only of a suitable primer pair: AI045075_DB71_seg6_F2 (SEQ ID NO: 261) forward primer; and AI045075_DB71_seg6_R2 (SEQ ID NO: 262) reverse primer.

The present invention also preferably encompasses any amplicon obtained through the use of any suitable primer pair; for example, for the above experiment, the following amplicon was obtained as a non-limiting illustrative example only of a suitable amplicon:

AI045075_DB71_seg6_F2R2 (SEQ ID NO: 263).

Forward primer (AI045075_DB71_seg6_F2 (SEQ ID NO: 261)):

GGGCCACAATGGAGCTCTAC

Reverse primer (AI045075_DB71_seg6_R2 (SEQ ID NO: 262)):

ACAGGTCTCATTCATGGAAAACTATG

Amplicon (AI045075_DB71_seg6_F2R2 (SEQ ID NO: 263)):

GGGCCACAATGGAGCTCTACAAAATCTCTCAGCGACTCAGAGCCCAGCGA
GGGCTGCCCTGCCTGGGAACATCAGCCTGAACTTCATCCTCATCCAGGAT
CAGAAGCAGCTACTCCTTGAAGGACCATAGTTTTCCATGAATGAGACCT
GT

### Example 3.3

### Expression of Interferon Stimulated Exonuclease 20 (ISG20), AI045075, Transcripts which are Detectable by Amplicon as Depicted in Sequence Name W64472_DB81_Seg2 in Kidney Tissues of Treated or Untreated Rats

Expression of Interferon stimulated exonuclease 20 (ISG20), AI045075, transcripts detectable by or according to seg2—W64472_DB81_seg2_F6R1 (SEQ ID NO: 329) amplicon and primers W64472_DB81_seg2_F6 (SEQ ID NO: 327) and W64472_DB81_seg2_R1 (SEQ ID NO: 328) was measured by real time PCR. The value of the expression was measured by Real-Time PCR and normalized relative to the expression of the house keeping genes, as described in section “RT Preparation and Real-Time RT-PCR Analysis” hereinabove and by the shuffled plate values, as described in section “Inter-Plate Normalization”.

The column entitled W64472_DB81_seg2 in Table 23 contains the normalized expression values of the above-indicated Interferon stimulated exonuclease 20 (ISG20), AI045075, transcript in treated or untreated kidney samples.

As is evident from the column entitled W64472_DB81_seg2 in Table 23, the level of expression of the Interferon stimulated exonuclease 20 (ISG20), AI045075, transcript detectable by the above amplicon was higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 1.0E⁻⁰⁵.

Primer pairs are also optionally and preferably encompassed within the present invention; for example, for the above experiment, the following primer pair was used as a non-limiting illustrative example only of a suitable primer pair: W64472_DB81_seg2_F6 (SEQ ID NO: 327) forward primer; and W64472_DB81_seg2_R1 (SEQ ID NO:328) reverse primer.

The present invention also preferably encompasses any amplicon obtained through the use of any suitable primer pair; for example, for the above experiment, the following amplicon was obtained as a non-limiting illustrative example only of a suitable amplicon:

W64472_DB81_seg2_F6R1 (SEQ ID NO: 329).

Following optimization described in Example 3 herein, the following primers were used to amplify W64472_DB81_seg2_F6R1 (SEQ ID NO: 329) amplicon:

Forward primer (W64472_DB81_seg2_F6 (SEQ ID NO: 327)):

ACAGCCTGATGCAGACAGC

Reverse primer (W64472_DB81_seg2_R1 (SEQ ID NO:328)):

TCTGGTTCATTATCAAGGGAAGTTG

Amplicon (W64472_DB81_seg2_F6R1 (SEQ ID NO: 329)):

ACAGCCTGATGCAGACAGCCCTGACTCAACCTGCCAGCCCCCTTACCTGG
CCAGCCTTGAGGAGATGGAACAGCCCAGGCTACAAGGCCTGCCCCCACT
CCTCAACTTCCCTTGATAATGAACCAGA

### Example 3.4

### Expression of Cyclin-G1 (CCNG1), 1131883, Transcripts which are Detectable by Amplicon as Depicted in Sequence Name H31883_DB71_Seg13 in Kidney Tissues of Treated or Untreated Rats

Expression of Cyclin-G1 (CCNG1) transcripts detectable by or according to seg13—H31883_DB71_seg13_F5R5 (SEQ ID NO: 332) amplicon and primers H31883_DB71_seg13_F5 (SEQ ID NO: 330) and H31883_DB71_seg13_R5 (SEQ ID NO: 331) was measured by real time PCR. The value of the expression was measured by Real-Time PCR and normalized relative to the expression of the house keeping genes, as described in section “RT Preparation and Real-Time RT-PCR Analysis” hereinabove and by the shuffled plate values, as described in section “Inter-Plate Normalization”.

The column entitled H31883_DB71_seg13 in Table 23 contains the normalized expression values of the above-indicated Cyclin-G1 (CCNG1) transcript in treated or untreated kidney samples.

As is evident from the column entitled H31883_DB71_seg13 in Table 23, the level of expression of the Cyclin-G1 (CCNG1) transcript detectable by the above amplicon was higher in the samples treated with toxic compounds (samples 1-58 and 76-105, in kidney tissue panel described in hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 2.1E⁻⁰⁵.

Primer pairs are also optionally and preferably encompassed within the present invention; for example, for the above experiment, the following primer pair was used as a non-limiting illustrative example only of a suitable primer pair: H31883_DB71_seg13_F5 (SEQ ID NO: 330) forward primer; and H31883_DB71_seg13_R5 (SEQ ID NO: 331) reverse primer.

The present invention also preferably encompasses any amplicon obtained through the use of any suitable primer pair; for example, for the above experiment, the following amplicon was obtained as a non-limiting illustrative example only of a suitable amplicon:

H31883_DB71_seg13_F5R5 (SEQ ID NO: 332).

Following optimization described in Example 3 herein, the following primers were used to amplify H31883_DB71_seg13_F5R5 (SEQ ID NO: 332) amplicon:

Forward primer (H31883_DB71_seg13_F5 (SEQ ID NO: 330)):

CAGAATTGCTGCCTCAATCTAGTCC

Reverse primer (H31883_DB71_seg13_R5 (SEQ ID NO: 331)):

GGTTTTGCAGATGTACTCGGTTCC

Amplicon (H31883_DB71_seg13_F5R5 (SEQ ID NO: 332)):

CAGAATTGCTGCCTCAATCTAGTCCCATTTGAGAAAATTTGTTTCTACTG
TCTCAATAACTGGATGAAATATCACTCTGAAAACTTGCCTATTGCACTAA
AGCTAGTTTAGGCTTGATAAAACACTCCAGGAGGTTTTTACCACAGACTG
TTTCTATTAAAACTGCTGCTTCTCATGTACAATTTTGTTTTAAAAGGAAC
CGAGTACATCTGCAAAACC

### Example 3.5

### Expression of Cyclin-G1 (CCNG1), H31883, Transcripts which are Detectable by Amplicon as Depicted in Sequence Name MUSCYCG1R_DB81_Seg19-20 in Kidney Tissues of Treated or Untreated Rats

Expression of Cyclin-G1 (CCNG1) transcripts detectable by or according to seg19-20—MUSCYCG1R_DB81_seg19-20_F1R1 (SEQ ID NO: 296) amplicon and primers MUSCYCG1R_DB81_seg19-20_F1 (SEQ ID NO: 294) and MUSCYCG1R_DB81_seg19-20_R1 (SEQ ID NO: 295) was measured by real time PCR. The value of the expression was measured by Real-Time PCR and normalized relative to the expression of the house keeping genes, as described in section “RT Preparation and Real-Time RT-PCR Analysis” hereinabove and by the shuffled plate values, as described in section “Inter-Plate Normalization”. Following optimization described in Example 3 herein, the annealing temperature of the PCR was 62° C.

The column entitled MUSCYCG1R_DB81_seg19-20 in Table 23 contains the normalized expression values of the above-indicated Cyclin-G1 (CCNG1) transcript in treated or untreated kidney samples.

As is evident from the column entitled MUSCYCG1R_DB81_seg19-20 in Table 23, the level of expression of the Cyclin-G1 (CCNG1) transcript detectable by the above amplicon was higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 1.3E⁻⁰⁴.

Primer pairs are also optionally and preferably encompassed within the present invention; for example, for the above experiment, the following primer pair was used as a non-limiting illustrative example only of a suitable primer pair: MUSCYCG1R_DB81_seg19-20_F1 (SEQ ID NO: 294) forward primer; and MUSCYCG1R_DB81_seg19-20_R1 (SEQ ID NO: 295) reverse primer.

The present invention also preferably encompasses any amplicon obtained through the use of any suitable primer pair; for example, for the above experiment, the following amplicon was obtained as a non-limiting illustrative example only of a suitable amplicon: MUSCYCG1R_DB81_seg19-20_F1R1 (SEQ ID NO: 296).

Forward primer (MUSCYCG1R_DB81_seg19-20_F1 (SEQ ID NO: 294)):

GAGATCCAAGCACTGAAGTATGTAGAGT

Reverse primer (MUSCYCG1R_DB81_seg19-20_R1 (SEQ ID NO: 295)):

TCAGGAGTACAGTGGATACATTTCTCTT

Amplicon (MUSCYCG1R_DB81_seg19-20_F1R1 (SEQ ID NO: 296)):

GAGATCCAAGCACTGAAGTATGTAGAGTTAACAGAAGGAGTAGAATGT
ATTCAGAAACATTCCAAGGTATGCCAAGGTGATAGCATTGATCCTATT
AGCAAGCTACAAGAGAAATGTATCCACTGTACTCCTGA

### Example 3.6

### Expression Etoposide Induced 2.4 (EI24) mRNA, 1131799, Transcripts which are Detectable by Amplicon as Depicted in Sequence Name W83813_DB81_Seg27 in Kidney Tissues of Treated or Untreated Rats

Expression of Etoposide induced 2.4 mRNA (EI24) transcripts detectable by or according to seg27—W83813_DB81_seg27_F1R1 (SEQ ID NO: 287) amplicon and primers W83813_DB81_seg27_F1 (SEQ ID NO: 285) and W83813_DB81_seg27_R1 (SEQ ID NO: 286) was measured by real time PCR. The value of the expression was measured by Real-Time PCR and normalized relative to the expression of the house keeping genes, as described in section “RT Preparation and Real-Time RT-PCR Analysis” hereinabove and by the shuffled plate values, as described in section “Inter-Plate Normalization”.

The column entitled W83813_DB81_seg27 in Table 23 contains the normalized expression values of the above-indicated Etoposide induced 2.4 mRNA (EI24) transcript in treated or untreated kidney samples.

As is evident from the column entitled W83813_DB81_seg27 in Table 23, the level of expression of the Etoposide induced 2.4 mRNA (EI24) transcript detectable by the above amplicon was higher in the samples treated with toxic compounds (samples 1-58 and 76-105, in kidney tissue panel described in Table 9 hereinabove) than in the control samples (naïve, saline and valproic acid treated samples; samples numbers—59-75 and 106-129, in kidney tissue panel described in Table 9 hereinabove). Statistical analysis was applied to verify the significance of these results, P-value for day 5: 0.04.

Primer pairs are also optionally and preferably encompassed within the present invention; for example, for the above experiment, the following primer pair was used as a non-limiting illustrative example only of a suitable primer pair: W83813_DB81_seg27_F1 (SEQ ID NO: 285) forward primer; and W83813_DB81_seg27_R1 (SEQ ID NO: 286) reverse primer.

The present invention also preferably encompasses any amplicon obtained through the use of any suitable primer pair; for example, for the above experiment, the following amplicon was obtained as a non-limiting illustrative example only of a suitable amplicon W83813_DB81_seg27_F1R1 (SEQ ID NO: 287):

Forward primer (W83813_DB81_seg27_F1 (SEQ ID NO: 285)):

AATCTAGGCTGCCTCCTGGAG

Reverse primer (W83813_DB81_seg27_R1 (SEQ ID NO: 286)):

AGGTCAATTATACAAGGCATGACTTTAG

Amplicon (W83813_DB81_seg27_F1R1 (SEQ ID NO: 287)):

AATCTAGGCTGCCTCCTGGAGGAAGATACTTAGGAGTTCAGAAGTGAA
GAGATGAGGCTTATAATACTTTTTCCTAAAGTCATGCCTTGTATAATTG
ACCT

Classification Using qRT-PCR Data

Given the qRT-PCR panel for the selected genes detailed in Table 23, the optimal classifier was built based on the measured expression levels of the labeled samples, as described in Table 9. This was performed again to achieve a more accurate and controlled signature.

Two equivalent optimal signatures for prediction of renal damage at day-5, as derived in the validation stage analysis of the labeled samples, are given in Table 26 (four amplicons of three genes) and Table 27 (six amplicons of four genes). Table 28 summarizes the performance of the 4-genes signature on the various groups of the labeled samples.

TABLE 26
Optimal Signature Day 5 (Validation Stage Analysis)

| | | Splice Variant/ | | |
|---|---|---|---|---|
| Contig | Gene name | Wild Type | Internal name | Probeset |
| AA686189 | TNFRSF12A | SV | W41270_DB81_seg11 | 1371785_at |
| AI045075 | ISG20 | SV | W64472_DB81_seg2 | 1390507_at |
| AI045075 | ISG20 | WT | AI045075_DB71_seg6 | 1390507_at |
| H31883 | CCNG1 | WT | H31883_DB71_seg13 | 1367764_at |

TABLE 27
Optimal Signature Day 5 (Validation Stage Analysis)

| | | Splice | | |
|---|---|---|---|---|
| | | Variant/ | | |
| Contig | Gene name | Wild Type | Internal name | Probeset |
| AA686189 | TNFRSF12A | SV | W41270_DB81_seg11 | 1371785_at |
| AI045075 | ISG20 | SV | W64472_DB81_seg2 | 1390507_at |
| AI045075 | ISG20 | WT | AI045075_DB71_seg6 | 1390507_at |
| H31883 | CCNG1 | WT | H31883_DB71_seg13 | 1367764_at |
| H31883 | CCNG1 | SV | MUSCYCG1R_DB81_seg19-20 | 1367764_at |
| H31799 | EI24 | SV | W83813_DB81_seg27 | 1388642_at |

TABLE 28
Performance of the 4-genes signature on the
various groups of the labeled samples

| | | | Standard |
|---|---|---|---|
| | | Average | Deviation |
| Group (Compound, Period, | No. of | Toxicity | of Toxicity |
| Gender) | samples | Score | Score |
| Doxorubicin Day5 Male | 2 | 0.77 | 0.07 |
| Gentamycin Day5 Female | 3 | 0.73 | 0.12 |
| Cisplatin Day5 Female | 3 | 0.69 | 0.14 |
| Gentamycin Day5 Male | 3 | 0.63 | 0.03 |
| Cisplatin Day5 Male | 3 | 0.63 | 0.27 |
| Tobramycin Day5 Female | 3 | 0.61 | 0.28 |
| Doxorubicin Day5 Female | 3 | 0.61 | 0.19 |
| Tobramycin Day5 Male | 3 | 0.59 | 0.18 |
| Valproic-Acid Day1 Male | 3 | 0.57 | 0.42 |
| Saline Day1 Male | 3 | 0.51 | 0.45 |
| Naïve Male | 3 | 0.41 | 0.42 |
| Valproic-Acid Day1 Female | 3 | 0.33 | 0.51 |
| Valproic-Acid Day5 Female | 3 | 0.18 | 0.3 |
| Naïve Female | 3 | 0.14 | 0.24 |
| Saline Day1 Female | 3 | 0.11 | 0.14 |
| Valproic-Acid Day28 Female | 3 | 0.07 | 0.12 |
| Valproic-Acid Day5 Male | 3 | 0.02 | 0.02 |
| Valproic-Acid Day28 Male | 3 | 0.01 | 0 |
| Saline Day5 Female | 2 | 0.01 | 0.01 |
| Saline Day28 Female | 3 | 0.01 | 0.01 |
| Saline Day5 Male | 3 | 0 | 0 |
| Saline Day28 Male | 3 | 0 | 0 |

The two optimal classifiers (signatures described in Tables 26 and 27) were applied to the qRT-PCR reads of the un-labeled samples and their calls was used to assign a level of toxicity to each group and, eventually, each compound.

### Results

qRT-PCR Data Analysis of Labeled Samples Described in Table 9 and Plate 1-3 in Table 25.

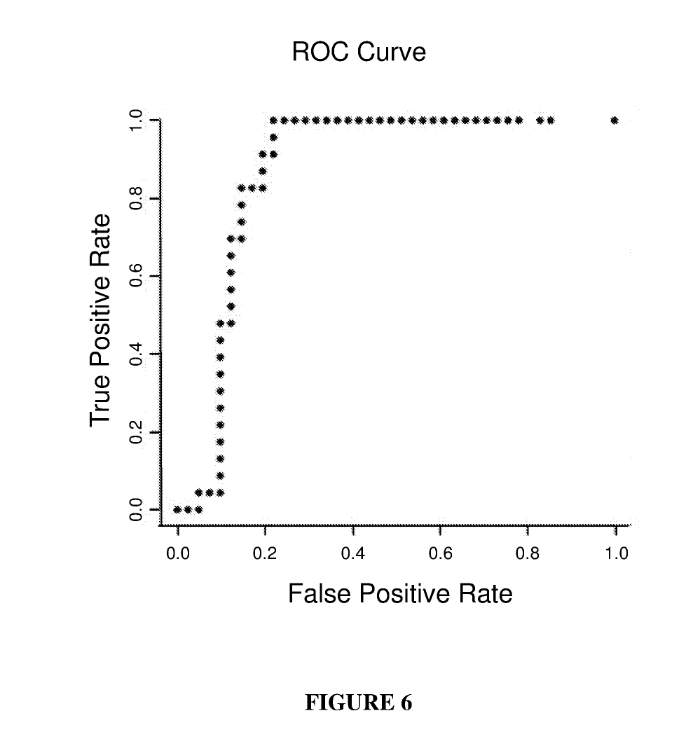

Transcripts abundance was measured using qRT-PCR for 8 amplicons from the 4 genes disclosed in Table 13 hereinabove. As in Example 1, an optimal signature for day-5 was identified, distinguishing the day-5 “Toxic” samples from all control (and naïve rats) samples. Iterative feature selection and Random-Forest classifier were used as in the discovery stage, described in Example 1 hereinabove. The input data was the new qRT-PCR measurements and the gender of the rat as an extra two-values features. The same parameters as in the discovery stage were used again, performing cross validation by leaving-out randomly selected samples—3 control samples and 3 toxic samples and repeating 500 times, choosing the bound on ‘toxicity’ as to maximize the accuracy. Two equivalent (but not identical in results) signatures were found, presented in Tables 26 and 27, consisting of 4 and 6 amplicons, coming from 3 and 4 genes, respectively. The cross-validation performance evaluations of these signatures show 80% sensitivity at about 85% specificity. The Out-Of-Bag OOB performance evaluation gave the confusion matrix presented in Table 29 (overall accuracy of 83%) and ROC curve presented in FIG. 6.

TABLE 29
Confusion matrix

| | Normal | Toxic |
|---|---|---|
| Predicted as Normal | 34 | 4 |
| Predicted as Toxic | 7 | 19 |

It was possible to find a bound on the Random-Forest call—30% of the trees in the forests giving a “Toxic” call—such that all “Toxic” samples pass that bound while only one group of control samples (Naïve males) has more than 50% (2 out of 3) of its members pass that bound. See Tables 26 and 27 for details on the signatures and Table 28 for their performance

qRT-PCR Data Analysis of the Un-Labeled Samples, Described in Plates 4-7 in Table 25

The classifiers described in Tables 26 and 27, were used for testing the un-labeled samples. Both classifiers were applied to the measurements of each sample, used the 30% bound from above, and the number of samples pass that bound in each group were summarized. The results are given in the following Table 30

TABLE 30

| | | ‘Toxic’ by | ‘Toxic’ by | ‘Toxic’ by | ‘Toxic’ by | |
|---|---|---|---|---|---|---|
| Group (Compound, | | 4-genes | 6-genes | 4-genes | 6-genes | Average |
| Dose, Period, | Animals | Classifier | Classifier | Classifier | Classifier | ‘Toxic’ |
| Gender) | (#) | (#) | (#) | (%) | (#) | (%) |
| T2 Ds1 Day5 Male | 6 | 5 | 5 | 83 | 83 | 0.83 |
| T1 Ds2 Day5 Female | 6 | 5 | 3 | 83 | 50 | 0.665 |
| control Day5 Male | 5 | 3 | 3 | 60 | 60 | 0.6 |
| T2 Ds2 Day1 Female | 6 | 4 | 3 | 67 | 50 | 0.585 |
| T3 Ds1 Day5 Male | 5 | 3 | 2 | 60 | 40 | 0.5 |
| T2 Ds2 Day5 Female | 4 | 2 | 2 | 50 | 50 | 0.5 |
| T1 Ds1 Day5 Male | 6 | 3 | 3 | 50 | 50 | 0.5 |
| T1 Ds1 Day5 Female | 6 | 3 | 3 | 50 | 50 | 0.5 |
| control Day5 Female | 6 | 3 | 2 | 50 | 33 | 0.415 |
| T3 Ds2 Day5 Male | 4 | 2 | 1 | 50 | 25 | 0.375 |
| control Day1 Male | 6 | 2 | 2 | 33 | 33 | 0.33 |
| T1 Ds2 Day5 Male | 6 | 2 | 2 | 33 | 33 | 0.33 |
| T3 Ds2 Day1 Male | 5 | 2 | 1 | 40 | 20 | 0.3 |
| T2 Ds2 Day5 Male | 5 | 1 | 2 | 20 | 40 | 0.3 |
| T2 Ds1 Day5 Female | 5 | 2 | 1 | 40 | 20 | 0.3 |
| T2 Ds1 Day1 Male | 6 | 2 | 1 | 33 | 17 | 0.25 |
| T3 Ds2 Day5 Female | 5 | 1 | 1 | 20 | 20 | 0.2 |
| T3 Ds2 Day1 Female | 5 | 1 | 1 | 20 | 20 | 0.2 |
| T3 Ds1 Day1 Female | 5 | 1 | 1 | 20 | 20 | 0.2 |
| T2 Ds1 Day1 Female | 5 | 1 | 1 | 20 | 20 | 0.2 |
| control Day1 Female | 6 | 1 | 1 | 17 | 17 | 0.17 |
| T1 Ds1 Day1 Male | 6 | 1 | 1 | 17 | 17 | 0.17 |
| T3 Ds1 Day5 Female | 6 | 1 | 0 | 17 | 0 | 0.085 |
| T3 Ds1 Day1 Male | 6 | 0 | 0 | 0 | 0 | 0 |
| T2 Ds2 Day1 Male | 6 | 0 | 0 | 0 | 0 | 0 |
| T1 Ds2 Day1 Male | 6 | 0 | 0 | 0 | 0 | 0 |
| T1 Ds2 Day1 Female | 6 | 0 | 0 | 0 | 0 | 0 |
| T1 Ds1 Day1 Female | 5 | 0 | 0 | 0 | 0 | 0 |

The first two groups—“T2 Ds1 Day5 Male” and “T1 Ds2 Day5 Female” showed a clear nephrotoxic effect (more than 66% of samples were marked as “Toxic” when averaging the two classifiers), while the following six groups—“control Day5 Male”, “T2 Ds2 Day1 Female”, “T3 Ds1 Day5 Male”, “T2 Ds2 Day5 Female”, “T1 Ds1 Day5 Male”, “T1 Ds1 Day5 Female” were also suspicious of showing the effect (at least 50% of samples predicted as ‘Toxic’ on average). Therefore, consistently with the information available for the compounds T1 and T2, the results demonstrated that T1 and T2 are nephrotoxic agents. The T3 and control groups showing ‘possible’ toxic effect are either real false-positive of the classifier, or a result of some technical problem.

Using the classifier the toxicity was established after the rats were treated for 5 days only, while the visible pathological damage was seen only after 28 days for T1 and after 7 days for T2, as shown in FIGS. 3-5.

Therefore, combinations of biomarkers according to the present invention can be used for early detection of drug-induced nephrotoxicity. These signatures predict the future occurrence of drug induced renal toxicity before it is detected using the traditional endpoint analysis such as histopathology or clinical chemistry.

According to the present invention, an optimized assay based on these signatures can be implemented in pre-clinical studies. The markers of the invention can be used alone or in combination with other known markers for renal damage identification.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-32. (canceled)

33. A kit for assaying a sample for gene sets predictive of onset of renal injury, comprising:
a set of probes and/or pairs of primers that hybridize to at least two polynucleotide markers, wherein said at least two polynucleotide markers are selected from the group consisting of: SEQ ID NO:1, 3, 48, 6, 12, 52 or a fragment thereof and sequences homologous thereto;
reagents for detecting the at least two polynucleotide markers and for measuring the expression level of said at least two polynucleotide markers; and
instructional material directing the correlation of said expression level of said at least two polynucleotide markers with onset of renal injury using a classifier capable of classifying renal injury using said expression level of said at least two polynucleotide markers.

34. The kit of claim 33, said kit comprises a plurality of probes and/or pairs of primers for detecting a set of four polynucleotide markers, wherein the set of four polynucleotide markers comprises:
a first polynucleotide having the nucleic acid sequence set forth in SEQ ID NO:1, or having a node consisting of the nucleic acid sequence set forth in SEQ ID NO:2, or having an amplicon consisting of the nucleic acid sequence set forth in SEQ ID NO:254;
a second polynucleotide having the nucleic acid sequence set forth in SEQ ID NO:3, or having a node consisting of the nucleic acid sequence set forth in any one of SEQ ID NOs:4 and 5, or having an amplicon consisting of the nucleic acid sequence set forth in any one of SEQ ID NO:266 and 329;
a third polynucleotide having the nucleic acid sequence set forth in SEQ ID NO:6, or having a node consisting of the nucleic acid sequence set forth in any one of SEQ ID NOS:7 and 8, or having an amplicon consisting of the nucleic acid sequence set forth in SEQ ID NO:287; and
a fourth polynucleotide having the nucleic acid sequence set forth in SEQ ID NO:12, or having a node consisting of the nucleic acid sequence set forth in any one of SEQ ID NOS:13 and 14, or having an amplicon consisting of the nucleic acid sequence set forth in SEQ ID NO:296, and
sequences homologous thereto.

35. The kit of claim 33, said kit comprises a plurality of probes and/or pairs of primers for detecting a set of four polynucleotide markers, wherein the set of four polynucleotide markers comprises:
a first polynucleotide having the nucleic acid sequence set forth in SEQ ID NO:1, or having a node consisting of the nucleic acid sequence set forth in SEQ ID NO:2, or having an amplicon consisting of the nucleic acid sequence set forth in SEQ ID NO:254;
a second polynucleotide having the nucleic acid sequence set forth in SEQ ID NO:3, or having a node consisting of the nucleic acid sequence set forth in any one of SEQ ID NOS:4 and 5, or having an amplicon consisting of the nucleic acid sequence set forth in any one of SEQ ID NOS:266 and 329;
a third polynucleotide having the nucleic acid sequence set forth in SEQ ID NO:48, or having a node consisting of the nucleic acid sequence set forth in SEQ ID NO:49, or having an amplicon consisting of the nucleic acid sequence set forth in SEQ ID NO:263; and
a fourth polynucleotide having the nucleic acid sequence set forth in SEQ ID NO:52 or having a node consisting of the nucleic acid sequence set forth in SEQ ID NO:53 or having an amplicon consisting of the nucleic acid sequence set forth in any one of SEQ ID NOS:290 and 332, and
sequences homologous thereto.

36. The kit of claim 33, said kit comprises a plurality of probes and/or pairs of primers for detecting a set of six polynucleotide markers, wherein the set of six polynucleotide markers comprises:
a first polynucleotide the nucleic acid sequence set forth in SEQ ID NO:1, or having a node consisting of the nucleic acid sequence set forth in SEQ ID NO:2, or having an amplicon consisting of the nucleic acid sequence set forth in SEQ ID NO:254;
a second polynucleotide having the nucleic acid sequence set forth in SEQ ID NO:3, or having a node consisting of the nucleic acid sequence set forth in any one of SEQ ID NOS:4 and 5, or having an amplicon consisting of the nucleic acid sequence set forth in any one of SEQ ID NOS:266 and 329;
a third polynucleotide having the nucleic acid sequence set forth in SEQ ID NO:48, or having a node consisting of the nucleic acid sequence set forth in SEQ ID NO:49, or having an amplicon consisting of the nucleic acid sequence set forth in SEQ ID NO:263;
a fourth polynucleotide having the nucleic acid sequence set forth in SEQ ID NO:6, or having a node consisting of the nucleic acid sequence set forth in any one of SEQ ID NOS:7 and 8, or having an amplicon consisting of the nucleic acid sequence set forth in SEQ ID NO:287;
a fifth polynucleotide having the nucleic acid sequence set forth in SEQ ID NO:12, or having a node consisting of the nucleic acid sequence set forth in any one of SEQ ID NOS:13 and 14, or having an amplicon consisting of the nucleic acid sequence set forth in SEQ ID NO:296; and
a sixth polynucleotide having the nucleic acid sequence set forth in SEQ ID NO:52, or having a node consisting of the nucleic acid sequence set forth in SEQ ID NO:53, or having an amplicon consisting of the nucleic acid sequence set forth in any one of SEQ ID NOS:290 and 332, and
sequences homologous thereto.

37. The kit of claim 33, wherein the plurality of pairs of primers is selected from the group consisting of:
SEQ ID NOS:252 and 253,
SEQ ID NOS:261 and 262,
SEQ ID NOS:264 and 265,
SEQ ID NOS:327 and 328,
SEQ ID NOS:285 and 286,
SEQ ID NOS:288 and 289,
SEQ ID NOS:294 and 295, and
SEQ ID NOS:330 and 331.

38. The kit of claim 33, wherein the reagent for detecting the plurality of polynucleotide markers and for measuring their expression level are reagent for employing at least one NAT-based technology selected from the group consisting of: a PCR; Real-Time PCR; LCR; Self-Sustained Synthetic Reaction; Q-Beta Replicase; Cycling Probe Reaction; Branched DNA; RFLP analysis; DGGE/TGGE; Single-Strand Conformation Polymorphism; Dideoxy Fingerprinting; Microarrays; Fluorescence In Situ Hybridization; and Comparative Genomic Hybridization.

39. The kit of claim 33, wherein the classifier is selected from the group consisting of: linear classifier and non-linear classifier.

40. The kit of claim 33, wherein the classifier has a training accuracy of at least 83%.

41. A method for predicting onset of renal injury caused by treatment with a compound, comprising:
(i) administering a dose of the compound to at least one test subject;
(ii) obtaining a biological sample from the at least one test subject; and
(iii) determining whether the biological sample is in the positive class for onset of renal injury using the kit of claim 33.

42. The method of claim 41, wherein the biological sample is obtained from the at least one test subject after a time period selected from the group consisting of: 1 day after administering the dose of the compound; 5 days after administering the dose of the compound; 7 days after administering the dose of the compound; 14 days after administering the dose of the compound; 21 days after administering the dose of the compound; and 28 days after administering the compound.

43. A method for predicting onset of renal injury caused by treatment with a compound, comprising:
(i) administering a dose of the compound to at least one test subject;
(ii) obtaining a biological sample from the at least one test subject;
(iii) measuring the expression level of at least two polynucleotide markers selected from the group consisting of: SEQ ID NO:1, 3, 48, 6, 12, 52 and sequences homologous thereto; and
(iv) determining whether the biological sample is in the positive class for onset of renal injury.

44. The method of claim 43, wherein the polynucleotide markers comprises a set of four polynucleotide markers comprising:
a first polynucleotide having the nucleic acid sequence set forth in SEQ ID NO:1 or having a node consisting of the nucleic acid sequence set forth in SEQ ID NO:2;
a second polynucleotide having the nucleic acid sequence set forth in SEQ ID NO:3 or having a node consisting of the nucleic acid sequence set forth in any one of SEQ ID NOS:4 and 5;
a third polynucleotide having the nucleic acid sequence set forth in SEQ ID NO:6 or having a node consisting of the nucleic acid sequence set forth in any one of SEQ ID NOS:7 and 8; and
a fourth polynucleotide having the nucleic acid sequence set forth in SEQ ID NO:12 or having a node consisting of the nucleic acid sequence set forth in any one of SEQ ID NOS:13 and 14,
and sequences homologous thereto.

45. The method of claim 44, wherein the expression of the four polynucleotide markers is measured using a set of probes that hybridize to:
a nucleic acid sequence set form in SEQ ID NO:254;
a nucleic acid sequence set forth in any one of SEQ ID NO:266 and 329;
a nucleic acid sequence set forth in SEQ ID NO:287; and
a nucleic acid sequence set forth in SEQ ID NO:296, and
sequences homologous thereto.

46. The method of claim 43, wherein the set of probes hybridizes a set of four polynucleotide markers, wherein the set of four polynucleotide markers comprises:
a first polynucleotide having the nucleic acid sequence set forth in SEQ ID NO:1 or having a node consisting of the nucleic acid sequence set forth in SEQ ID NO:2;
a second polynucleotide having the nucleic acid sequence set forth in SEQ ID NO:3 or having a node consisting of the nucleic acid sequence set forth in any one of SEQ ID NOS:4 and 5;
a third polynucleotide having the nucleic acid sequence set forth in SEQ ID NO:48 or having a node consisting of the nucleic acid sequence set forth in SEQ ID NO:49; and
a fourth polynucleotide having the nucleic acid sequence set forth in SEQ ID NO:52 or having a node consisting of the nucleic acid sequence set forth in SEQ ID NO:53, and
sequences homologous thereto.

47. The method of claim 46, wherein the expression of the four polynucleotide markers is measured using a set of probes that hybridize to:
a nucleic acid sequence set form in SEQ ID NO:254;
a nucleic acid sequence set forth in any one of SEQ ID NO:266 and 329;
a nucleic acid sequence set forth in SEQ ID NO:263; and
a nucleic acid sequence set forth in in any one of SEQ ID NOS:290 and 332, and
sequences homologous thereto.

48. The method of claim 43, wherein the set of probes hybridizes to a set of six polynucleotide markers, wherein the set of six polynucleotide markers comprises:
a first polynucleotide the nucleic acid sequence set forth in SEQ ID NO:1 or having a node consisting of the nucleic acid sequence set forth in SEQ ID NO:2;
a second polynucleotide having the nucleic acid sequence set forth in SEQ ID NO:3 or having a node consisting of the nucleic acid sequence set forth in any one of SEQ ID NOS:4 and 5;
a third polynucleotide having the nucleic acid sequence set forth in SEQ ID NO:48 or having a node consisting of the nucleic acid sequence set forth in SEQ ID NO:49;
a fourth polynucleotide having the nucleic acid sequence set forth in SEQ ID NO:6 or having a node consisting of the nucleic acid sequence set forth in any one of SEQ ID NOS:7 and 8;
a fifth polynucleotide having the nucleic acid sequence set forth in SEQ ID NO:12 or having a node consisting of the nucleic acid sequence set forth in any one of SEQ ID NOS:13 and 14; and
a sixth polynucleotide having the nucleic acid sequence set forth in SEQ ID NO:52 or having a node consisting of the nucleic acid sequence set forth in SEQ ID NO:53, and
sequences homologous thereto.

49. The method of claim 48, wherein the expression of the six polynucleotide markers is measured using a set of probes that hybridize to:
a nucleic acid sequence set form in SEQ ID NO:254;
a nucleic acid sequence set forth in any one of SEQ ID NO:266 and 329;
a nucleic acid sequence set forth in SEQ ID NO:263;
a nucleic acid sequence set forth in SEQ ID NO:287;
a nucleic acid sequence set forth in SEQ ID NO:296; and
a nucleic acid sequence set forth in in any one of SEQ ID NOS:290 and 332, and
sequences homologous thereto.

50. The method of claim 43, wherein the expression of the plurality of polynucleotide markers is measured using a set of probes selected from the group consisting of:
SEQ ID NO:252 and/or SEQ ID NO:253,
SEQ ID NO:261 and/or SEQ ID NO:262,
SEQ ID NO:264 and/or SEQ ID NO:265,
SEQ ID NO:327 and/or SEQ ID NO:328,
SEQ ID NO:285 and/or SEQ ID NO:286,
SEQ ID NO:288 and/or SEQ ID NO:289,
SEQ ID NO:294 and/or SEQ ID NO:295, and
SEQ ID NO:330 and/or SEQ ID NO:331.

51. The kit of claim 33, wherein the set of probes is selected from the group consisting of:
SEQ ID NO:252 and/or SEQ ID NO:253,
SEQ ID NO:261 and/or SEQ ID NO:262,
SEQ ID NO:264 and/or SEQ ID NO:265,
SEQ ID NO:327 and/or SEQ ID NO:328,
SEQ ID NO:285 and/or SEQ ID NO:286,
SEQ ID NO:288 and/or SEQ ID NO:289,
SEQ ID NO:294 and/or SEQ ID NO:295, and
SEQ ID NO:330 and/or SEQ ID NO:331, and
sequences homologous thereto.

52. The kit of claim 33, wherein the set of probes hybridizes to a set of four polynucleotide markers, wherein the set of four polynucleotide markers comprises:
a first polynucleotide having a node consisting of the nucleic acid sequence set forth in SEQ ID NO:2 or the nucleic acid sequence set forth in SEQ ID NO:254;
a second polynucleotide having a node consisting of the nucleic acid sequence set forth in any one of SEQ ID NOS:4 and 5 or the nucleic acid sequence set forth in any one of SEQ ID NO:266 and 329;
a third polynucleotide having a node consisting of the nucleic acid sequence set forth in any one of SEQ ID NOS:7 and 8 or having the nucleic acid sequence set forth in SEQ ID NO:287; and
a fourth polynucleotide having a node consisting of the nucleic acid sequence set forth in any one of SEQ ID NOS:13 and 14 or having the nucleic acid sequence set forth in SEQ ID NO:296, and
sequences homologous thereto.

53. The kit of claim 33, wherein the set of probes hybridizes to a set of four polynucleotide markers, wherein the set of four polynucleotide markers comprises:
a first polynucleotide having a node consisting of the nucleic acid sequence set forth in SEQ ID NO:2 or having the nucleic acid sequence set forth in SEQ ID NO:254;
a second polynucleotide having a node consisting of the nucleic acid sequence set forth in any one of SEQ ID NOS:4 and 5 or having the nucleic acid sequence set forth in any one of SEQ ID NOS:266 and 329;
a third polynucleotide having a node consisting of the nucleic acid sequence set forth in SEQ ID NO:49 or having the nucleic acid sequence set forth in SEQ ID NO:263; and
a fourth polynucleotide having a node consisting of the nucleic acid sequence set forth in SEQ ID NO:53 or having the nucleic acid sequence set forth in any one of SEQ ID NOS:290 and 332, and
sequences homologous thereto.

54. The kit of claim 33, wherein the set of probes hybridizes to a set of six polynucleotide markers, wherein the set of six polynucleotide markers comprises:
a first polynucleotide the nucleic acid sequence set forth in SEQ ID NO:1;
a second polynucleotide having the nucleic acid sequence set forth in SEQ ID NO:3;
a third polynucleotide having the nucleic acid sequence set forth in SEQ ID NO:48;
a fourth polynucleotide having the nucleic acid sequence set forth in SEQ ID NO:6;
a fifth polynucleotide having the nucleic acid sequence set forth in SEQ ID NO:12; and
a sixth polynucleotide having the nucleic acid sequence set forth in SEQ ID NO:52, and
sequences homologous thereto.
